SEQUENCE LISTING (116 sequences)

SEQ ID NO: 1 - DNA, 51 bp, Homo sapiens
gaattgttatcaatatagtaagttasgtatcttctcattttataaccagga

SEQ ID NO: 2 - DNA, 51 bp, Homo sapiens
aggcatctgcagtccctgggggccaygctgcgtgagctgagcagtgccttc

SEQ ID NO: 3 - DNA, 51 bp, Homo sapiens
tgggtggaggtggagacaggtttgcrggaaggagtgaaagacaaccccacc

SEQ ID NO: 4 - DNA, 51 bp, Homo sapiens
cctgtgggcagttccctgaaggcctygccggctccatttgccttattgcac

SEQ ID NO: 5 - DNA, 51 bp, Homo sapiens
tcaagaacttttcctgtgcattctcracttggctacctggtacaagagtcc

SEQ ID NO: 6 - DNA, 51 bp, Homo sapiens
ctggaggtggttgcagttcacagttkctctgcccaacttcttcttcttgct

SEQ ID NO: 7 - DNA, 51 bp, Homo sapiens
gggctcagtttccacagtagcgtggragttcccggctagacaaaggggatg

SEQ ID NO: 8 - DNA, 20 bp, Artificial Sequence
  Description of Artificial Sequence Synthetic primer
gccttgggcaagggtactta

SEQ ID NO: 9 - DNA, 20 bp, Artificial Sequence
  Description of Artificial Sequence Synthetic primer
cccacgtctgcctatctgtc

SEQ ID NO: 10 - DNA, 20 bp, Artificial Sequence
  Description of Artificial Sequence Synthetic primer
ccgcatcctggctaaaaata

SEQ ID NO: 11 - DNA, 19 bp, Artificial Sequence
  Description of Artificial Sequence Synthetic primer
ccattccgctgtgaatttg

SEQ ID NO: 12 - DNA, 20 bp, Artificial Sequence
  Description of Artificial Sequence Synthetic primer
tgggggacactcagtagagg

SEQ ID NO: 13 - DNA, 20 bp, Artificial Sequence
  Description of Artificial Sequence Synthetic primer
aacgtgtatggtggggttgt

SEQ ID NO: 14 - DNA, 18 bp, Artificial Sequence
  Description of Artificial Sequence Synthetic primer
ctgcctcctgtgctccat

SEQ ID NO: 15 - DNA, 20 bp, Artificial Sequence
  Description of Artificial Sequence Synthetic primer
tgggaaatgactgagctgtg

SEQ ID NO: 16 - DNA, 21 bp, Artificial Sequence
  Description of Artificial Sequence Synthetic primer
ccagaccactcaagctttctc

SEQ ID NO: 17 - DNA, 20 bp, Artificial Sequence
  Description of Artificial Sequence Synthetic primer
agtcatgttgaaagccagca

SEQ ID NO: 18 - DNA, 23 bp, Artificial Sequence
  Description of Artificial Sequence Synthetic primer
gaaggcagacttcttagcagaat

SEQ ID NO: 19 - DNA, 20 bp, Artificial Sequence
  Description of Artificial Sequence Synthetic primer
atacagttgggctcctggtg

SEQ ID NO: 20 - DNA, 20 bp, Artificial Sequence
  Description of Artificial Sequence Synthetic primer
ccagtttgtgctaagcatcg

SEQ ID NO: 21 - DNA, 20 bp, Artificial Sequence
  Description of Artificial Sequence Synthetic primer
acacctggtgggaaaacttg

SEQ ID NO: 22 - DNA, 182 bp, Artificial Sequence
  Description of Artificial Sequence Synthetic polynucleotide
gccttgggcaagggtacttaatcttttctcaacctcaatttcctggttataaaatgagaagatacstaacttactatattgataacaattcagtgattttatatactgtgtgtatgtacacacagatacacatacatacatatagagagagacagacagacagacagataggcagacgtggg

SEQ ID NO: 23 - DNA, 178 bp, Artificial Sequence
  Description of Artificial Sequence Synthetic polynucleotide
ccgcatcctggctaaaaatacgagctcaggggtgcaaccctgccttggtgctcacctggttgatgcacagcacagggctctggaaggcactgctcagctcacgcagcrtggcccccagggactgcagatgcctggccctgggggcggaggcctggctgtcaaattcacagcggaatgg

SEQ ID NO: 24 - DNA, 190 bp, Artificial Sequence
  Description of Artificial Sequence Synthetic polynucleotide
tgggggacactcagtagagggagggcaaaaggagagtccctgctcccagagctgagaggagagatggctcaggaggggctcccaggctgaggggatagtgactgtagtggaggggggcgccaagcttacttgggtggaggtggagacaggtttgcrggaaggagtgaaagacaaccccaccatacacgtt

SEQ ID NO: 25 - DNA, 125 bp, Artificial Sequence
  Description of Artificial Sequence Synthetic polynucleotide
ctgcctcctgtgctccattcccaggctgcctgcccattctgtccccacctccgctctcctgtgggcagttccctgaaggcctygccggctccatttgccttattgcacagctcagtcatttccca

SEQ ID NO: 26 - DNA, 160 bp, Artificial Sequence
  Description of Artificial Sequence Synthetic polynucleotide
ccagaccactcaagctttctccatatcagcaataaggctgtttcatttctcatttgtagcatttttatacttccttcaagaacttttcctgtgcattctcracttggctacctggtacaagagtcctcactttcagcttatgctggctttcaacatgact

SEQ ID NO: 27 - DNA, 175 bp, Artificial Sequence
  Description of Artificial Sequence Synthetic polynucleotide
gaaggcagacttcttagcagaataaaatactttgatttatttcctttttattccaaatggacttaccagtaatagagcaagaagaagaagttgggcagagmaactgtgaactgcaaccacctccagtgaggaagtgcgtaagccaccccagctagcaccaggagcccaactgtat

SEQ ID NO: 28 - DNA, 130 bp, Artificial Sequence
  Description of Artificial Sequence Synthetic polynucleotide
ccagtttgtgctaagcatcgtaacctggggctcagtttccacagtagcgtggragttcccggctagacaaaggggatgttgcaaatcagtcttttcaaaacttttaggaccaagttttcccaccaggtgt

SEQ ID NO: 29 - DNA, 51 bp, Homo sapiens
gaattgttatcaatatagtaagttacgtatcttctcattttataaccagga

SEQ ID NO: 30 - DNA, 51 bp, Homo sapiens
gaattgttatcaatatagtaagttaggtatcttctcattttataaccagga

SEQ ID NO: 31 - DNA, 51 bp, Homo sapiens
aggcatctgcagtccctgggggccacgctgcgtgagctgagcagtgccttc

SEQ ID NO: 32 - DNA, 51 bp, Homo sapiens
aggcatctgcagtccctgggggccatgctgcgtgagctgagcagtgccttc

SEQ ID NO: 33 - DNA, 51 bp, Homo sapiens
tgggtggaggtggagacaggtttgcaggaaggagtgaaagacaaccccacc

SEQ ID NO: 34 - DNA, 51 bp, Homo sapiens
tgggtggaggtggagacaggtttgcgggaaggagtgaaagacaaccccacc

SEQ ID NO: 35 - DNA, 51 bp, Homo sapiens
cctgtgggcagttccctgaaggcctcgccggctccatttgccttattgcac

SEQ ID NO: 36 - DNA, 51 bp, Homo sapiens
cctgtgggcagttccctgaaggccttgccggctccatttgccttattgcac

SEQ ID NO: 37 - DNA, 51 bp, Homo sapiens
tcaagaacttttcctgtgcattctcaacttggctacctggtacaagagtcc

SEQ ID NO: 38 - DNA, 51 bp, Homo sapiens
tcaagaacttttcctgtgcattctcgacttggctacctggtacaagagtcc

SEQ ID NO: 39 - DNA, 51 bp, Homo sapiens
ctggaggtggttgcagttcacagttgctctgcccaacttcttcttcttgct

SEQ ID NO: 40 - DNA, 51 bp, Homo sapiens
ctggaggtggttgcagttcacagtttctctgcccaacttcttcttcttgct

SEQ ID NO: 41 - DNA, 51 bp, Homo sapiens
gggctcagtttccacagtagcgtggaagttcccggctagacaaaggggatg

SEQ ID NO: 42 - DNA, 51 bp, Homo sapiens
gggctcagtttccacagtagcgtgggagttcccggctagacaaaggggatg

SEQ ID NO: 43 - DNA, 20 bp, Artificial Sequence
  Description of Artificial Sequence Synthetic primer
ccagagtgggcagagaatgt

SEQ ID NO: 44 - DNA, 20 bp, Artificial Sequence
  Description of Artificial Sequence Synthetic primer
aaccacagtcgggaaacaag

SEQ ID NO: 45 - DNA, 22 bp, Artificial Sequence
  Description of Artificial Sequence Synthetic primer
gcttgaatgttttcatcactgg

SEQ ID NO: 46 - DNA, 21 bp, Artificial Sequence
  Description of Artificial Sequence Synthetic primer
cagtgagcacaattagccgta

SEQ ID NO: 47 - DNA, 22 bp, Artificial Sequence
  Description of Artificial Sequence Synthetic primer
ggaaccaaatgatactgatcca

SEQ ID NO: 48 - DNA, 20 bp, Artificial Sequence
  Description of Artificial Sequence Synthetic primer
accattcacaggccaaagac

SEQ ID NO: 49 - DNA, 20 bp, Artificial Sequence
  Description of Artificial Sequence Synthetic primer
ctcacctggttgatgcacag

SEQ ID NO: 50 - DNA, 20 bp, Artificial Sequence
  Description of Artificial Sequence Synthetic primer
catgtgcctctgctcaaaaa

SEQ ID NO: 51 - DNA, 20 bp, Artificial Sequence
  Description of Artificial Sequence Synthetic primer
gatccaaggcttacctgcaa

SEQ ID NO: 52 - DNA, 20 bp, Artificial Sequence
  Description of Artificial Sequence Synthetic primer
cactgccataccaccacttg

SEQ ID NO: 53 - DNA, 20 bp, Artificial Sequence
  Description of Artificial Sequence Synthetic primer
gattacaggcgtgagccatt

SEQ ID NO: 54 - DNA, 20 bp, Artificial Sequence
  Description of Artificial Sequence Synthetic primer
tctgggaccagagagagagg

SEQ ID NO: 55 - DNA, 20 bp, Artificial Sequence
  Description of Artificial Sequence Synthetic primer
tccagtccatttctccttgc

SEQ ID NO: 56 - DNA, 20 bp, Artificial Sequence
  Description of Artificial Sequence Synthetic primer
agaccaaaggggtggagtct

SEQ ID NO: 57 - DNA, 20 bp, Artificial Sequence
  Description of Artificial Sequence Synthetic primer
tccattctccctttgtcagg

SEQ ID NO: 58 - DNA, 20 bp, Artificial Sequence
  Description of Artificial Sequence Synthetic primer
agctgggaactgcaactcat

SEQ ID NO: 59 - DNA, 19 bp, Artificial Sequence
  Description of Artificial Sequence Synthetic primer
cgcctcacacactcacctc

SEQ ID NO: 60 - DNA, 21 bp, Artificial Sequence
  Description of Artificial Sequence Synthetic primer
agcagaacacaacctatctgc

SEQ ID NO: 61 - DNA, 20 bp, Artificial Sequence
  Description of Artificial Sequence Synthetic primer
cagctcctttgcagttgatg

SEQ ID NO: 62 - DNA, 21 bp, Artificial Sequence
  Description of Artificial Sequence Synthetic primer
tgggctatcaatggaagaaaa

SEQ ID NO: 63 - DNA, 20 bp, Artificial Sequence
  Description of Artificial Sequence Synthetic primer
aggtgtgagccacagcctat

SEQ ID NO: 64 - DNA, 20 bp, Artificial Sequence
  Description of Artificial Sequence Synthetic primer
ggcttggaagttcaatcagg

SEQ ID NO: 65 - DNA, 20 bp, Artificial Sequence
  Description of Artificial Sequence Synthetic primer
ttcattcccaggtagcatcc

SEQ ID NO: 66 - DNA, 20 bp, Artificial Sequence
  Description of Artificial Sequence Synthetic primer
accaatgaggagcagcagat

SEQ ID NO: 67 - DNA, 18 bp, Artificial Sequence
  Description of Artificial Sequence Synthetic primer
ctgattggctggggagtc

SEQ ID NO: 68 - DNA, 20 bp, Artificial Sequence
  Description of Artificial Sequence Synthetic primer
ttctgggaagggacagaaga

SEQ ID NO: 69 - DNA, 20 bp, Artificial Sequence
  Description of Artificial Sequence Synthetic primer
gaagacccaggtccagatga

SEQ ID NO: 70 - DNA, 20 bp, Artificial Sequence
  Description of Artificial Sequence Synthetic primer
gcctttgaacgctcaatctt

SEQ ID NO: 71 - DNA, 20 bp, Artificial Sequence
  Description of Artificial Sequence Synthetic primer
aactctctgggacaggctga

SEQ ID NO: 72 - DNA, 20 bp, Artificial Sequence
  Description of Artificial Sequence Synthetic primer
agctgaccccctgaaagtct

SEQ ID NO: 73 - DNA, 20 bp, Artificial Sequence
  Description of Artificial Sequence Synthetic primer
agcaatgaaggcaggagaaa

SEQ ID NO: 74 - DNA, 20 bp, Artificial Sequence
  Description of Artificial Sequence Synthetic primer
actctgggtccccaaatacc

SEQ ID NO: 75 - DNA, 20 bp, Artificial Sequence
  Description of Artificial Sequence Synthetic primer
gtgccgacattgacattcat

SEQ ID NO: 76 - DNA, 20 bp, Artificial Sequence
  Description of Artificial Sequence Synthetic primer
ccagactgcgagggaataaa

SEQ ID NO: 77 - DNA, 20 bp, Artificial Sequence
  Description of Artificial Sequence Synthetic primer
tgtttcccacagatgtccaa

SEQ ID NO: 78 - DNA, 18 bp, Artificial Sequence
  Description of Artificial Sequence Synthetic primer
gtccgtcagaacccatgc

SEQ ID NO: 79 - DNA, 20 bp, Artificial Sequence
  Description of Artificial Sequence Synthetic primer
gtgtctcggtgacaaagtcg

SEQ ID NO: 80 - DNA, 20 bp, Artificial Sequence
  Description of Artificial Sequence Synthetic primer
tggctgacttctgctgtctc

SEQ ID NO: 81 - DNA, 20 bp, Artificial Sequence
  Description of Artificial Sequence Synthetic primer
aagatgtagagcgggccttt

SEQ ID NO: 82 - DNA, 20 bp, Artificial Sequence
  Description of Artificial Sequence Synthetic primer
gaagggctgtatttcgaacg

SEQ ID NO: 83 - DNA, 20 bp, Artificial Sequence
  Description of Artificial Sequence Synthetic primer
ataatggcatcctccagcag

SEQ ID NO: 84 - DNA, 182 bp, Artificial Sequence
  Description of Artificial Sequence Synthetic polynucleotide
gccttgggcaagggtacttaatcttttctcaacctcaatttcctggttataaaatgagaagatacctaacttactatattgataacaattcagtgattttatatactgtgtgtatgtacacacagatacacatacatacatatagagagagacagacagacagacagataggcagacgtggg

SEQ ID NO: 85 - DNA, 182 bp, Artificial Sequence
  Description of Artificial Sequence Synthetic polynucleotide
gccttgggcaagggtacttaatcttttctcaacctcaatttcctggttataaaatgagaagatacgtaacttactatattgataacaattcagtgattttatatactgtgtgtatgtacacacagatacacatacatacatatagagagagacagacagacagacagataggcagacgtggg

SEQ ID NO: 86 - DNA, 190 bp, Artificial Sequence
  Description of Artificial Sequence Synthetic polynucleotide
tgggggacactcagtagagggagggcaaaaggagagtccctgctcccagagctgagaggagagatggctcaggaggggctcccaggctgaggggatagtgactgtagtggaggggggcgccaagcttacttgggtggaggtggagacaggtttgcaggaaggagtgaaagacaaccccaccatacacgtt

SEQ ID NO: 87 - DNA, 190 bp, Artificial Sequence
  Description of Artificial Sequence Synthetic polynucleotide
tgggggacactcagtagagggagggcaaaaggagagtccctgctcccagagctgagaggagagatggctcaggaggggctcccaggctgaggggatagtgactgtagtggaggggggcgccaagcttacttgggtggaggtggagacaggtttgcgggaaggagtgaaagacaaccccaccatacacgtt

SEQ ID NO: 88 - DNA, 125 bp, Artificial Sequence
  Description of Artificial Sequence Synthetic polynucleotide
ctgcctcctgtgctccattcccaggctgcctgcccattctgtccccacctccgctctcctgtgggcagttccctgaaggcctcgccggctccatttgccttattgcacagctcagtcatttccca

SEQ ID NO: 89 - DNA, 125 bp, Artificial Sequence
  Description of Artificial Sequence Synthetic polynucleotide
ctgcctcctgtgctccattcccaggctgcctgcccattctgtccccacctccgctctcctgtgggcagttccctgaaggccttgccggctccatttgccttattgcacagctcagtcatttccca

SEQ ID NO: 90 - DNA, 160 bp, Artificial Sequence
  Description of Artificial Sequence Synthetic polynucleotide
ccagaccactcaagctttctccatatcagcaataaggctgtttcatttctcatttgtagcatttttatacttccttcaagaacttttcctgtgcattctcaacttggctacctggtacaagagtcctcactttcagcttatgctggctttcaacatgact

SEQ ID NO: 91 - DNA, 160 bp, Artificial Sequence
  Description of Artificial Sequence Synthetic polynucleotide
ccagaccactcaagctttctccatatcagcaataaggctgtttcatttctcatttgtagcatttttatacttccttcaagaacttttcctgtgcattctcgacttggctacctggtacaagagtcctcactttcagcttatgctggctttcaacatgact

SEQ ID NO: 92 - DNA, 175 bp, Artificial Sequence
  Description of Artificial Sequence Synthetic polynucleotide
gaaggcagacttcttagcagaataaaatactttgatttatttcctttttattccaaatggacttaccagtaatagagcaagaagaagaagttgggcagagaaactgtgaactgcaaccacctccagtgaggaagtgcgtaagccaccccagctagcaccaggagcccaactgtat

SEQ ID NO: 93 - DNA, 175 bp, Artificial Sequence
  Description of Artificial Sequence Synthetic polynucleotide
gaaggcagacttcttagcagaataaaatactttgatttatttcctttttattccaaatggacttaccagtaatagagcaagaagaagaagttgggcagagcaactgtgaactgcaaccacctccagtgaggaagtgcgtaagccaccccagctagcaccaggagcccaactgtat

SEQ ID NO: 94 - DNA, 130 bp, Artificial Sequence
  Description of Artificial Sequence Synthetic polynucleotide
ccagtttgtgctaagcatcgtaacctggggctcagtttccacagtagcgtggaagttcccggctagacaaaggggatgttgcaaatcagtcttttcaaaacttttaggaccaagttttcccaccaggtgt

SEQ ID NO: 95 - DNA, 130 bp, Artificial Sequence
  Description of Artificial Sequence Synthetic polynucleotide
ccagtttgtgctaagcatcgtaacctggggctcagtttccacagtagcgtgggagttcccggctagacaaaggggatgttgcaaatcagtcttttcaaaacttttaggaccaagttttcccaccaggtgt

SEQ ID NO: 96 - DNA, 178 bp, Artificial Sequence
  Description of Artificial Sequence Synthetic polynucleotide
ccgcatcctggctaaaaatacgagctcaggggtgcaaccctgccttggtgctcacctggttgatgcacagcacagggctctggaaggcactgctcagctcacgcagcatggcccccagggactgcagatgcctggccctgggggcggaggcctggctgtcaaattcacagcggaatgg

SEQ ID NO: 97 - DNA, 178 bp, Artificial Sequence
  Description of Artificial Sequence Synthetic polynucleotide
ccgcatcctggctaaaaatacgagctcaggggtgcaaccctgccttggtgctcacctggttgatgcacagcacagggctctggaaggcactgctcagctcacgcagcgtggcccccagggactgcagatgcctggccctgggggcggaggcctggctgtcaaattcacagcggaatgg

SEQ ID NO: 98 - DNA, 128 bp, Artificial Sequence
  Description of Artificial Sequence Synthetic polynucleotide
ctcacctggttgatgcacagcacagggctctggaaggcactgctcagctcacgcagcrtggcccccagggactgcagatgcctggccctgggggcggaggcctggctgtcaaattcacagcggaatgg

SEQ ID NO: 99 - DNA, 128 bp, Artificial Sequence
  Description of Artificial Sequence Synthetic polynucleotide
ctcacctggttgatgcacagcacagggctctggaaggcactgctcagctcacgcagcatggcccccagggactgcagatgcctggccctgggggcggaggcctggctgtcaaattcacagcggaatgg

SEQ ID NO: 100 - DNA, 128 bp, Artificial Sequence
  Description of Artificial Sequence Synthetic polynucleotide
ctcacctggttgatgcacagcacagggctctggaaggcactgctcagctcacgcagcgtggcccccagggactgcagatgcctggccctgggggcggaggcctggctgtcaaattcacagcggaatgg

SEQ ID NO: 101 - DNA, 20 bp, Artificial Sequence
  Description of Artificial Sequence Synthetic primer
gcagaccagtgggtaggaga

SEQ ID NO: 102 - DNA, 20 bp, Artificial Sequence
  Description of Artificial Sequence Synthetic primer
agccactccaggaggaagtc

SEQ ID NO: 103 - DNA, 20 bp, Artificial Sequence
  Description of Artificial Sequence Synthetic primer
tgggggacactcagtagagg

SEQ ID NO: 104 - DNA, 20 bp, Artificial Sequence
  Description of Artificial Sequence Synthetic primer
aacgtgtatggtggggttgt

SEQ ID NO: 105 - DNA, 18 bp, Artificial Sequence
  Description of Artificial Sequence Synthetic primer
ctgcctcctgtgctccat

SEQ ID NO: 106 - DNA, 20 bp, Artificial Sequence
  Description of Artificial Sequence Synthetic primer
tgggaaatgactgagctgtg

SEQ ID NO: 107 - DNA, 20 bp, Artificial Sequence
  Description of Artificial Sequence Synthetic primer
ctggtcctgtggctgtctct

SEQ ID NO: 108 - DNA, 20 bp, Artificial Sequence
  Description of Artificial Sequence Synthetic primer
gccccacaactagcactcac

SEQ ID NO: 109 - DNA, 20 bp, Artificial Sequence
  Description of Artificial Sequence Synthetic primer
ccagtttgtgctaagcatcg

SEQ ID NO: 110 - DNA, 20 bp, Artificial Sequence
  Description of Artificial Sequence Synthetic primer
acacctggtgggaaaacttg

SEQ ID NO: 111 - DNA, 20 bp, Artificial Sequence
  Description of Artificial Sequence Synthetic primer
caggcatcactaagctgcaa

SEQ ID NO: 112 - DNA, 24 bp, Artificial Sequence
  Description of Artificial Sequence Synthetic primer
ttctctcctttctgcattaatttg

SEQ ID NO: 113 - DNA, 23 bp, Artificial Sequence
  Description of Artificial Sequence Synthetic primer
gaaggcagacttcttagcagaat

SEQ ID NO: 114 - DNA, 20 bp, Artificial Sequence
  Description of Artificial Sequence Synthetic primer
atacagttgggctcctggtg

SEQ ID NO: 115 - DNA, 21 bp, Artificial Sequence
  Description of Artificial Sequence Synthetic primer
ccagaccactcaagctttctc

SEQ ID NO: 116 - DNA, 20 bp, Artificial Sequence
  Description of Artificial Sequence Synthetic primer
agtcatgttgaaagccagca

(19) United States
(12) Patent Application Publication
(10) Pub. No.: US 2019/0233884 A1
(43) Pub. Date: Aug. 1, 2019

(54) HENT1 PLUS MATE1/OCT2 POLYMORPHISMS PREDICT EFFICACY AND TOXICITY OF TAS-102 IN METASTATIC COLORECTAL CANCER PATIENTS

(71) Applicant: University of Southern California, Los Angeles, CA (US)

(72) Inventor: Heinz-Josef Lenz, Los Angeles, CA (US)

(21) Appl. No.: 16/159,373

(22) Filed: Oct. 12, 2018

Related U.S. Application Data
(63) Continuation of application No. PCT/US2017/028997, filed on Apr. 21, 2017.
(60) Provisional application No. 62/573,267, filed on Oct. 17, 2017.
(60) Provisional application No. 62/326,611, filed on Apr. 22, 2016.

(51) Int. Cl. C12Q 1/6827; C12Q 1/6886; C12Q 1/6837; C12Q 1/6806; C12Q 1/686

(52) U.S. Cl. CPC C12Q 1/6827; C12Q 1/6886; C12Q 1/6837; C12Q 2600/156; C12Q 1/686; C12Q 2600/118; C12Q 2600/112; C12Q 1/6806

(57) ABSTRACT

Methods and compositions for the diagnosis and treatment of colorectal cancer are provided.

### CROSS REFERENCE TO RELATED APPLICATIONS

This application is a continuation-in-part application of International Application No. PCT/US2017/028997, filed Apr. 21, 2017, which in turn claims priority under 35 U.S.C. § 119(e) to U.S. Provisional Application No. 62/326,611, filed Apr. 22, 2016. This application also claims priority under 35 U.S.C. § 119(e) to U.S. Provisional Application No. 62/573,267, filed Oct. 17, 2017. The contents of each of these applications are hereby incorporated by reference in their entireties.

### STATEMENT OF GOVERNMENT SUPPORT

This invention was made with government support under Grant No. P30CA014089-27S1 awarded by National Institutes of Health (NIH). The government has certain rights in the invention.

### BACKGROUND

In nature, organisms of the same species usually differ from each other in some aspects, e.g., their appearance. The differences are genetically determined and are referred to as polymorphism. Genetic polymorphism is the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Polymorphism also plays a role in determining differences in an individual's response to drugs. Pharmacogenetics and pharmacogenomics are multidisciplinary research efforts to study the relationship between genotype, gene expression profiles, and phenotype, as expressed in variability between individuals in response to or toxicity from drugs. Indeed, it is now known that cancer chemotherapy is limited by the predisposition of specific populations to drug toxicity or poor drug response.

Although considerable research correlating gene expression and/or polymorphisms has been reported, much work remains to be done. This disclosure supplements the existing body of knowledge and provides related advantages as well.

### SUMMARY OF THE DISCLOSURE

It is described herein that colorectal cancer patients and metastatic colorectal cancer (collectively “CRC”) harboring certain genotypes are likely to experience more desirable clinical outcomes when treated with a therapy comprising, or consisting essentially of, or yet further consisting of, TAS-102, as compared to those not having the genotype. More desirable clinical outcomes for a cancer patient following a therapy include, without limitation, higher likelihood to respond to the therapy, relatively longer progression free survival (PFS), relatively longer overall survival (OS), relatively longer time to tumor recurrence (TTR), lower likelihood to experience an adverse effect or toxicity, or relatively milder adverse effect or toxicity.

TAS-102 is an oral combination of trifluridine (FTD) and tipiracil hydrochloride approved for the treatment of colorectal cancer. TAS-102's primary anti-tumor mechanism of action is incorporating the novel nucleoside analog, FTD, into DNA. Trifluridine (FTD) is an active cytotoxic component of TAS-102, and thymidine phospholyrase inhibitor (TPI) inhibits rapid degradation of FTD by TP. It has been tested whether single nucleotide polymorphisms (SNPs) in genes involved in FTD metabolism and TPI excretion will predict outcome in patients with refractory metastatic colorectal cancer (mCRC) treated with TAS-102.

The findings of the present disclosure are summarized in the following Table A:

| | | Clinical | Favorable | Unfavorable |
|---|---|---|---|---|
| Polymorphism(s) | Gene | Endpoint | Genotype | Genotype |
| rs760370 | hENT1 | PFS, OS | GG; GA | AA |
| rs9394992 | hENT | PFS, OS | TT or TC | CC |
| rs2289669 | MATE1 | PFS | GA *see | — |
| | | | below for | |
| | | | interactions | |

| | | Clinical | Favorable | Unfavorable |
|---|---|---|---|---|
| Polymorphism(s) | Gene(s) | Endpoint | Genotype | Genotype |
| rs2289669 | MATE1 | PFS, OS | 1. rs2289669 (GG) and | — |
| (MATE1) and | and OCT2 | | rs316019 (CC); | |
| rs316019 | | | 2. rs2289669 (GA) and | |
| (OCT2) | | | rs316019 (CC); | |
| | | | 3. rs2289669 (GA) and | |
| | | | rs316019 (CA); | |
| | | | 4. rs2289669 (GA) and | |
| | | | rs316019 (AA); or | |
| | | | 5. rs2289669 (AA) and | |
| | | | rs316019 (AA). | |
| rs316019 | OCT2, | PFS, OS | rs760370 (GG or GA) and | — |
| (OCT2), and | MATE1 and | | 1. rs316019/rs2289669: | |
| rs2289669 | hENT1 | | (CC/GG), (CC/GA), | |
| (MATE1), and | | | (CA/GA), (AA/GA) | |
| rs760370 | | | or (AA/AA). | |
| (hENT1) | | | | |
| rs609429 | ATM | PFS, OS | GC or GG | CC |
| rs861539 | XRCC3 | PFS, OS | AG or AA | GG |

*The genotypes noted here only refer to one DNA strand; for instance, genotype CG is equivalent to GC on the opposite strand and should be understood to encompass both strands.
** Findings with respect to the interaction between MATE1 rs2289669 and OCT2 rs316019 polymorphisms on PFS and OS are further described herein (see Examples).

This disclosure relates to methods and kits for one or more of: screening a biological sample for a relevant polymorphism, classifying a patient as eligible for TAS-102 therapy, identifying patients likely to respond to TAS-102 therapy, treating a patient based on the patient's genotype, and increasing survival of a patient. This disclosure provides an in vitro method of detecting a polymorphism in a patient with cancer or a patient suspected of having cancer, e.g., GI cancer, colon cancer, rectal cancer, or colorectal cancer, the method comprising, or alternatively consisting essentially of, or yet further consisting of screening a biological sample from the patient to detect the genotypes of (GC) or (GG) for rs609429, (AG) or (AA) for rs861539, (AG) or (GG) for rs760370, or (CT) or (TT) for rs9394992. In one aspect, the patient is known to have the cancer. In another aspect the patient is suspected of having the cancer.

Also provided by this disclosure is a method for selecting a cancer patient, e.g., a GI cancer patient, a colon cancer patient, a rectal cancer patient, or a colorectal cancer patient for a therapy comprising, or alternatively consisting essentially of, or yet further consisting of administration of an effective amount of TAS-102, the method comprising, or alternatively consisting essentially of, or yet further consisting of screening a biological sample isolated from the patient for a polymorphism, and selecting the patient for the therapy if the genotype of (GC) or (GG) for rs609429, (AG) or (AA) for rs861539, (AG) or (GG) for rs760370, or (CT) or (TT) for rs9394992 is present in the sample. In one aspect, the patient is known to have the cancer. In another aspect the patient is suspected of having the cancer. The TAS-102 therapy can be first line, second line, third line, fourth line or fifth line therapy and can be administered alone or in combination with other therapies, e.g., surgical resection, radiation therapy or other chemical or biological based therapies.

Further provided by this disclosure is a method for classifying a cancer patient, e.g., a GI cancer patient, a colon cancer patient, a rectal cancer patient, or a colorectal cancer patient as eligible for a therapy comprising, or alternatively consisting essentially of, or yet further consisting of administration of an effective amount of TAS-102, the method comprising, or alternatively consisting essentially of, or yet further consisting of screening a biological sample isolated from the patient for a polymorphism, and classifying the patient as eligible for the therapy if the genotype of (GC) or (GG) for rs609429, (AG) or (AA) for rs861539, (AG) or (GG) for rs760370, or (CT) or (TT) for rs9394992 is present in the sample. In one aspect, the patient is known to have the cancer. In another aspect the patient is suspected of having the cancer. The TAS-102 therapy can be first line, second line, third line, fourth line or fifth line therapy and can be administered alone or in combination with other therapies, e.g., surgical resection, radiation therapy or other chemical or biological based therapies.

Also provided herein is a method for identifying whether a cancer patient, e.g., a GI cancer patient, a colon cancer patient, a rectal cancer patient, or a colorectal cancer patient is likely to experience a relatively longer or shorter progression free survival following a therapy comprising, or alternatively consisting essentially of, or yet further consisting of administration of an effective amount of TAS-102, the method comprising, or alternatively consisting essentially of, or yet further consisting of screening a biological sample isolated from the patient for a polymorphism, and identifying that the patient is likely to experience a longer progression free survival if the genotype of (GC) or (GG) for rs609429, (AG) or (AA) for rs861539, (AG) or (GG) for rs760370, or (CT) or (TT) for rs9394992 is present in the sample, relative to a corresponding cancer patient not having the genotype, e.g., for a colorectal cancer patient the corresponding cancer patient would be another colorectal cancer patient. In one aspect, the patient is known to have the cancer. In another aspect the patient is suspected of having the cancer. The TAS-102 therapy can be first line, second line, third line, fourth line or fifth line therapy and can be administered alone or in combination with other therapies, e.g., surgical resection, radiation therapy or other chemical or biological based therapies.

Further provided herein is a method for identifying whether a cancer patient, e.g., a GI cancer patient, a colon cancer patient, a rectal cancer patient, or a colorectal cancer patient is likely to experience TAS-102 related toxicity following a therapy comprising, or alternatively consisting essentially of, or yet further consisting of administration of an effective amount of TAS-102, the method comprising, or alternatively consisting essentially of, or yet further consisting of screening a biological sample isolated from the patient for a polymorphism, and identifying that the patient is likely to experience toxicity if the genotype of (GC) or (GG) for rs609429 is present in the sample, relative to a corresponding cancer patient not having the genotype. In one aspect, the patient is known to have the cancer. In another aspect the patient is suspected of having the cancer. The TAS-102 therapy can be first line, second line, third line, fourth line or fifth line therapy and can be administered alone or in combination with other therapies, e.g., surgical resection, radiation therapy or other chemical or biological based therapies.

Yet further provided herein is a method for treating a cancer patient, e.g., a GI cancer patient, a colon cancer patient, a rectal cancer patient, or a colorectal cancer patient selected for treatment based on the presence of the genotype of (GC) or (GG) for rs609429, (AG) or (AA) for rs861539, (AG) or (GG) for rs760370, or (CT) or (TT) for rs9394992 in a biological sample from the patient, comprising, or alternatively consisting essentially of, or yet further consisting of administering to the patient a therapy comprising, or alternatively consisting essentially of, or yet further consisting of an effective amount of TAS-102. In one aspect, the patient is known to have the cancer. In another aspect the patient is suspected of having the cancer. The TAS-102 therapy can be first line, second line, third line, fourth line or fifth line therapy and can be administered alone or in combination with other therapies, e.g., surgical resection, radiation therapy or other chemical or biological based therapies.

Also provided herein is a method for increasing the progression-free and/or overall survival of a cancer patient, e.g., a GI cancer patient, a colon cancer patient, a rectal cancer patient, or a colorectal cancer patient, the method comprising, or alternatively consisting essentially of, or yet further consisting of screening a biological sample isolated from the patient for a polymorphism, and classifying the patient as eligible for the therapy with an effective amount of TAS-102 if the genotype of (GC) or (GG) for rs609429, (AG) or (AA) for rs861539, (AG) or (GG) for rs760370, or (CT) or (TT) for rs9394992 is present in the sample or not eligible for the therapy comprising, or alternatively consisting essentially of, or yet further consisting of administration of an effective amount of TAS-102 if the genotype of (GC) or (GG) for rs609429, (AG) or (AA) for rs861539, (AG) or (GG) for rs760370, or (CT) or (TT) for rs9394992 is not present in the sample. In one aspect, the patient is known to have the cancer. In another aspect the patient is suspected of having the cancer. The TAS-102 therapy can be first line, second line, third line, fourth line or fifth line therapy and can be administered alone or in combination with other therapies, e.g., surgical resection, radiation therapy or other chemical or biological based therapies.

Yet further provided are kits for performing the methods described herein, comprising or alternatively consisting essentially of, or yet further consisting of reagents to identify or determine the genotype of the sample or a patient and instructions for use.

### BRIEF DESCRIPTION OF THE DRAWINGS

The drawings as presented herein set forth various aspects of the invention as further described herein.

FIGS. 1A-1B Progression-free survival (PFS) and overall survival (OS) by hENT1 rs760370 variants, AA or any G in the training cohort (FIG. 1A) and the testing cohort (FIG. 1B) treated with TAS-102.

FIGS. 2A-2C (FIG. 2A) OCT2 and MATE1 gene-gene interaction and CLsec (secretory) of metformin, based on data of a previous pharmacokinetic study. (FIG. 2B) Combination of MATE1 rs2289669 and OCT2 rs316019 and expected clinical outcome in the training cohort. Based upon the expected secretory clearance of TPI by gene-gene interaction of MATE1 rs2289669 and OCT2 rs316019, the all nine assemblies of the genetic variants were divided into two categories, five ‘Good’ and four ‘Poor’. (FIG. 2C) A novel classification composed of hENT1 rs760370 and OCT2 rs316019/MATE1 rs2289669 based on the association of SNPs and clinical outcome in the training cohort. Excellent and Good groups were combined as one group to increase the sample size.

FIGS. 3A-3B Progression-free survival (PFS) and overall survival (OS) by combination of hENT1 rs760370 and OCT2 rs316000/MATE1 rs2289669 in the training cohort (FIG. 3A) and the testing cohort (FIG. 3B) treated with TAS-102: Poor, Fair and Good or Excellent.

FIG. 4 FTD metabolism and TPI excretion.

FIG. 5 Scheme of mediation analysis for SNPs.

FIG. 6 Treatment recommendation for TAS-102 in patients with metastatic colorectal cancer.

FIG. 7A-7D illustrate data for overall survival (OS) between the (C/C) and (C/G) or (G/G) genotypes for ATM rs609429 following TAS-102 treatment. FIG. 7A depicts OS by combination of ATM rs609429 in the evaluation cohort. FIG. 7B depicts OS by combination of ATM rs609429 in the control cohort. FIG. 7C depicts PFS by combination of ATM rs609429 in the validation cohort. FIG. 7D depicts OS by combination of ATM rs609429 in the validation cohort. The light gray line represents the Any G genotype (C/G or G/G) and the dark gray line depicts the (C/C) genotype.

FIG. 8 illustrates data for progression-free survival (PFS) and overall survival (OS) between the (G/G) and (G/A) genotypes for XRCC3 rs861539 following TAS-102 treatment. The light gray line represents the (G/A) genotype (n=17) and the dark gray line depicts the (G/G) genotype (n=35). The log-rank P values are 0.024 for PFS and 0.012 for OS.

FIG. 9 illustrates data for overall survival (OS) or survival functions between the (C/C) and (C/G) or (G/G) genotypes for ATM rs609429 following TAS-102 treatment (JPN TAS-102 cohort) versus treatment with the multi-kinase inhibitor Regorafenib (ITA REGORA cohort). The light gray line represents the (C/C) genotype (n=14) and the dark gray line depicts the (C/G) or (G/G) genotypes (n=38).

FIG. 10 illustrates data for progression-free survival (PFS) between the (G/G) and (G/A) genotypes for XRCC3 rs861539 following TAS-102 treatment (JPN TAS-102 cohort) versus treatment with the multi-kinase inhibitor Regorafenib (ITA REGORA cohort). The light gray line represents the (G/A) genotype and the dark gray line represents the (G/G) genotype.

FIG. 11 illustrates data for overall survival (OS) between the (G/G) and (G/A) genotypes for XRCC3 rs861539 following TAS-102 treatment (JPN TAS-102 cohort) versus treatment with the multi-kinase inhibitor Regorafenib (ITA REGORA cohort). The light gray line represents the (G/A) genotype and the dark gray line represents the (G/G) genotpe.

FIG. 12 illustrates data for progression-free survival (PFS) and overall survival (OS) between the (A/A) and (A/G) or (G/G) genotypes for hENT rs760370 following TAS-102 treatment. The light gray line represents the (A/G) or (G/G) genotypes and the dark gray line represents the (A/A) genotype.

FIG. 13 illustrates data for progression-free survival (PFS) and overall survival (OS) between the (C/C) and (C/T) or (T/T) genotypes for hENT rs9394992 following TAS-102 treatment. The light gray line represents the (C/T) or (T/T) genotypes and the dark gray line represents the (C/C) genotype.

FIG. 14 illustrates data for progression-free survival (PFS) and overall survival (OS) among the (G/G), (G/A) and (A/A) genotypes for MATE1 rs2289669 following TAS-102 treatment. The light gray line represents the (G/A) genotype, the medium gray line represents the (A/A) genotype, and the dark gray line represents the (G/G) genotype.

FIG. 15 illustrates the expected OCT2 and MATE1 gene-gene interaction of the TAS-102 cohort. The dark gray squares represent good and the light gray squares represent poor.

FIG. 16 illustrates data for progression-free survival (PFS) and overall survival (OS) or the combination of OCT2 rs316000 MATE1 rs2289669 genotypes following TAS-102 treatment. The dark gray lines represent poor and the light gray lines represent good.

FIG. 17 illustrates combined hENT1 and OCT2/MATE1 gene variants analysis of PFS in the TAS-102 cohort (n=52).

FIG. 18 illustrates combined hENT1 and OCT2/MATE1 gene variants analysis of OS in the TAS-102 cohort (n=52).

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this disclosure pertains.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1.

It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used herein, the term “cancer” intends a malignant phenotype characterized by the uncontrolled proliferation of malignant cells. The methods and compositions of this disclosure are useful for the treatment and diagnosis of cancer of the gastrointestinal (GI) tract, such as gastric cancer, GI cancer, colon cancer, rectal cancer, and/or colorectal cancer. The cancer can be metastatic, non-metastatic and pre-clinical.

As used herein, the terms “corresponding cancer” when referring to a comparison or relative term indicates that the measured or detected polymorphism is compared to a patient having the same cancer type, e.g., colorectal cancer is compared to colorectal cancer.

The term “chemotherapy” encompasses cancer therapies that employ chemical or biological agents or other therapies, such as radiation therapies, e.g., a small molecule drug or a large molecule, such as antibodies, RNAi and gene therapies. Non-limiting examples of chemotherapies are provided below. Unless specifically excluded, when a specific therapy is recited, equivalents of the therapy are within the scope of this invention.

Trifluridine/tipiracil (TAS-102, CAS Number 733030-01-8) is sold under the trade name of Lonsurf. It is a combination of two active pharmaceutical ingredients: trifluridine, a nucleoside analog, and tipiracil hydrochloride, a thymidine phosphorylase inhibitor. Trifluridine has the chemical formula C₁₀H₁₁F₃N₂O₅ and is also known as α,α,α-trifluorothymidine; 5-trifluromethyl-2′-deoxyuridine; and FTD5-trifluoro-2′-deoxythymidine (CAS number 70-00-8). Tipiracil has the chemical formula C₉H₁₁ClN₄O₂ and inhibits the enzyme thymidine phosphorylase, preventing rapid metabolism of trifluridine, increasing the bioavailability of trifluridine. Although not always explicitly stated, when the term TAS-102 is used in terms of therapy, Applicant intends not only Lonsurf, but also equivalents thereof. Equivalents thereof include trifluridine alone, trifluridine that modified to increase its halflife and/or resistance to metabolism by thymidine phosphorylase, or substitution of one or both of trifluridine and/or tipiracil hydrochloride with a chemical equivalent. Non-limiting examples of chemical equivalents include pharmaceutically acceptable salts or solvates of the active ingredients. In one aspect of each of the disclosed methods, TAS-102 intends the combination therapy marketed by Taiho under the tradename Lonsurf. In one aspect, TAS-102 is part of a combination therapy including TAS-102 or an equivalent thereof and one or more of surgical resection, radiation therapy or other line therapies.

Cancer chemotherapy has been used for the treatment of cancers for many decades. Alternatives to TAS-102 include those known in the art, e.g., 5-FU, platinum-based therapies, radiation therapy, surgical resection. Non-limiting examples are disclosed herein.

Irinotecan (CPT-11) is sold under the trade name of Camptosar®. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation. An equivalent of irinotecan is a composition that inhibits a topoisomerase. Non-limiting examples of an equivalent of irinotecan include topotecan, camptothecin and lamellarin D, etoposide, or doxorubicin.

Oxaliplatin (trans−/−diaminocyclohexane oxalatoplatinum; L-OHP; CAS No. 61825-94-3) is sold under the trade name of Elotaxin. It is a platinum derivative that causes cell cytotoxicity. Oxaliplatin forms both inter- and intra-strand cross links in DNA, which prevent DNA replication and transcription, causing cell death. Non-limiting examples of an equivalent of oxaliplatin include carboplatin and cisplatin.

Topoisomerase inhibitors are agents designed to interfere with the action of topoisomerase enzymes (topoisomerase I and II), which are enzymes that control the changes in DNA structure by catalyzing the breaking and rejoining of the phosphodiester backbone of DNA strands during the normal cell cycle. In one aspect, topoisomerase inhibitors include irinotecan, topotecan, camptothecin and lamellarin D, or compounds targeting topoisomerase IA. In another aspect, topoisomerase inhibitors include etoposide, doxorubicin or compounds targeting topoisomerase II.

Pyrimidine antimetabolite includes, without limitation, fluorouracil (5-FU), its equivalents and prodrugs. In one embodiment, a pyrimidine antimetabolite is a chemical that inhibits the use of a pyrimidine. The presence of antimetabolites can have toxic effects on cells, such as halting cell growth and cell division, so these compounds can be used as chemotherapy for cancer.

Fluorouracil (5-FU) belongs to the family of therapy drugs called pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluroidine), 1-tetrahydrofuranyl-5-fluorouracil (ftorafur), Capecitabine (Xeloda), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487.

“5-FU based adjuvant therapy” refers to 5-FU alone or alternatively the combination of 5-FU with other treatments, that include, but are not limited to radiation, methyl-CCNU, leucovorin, oxaliplatin, irinotecin, mitomycin, cytarabine, levamisole. Specific treatment adjuvant regimens are known in the art as FOLFOX, FOLFOX4, FOLFIRI, MOF (semustine (methyl-CCNU), vincrisine (Oncovin) and 5-FU). For a review of these therapies see Beaven and Goldberg (2006) Oncology 20(5):461-470. An example of such is an effective amount of 5-FU and Leucovorin. Other chemotherapeutics can be added, e.g., oxaliplatin or irinotecan.

Capecitabine is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

A therapy comprising, or alternatively consisting essentially of, or yet further consisting of a pyrimidine antimetabolite includes, without limitation, a pyrimidine antimetabolite alone or alternatively the combination of a pyrimidine antimetabolite with other treatments, that include, but are not limited to, radiation, methyl-CCNU, leucovorin, oxaliplatin, irinotecin, mitomycin, cytarabine, levamisole. Specific treatment adjuvant regimens are known in the art as FOLFOX, FOLFOX4, FOLFOX6, FOLFIRI, MOF (semustine (methyl-CCNU), vincrisine (Oncovin) and 5-FU). For a review of these therapies see Beaven and Goldberg (2006) Oncology 20(5):461-470. An example of such is an effective amount of 5-FU and Leucovorin. Other chemotherapeutics can be added, e.g., oxaliplatin or irinotecan.

FOLFIRI is a chemotherapy regimen for treatment of colorectal cancer. It is made up of the following drugs: FOL—folinic acid (leucovorin), a vitamin B derivative used as a “rescue” drug for high doses of the drug methotrexate and that modulates/potentiates/reduces the side effects of fluorouracil; F—fluorouracil (5-FU), a pyrimidine analog and antimetabolite which incorporates into the DNA molecule and stops synthesis; and IRI—irinotecan (Camptosar), a topoisomerase inhibitor, which prevents DNA from uncoiling and duplicating.

FOLFOX is a chemotherapy regimen for treatment of colorectal cancer. is made up of the following drugs: FOL—folinic acid (leucovorin), F—fluorouracil (5-FU), and OX-oxaliplatin.

FOLFOXFIRI is a chemotherapy regimen for treatment of colorectal cancer. is made up of the following drugs: FOL—folinic acid (leucovorin), F—fluorouracil (5-FU), OX-oxaliplatin and IRI—irinotecan (Camptosar).

Bevacizumab (BV) is sold under the trade name Avastin® by Genentech. It is a humanized monoclonal antibody that binds to and inhibits the biologic activity of human vascular endothelial growth factor (VEGF). Biological equivalent antibodies are identified herein as modified antibodies which bind to the same epitope of the antigen, prevent the interaction of VEGF to its receptors (Flt01, KDR a.k.a. VEGFR2) and produce a substantially equivalent response, e.g., the blocking of endothelial cell proliferation and angiogenesis. Bevacizumab is also in the class of cancer drugs that inhibit angiogenesis (angiogenesis inhibitors).

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at cancer.gov. Typically, a patient is given a subsequent chemotherapy regimen because the patient did not shown a positive clinical or sub-clinical response to the first line therapy or the first line therapy has stopped.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

In one aspect, the term “equivalent” of “chemical equivalent” of a chemical means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

As used herein, the term “determining the genotype of a cell or tissue sample” intends to identify the genotypes of polymorphic loci of interest in the cell or tissue sample. In one aspect, a polymorphic locus is a single nucleotide polymorphic (SNP) locus. If the allelic composition of a SNP locus is heterozygous, the genotype of the SNP locus will be identified as “X/Y” or “XX” wherein X and Y are two different nucleotides. If the allelic composition of a SNP locus is heterozygous, the genotype of the SNP locus will be identified as “X/X” or “XX” wherein X identifies the nucleotide that is present at both alleles.

As used herein, a “synonymous” SNP refers to a SNP that does not cause a change in the polypeptide encoded by the gene. A “non-synonymous SNP” is a SNP that does result in a change in the polypeptide encoded by the gene. For example, a non-synonymous SNP may result in an amino acid substitution or the introduction of a premature stop codon.

The term “genetic marker” refers to an allelic variant of a polymorphic region of a gene of interest and/or the expression level of a gene of interest.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region can be a single nucleotide, the identity of which differs in different alleles.

The term “genotype” refers to the specific allelic composition of an entire cell or a certain gene and in some aspects a specific polymorphism associated with that gene, whereas the term “phenotype” refers to the detectable outward manifestations of a specific genotype.

The KRAS gene (NM_004985, NM_033360) is a proto-oncogene that encodes a GTPase important in in signal transduction. Mutations in the KRAS gene are found at high rates in cancers, including but not limited to colorectal cancers. Common KRAS mutations in colorectal cancer include but are not limited to mutations at codons 12 or 13 of exon 2 that result in amino acid substitutions in the protein sequence such as Gly12Asp [GGT>GAT] G12D, Gly12Val [GGT>GAC] G12V, Gly12Cys [GGT>TGT] G12C, Gly12Ser [GGT>AGT] G12S, Gly12Ala [GGT>GCT] G12A, Gly12Arg [GGT>CGT] G12R, Gly13Asp [GGC>GAC] G13D. The BRAF gene (NM_004333) is a proto-oncogene that is often mutated in colorectal cancers. The BRAF gene encodes a signal transduction kinase of the Raf family. Common mutations of the BRAF gene that are relevant to cancer result in amino acid substitutions in the protein sequence including but not limited to V600E, R461I, I462S, G463E, G463V, G465A, G465E, G465V, G468A, G468E, N580S, E585K, D593V, F594L, G595R, L596V, T598I, V599D, V599E, V599K, V599R, V600K, A727V. The terms “KRAS wild-type” and “BRAF wild-type” refers to a genotype of a cell or patient in which no mutation is detected in the corresponding gene. In some aspects, no mutation is detected that affects the function or activity of the gene.

The rs760370 polymorphism is located at chromosome position 44173216 on chromosome 6 according to the Genome Reference Consortium Human Build 38 patch release 2 (GRCh38.p2, NCBI). The rs760370 polymorphism is located within the human equilibrative nucleoside transporter 1 (hENT1, mRNA: NM_001078174, NM_001078175, NM_001078176, NM_001078177, NM_001304462, NM_001304463, NM_001304465, NM_001304466, NM_004955) gene (also known as SLC29A1, solute carrier family 29 member 1). The following nucleotide sequence represents a region of human DNA comprising the rs760370 polymorphism: TGGGTGGAGGTGGAGACAGGTTTGC[A/G]GGAAGGAGTGAAAGACAACCCCACC (SEQ ID NO:3). Thus, the partial sequence of the (A) allele of the rs760370 polymorphism is: TGGGTGGAGGTGGAGACAGGTTTGCAGGAAGGAGTGAAAGACAACCCCACC (SEQ ID NO:33) and the partial sequence of the (G) allele is: TGGGTGGAGGTGGAGACAGGTTT GCGGGAAGGAGTGAAAGACAACCCCACC (SEQ ID NO:34).

The rs609429 polymorphism (also named IVS48+238C>G) is located at chromosome position 20322886 on chromosome 11 according to the Genome Reference Consortium Human Build 38 patch release 2 (GRCh38.p2, NCBI). The rs609429 polymorphism is and intron polymorphism located within the ataxia telangiectasia (ATM, mRNA: NM_000051, NM_138292, NM_138293) gene. ATM, a serine/threonine protein kinase, is a key activator of the cellular response to DNA double-strand breaks managing subsequent phosphorylation of proteins such as p53, Chk2, and BRCA1. The minor allele (G) generates a weak 5′ splice site and decreases gene expression. The following nucleotide sequence represents a region of human DNA comprising the rs609429 polymorphism: GAATTGTTATCAATATAGTAAGTTA[C/G] GTATCTTCTCATTTTATAACCAGGA (SEQ ID NO:1). Thus, the partial sequence of the (C) allele of rs609429 is: GAATTGTTATCAATATAGTAAGTTACGTATCTTCTCATTTTATA ACCAGGA (SEQ ID NO:29) and the partial sequence of the (G) allele of rs609429 is: GAATT GTTATCAATATAGTAAGTTAGGTATCTTCTCATTTTATAACCAGGA (SEQ ID NO:30). The rs861539 polymorphism is located at chromosome position 85475893 on chromosome 14 according to the Genome Reference Consortium Human Build 38 patch release 2 (GRCh38.p2, NCBI). The rs861539 polymorphism is located within the X-ray repair complementing defective repair in Chinese hamster cells 3 (XRCC3, mRNA: NM_001100118, NM_001100119, NM_005432) gene. XRCC3 is a member of the DNA repair genes encoding the RecA/RAD51-related protein family for the maintenance of genome stability in homologous recombination repair for DNA double-strand breaks. The polymorphism is in the coding region of the XRCC3 gene and results in a T241M change in the XRCC3 protein. The following nucleotide sequence represents a region of human DNA comprising the rs861539 polymorphism: AGGCATCTGCAGTCCCTGGGGGCCA[C/T]GCTGCGTGAGCTGAGCAGTGCCTTC (SEQ ID NO:2). Thus, the partial sequence of the (C) allele of rs861539 is: AGGCATCTGCAGTCCCTGGGGGCCACGCTGCGTGAGCTGAGCAGTGCCTTC (SEQ ID NO:31) and the partial sequence of the (T) allele is AGGCATCTGC AGTCCCTGGGGGCCATGCTGCGTGAGCTGAGCAGTGCCTTC (SEQ ID NO:32).

The rs9394992 polymorphism is located at chromosome position 44168255 on chromosome 6 according to the Genome Reference Consortium Human Build 38 patch release 2 (GRCh38.p2, NCBI), which is also within the hENT1 gene. The following nucleotide sequence represents a region of human DNA comprising the rs9394992 polymorphism: CCTGTGGGCAG TTCCCTGAAGGCCT[C/T]GCCGGCTCCATTTGCCTTATTGCAC (SEQ ID NO:4). Thus, the partial sequence of the (C) allele of rs9394992 is: CCTGTGGGCAGTTCCCTGAAGGCCT CGCCGGCTCCATTTGCCTTATTGCAC (SEQ ID NO:35) and the partial sequence of the (T) allele is: CCTGTGGGCAGTTCCCTGAAGGCCTTGCCGGCTCCATTTGCCTTATTGCAC (SEQ ID NO:36).

The OCT2 rs316019 polymorphism is located at chromosome position 1000019316 on chromosome 6 according to the Genome Reference Consortium Human Build 38 patch release 2 (GRCh38.p2, NCBI). The following nucleotide sequence represents a region of human DNA comprising the rs316019 polymorphism: CTGGAGGTGGTTGCAGTTCACAGTT[G/T] CTCTGCCCAACTTCTTCTTCTTGCT (SEQ ID NO:6). Thus, the partial sequence of the (G) allele of rs316019 is CTGGAGGTGGTTGCAGTTCACAGTTGCTCTGCCCAACTTCTTC TTCTTGCT (SEQ ID NO:39) and the partial sequence of the (T) allele of rs316019 is: CTGGAGGTGGTTGCAGTTCACAGTTTCTCTGCCCAACTTCTTCTTCTTGCT (SEQ ID NO:40).

The MATE1 rs2289669 polymorphism is located at chromosome position 19071043 on chromosome 17 according to the Genome Reference Consortium Human Build 38 patch release 2 (GRCh38.p2, NCBI). The following nucleotide sequence represents a region of human DNA comprising the rs2289669 polymorphism: GGGCTCAGTTTCCACAGTAGCGTGG[A/G] AGTTCCCGGCTAGACAAAGGGGATG (SEQ ID NO:7). Thus, the partial sequence of the (A) allele of rs2289669 is: GGGCTCAGTTTCCACAGTAGCGTGGAAGTTCCCGGCTAGAC AAAGGGGATG (SEQ ID NO:41) and the partial sequence for the (G) allele is GGGCTCAG TTTCCACAGTAGCGTGGGAGTTCCCGGCTAGACAAAGGGGATG (SEQ ID NO:42).

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs.

“An effective amount” or “therapeutically effect amount” intends to indicate the amount of a compound or agent administered or delivered to the patient which is most likely to result in the desired response to treatment. The amount is empirically determined by the patient's clinical parameters including, but not limited to the Stage of disease, age, gender, histology, and likelihood for tumor recurrence.

A “patient” as used herein intends an animal patient, a mammal patient or yet further a human patient. For the purpose of illustration only, a mammal includes but is not limited to a simian, a murine, a bovine, an equine, a porcine or an ovine subject.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity or side effect.

The term “TAS-102 related toxicity” refers to harmful, toxic, or adverse effects or events caused by treatment with TAS-102. Such effects include but are not limited to febrile neutropenia, leukopenia, stomatitis, neutropenia, hand-foot syndrome, cardiac ischemia, thrombocytopenia, increase in alanine aminotransferase level, increase in aspartate aminotransferase level, increase in total bilirubin, increase in alkaline phosphatase level, increase in creatinine level, anemia, anorexia, nausea, vomiting, decreased appetite, fatigue, abdominal pain, fever, asthenia, and diarrhea. The comparative reference for relative terms such as “increased” or “decreased” in the preceding list is the levels in the patient or subject prior to TAS-102 treatment or the levels in a group of treated patients or subjects compared to untreated control patients or subjects. TAS-102 related toxic effects are detailed in Mayer et al. New England J Med., 372:1909-1919 (May 2015). As used generally, “acute” drug toxicity involves harmful or adverse effects in an organism through a single or short-term exposure to a drug substance or other therapeutic biological products. “Subchronic” toxicity is the ability of a drug substance to cause harmful or adverse effects for more than one year but less than the lifetime of the exposed organism. “Chronic” toxicity is the ability of a drug substance or combination therapy to cause harmful effects over an extended period, usually upon repeated or continuous exposure, sometimes lasting for the entire life of the exposed organism. Adverse events are classified and graded by severity. Grade 1 adverse events: mild; asymptomatic or mild symptoms; clinical or diagnostic observations only; intervention not indicated. Grade 2 adverse events: moderate; minimal, local or noninvasive intervention indicated; limiting age-appropriate instrumental activities of daily living (ADL). Grade 3 adverse events: severe or medically significant but not immediately life-threatening; hospitalization or prolongation of hospitalization indicated; disabling; limiting self care activities of daily living. Grade 4 adverse events: life-threatening consequences; urgent intervention indicated. Grade 5 adverse event: death related to AE (Department of Health and Human Services, National Institutes of Health, National Cancer Institute. Common terminology criteria for adverse events (CTCAE) v4.03. 2010).

TAS-102 also known as Trifluridine/tipiracil (trade name Lonsurf™) is a combination drug for the treatment of metastatic colorectal cancer. It is a combination of two active pharmaceutical ingredients: trifluridine, a nucleoside analog, and tipiracil, a thymidine phosphorylase inhibitor. Tipiracil prevents rapid metabolism of trifluridine, increasing the bioavailability of trifluridine.

The term “suitable for a therapy” or “suitably treated with a therapy” shall mean that the patient is likely to exhibit one or more desirable clinical outcomes as compared to patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In further another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, can exhibit more than one more desirable clinical outcomes as compared to patients having the same disease and receiving the same therapy but not possessing the characteristic. As defined herein, the patient is considered suitable for the therapy. In another such aspect, a patient possessing a characteristic can exhibit one or more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

Response criteria herein are based on the RECIST criteria (Therasse and Arbuck et al., 2000, New Guidelines to Evaluate Response to Treatment in Solid Tumors, J Natl Cancer Inst, 92:205-16). A “complete response” (CR) to a therapy refers to the clinical status of a patient with evaluable but non-measurable disease, whose tumor and all evidence of disease have disappeared following administration of the therapy. In this context, a “partial response” (PR) refers to a response that is anything less than a complete response. “Stable disease” (SD) indicates that the patient is stable following the therapy. “Progressive disease” (PD) indicates that the tumor has grown (i.e. become larger) or spread (i.e. metastasized to another tissue or organ) or the overall cancer has gotten worse following the therapy. For example, tumor growth of more than 20 percent since the start of therapy typically indicates progressive disease. “Non-response” (NR) to a therapy refers to status of a patient whose tumor or evidence of disease has remained constant or has progressed.

“Overall Survival” (OS) refers to the length of time of a cancer patient remaining alive following a cancer therapy.

“Progression free survival” (PFS) or “Time to Tumor Progression” (TTP) refers to the length of time following a therapy, during which the tumor in a cancer patient does not grow. Progression-free survival includes the amount of time a patient has experienced a complete response, partial response or stable disease.

“Disease free survival” refers to the length of time following a therapy, during which a cancer patient survives with no signs of the cancer or tumor.

“Time to Tumor Recurrence (TTR)” refers to the length of time, following a cancer therapy such as surgical resection or chemotherapy, until the tumor has reappeared (come back). The tumor may come back to the same place as the original (primary) tumor or to another place in the body.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

“Objective response rate” refers to the proportion of responders (patients with either a partial (PR) or complete response (CR)) compared to nonresponders (patients with either SD or PD). Response duration can be measured from the time of initial response until documented tumor progression.

The term “identify” or “identifying” is to associate or affiliate a patient closely to a group or population of patients who likely experience the same or a similar clinical response to a therapy.

The term “selecting” a patient for a therapy refers to making an indication that the selected patient is suitable for the therapy. Such an indication can be made in writing by, for instance, a handwritten prescription or a computerized report making the corresponding prescription or recommendation.

When a genetic marker or polymorphism “is used as a basis” for identifying or selecting a patient for a treatment described herein, the genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

“Having the same cancer” is used when comparing one patient to another or alternatively, one patient population to another patient population. For example, the two patients or patient population will each have or be suffering from colon cancer.

A “normal cell corresponding to the tumor tissue type” refers to a normal cell from a same tissue type as the tumor tissue. A non-limiting examples is a normal lung cell from a patient having lung tumor, or a normal colon cell from a patient having colon tumor.

The term “amplification” or “amplify” as used herein means one or more methods known in the art for copying a target nucleic acid, thereby increasing the number of copies of a selected nucleic acid sequence. Amplification can be exponential or linear. A target nucleic acid can be either DNA or RNA. The sequences amplified in this manner form an “amplicon.” While the exemplary methods described hereinafter relate to amplification using the polymerase chain reaction (“PCR”), numerous other methods are known in the art for amplification of nucleic acids (e.g., isothermal methods, rolling circle methods, etc.). The skilled artisan will understand that these other methods can be used either in place of, or together with, PCR methods.

The term “complement” as used herein means the complementary sequence to a nucleic acid according to standard Watson/Crick base pairing rules. A complement sequence can also be a sequence of RNA complementary to the DNA sequence or its complement sequence, and can also be a cDNA. The term “substantially complementary” as used herein means that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target or marker sequence, positioned 3′ or 5′ to a contiguous sequence of bases that hybridize under stringent hybridization conditions to a target or marker sequence.

As used herein, the term “hybridize” or “specifically hybridize” refers to a process where two complementary nucleic acid strands anneal to each other under appropriately stringent conditions. Hybridizations are typically conducted with probe-length nucleic acid molecules. Nucleic acid hybridization techniques are well known in the art. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

“Primer” as used herein refers to an oligonucleotide that is capable of acting as a point of initiation of synthesis when placed under conditions in which primer extension is initiated (e.g., primer extension associated with an application such as PCR). The primer is complementary to a target nucleotide sequence and it hybridizes to a substantially complementary sequence in the target and leads to addition of nucleotides to the 3′-end of the primer in the presence of a DNA or RNA polymerase. The 3′-nucleotide of the primer should generally be complementary to the target sequence at a corresponding nucleotide position for optimal expression and amplification. An oligonucleotide “primer” can occur naturally, as in a purified restriction digest or can be produced synthetically. The term “primer” as used herein includes all forms of primers that can be synthesized including, peptide nucleic acid primers, locked nucleic acid primers, phosphorothioate modified primers, labeled primers, and the like.

Primers are typically between about 5 and about 100 nucleotides in length, such as between about 15 and about 60 nucleotides in length, such as between about 20 and about 50 nucleotides in length, such as between about 25 and about 40 nucleotides in length. In some embodiments, primers can be at least 8, at least 12, at least 16, at least 20, at least 25, at least 30, at least 35, at least 40, at least 45, at least 50, at least 55, at least 60 nucleotides in length. An optimal length for a particular primer application can be readily determined in the manner described in H. Erlich, PCR Technology. Principles and Application for DNA Amplification (1989).

“Probe” as used herein refers to nucleic acid that interacts with a target nucleic acid via hybridization. A probe can be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe can specifically hybridize to a target nucleic acid.

Probes can be DNA, RNA or a RNA/DNA hybrid. Probes can be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes can comprise modified nucleobases, modified sugar moieties, and modified internucleotide linkages. A probe can be fully complementary to a target nucleic acid sequence or partially complementary. A probe can be used to detect the presence or absence of a target nucleic acid. Probes are typically at least about 10, 15, 21, 25, 30, 35, 40, 50, 60, 75, 100 nucleotides or more in length.

“Detecting” as used herein refers to determining the presence of a nucleic acid of interest in a sample or the presence of a protein of interest in a sample. Detection does not require the method to provide 100% sensitivity and/or 100% specificity.

“Detectable label” as used herein refers to a molecule or a compound or a group of molecules or a group of compounds used to identify a nucleic acid or protein of interest. In some cases, the detectable label can be detected directly. In other cases, the detectable label can be a part of a binding pair, which can then be subsequently detected. Signals from the detectable label can be detected by various means and will depend on the nature of the detectable label. Detectable labels can be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluorescence, or chemiluminescence, or any other appropriate means.

“TaqMan® PCR detection system” as used herein refers to a method for real time PCR. In this method, a TaqMan® probe which hybridizes to the nucleic acid segment amplified is included in the PCR reaction mix. The TaqMan® probe comprises a donor and a quencher fluorophore on either end of the probe and in close enough proximity to each other so that the fluorescence of the donor is taken up by the quencher. However, when the probe hybridizes to the amplified segment, the 5′-exonuclease activity of the Taq polymerase cleaves the probe thereby allowing the donor fluorophore to emit fluorescence which can be detected.

As used herein, the term “sample” or “test sample” refers to any liquid or solid material containing nucleic acids. In suitable embodiments, a test sample is obtained from a biological source (i.e., a “biological sample”), such as cells in culture or a tissue sample from an animal, preferably, a human. In an exemplary embodiment, the sample is a biopsy sample.

“Target nucleic acid” as used herein refers to segments of a chromosome, a complete gene with or without intergenic sequence, segments or portions a gene with or without intergenic sequence, or sequence of nucleic acids to which probes or primers are designed. Target nucleic acids can include wild type sequences, nucleic acid sequences containing mutations, deletions or duplications, tandem repeat regions, a gene of interest, a region of a gene of interest or any upstream or downstream region thereof. Target nucleic acids can represent alternative sequences or alleles of a particular gene. Target nucleic acids can be derived from genomic DNA, cDNA, or RNA. As used herein, target nucleic acid can be native DNA or a PCR-amplified product.

As used herein the term “stringency” is used in reference to the conditions of temperature, ionic strength, and the presence of other compounds, under which nucleic acid hybridizations are conducted. With high stringency conditions, nucleic acid base pairing will occur only between nucleic acids that have sufficiently long segments with a high frequency of complementary base sequences. Exemplary hybridization conditions are as follows. High stringency generally refers to conditions that permit hybridization of only those nucleic acid sequences that form stable hybrids in 0.018 M NaCl at 65° C. High stringency conditions can be provided, for example, by hybridization in 50% formamide, 5×Denhardt's solution, 5×SSC (saline sodium citrate) 0.2% SDS (sodium dodecyl sulfate) at 42° C., followed by washing in 0.1×SSC, and 0.1% SDS at 65° C. Moderate stringency refers to conditions equivalent to hybridization in 50% formamide, 5×Denhardt's solution, 5×SSC, 0.2% SDS at 42° C., followed by washing in 0.2×SSC, 0.2% SDS, at 65° C. Low stringency refers to conditions equivalent to hybridization in 10% formamide, 5×Denhardt's solution, 6×SSC, 0.2% SDS, followed by washing in 1° SSC, 0.2% SDS, at 50° C.

As used herein the term “substantially identical” refers to a polypeptide or nucleic acid exhibiting at least 50%, 75%, 85%, 90%, 95%, or even 99% identity to a reference amino acid or nucleic acid sequence over the region of comparison. For polypeptides, the length of comparison sequences will generally be at least 20, 30, 40, or 50 amino acids or more, or the full length of the polypeptide. For nucleic acids, the length of comparison sequences will generally be at least 10, 15, 20, 25, 30, 40, 50, 75, or 100 nucleotides or more, or the full length of the nucleic acid.

### Descriptive Embodiments

The disclosure further provides diagnostic, prognostic and therapeutic methods, which are based, at least in part, on determination of the identify of a genotype of interest identified herein.

For example, information obtained using the diagnostic assays described herein is useful for determining if a subject is suitable for cancer treatment of a given type. Based on the prognostic information, a doctor can recommend a therapeutic protocol, useful for reducing the malignant mass or tumor in the patient or treat cancer in the individual.

A patient's likely clinical outcome following a clinical procedure such as a therapy or surgery can be expressed in relative terms. For example, a patient having a particular genotype or expression level can experience relatively longer overall survival than a patient or patients not having the genotype or expression level. The patient having the particular genotype or expression level, alternatively, can be considered as likely to survive. Similarly, a patient having a particular genotype or expression level can experience relatively longer progression free survival, or time to tumor progression, than a patient or patients not having the genotype or expression level. The patient having the particular genotype or expression level, alternatively, can be considered as not likely to suffer tumor progression. Further, a patient having a particular genotype or expression level can experience relatively shorter time to tumor recurrence than a patient or patients not having the genotype or expression level. The patient having the particular genotype or expression level, alternatively, can be considered as not likely to suffer tumor recurrence. Yet in another example, a patient having a particular genotype or expression level can experience relatively more complete response or partial response than a patient or patients not having the genotype or expression level. The patient having the particular genotype or expression level, alternatively, can be considered as likely to respond. Accordingly, a patient that is likely to survive, or not likely to suffer tumor progression, or not likely to suffer tumor recurrence, or likely to respond following a clinical procedure is considered suitable for the clinical procedure.

It is to be understood that information obtained using the diagnostic assays described herein can be used alone or in combination with other information, such as, but not limited to, genotypes or expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis or treatment.

The methods are useful in the assistance of an animal, a mammal or yet further a human patient. For the purpose of illustration only, a mammal includes but is not limited to a human, a simian, a murine, a bovine, an equine, a porcine or an ovine subject.

### Diagnostic Methods

The prediction of treatment outcomes based on presence of certain polymorphisms in colorectal cancer patients administered TAS-102 is based on a study conducted by the Applicant. In this study genomic DNA extracted from 233 blood samples of three different cohorts: a training cohort (n=52) and a testing cohort (n=129) both receiving TAS-102; a control cohort (n=52) receiving regorafenib. SNPs of TK1, hENT1, hCNT1, MATE1, MATE2 and OCT2 genes were analyzed by PCR-based direct DNA sequencing.

In the training cohort, patients with any hENT1 rs760370 G allele had longer progression free survival (PFS) (3.5 vs. 2.1 months, HR 0.44, P=0.004) and overall survival (OS) (8.7 vs. 5.3 months, HR 0.27, P=0.003) than those carrying the A/A genotype. These findings were validated in the testing cohort (P=0.021 and 0.009 for PFS and OS, respectively). Additionally, the combination of hENT1 rs760370, MATE1 rs2289669 with OCT2 rs316019 polymorphisms significantly stratified patients with the risk of PFS and OS in both cohorts (P<0.001 for PFS and OS in training cohort; P=0.025 and 0.053 for PFS and OS respectively in testing cohort). In multivariable analysis, these correlations remained significant in both cohorts. No significant differences were observed in the control group.

Thus, the combination of hENT1, MATE1 and OCT2 gene polymorphisms could serve as predictive and prognostic markers in metastatic colorectal cancer (mCRC) patients treated with TAS-102.

TAS-102 consists of a cytotoxic component, trifluridine (FTD), and of thymidine phospholyrase inhibitor (TPI) that inhibits the rapid degradation of FTD. Although FTD incorporation into DNA of the tumor cells is identified as anti-tumor mechanism, the role of TPI remains unknown. The study focused on the finding that TPI is mainly excreted in urine without being metabolized, and hypothesized that circulating unchanged TPI in blood might re-induce inhibition of TP to maintain FTD concentration in tumor cells. The findings show for the first time that germline polymorphism of nucleoside transporter hENT1 regulating FTD intracellular influx/efflux significantly correlates with outcomes alone, and clearly classifies the risk of outcomes when combining gene polymorphisms of other transporters MATE1 and OCT2 involved in TPI excretion. The suggested classification consisting of three transporters gene polymorphisms may serve as predictive and prognostic marker in patients with refractory metastatic colorectal cancer treated with TAS-102.

Provided, in one embodiment, is a method for selecting a colorectal cancer patient for a therapy comprising, or alternatively consisting essentially of, or yet further consisting of TAS-102 or an equivalent thereof, comprising, or alternatively consisting essentially of, or yet further consisting of screening a biological sample isolated from the patient for one or more polymporphism(s) identified in the above Table A or as identified herein as favorable, and selecting the patient for the therapy if the genotype as identified in the favorable genotype(s) column of Table A is present in the sample. In some aspects, the patient is not selected for the therapy if the favorable genotype of Table A is not present in the sample.

Also provided is a method for identifying whether a colorectal cancer patient is likely to experience a relatively longer or shorter progression free survival (PFS) or overall survival (OS) following a therapy comprising, or alternatively consisting essentially of, or yet further consisting of TAS102, comprising, or alternatively consisting essentially of, or yet further consisting of screening a biological sample isolated from the patient for one or more polymorphism(s) identified in the above Table A, and identifying that the patient is likely to experience a longer progression free survival if genotype identified by the method is identified in the favorable genotype(s) column of Table A is present in the sample, relative to a colorectal cancer patient not having the genotype.

Yet further provided is a method for detecting a biomarker identified in Table A in a biological sample isolated from a patient, comprising, or alternatively consisting essentially of, or yet further consisting of screening the biological sample for one more polymorphism(s) identified in Table A.

In some embodiments, provided is an in vitro method of detecting a polymorphism in a patient with cancer or a patient suspected of having cancer, e.g., GI cancer, colon cancer, rectal cancer, or colorectal cancer, the method comprising, or alternatively consisting essentially of, or yet further consisting of screening a biological sample from the patient to detect the genotypes of (G/C) or (G/G) for rs609429, (A/G) or (A/A) for rs861539, (A/G) or (G/G) for rs760370, or (C/T) or (T/T) for rs9394992. In some embodiments, the patient is known to have the cancer. In other embodiments, the patient is suspected of having the cancer. In some embodiments, the screening comprises, or alternatively consists essentially of, or yet further consists of detecting the genotypes of (G/C) or (G/G) for rs609429 in the sample. In some embodiments, the screening comprises, or alternatively consists essentially of, or yet further consists of detecting the genotypes of (A/G) or (A/A) for rs861539 in the sample. In some embodiments, the screening comprises, or alternatively consists essentially of, or yet further consists of detecting the genotypes of (A/G) or (G/G) for rs760370 in the sample. In some embodiments, the screening comprises, or alternatively consists essentially of, or yet further consists of detecting the genotypes of (C/T) or (T/T) for rs9394992 in the sample. In some embodiments, the method further comprises, or alternatively consists essentially of, or yet further consists of, administering a therapy comprising, or alternatively consisting essentially of, or yet further consisting of administration of an effective amount of TAS-102. The therapy can be first line, second line, third line, fourth line of fifth line therapy and is some aspects, can be administered alone or in combination with other therapies, e.g., surgical resection, radiation therapy or other chemical or biological based therapies.

Provided, in one embodiment, is a method for selecting a cancer patient, e.g., a GI cancer patient, a colon cancer patient, a rectal cancer patient, or a colorectal cancer patient for a therapy comprising, or alternatively consisting essentially of, or yet further consisting of administration of an effective amount of TAS-102, the method comprising, or alternatively consisting essentially of, or yet further consisting of screening a biological sample isolated from the patient for an rs861539 polymorphism, and selecting the patient for the therapy if the genotype of (G/C) or (G/G) for rs609429, (A/G) or (A/A) for rs861539, (A/G) or (G/G) for rs760370, or (C/T) or (T/T) for rs9394992 is present in the sample. In some aspects, the patient is not selected for the therapy if the genotype of (G/C) or (G/G) for rs609429, (A/G) or (A/A) for rs861539, (A/G) or (G/G) for rs760370, or (C/T) or (T/T) for rs9394992 is not present in the sample. In some aspects, the patient is not selected for the therapy if the genotype of (C/C) for rs609429, (G/G) for rs861539, (A/A) for rs760370, or (G/G) for rs9394992 is present in the sample. In some embodiments, the patient is selected for a TAS-102-free therapy if the genotype of (G/C) or (G/G) for rs609429, (A/G) or (A/A) for rs861539, (A/G) or (G/G) for rs760370, or (C/T) or (T/T) for rs9394992 is not present in the sample. Alternative therapies are known in the art, some of which are described herein. In some embodiments, the patient is selected for a TAS-102-free therapy if the genotype of (C/C) for rs609429, (G/G) for rs861539, (A/A) for rs760370, or (G/G) for rs9394992 is present in the sample.

In some embodiments, provided is a method for selecting a cancer patient, e.g. a GI cancer, colon cancer, a rectal cancer or a colorectal cancer patient for a therapy comprising, or alternatively consisting essentially of, or yet further consisting of administration of an effective amount of TAS-102, the method comprising, or alternatively consisting essentially of, or yet further consists of, screening a biological sample isolated from the patient for an rs609429 polymorphism, and selecting the patient for the therapy if the genotype of (G/C) or (G/G) for rs609429 is present in the sample. In some embodiments, the patient is not selected for a therapy comprising, or alternatively consisting essentially of, or yet further consists of, a therapeutically effective amount of TAS-102 if the genotype of (G/C) or (G/G) for rs609429 is not present in the sample. In some embodiments, the patient is not selected for a therapy comprising, or alternatively consisting essentially of, or yet further consists of, TAS-102 if the genotype of (C/C) for rs609429 is present in the sample. In some embodiments, the patient is selected for a TAS-102-free therapy if the genotype of (G/C) or (G/G) for rs609429 is not present in the sample. Non-limiting examples of other cancer therapies are known in the art and described herein. In some embodiments, the patient is selected for a TAS-102-free therapy if the genotype of (C/C) for rs609429 is present in the sample. The therapy can be first line, second line, third line, fourth line of fifth line therapy and is some aspects, can be administered alone or in combination with other therapies, e.g., surgical resection, radiation therapy or other chemical or biological based therapies.

Also provided is a method for classifying a cancer patient, e.g. a GI cancer, colon cancer, a rectal cancer, or a colorectal cancer patient as eligible for a therapy comprising, or alternatively consisting essentially of, or yet further consists of, administration of an effective amount of TAS-102, the method comprising, or alternatively consisting essentially of, or yet further consists of, screening a biological sample isolated from the patient for an rs609429 polymorphism, and classifying the patient as eligible for the therapy if the genotype of (G/C) or (G/G) for rs609429 is present in the sample. In some embodiments, the method comprises, or consists essentially of, or yet further consists of, classifying the patient as not eligible for the therapy comprising, or alternatively consisting essentially of, or yet further consists of, administration of an effective amount of TAS-102 if the genotype of (G/C) or (G/G) for rs609429 is not present in the sample. In some embodiments, the patient is classified as not eligible for the therapy comprising, or alternatively consisting essentially of, or yet further consisting of TAS-102 if the genotype of (C/C) for rs609429 is present in the sample. In some embodiments, the method further comprises, or consists essentially of, or yet further consists of, administering a therapy comprising, or alternatively consisting essentially of, or yet further consists of, a therapeutically effective amount of TAS-102. The therapy can be first line, second line, third line, fourth line of fifth line therapy and is some aspects, can be administered alone or in combination with other therapies, e.g., surgical resection, radiation therapy or other chemical or biological based therapies.

Also provided is a method for increasing the progression-free and/or overall survival of a cancer patient, e.g. a GI cancer, colon cancer, a rectal cancer or a colorectal cancer patient, comprising, or alternatively consisting essentially of, or yet further consists of, screening a biological sample isolated from the patient for an rs609429 polymorphism, and classifying the patient as eligible for a therapy comprising, or alternatively consisting essentially of, or yet further consisting of administration of an effective amount of TAS-102 if the genotype of (G/C) or (G/G) for rs609429 is present in the sample, or not eligible for the therapy comprising, or alternatively consisting essentially of, or yet further consists of, TAS-102 if the genotype of (G/C) or (G/G) for rs609429 is not present in the sample. In some embodiments, the patient is classified as not eligible for the therapy comprising, or alternatively consisting essentially of, or yet further consists of, TAS-102 if the genotype of (C/C) for rs609429 is present in the sample. In some embodiments, the method further comprises, or consists essentially of, or yet further consists of, administering a therapy comprising, or alternatively consisting essentially of, or yet further consists of, an effective amount of TAS-102 or a TAS-102-free therapy in accordance with the classification. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, are methods for identifying whether a cancer patient, e.g. a GI cancer, colon cancer, a rectal cancer or a colorectal cancer patient is likely to experience a relatively longer or shorter progression free survival (PFS) following a therapy comprising, or alternatively consisting essentially of, or yet further consisting of administration of an effective amount of TAS-102, the method comprising, or alternatively consisting essentially of, or yet further consists of, screening a biological sample isolated from the patient for an rs609429 polymorphism, and identifying that the patient as likely to experience a longer progression free survival if the genotype of (G/C) or (G/G) for rs609429 is present in the sample, relative to a corresponding cancer patient not having the genotype. In some embodiments, the method comprises, or consists essentially of, or yet further consists of, identifying that the patient is likely to experience a shorter progression free survival if the genotype of (G/C) or (G/G) for rs609429 is not present in the sample, relative to a corresponding cancer patient having the genotype or relative to a corresponding cancer patient having the genotype of (C/C) for rs609429. In some embodiments, the method comprises, or consists essentially of, or yet further consists of, identifying that the patient is likely to experience a shorter progression free survival if the genotype of (C/C) for rs609429 is present in the sample, relative to a corresponding cancer patient not having the genotype or relative to a colorectal cancer patient having the genotype of (G/C) or (G/G) for rs609429. The therapy can be first line, second line, third line, fourth line of fifth line therapy and is some aspects, can be administered alone or in combination with other therapies, e.g., surgical resection, radiation therapy or other chemical or biological based therapies.

Further provided is a method for identifying whether a cancer, patient, e.g., a GI cancer patient, a colon cancer patient, a rectal cancer patient, or a colorectal cancer patient is likely to experience TAS-102 related toxicity following a therapy, the therapy comprising, or alternatively consisting essentially of, or yet further consisting of administration of an effective amount of TAS-102, the method comprising, or alternatively consisting essentially of, or yet further consisting of screening a biological sample isolated from the patient for a polymorphism, and identifying that the patient is likely to experience toxicity if the genotype of (G/C) or (G/G) for rs609429 is present in the sample, relative to a corresponding patient not having the genotype. In some aspects, the TAS-102 related toxicity comprises, or alternatively consists essentially of, or yet further consists of one or more of the group of: febrile neutropenia, leukopenia, stomatitis, neutropenia, hand-foot syndrome, cardiac ischemia, thrombocytopenia, increase in alanine aminotransferase level, increase in aspartate aminotransferase level, increase in total bilirubin, increase in alkaline phosphatase level, increase in creatinine level, anemia, anorexia, nausea, vomiting, alopecia, decreased appetite, fatigue, abdominal pain, fever, asthenia, or diarrhea. In yet further aspects, the toxicity comprises, or consists essentially of, or yet further consists of, neutropenia. In some aspects, the toxicity is limited to adverse events of grade 3 severity or higher. In other aspects, the toxicity is limited to adverse events of grade 3 severity or lower.

Also provided, in some embodiments, is a method for treating a cancer patient, e.g. a GI cancer, colon cancer, a rectal cancer or a colorectal cancer patient selected for treatment based on the presence of the genotype of (G/C) or (G/G) for rs609429 in a biological sample from the patient, the method comprising, or alternatively consisting essentially of, or yet further consists of, administering to the patient a therapy comprising, or alternatively consisting essentially of, or yet further consists of, an effective amount of TAS-102. Also provided is a method for treating a cancer patient, e.g. a GI cancer, colon cancer, a rectal cancer or a colorectal cancer patient selected for treatment based on the absence of the genotype of (C/C) for rs609429 in a biological sample from the patient, the method comprising, or alternatively consisting essentially of, or yet further consists of, administering to the patient a therapy comprising, or alternatively consisting essentially of, or yet further consists of, an effective amount of TAS-102. The therapy can be first line, second line, third line, fourth line of fifth line therapy and is some aspects, can be administered alone or in combination with other therapies, e.g., surgical resection, radiation therapy or other chemical or biological based therapies.

In some embodiments, the methods noted above further comprise screening a biological sample isolated from the patient for the rs609429 polymorphism. Thus, also provided, in some embodiments, is a method for treating a cancer patient, e.g. a GI cancer, colon cancer, a rectal cancer or a colorectal cancer patient, the method comprising, or alternatively consisting essentially of, or yet further consists of, screening a biological sample isolated from the patient for the rs609429 polymorphism and administering to the patient a therapy comprising, or alternatively consisting essentially of, or yet further consists of, an effective amount of TAS-102 if the sample has the genotype of (G/C) or (G/G) for rs609429.

Also provided, in some embodiments, is a method for modifying the treatment of a cancer patient, e.g. a GI cancer, colon cancer, a rectal cancer or a colorectal cancer patient receiving a therapy, the therapy comprising, or alternatively consisting essentially of, or yet further consists of, an effective amount of TAS-102 based on the presence of the genotype of (G/C) or (G/G) for rs609429 in a biological sample from the patient. For example, provided are methods for modifying the treatment of the patient receiving a therapy comprising, or alternatively consisting essentially of, or yet further consists of, an effective amount of TAS-102. The method further comprises, or consists essentially of, or yet further consists of, screening a biological sample isolated from the patient for an rs609429 polymorphism, and modifying the dosage or frequency of the therapy based on the genotype for rs609429. In some embodiments, the dosage or frequency of the therapy, or components thereof (e.g., one or more therapeutic agents of the therapy), is increased if the genotype of (G/C) or (G/G) for rs609429 is not present in the sample. In some embodiments, the dosage or frequency of the therapy, or components thereof, is increased if the genotype of (C/C) for rs609429 is present in the sample. In some embodiments, the therapy is discontinued if the genotype of (G/C) or (G/G) for rs609429 is not present in the sample. In some embodiments, the therapy is discontinued if the genotype of (C/C) for rs609429 is present in the sample. In some embodiments, the therapy is continued if the genotype of (G/C) or (G/G) for rs609429 is present in the sample. The therapy can be first line, second line, third line, fourth line of fifth line therapy and is some aspects, can be administered alone or in combination with other therapies, e.g., surgical resection, radiation therapy or other chemical or biological based therapies.

In the above methods any appropriate technique can be used for screening the biological sample isolated from the patient for an rs609429 polymorphism. A non-limiting example comprises, or consists essentially of, or yet further consists of contacting the biological sample with a nucleic acid probe that specifically binds to nucleic acid containing the rs609429 polymorphism and overlaps the polymorphic site. For example, in some embodiments, the nucleic acid specifically binds to a nucleic acid having the sequence of SEQ ID NO:1, SEQ ID NO:29, or SEQ ID NO:30 and overlaps the polymorphic site. In some embodiments, the a nucleic acid is labeled with a detectable moiety, having about 5, about 10, about 15, about 20, about 25, about 30, about 35, or about 40 nucleotides upstream and/or downstream of the polymorphic region. In another aspect, whole genome sequencing can be used to determine the identity of the genome at the site of interest.

In some embodiments, screening a biological sample isolated from the patient for an rs609429 polymorphism comprises, or consists essentially of, or yet further consists of amplifying nucleic acid containing the rs609429 polymorphism. In some embodiments, nucleic acid containing the rs609429 polymorphism is amplified using a forward primer comprising, or alternatively consisting essentially of, or yet further consisting of nucleic acid having the sequence of SEQ ID NO:8 and a reverse primer comprising, or alternatively consisting essentially of, or yet further consisting of nucleic acid having the sequence of SEQ ID NO:9.

In some embodiments, provided are methods for selecting a cancer patient, e.g. a GI cancer, colon cancer, a rectal cancer or a colorectal cancer patient for a therapy comprising, or alternatively consisting essentially of, or yet further consists of, administration of an effective amount of TAS-102. The selection method comprises, or alternatively consists essentially of, or yet further consists of, screening a biological sample isolated from the patient for an rs861539 polymorphism, and selecting the patient for the therapy if the genotype of (A/G) or (A/A) for rs861539 is present in the sample. In some embodiments, the patient is not selected for the therapy if the genotype of (A/G) or (A/A) for rs861539 is not present in the sample. In some embodiments, the patient is not selected for the therapy if the genotype of (G/G) for rs861539 is present in the sample. In some embodiments, the patient is selected for a TAS-102-free therapy if the genotype of (A/G) or (A/A) for rs861539 is not present in the sample. In some embodiments, the patient is selected for a TAS-102-free therapy if the genotype of (G/G) for rs861539 is present in the sample. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, are methods for classifying a cancer patient, e.g. a GI cancer, colon cancer, a rectal cancer or a colorectal cancer patient as eligible for a therapy, the therapy comprising, or alternatively consisting essentially of, or yet further consists of, administration of an effective amount of TAS-102, comprising, or alternatively consisting essentially of, or yet further consisting of screening a biological sample isolated from the patient for an rs861539 polymorphism, and classifying the patient as eligible for the therapy if the genotype of (A/G) or (A/A) for rs861539 is present in the sample. In some embodiments, the method comprises, or consists essentially of, or yet further consists of classifying the patient as not eligible for the therapy comprising, or alternatively consisting essentially of, or yet further consisting of TAS-102 if the genotype of (A/G) or (A/A) for rs861539 is not present in the sample. In some embodiments, the patient is classified as not eligible for the therapy comprising, or alternatively consisting essentially of, or yet further consisting of administration of an effective amount of TAS-102 if the genotype of (G/G) for rs861539 is present in the sample. In some embodiments, the method further comprises, or alternatively consists essentially of, or yet further consists of, administering a therapy comprising, or alternatively consisting essentially of, or yet further consisting of administration of an effective amount of TAS-102. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, are methods for increasing the progression-free and/or overall survival of a cancer patient, e.g. a GI cancer, colon cancer, a rectal cancer or a colorectal cancer patient, comprising, or alternatively consisting essentially of, or yet further consists of, screening a biological sample isolated from the patient for an rs861539 polymorphism, and classifying the patient as eligible for the therapy with TAS-102 if the genotype of (A/G) or (A/A) for rs861539 is present in the sample or not eligible for the therapy comprising, or alternatively consisting essentially of, or yet further consists of, administration of an effective amount of TAS-102 if the genotype of (A/G) or (A/A) for rs861539 is not present in the sample. In some embodiments, the patient is classified as not eligible for the therapy comprising, or alternatively consisting essentially of, or yet further consists of, administration of an effective amount of TAS-102 if the genotype of (G/G) for rs861539 is present in the sample. In some embodiments, the method further comprises, or consists essentially of, or yet further consists of administering a therapy comprising, or alternatively consisting essentially of, or yet further consisting of an effective amount of TAS-102 or a TAS-102-free therapy in accordance with the classification. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, are methods for identifying whether a cancer patient, e.g. a GI cancer, colon cancer, a rectal cancer or a colorectal cancer patient is likely to experience a relatively longer or shorter progression free survival (PFS) following a therapy comprising, or alternatively consisting essentially of, or yet further consists of, an administration of an effective amount of TAS-102, comprising, or alternatively consisting essentially of, or yet further consists of, screening a biological sample isolated from the patient for an rs861539 polymorphism, and identifying that the patient is likely to experience a longer progression free survival if the genotype of (A/G) or (A/A) for rs861539 is present in the sample, relative to the patient not having the genotype. In some embodiments, the method comprises, or consists essentially of, or yet further consists of identifying that the patient is likely to experience a shorter progression free survival if the genotype of (A/G) or (A/A) for rs861539 is not present in the sample, relative to a patient having the genotype or relative to a patient having the genotype of (G/G) for rs861539. In some embodiments, the method comprises, or consists essentially of, or yet further consists of identifying that the patient is likely to experience a shorter progression free survival if the genotype of (G/G) for rs861539 is present in the sample, relative to the patient not having the genotype or relative to the patient having the genotype of (A/G) or (A/A) for rs861539. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, are methods for treating a cancer patient, e.g. a GI cancer, colon cancer, a rectal cancer or a colorectal cancer patient selected for treatment based on the presence of the genotype of (A/G) or (A/A) for rs861539 in a biological sample from the patient, comprising, or alternatively consisting essentially of, or yet further consists of, administering to the patient a therapy comprising, or alternatively consisting essentially of, or yet further consists of, an effective amount of TAS-102. Also provided, in some embodiments, are methods for treating a cancer patient, e.g. a GI cancer, colon cancer, a rectal cancer or a colorectal cancer patient selected for treatment based on the absence of the genotype of (G/G) for rs861539 in a biological sample from the patient, comprising, or alternatively consisting essentially of, or yet further consists of, administering to the patient a therapy comprising, or alternatively consisting essentially of, or yet further consisting of an effective amount of TAS-102. The therapy can be first line, second line, third line, fourth line of fifth line therapy and is some aspects, can be administered alone or in combination with other therapies, e.g., surgical resection, radiation therapy or other chemical or biological based therapies. In some embodiments, the patient is treated with a TAS-102-free therapy if the genotype of (G/C) or (G/G) for rs609429, (A/G) or (A/A) for rs861539, (A/G) or (G/G) for rs760370, or (C/T) or (T/T) for rs9394992 is not present in the sample. In some embodiments, the patient is treated with a TAS-102-free therapy if the genotype of (C/C) for rs609429, (G/G) for rs861539, (A/A) for rs760370, or (G/G) for rs9394992 is present in the sample. Alternative therapies are known in the art, non-limiting examples of such are described herein.

In some embodiments, the method further comprises, or consists essentially of, or yet further consists of, screening a biological sample isolated from the patient for the rs861539 polymorphism. Thus, also provided, in some embodiments, are methods for treating a cancer patient, e.g. a GI cancer, colon cancer, a rectal cancer or a colorectal cancer patient, comprising, or alternatively consisting essentially of, or yet further consists of, screening a biological sample isolated from the patient for the rs861539 polymorphism and administering to the patient a therapy comprising, or alternatively consisting essentially of, or yet further consists of, an effective amount of TAS-102 if the sample has the genotype of (A/G) or (A/A) for rs861539. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, are methods for modifying the treatment of a cancer patient, e.g. a GI cancer, colon cancer, a rectal cancer or a colorectal cancer patient receiving a therapy, the therapy comprising, or alternatively consisting essentially of, or yet further consists of, an effective amount of TAS-102 based on the presence of the genotype of (A/G) or (A/A) for rs861539 in a biological sample from the patient. For example, provided are methods for modifying the treatment of the patient receiving a therapy comprising, or alternatively consisting essentially of, or yet further consists of, an effective amount of TAS-102, the method comprising, or alternatively consisting essentially of, or yet further consisting of screening a biological sample isolated from the patient for an rs861539 polymorphism, and modifying the dosage or frequency of the therapy comprising, or alternatively consisting essentially of, or yet further consists of, an effective amount of TAS-102 based on the genotype for rs861539. In some embodiments, the dosage or frequency of the therapy, or components thereof (e.g., one or more therapeutic agents of the therapy), is increased if the genotype of (A/G) or (A/A) for rs861539 is not present in the sample. In some embodiments, the dosage or frequency of the therapy, or components thereof, is increased if the genotype of (G/G) for rs861539 is present in the sample. In some embodiments, the therapy is discontinued if the genotype of (A/G) or (A/A) for rs861539 is not present in the sample. In some embodiments, the therapy is discontinued if the genotype of (G/G) for rs861539 is present in the sample. In some embodiments, the therapy is continued if the genotype of (A/G) or (A/A) for rs861539 is present in the sample. The therapy can be first line, second line, third line, fourth line of fifth line therapy and is some aspects, can be administered alone or in combination with other therapies, e.g., surgical resection, radiation therapy or other chemical or biological based therapies.

In some embodiments, screening a biological sample isolated from the patient for an rs861539 polymorphism comprises, or consists essentially of, or yet further consists of contacting the biological sample with a nucleic acid probe that specifically binds to nucleic acid containing the rs861539 polymorphism and overlaps the polymorphic site. For example, in some embodiments, the nucleic acid specifically binds to a nucleic acid having the sequence of SEQ ID NO:2, SEQ ID NO:31, or SEQ ID NO:32 and overlaps the polymorphic site. In some embodiments, the a nucleic acid is labeled with a detectable moiety, having about 5, about 10, about 15, about 20, about 25, about 30, about 35, or about 40 nucleotides upstream and/or downstream of the polymorphic region. In other aspects, it comprises, or consists essentially of, or yet further consists of whole genome sequencing of the patient's genotype.

In some embodiments, screening a biological sample isolated from the patient for an rs861539 polymorphism comprises, or consists essentially of, or yet further consists of amplifying nucleic acid containing the rs861539 polymorphism. In some embodiments, nucleic acid containing the rs861539 polymorphism is amplified using a forward primer comprising, or alternatively consisting essentially of, or yet further consisting of nucleic acid having the sequence of SEQ ID NO:10 and a reverse primer comprising, or alternatively consisting essentially of, or yet further consisting of nucleic acid having the sequence of SEQ ID NO:11. In other embodiments, nucleic acid containing the rs861539 polymorphism is amplified using a forward primer comprising, or alternatively consisting essentially of, or yet further consisting of nucleic acid having the sequence of SEQ ID NO:49 and a reverse primer comprising, or alternatively consisting essentially of, or yet further consisting of nucleic acid having the sequence of SEQ ID NO:11.

In some embodiments, provided are methods for selecting a cancer, patient, e.g., a GI cancer patient, a colon cancer patient, a rectal cancer patient, or a colorectal cancer patient for a therapy comprising, or alternatively consisting essentially of, or yet further consists of, administration of an effective amount of TAS-102, the method comprising, or alternatively consisting essentially of, or yet further consisting of screening a biological sample isolated from the patient for an rs760370 polymorphism, and selecting the patient for the therapy if the genotype of (A/G) or (G/G) for rs760370 is present in the sample. In some embodiments, the patient is not selected for the therapy comprising, or alternatively consisting essentially of, or yet further consists of, administration of an effective amount of TAS-102 if the genotype of (A/G) or (G/G) for rs760370 is not present in the sample. In some embodiments, the patient is not selected for a therapy comprising, or alternatively consisting essentially of, or yet further consisting of administration of an effective amount of TAS-102 if the genotype of (A/A) for rs760370 is present in the sample. In some embodiments, the patient is selected for a TAS-102-free therapy if the genotype of (A/G) or (G/G) for rs760370 is not present in the sample. In some embodiments, the patient is selected for a TAS-102-free therapy if the genotype of (A/A) for rs760370 is present in the sample. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, are methods for classifying a cancer, patient, e.g., a GI cancer patient, a colon cancer patient, a rectal cancer patient, or a colorectal cancer patient as eligible for a therapy, the therapy comprising, or alternatively consisting essentially of, or yet further consists of, administration of TAS-102, the method comprising, or alternatively consisting essentially of, or yet further consists of, screening a biological sample isolated from the patient for an rs760370 polymorphism, and classifying the patient as eligible for the therapy if the genotype of (A/G) or (G/G) for rs760370 is present in the sample. In some embodiments, the method comprises, or consists essentially of, or yet further consists of classifying the patient as not eligible for the therapy comprising, or alternatively consisting essentially of, or yet further consists of, TAS-102 if the genotype of (A/G) or (G/G) for rs760370 is not present in the sample. In some embodiments, the patient is classified as not eligible for the therapy comprising, or alternatively consisting essentially of, or yet further consists of, TAS-102 if the genotype of (A/A) for rs760370 is present in the sample. In some embodiments, the method further comprises, or consists essentially of, or consists of, administering a therapy comprising, or alternatively consisting essentially of, or yet further consisting of an effective amount of TAS-102. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, are methods for increasing the progression-free and/or overall survival of a cancer, patient, e.g., a GI cancer patient, a colon cancer patient, a rectal cancer patient, or a colorectal cancer patient, comprising, or alternatively consisting essentially of, or yet further consists of, screening a biological sample isolated from the patient for an rs760370 polymorphism, and classifying the patient as eligible for the therapy with TAS-102 if the genotype of (A/G) or (G/G) for rs760370 is present in the sample or not eligible for the therapy comprising, or alternatively consisting essentially of, or yet further consists of, administration of an effective amount of TAS-102 if the genotype of (A/G) or (G/G) for rs760370 is not present in the sample. In some embodiments, the patient is classified as not eligible for the therapy comprising, or alternatively consisting essentially of, or yet further consisting of TAS-102 if the genotype of (A/A) for rs760370 is present in the sample. In some embodiments, the method further comprises, or consists essentially of, or consists of, administering a therapy comprising, or alternatively consisting essentially of, or yet further consists of, an effective amount of TAS-102 or a TAS-102-free therapy in accordance with the classification. The therapy can be first line, second line, third line, fourth line of fifth line therapy and is some aspects, can be administered alone or in combination with other therapies, e.g., surgical resection, radiation therapy or other chemical or biological based therapies. Also provided, in some embodiments, are methods for identifying whether a cancer, patient, e.g., a GI cancer patient, a colon cancer patient, a rectal cancer patient, or a colorectal cancer patient is likely to experience a relatively longer or shorter progression free survival (PFS) following a therapy, the therapy comprising, or alternatively consisting essentially of, or yet further consists of, an effective amount of TAS-102, comprising, or alternatively consisting essentially of, or yet further consists of, screening a biological sample isolated from the patient for an rs760370 polymorphism, and identifying that the patient is likely to experience a longer progression free survival if the genotype of (A/G) or (G/G) for rs760370 is present in the sample, relative to a corresponding cancer patient not having the genotype. In some embodiments, the method comprises, or consists essentially of, or yet further consists of identifying that the patient is likely to experience a shorter progression free survival if the genotype of (A/G) or (G/G) for rs760370 is not present in the sample, relative to a corresponding cancer patient having the genotype or relative to a cancer patient having the genotype of (A/A) for rs760370. In some embodiments, the method comprises, or consists essentially of, or yet further consists of identifying that the patient is likely to experience a shorter progression free survival if the genotype of (A/A) for rs760370 is present in the sample, relative to a corresponding cancer patient not having the genotype or relative to a corresponding cancer patient having the genotype of (A/G) or (G/G) for rs760370. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, are methods for treating a cancer, patient, e.g., a GI cancer patient, a colon cancer patient, a rectal cancer patient, or a colorectal cancer patient selected for treatment based on the presence of the genotype of (A/G) or (G/G) for rs760370 in a biological sample from the patient, comprising, or alternatively consisting essentially of, or yet further consists of, administering to the patient a therapy comprising, or alternatively consisting essentially of, or yet further consists of, administration of an effective amount of TAS-102. Also provided, in some embodiments, are methods for treating the cancer patient selected for treatment based on the absence of the genotype of (A/A) for rs760370 in a biological sample from the patient, comprising, or alternatively consisting essentially of, or yet further consists of, administering to the patient a therapy comprising, or alternatively consisting essentially of, or yet further consisting of an effective amount of TAS-102. The therapy can be first line, second line, third line, fourth line of fifth line therapy and is some aspects, can be administered alone or in combination with other therapies, e.g., surgical resection, radiation therapy or other chemical or biological based therapies.

In some embodiments, the method further comprises, or consists essentially of, or yet further consists of screening a biological sample isolated from the patient for the rs760370 polymorphism. Thus, also provided, in some embodiments, are methods for treating a cancer, patient, e.g., a GI cancer patient, a colon cancer patient, a rectal cancer patient, or a colorectal cancer patient, comprising, or alternatively consisting essentially of, or yet further consists of, screening a biological sample isolated from the patient for the rs760370 polymorphism and administering to the patient a therapy comprising, or alternatively consisting essentially of, or yet further consists of, an effective amount of a TAS-102 if the sample has the genotype of (A/G) or (G/G) for rs760370. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, are methods for modifying the treatment of patient receiving a therapy, the therapy comprising, or alternatively consisting essentially of, or yet further consists of, administration of an effective amount of TAS-102 based on the presence of the genotype of (A/G) or (G/G) for rs760370 in a biological sample from the patient. For example, provided are methods for modifying the treatment of patient receiving a therapy comprising, or alternatively consisting essentially of, or yet further consists of, administration of an effective amount of TAS-102, comprising, or alternatively consisting essentially of, or yet further consists of, screening a biological sample isolated from the patient for an rs760370 polymorphism, and modifying the dosage or frequency of the therapy comprising, or alternatively consisting essentially of, or yet further consists of, an effective amount of TAS-102 based on the genotype for rs760370. In some embodiments, the dosage or frequency of the therapy, or components thereof (e.g., one or more therapeutic agents of the therapy), is increased if the genotype of (A/G) or (G/G) for rs760370 is not present in the sample. In some embodiments, the dosage or frequency of the therapy, or components thereof, is increased if the genotype of (A/A) for rs760370 is present in the sample. In some embodiments, the therapy is discontinued if the genotype of (A/G) or (G/G) for rs760370 is not present in the sample. In some embodiments, the therapy is discontinued if the genotype of (A/A) for rs760370 is present in the sample. In some embodiments, the therapy is continued if the genotype of (A/G) or (G/G) for rs760370 is present in the sample. The therapy can be first line, second line, third line, fourth line of fifth line therapy and is some aspects, can be administered alone or in combination with other therapies, e.g., surgical resection, radiation therapy or other chemical or biological based therapies.

In some embodiments, screening a biological sample isolated from the patient for an rs760370 polymorphism comprises, or consists essentially of, or yet further consists of contacting the biological sample with a nucleic acid probe that specifically binds to nucleic acid containing the rs760370 polymorphism and overlaps the polymorphic site. For example, in some embodiments, the nucleic acid specifically binds to a nucleic acid having the sequence of SEQ ID NO:3, SEQ ID NO:33, or SEQ ID NO:34 and overlaps the polymorphic site. In some embodiments, the a nucleic acid is labeled with a detectable moiety, having about 5, about 10, about 15, about 20, about 25, about 30, about 35, or about 40 nucleotides upstream and/or downstream of the polymorphic region. In a further aspect, the genotype is determined by a method comprising, or alternatively consisting essentially of, or yet further consisting of whole genome sequencing.

In some embodiments, screening a biological sample isolated from the patient for an rs760370 polymorphism comprises, or consists essentially of, or yet further consists of amplifying nucleic acid containing the rs760370 polymorphism. In some embodiments, nucleic acid containing the rs760370 polymorphism is amplified using a forward primer comprising, or alternatively consisting essentially of, or yet further consisting of nucleic acid having the sequence of SEQ ID NO:12 and a reverse primer comprising, or alternatively consisting essentially of, or yet further consisting of nucleic acid having the sequence of SEQ ID NO:13.

In some embodiments, provided are methods for selecting a cancer, patient, e.g., a GI cancer patient, a colon cancer patient, a rectal cancer patient, or a colorectal cancer patient for a therapy comprising, or alternatively consisting essentially of, or yet further consists of, administration of an effective amount of TAS-102, the method comprising, or alternatively consisting essentially of, or yet further consists of, screening a biological sample isolated from the patient for an rs9394992 polymorphism, and selecting the patient for the therapy if the genotype of (C/T) or (T/T) for rs9394992 is present in the sample. In some embodiments, the patient is not selected for a therapy comprising, or alternatively consisting essentially of, or yet further consists of, administration of an effective amount of TAS-102 if the genotype of (C/T) or (T/T) for rs9394992 is not present in the sample. In some embodiments, the patient is not selected for a therapy comprising, or alternatively consisting essentially of, or yet further consists of, administration of an effective amount TAS-102 if the genotype of (G/G) for rs9394992 is present in the sample. In some embodiments, the patient is selected for a TAS-102-free therapy if the genotype of (C/T) or (T/T) for rs9394992 is not present in the sample. In some embodiments, the patient is selected for a TAS-102-free therapy if the genotype of (G/G) for rs9394992 is present in the sample. The therapy can be first line, second line, third line, fourth line of fifth line therapy and is some aspects, can be administered alone or in combination with other therapies, e.g., surgical resection, radiation therapy or other chemical or biological based therapies. Also provided, in some embodiments, are methods for classifying a colorectal cancer patient as eligible for a therapy, the therapy comprising, or alternatively consisting essentially of, or yet further consists of, administration of an effective amount of TAS-102, comprising, or alternatively consisting essentially of, or yet further consisting of screening a biological sample isolated from the patient for an rs9394992 polymorphism, and classifying the patient as eligible for the therapy if the genotype of (C/T) or (T/T) for rs9394992 is present in the sample. In some embodiments, the method comprises, or consists essentially of, or yet further consists of classifying the patient as not eligible for the therapy comprising, or alternatively consisting essentially of, or yet further consisting of TAS-102 if the genotype of (C/T) or (T/T) for rs9394992 is not present in the sample. In some embodiments, the patient is classified as not eligible for the therapy comprising, or alternatively consisting essentially of, or yet further consisting of administration an effective amount of TAS-102 if the genotype of (G/G) for rs9394992 is present in the sample. In some embodiments, the method further comprises, or consists essentially of, or consists of, administering a therapy comprising, or alternatively consisting essentially of, or yet further consisting of administration of an effective amount of TAS-102. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, are methods for increasing the progression-free and/or overall survival of a cancer, patient, e.g., a GI cancer patient, a colon cancer patient, a rectal cancer patient, or a colorectal cancer patient, the method comprising, or alternatively consisting essentially of, or yet further consists of, screening a biological sample isolated from the patient for an rs9394992 polymorphism, and classifying the patient as eligible for the therapy comprising, or alternatively consisting essentially of, or yet further consisting of administration of an effective amount of TAS-102 if the genotype of (C/T) or (T/T) for rs9394992 is present in the sample or not eligible for the therapy comprising, or alternatively consisting essentially of, or yet further consisting of, or alternatively consisting essentially of, or yet further consists of, TAS-102 if the genotype of (C/T) or (T/T) for rs9394992 is not present in the sample. In some embodiments, the patient is classified as not eligible for the therapy comprising, or alternatively consisting essentially of, or yet further consists of, administration of an effective amount of TAS-102 if the genotype of (G/G) for rs9394992 is present in the sample. In some embodiments, the method further comprises, or consists essentially of, or yet further consists of, administering a therapy comprising, or alternatively consisting essentially of, or yet further consists of, an effective amount of TAS-102 or a TAS-102-free therapy in accordance with the classification. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, are methods for identifying whether a cancer, patient, e.g., a GI cancer patient, a colon cancer patient, a rectal cancer patient, or a colorectal cancer patient is likely to experience a relatively longer or shorter progression free survival (PFS) following a therapy, the therapy comprising, or alternatively consisting essentially of, or yet further consists of, administration of an effective amount of TAS-102, the method comprising, or alternatively consisting essentially of, or yet further consists of, screening a biological sample isolated from the patient for an rs9394992 polymorphism, and identifying that the patient is likely to experience a longer progression free survival if the genotype of (C/T) or (T/T) for rs9394992 is present in the sample, relative to a corresponding cancer patient not having the genotype. In some embodiments, the method comprises, or consists essentially of, or yet further consists of identifying that the patient is likely to experience a shorter progression free survival if the genotype of (C/T) or (T/T) for rs9394992 is not present in the sample, relative to a corresponding cancer patient having the genotype or relative to the cancer patient having the genotype of (G/G) for rs9394992. In some embodiments, the method comprises, or consists essentially of, or yet further consists of identifying that the patient is likely to experience a shorter progression free survival if the genotype of (G/G) for rs9394992 is present in the sample, relative to the corresponding cancer patient not having the genotype or relative to the cancer patient having the genotype of (C/T) or (T/T) for rs9394992. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, are methods for treating a cancer, patient, e.g., a GI cancer patient, a colon cancer patient, a rectal cancer patient, or a colorectal cancer patient selected for treatment based on the presence of the genotype of (C/T) or (T/T) for rs9394992 in a biological sample isolated from the patient, comprising, or alternatively consisting essentially of, or yet further consists of, administering to the patient a therapy comprising, or alternatively consisting essentially of, or yet further consisting of administration of an effective amount of TAS-102. Also provided, in some embodiments, are methods for treating the cancer patient selected for treatment based on the absence of the genotype of (G/G) for rs9394992 in a biological sample from the patient, comprising, or alternatively consisting essentially of, or yet further consists of, administering to the patient a therapy comprising, or alternatively consisting essentially of, or yet further consisting of administration of an effective amount of TAS-102. The therapy can be first line, second line, third line, fourth line of fifth line therapy and is some aspects, can be administered alone or in combination with other therapies, e.g., surgical resection, radiation therapy or other chemical or biological based therapies.

In some embodiments, the method further comprises, or consists essentially of, or consists of, screening a biological sample isolated from the patient for the rs9394992 polymorphism. Thus, also provided, in some embodiments, are methods for treating a cancer, patient, e.g., a GI cancer patient, a colon cancer patient, a rectal cancer patient, or a colorectal cancer patient, the method comprising, or alternatively consisting essentially of, or yet further consists of, screening a biological sample isolated from the patient for the rs9394992 polymorphism and administering to the patient a therapy comprising, or alternatively consisting essentially of, or yet further consists of, an effective amount of TAS-102 if the sample has the genotype of (C/T) or (T/T) for rs9394992.

Also provided, in some embodiments, are methods for modifying the treatment of patient receiving a therapy comprising, or alternatively consisting essentially of, or yet further consists of, administration of an effective amount of TAS-102 based on the presence of the genotype of (C/T) or (T/T) for rs9394992 in a biological sample from the patient. For example, provided are methods for modifying the treatment of patient receiving a therapy comprising, or alternatively consisting essentially of, or yet further consists of, administration of an effective amount of TAS-102, the method comprising, or alternatively consisting essentially of, or yet further consists of, screening a biological sample isolated from the patient for an rs9394992 polymorphism, and modifying the dosage or frequency of the therapy comprising, or alternatively consisting essentially of, or yet further consisting of an effective amount of TAS-102 based on the genotype for rs9394992. In some embodiments, the dosage or frequency of the therapy, or components thereof (e.g., one or more therapeutic agents of the therapy), is increased if the genotype of (C/T) or (T/T) for rs9394992 is not present in the sample. In some embodiments, the dosage or frequency of the therapy, or components thereof, is increased if the genotype of (G/G) for rs9394992 is present in the sample. In some embodiments, the therapy is discontinued if the genotype of (C/T) or (T/T) for rs9394992 is not present in the sample. In some embodiments, the therapy is discontinued if the genotype of (G/G) for rs9394992 is present in the sample. In some embodiments, the therapy is continued if the genotype of (C/T) or (T/T) for rs9394992 is present in the sample. The therapy can be first line, second line, third line, fourth line of fifth line therapy and is some aspects, can be administered alone or in combination with other therapies, e.g., surgical resection, radiation therapy or other chemical or biological based therapies.

In some embodiments, screening a biological sample isolated from the patient for an rs9394992 polymorphism comprises, or consists essentially of, or yet further consists of contacting the biological sample with a nucleic acid probe that specifically binds to nucleic acid containing the rs9394992 polymorphism and overlaps the polymorphic site. For example, in some embodiments, the nucleic acid specifically binds to a nucleic acid having the sequence of SEQ ID NO:4, SEQ ID NO:35, or SEQ ID NO:36 and overlaps the polymorphic site. In some embodiments, the a nucleic acid is labeled with a detectable moiety, having about 5, about 10, about 15, about 20, about 25, about 30, about 35, or about 40 nucleotides upstream and/or downstream of the polymorphic region. In another aspect, the sample is screened by a method comprising, or alternatively consisting essentially of, or yet further consisting of whole genome sequencing of the region comprising, or alternatively consisting essentially of, or yet further consisting of the polymorphism.

In some embodiments, screening a biological sample isolated from the patient for an rs9394992 polymorphism comprises, or consists essentially of, or yet further consists of amplifying nucleic acid containing the rs9394992 polymorphism. In some embodiments, nucleic acid containing the rs9394992 polymorphism is amplified using a forward primer comprising, or alternatively consisting essentially of, or yet further consisting of nucleic acid having the sequence of SEQ ID NO:14 and a reverse primer comprising, or alternatively consisting essentially of, or yet further consisting of nucleic acid having the sequence of SEQ ID NO:15.

Also provided are methods for provided for screening a biological sample isolated from a patient for an OCT2 rs316000 or OCT2 rs316019 polymorphism and a MATE1 rs2289669 polymorphism. The OCT2 rs316000 polymorphism is located at chromosome position 99991562 on chromosome 6 according to the Genome Reference Consortium Human Build 38 patch release 2 (GRCh38.p2, NCBI). The following nucleotide sequence represents a region of human DNA comprising, or alternatively consisting essentially of, or yet further consisting of the rs316000 polymorphism: tcaagaacttttcctgtgcattctc[A/G]acttggctacctggtacaagagtcc (SEQ ID NO:5). Thus, the partial sequence of the (A) allele of the rs3166000 polymorphism is: tcaagaacttttccattctcAacttggctacctggtacaagagtcc (SEQ ID NO:37) and the partial sequence of the (G) allele is: tcaagaacttttcc (SEQ ID NO:38).

Provided are methods for provided are methods for selecting a cancer, patient, e.g., a GI cancer patient, a colon cancer patient, a rectal cancer patient, or a colorectal cancer patient for a therapy, the therapy comprising, or alternatively consisting essentially of, or yet further consisting of, or alternatively consisting essentially of, or yet further consists of, administration of an effective amount of TAS-102, the method comprising, or alternatively consisting essentially of, or yet further consists of, screening a biological sample isolated from the patient for an OCT2 rs316000 or OCT2 rs316019 polymorphism and a MATE1 rs2289669 polymorphism. Also provided, in some embodiments, are methods for classifying a cancer, patient, e.g., a GI cancer patient, a colon cancer patient, a rectal cancer patient, or a colorectal cancer patient as eligible for a therapy comprising, or alternatively consisting essentially of, or yet further consists of, administration of an effective amount of TAS-102, the method comprising, or alternatively consisting essentially of, or yet further consists of, screening a biological sample isolated from the patient for an OCT2 rs316000 or OCT2 rs316019 polymorphism and a MATE1 rs2289669 polymorphism. Also provided, in some embodiments, are methods for increasing the progression-free and/or overall survival of a cancer, patient, e.g., a GI cancer patient, a colon cancer patient, a rectal cancer patient, or a colorectal cancer patient, the method comprising, or alternatively consisting essentially of, or yet further consists of, screening a biological sample isolated from the patient for an OCT2 rs316000 or OCT2 rs316019 polymorphism and a MATE1 rs2289669 polymorphism. Also provided, in some embodiments, are methods for identifying whether a cancer, patient, e.g., a GI cancer patient, a colon cancer patient, a rectal cancer patient, or a colorectal cancer patient is likely to experience a relatively longer or shorter progression free survival (PFS) following a therapy comprising, or alternatively consisting essentially of, or yet further consists of, administration of an effective amount of TAS-102, the method comprising, or alternatively consisting essentially of, or yet further consists of, screening a biological sample isolated from the patient for an OCT2 rs316000 or OCT2 rs316019 polymorphism and a MATE1 rs2289669 polymorphism. Also provided, in some embodiments, are methods for treating a cancer, patient, e.g., a GI cancer patient, a colon cancer patient, a rectal cancer patient, or a colorectal cancer patient selected for treatment based on the presence of the genotype of an OCT2 rs316000 or OCT2 rs316019 polymorphism and a MATE1 rs2289669 polymorphism. Also provided, in some embodiments, are methods for modifying the treatment of patient receiving a therapy comprising, or alternatively consisting essentially of, or yet further consists of, administration of an effective amount of TAS-102 based on the presence of the genotype of an OCT2 rs316000 or OCT2 rs316019 polymorphism and a MATE1 rs2289669 polymorphism. In some embodiments, the sample is also screened an hENT1 rs760370 or an hENT1 rs9394992 polymorphism. The therapy can be first line, second line, third line, fourth line of fifth line therapy and is some aspects, can be administered alone or in combination with other therapies, e.g., surgical resection, radiation therapy or other chemical or biological based therapies.

In some aspects, the patient has a wild-type KRAS and/or BRAF gene.

In some aspects, the patient for the methods described herein suffers colon cancer, non-metastatic colorectal cancer or metastatic colorectal cancer.

In some aspects, the biological sample is a tissue or a cell sample. In some aspects, the sample comprises, or consists essentially of, or yet further consists of at least one of a tumor cell, a normal cell adjacent to a tumor, a normal cell corresponding to the tumor tissue type, a blood cell, a peripheral blood lymphocyte, or combinations thereof.

In some aspects, the sample is at least one of blood, plasma, serum, an original sample recently isolated from the patient, a fixed tissue, a previously frozen tissue, a biopsy tissue, a resection tissue, a microdissected tissue, or combinations thereof.

In some aspects, the screening the polymorphism is by a method comprising, or alternatively consisting essentially of, or yet further consisting of PCR, RT-PCR, real-time PCR, PCR-RFLP, sequencing, or whole genome sequencing, a nucleic acid probe hybridization in solution or on a solid support, such as a chip or a microarray. In some aspects, the patient is a mammal, such as a human patient.

Also provided, in some embodiments, are kits for screening for selecting a cancer, patient, e.g., a GI cancer patient, a colon cancer patient, a rectal cancer patient, or a colorectal cancer patient for a therapy comprising, or alternatively consisting essentially of, or yet further consists of, TAS-102 or for classifying the cancer patient as eligible for a therapy comprising, or alternatively consisting essentially of, or yet further consisting of TAS-102. In some embodiments, the kit comprises, or consists essentially of, or yet further consists of primer for amplification of nucleic acid containing a polymorphism selected from among a rs609429, rs861539, rs760370, rs9394992, rs2289669, and rs316019 polymorphism. For example, in some embodiments, the kit comprises, or consists essentially of, or yet further consists of a forward primer having the sequence of SEQ ID NO:8 and a reverse primer having the sequence of SEQ ID NO:9, a forward primer having the sequence of SEQ ID NO:10 and a reverse primer having the sequence of SEQ ID NO:11, a forward primer having the sequence of SEQ ID NO:49 and a reverse primer having the sequence of SEQ ID NO: 11, a forward primer having the sequence of SEQ ID NO:12 and a reverse primer having the sequence of SEQ ID NO:13, a forward primer having the sequence of SEQ ID NO:14 and a reverse primer having the sequence of SEQ ID NO:15, a forward primer having the sequence of SEQ ID NO:16 and a reverse primer having the sequence of SEQ ID NO:17, a forward primer having the sequence of SEQ ID NO:18 and a reverse primer having the sequence of SEQ ID NO:19, or a forward primer having the sequence of SEQ ID NO:20 and a reverse primer having the sequence of SEQ ID NO:21. In some embodiments, the kit comprises, or consists essentially of, or yet further consists of a nucleic acid probe that specifically binds to nucleic acid containing the polymorphism and overlaps the polymorphic site. For example, in some embodiments, the nucleic acid probe specifically binds to a nucleic acid having the sequence of any of SEQ ID NO:1-7 or SEQ ID NO:29-42 and overlaps the polymorphic site. In some embodiments, the nucleic acid probe has about 5, about 10, about 15, about 20, about 25, about 30, about 35 or about 40 or more contiguous nucleotides of any of SEQ ID NO:1-7 or SEQ ID NO:29-42 and overlaps the polymorphic site.

The disclosure further provides diagnostic, prognostic and therapeutic methods, which are based, at least in part, on determination of the identify of a genotype of interest identified herein.

For example, information obtained using the diagnostic assays described herein is useful for determining if a subject is suitable for cancer treatment of a given type. Based on the prognostic information, a doctor can recommend a therapeutic protocol, useful for reducing the malignant mass or tumor in the patient or treat cancer in the individual.

A patient's likely clinical outcome following a clinical procedure such as a therapy or surgery can be expressed in relative terms. For example, a patient having a particular genotype or expression level can experience relatively longer overall survival than a patient or patients not having the genotype or expression level. The patient having the particular genotype or expression level, alternatively, can be considered as likely to survive. Similarly, a patient having a particular genotype or expression level can experience relatively longer progression free survival, or time to tumor progression, than a patient or patients not having the genotype or expression level. The patient having the particular genotype or expression level, alternatively, can be considered as not likely to suffer tumor progression. Further, a patient having a particular genotype or expression level can experience relatively shorter time to tumor recurrence than a patient or patients not having the genotype or expression level. The patient having the particular genotype or expression level, alternatively, can be considered as not likely to suffer tumor recurrence. Yet in another example, a patient having a particular genotype or expression level can experience relatively more complete response or partial response than a patient or patients not having the genotype or expression level. The patient having the particular genotype or expression level, alternatively, can be considered as likely to respond. Accordingly, a patient that is likely to survive, or not likely to suffer tumor progression, or not likely to suffer tumor recurrence, or likely to respond following a clinical procedure is considered suitable for the clinical procedure.

It is to be understood that information obtained using the diagnostic assays described herein can be used alone or in combination with other information, such as, but not limited to, genotypes or expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis or treatment.

The methods are useful in the assistance of an animal, a mammal or yet further a human patient. For the purpose of illustration only, a mammal includes but is not limited to a human, a simian, a murine, a bovine, an equine, a porcine or an ovine subject.

In some aspects, the patient suffers from non-metastatic colorectal cancer or metastatic colorectal cancer. In some aspects, the colorectal cancer is metastatic or non-metastatic colon cancer. In some aspects, the colorectal cancer is metastatic or non-metastatic rectal cancer.

Any suitable method for identifying the genotype in the patient sample can be used and the disclosures described herein are not to be limited to these methods. For the purpose of illustration only, the genotype is determined by a method comprising, or alternatively consisting essentially of, or yet further consisting of, sequencing, hybridization, nucleic acid amplification, including polymerase chain reaction (PCR), real-time PCR, reverse transcriptase PCR (RT-PCR), nested PCR, ligase chain reaction, or PCR-RFLP, or microarray. These methods as well as equivalents or alternatives thereto are described herein.

The methods are useful in the assistance of an animal, a mammal or yet further a human patient. For the purpose of illustration only, a mammal includes but is not limited to a human, a simian, a murine, a bovine, an equine, a porcine or an ovine subject.

Information obtained using the diagnostic assays described herein is useful for determining if a subject will likely, more likely, or less likely to respond to cancer treatment of a given type. Based on the prognostic information, a doctor can recommend a therapeutic protocol, useful for treating reducing the malignant mass or tumor in the patient or treat cancer in the individual.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. The identity of the genotype or expression patterns of individual patients can then be compared to the genotype or expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

### Biological Sample Collection and Preparation

The methods and compositions disclosed herein can be used to detect nucleic acids associated with the genetic polymorphisms identified herein and in particular those noted in Table A using a biological sample obtained from a patient. Biological samples can be obtained by standard procedures and can be used immediately or stored, under conditions appropriate for the type of biological sample, for later use. Any liquid or solid biological material obtained from the patient believed to contain nucleic acids comprising the region the polymorphic region can be an suitable sample.

Methods of obtaining test samples are known to those of skill in the art and include, but are not limited to, aspirations, tissue sections, swabs, drawing of blood or other fluids, surgical or needle biopsies.

In some aspects, the biological sample is a tissue or a cell sample. Suitable patient samples in the methods include, but are not limited to, blood, plasma, serum, a biopsy tissue, fine needle biopsy sample, amniotic fluid, plasma, pleural fluid, saliva, semen, serum, tissue or tissue homogenates, frozen or paraffin sections of tissue or combinations thereof. In some aspects, the biological sample comprises, or alternatively consisting essentially of, or yet further consisting of, at least one of a tumor cell, a normal cell adjacent to a tumor, a normal cell corresponding to the tumor tissue type, a blood cell, a peripheral blood lymphocyte, or combinations thereof. In some aspects, the biological sample is an original sample recently isolated from the patient, a fixed tissue, a frozen tissue, a resection tissue, or a microdissected tissue. In some aspects, the biological samples are processed, such as by sectioning of tissues, fractionation, purification, nucleic acid isolation, or cellular organelle separation.

In some embodiments, nucleic acid (DNA or RNA) is isolated from the sample according to any methods known to those of skill in the art. In some aspects, genomic DNA is isolated from the biological sample. In some aspects, RNA is isolated from the biological sample. In some aspects, cDNA is generated from mRNA in the sample. In some embodiments, the nucleic acid is not isolated from the biological sample (e.g., the polymorphism is detected directly from the biological sample).

### Detection of Polymorphisms

In some aspects, detection of polymorphisms can be accomplished by molecular cloning of the specified allele and subsequent sequencing of that allele using techniques known in the art, in some aspects, after isolation of a suitable nucleic acid sample. In some aspects, the gene sequences can be amplified directly from a genomic DNA preparation from the biological sample using PCR, and the sequence composition is determined by sequencing the amplified product (i.e., amplicon). Alternatively, the PCR product can be analyzed following digestion with a restriction enzyme, a method known as PCR-RFLP.

In some embodiments, the polymorphism is detected using allele specific hybridization using probes overlapping the polymorphic site. In some aspects, the nucleic acid probes are between 5 and 40 nucleotides in length. In some aspects, the nucleic acid probes are about 5, about 10, about 15, about 20, about 25, about 30, about 35, or about 40 or more nucleotides flanking the polymorphic site.

In another embodiment of the disclosure, several nucleic acid probes capable of hybridizing specifically to the nucleic acid containing the allelic variant are attached to a solid phase support, e.g., a “chip” or “microarray. Such gene chips or microarrays can be used to detect genetic variations by a number of techniques known to one of skill in the art. In one technique, oligonucleotides are arrayed on a gene chip for determining the DNA sequence by the sequencing by hybridization approach. The probes of the disclosure also can be used for fluorescent detection of a genetic sequence. A probe also can be affixed to an electrode surface for the electrochemical detection of nucleic acid sequences.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers can be detectably labeled thereby identifying the polymorphism in the gene(s) of interest. Alternatively, a chemical or biological reaction can be used to identify the probes or primers which hybridized with the DNA or RNA of the gene(s) of interest. The genetic profile of the patient is then determined with the aid of the aforementioned apparatus and methods.

In some aspects, whole genome sequencing, in particular with the “next generation sequencing” techniques, which employ massively parallel sequencing of DNA templates, can be used to obtain genotypes of relevant polymorphisms. Exemplary NGS sequencing platforms for the generation of nucleic acid sequence data include, but are not limited to, Illumina's sequencing by synthesis technology (e.g., Illumina MiSeq or HiSeq System), Life Technologies' Ion Torrent semiconductor sequencing technology (e.g., Ion Torrent PGM or Proton system), the Roche (454 Life Sciences) GS series and Qiagen (Intelligent BioSystems) Gene Reader sequencing platforms.

In some aspects, nucleic acid comprising, or alternatively consisting essentially of, or yet further consisting of the polymorphism is amplified to produce an amplicon containing the polymorphism. Nucleic acids can be amplified by various methods known to the skilled artisan. Nucleic acid amplification can be linear or exponential. Amplification is generally carried out using polymerase chain reaction (PCR) technologies. Alternative or modified PCR amplification methods can also be used and include, for example, isothermal amplification methods, rolling circle methods, Hot-start PCR, real-time PCR, Allele-specific PCR, Assembly PCR or Polymerase Cycling Assembly (PCA), Asymmetric PCR, Colony PCR, Emulsion PCR, Fast PCR, Real-Time PCR, nucleic acid ligation, Gap Ligation Chain Reaction (Gap LCR), Ligation-mediated PCR, Multiplex Ligation-dependent Probe Amplification, (MLPA), Gap Extension Ligation PCR (GEXL-PCR), quantitative PCR (Q-PCR), Quantitative real-time PCR (QRT-PCR), multiplex PCR, Helicase-dependent amplification, Intersequence-specific (ISSR) PCR, Inverse PCR, Linear-After-The-Exponential-PCR (LATE-PCR), Methylation-specific PCR (MSP), Nested PCR, Overlap-extension PCR, PAN-AC assay, Reverse Transcription PCR(RT-PCR), Rapid Amplification of cDNA Ends (RACE PCR), Single molecule amplification PCR(SMA PCR), Thermal asymmetric interlaced PCR (TAIL-PCR), Touchdown PCR, long PCR, nucleic acid sequencing (including DNA sequencing and RNA sequencing), transcription, reverse transcription, duplication, DNA or RNA ligation, and other nucleic acid extension reactions known in the art. The skilled artisan will understand that other methods can be used either in place of, or together with, PCR methods, including enzymatic replication reactions developed in the future. See, e.g., Saiki, “Amplification of Genomic DNA” in PCR Protocols, Innis et al., eds., Academic Press, San Diego, Calif., 13-20 (1990); Wharam, et al., 29(11) Nucleic Acids Res, E54-E54 (2001); Hafner, et al., 30(4) Biotechniques, 852-6, 858, 860 passim (2001).

In some aspects, nucleic acid comprising, or alternatively consisting essentially of, or yet further consisting of the polymorphism of interest is amplified to produce an amplicon. In some aspects, a nucleic acid containing the region of interest is amplified using a forward primer and a reverse primer the flank the region of interest. In some aspects, the amplicon containing the region of interest (e.g. an amplicon having the polymorphic sequence) is detected by hybridizing a nucleic acid probe containing the polymorphism or a complement thereof to the corresponding complementary strand of the amplicon and detecting the hybrid formed between the nucleic acid probe and the complementary strand of the amplicon. In some aspects, amplicon containing the region of interest is sequenced (e.g., dideoxy chain termination methods (Sanger method and variants thereof), Maxam & Gilbert sequencing, pyrosequencing, exonuclease digestion and next-generation sequencing methods).

In some embodiments, the amplification includes a labeled primer or probe, thereby allowing detection of the amplification products corresponding to that primer or probe. In particular embodiments, the amplification can include a multiplicity of labeled primers or probes; such primers can be distinguishably labeled, allowing the simultaneous detection of multiple amplification products.

In some embodiments, the amplification products are detected by any of a number of methods such as gel electrophoresis, column chromatography, hybridization with a nucleic acid probe, or sequencing the amplicon.

Detectable labels can be used to identify the primer or probe hybridized to a genomic nucleic acid or amplicon. Detectable labels include but are not limited to fluorophores, isotopes (e.g., ³²P, ³³P, ³⁵S, ³H, ¹⁴C, ¹²⁵I, ¹³¹I) electron-dense reagents (e.g., gold, silver), nano articles enzymes commonly used in an ELISA (e.g., horseradish peroxidase, beta-galactosidase, luciferase, alkaline phosphatase), chemiluminiscent compounds, colorimetric labels (e.g., colloidal gold), magnetic labels (e.g., Dynabeads®), biotin, digoxigenin, haptens, proteins for which antisera or monoclonal antibodies are available, ligands, hormones, oligonucleotides capable of forming a complex with the corresponding oligonucleotide complement.

In one embodiment, a primer or probe is labeled with a fluorophore that emits a detectable signal. The term “fluorophore” as used herein refers to a molecule that absorbs light at a particular wavelength (excitation frequency) and subsequently emits light of a longer wavelength (emission frequency). While a suitable reporter dye is a fluorescent dye, any reporter dye that can be attached to a detection reagent such as an oligonucleotide probe or primer is suitable for use in the methods described. Suitable fluorescent moieties include, but are not limited to, the following fluorophores working individually or in combination: 4-acetamido-4′-isothiocyanatostilbene-2,2′disulfonic acid; acridine and derivatives, e,g, acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET); fluorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; and tetramethyl rhodamine isothiocyanate (TRITC).

In some aspects, the primer or probe is further labeled with a quencher dye such as Tamra, Dabcyl, or Black Hole Quencher® (BHQ), especially when the reagent is used as a self-quenching probe such as a TaqMan® (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635).

In some aspects, methods for real time PCR use fluorescent primers/probes, such as the TaqMan® primers/probes (Heid, et al., Genome Res 6: 986-994, 1996), molecular beacons, and Scorpion™ primers/probes. Real-time PCR quantifies the initial amount of the template with more specificity, sensitivity and reproducibility, than other forms of quantitative PCR, which detect the amount of final amplified product. Real-time PCR does not detect the size of the amplicon. The probes employed in Scorpion®™ and TaqMan® technologies are based on the principle of fluorescence quenching and involve a donor fluorophore and a quenching moiety. The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety. The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Forster or fluorescence resonance energy transfer (“FRET”). Quenching by FRET is generally used in TaqMan® primers/probes while proximal quenching is used in molecular beacon and Scorpion™ type primers/probes.

The detectable label can be incorporated into, associated with or conjugated to a nucleic acid primer or probe. Labels can be attached by spacer arms of various lengths to reduce potential steric hindrance or impact on other useful or desired properties. See, e.g., Mansfield, Mol. Cell. Probes (1995), 9:145-156.

Detectable labels can be incorporated into nucleic acid probes by covalent or non-covalent means, e.g., by transcription, such as by random-primer labeling using Klenow polymerase, or nick translation, or, amplification, or equivalent as is known in the art. For example, a nucleotide base is conjugated to a detectable moiety, such as a fluorescent dye, e.g., Cy3™ or Cy5™ and then incorporated into nucleic acid probes during nucleic acid synthesis or amplification. Nucleic acid probes can thereby be labeled when synthesized using Cy3™- or Cy5™-dCTP conjugates mixed with unlabeled dCTP.

Nucleic acid probes can be labeled by using PCR or nick translation in the presence of labeled precursor nucleotides, for example, modified nucleotides synthesized by coupling allylamine-dUTP to the succinimidyl-ester derivatives of the fluorescent dyes or haptens (such as biotin or digoxigenin) can be used; this method allows custom preparation of most common fluorescent nucleotides, see, e.g., Henegariu et al., Nat. Biotechnol. (2000), 18:345-348,

Nucleic acid probes can be labeled by non-covalent means known in the art. For example, Kreatech Biotechnology's Universal Linkage System® (ULS®) provides a non-enzymatic labeling technology, wherein a platinum group forms a co-ordinative bond with DNA, RNA or nucleotides by binding to the N7 position of guanosine. This technology can also be used to label proteins by binding to nitrogen and sulfur containing side chains of amino acids. See, e.g., U.S. Pat. Nos. 5,580,990; 5,714,327; and 5,985,566; and European Patent No. 0539466.

Labeling with a detectable label also can include a nucleic acid attached to another biological molecule, such as a nucleic acid, e.g., an oligonucleotide, or a nucleic acid in the form of a stem-loop structure as a “molecular beacon” or an “aptamer beacon”. Molecular beacons as detectable moieties are described; for example, Sokol (Proc. Natl. Acad. Sci. USA (1998), 95:11538-11543) synthesized “molecular beacon” reporter oligodeoxynucleotides with matched fluorescent donor and acceptor chromophores on their 5′ and 3′ ends. In the absence of a complementary nucleic acid strand, the molecular beacon remains in a stem-loop conformation where fluorescence resonance energy transfer prevents signal emission. On hybridization with a complementary sequence, the stem-loop structure opens increasing the physical distance between the donor and acceptor moieties thereby reducing fluorescence resonance energy transfer and allowing a detectable signal to be emitted when the beacon is excited by light of the appropriate wavelength. See also, e.g., Antony (Biochemistry (2001), 40:9387-9395), describing a molecular beacon consist of a G-rich 18-mer triplex forming oligodeoxyribonucleotide. See also U.S. Pat. Nos. 6,277,581 and 6,235,504.

Aptamer beacons are similar to molecular beacons; see, e.g., Hamaguchi, Anal. Biochem. (2001), 294:126-131; Poddar, Mol. Cell. Probes (2001), 15:161-167; Kaboev, Nucleic Acids Res. (2000), 28:E94. Aptamer beacons can adopt two or more conformations, one of which allows ligand binding. A fluorescence-quenching pair is used to report changes in conformation induced by ligand binding. See also, e.g., Yamamoto et al., Genes Cells (2000), 5:389-396; Smimov et al., Biochemistry (2000), 39:1462-1468.

The nucleic acid primer or probe can be indirectly detectably labeled via a peptide. A peptide can be made detectable by incorporating predetermined polypeptide epitopes recognized by a secondary reporter (e.g., leucine zipper pair sequences, binding sites for secondary antibodies, transcriptional activator polypeptide, metal binding domains, epitope tags). A label can also be attached via a second peptide that interacts with the first peptide (e.g., S—S association).

As readily recognized by one of skill in the art, detection of the complex containing the nucleic acid from a sample hybridized to a labeled probe can be achieved through use of a labeled antibody against the label of the probe. In one example, the probe is labeled with digoxigenin and is detected with a fluorescent labeled anti-digoxigenin antibody. In another example, the probe is labeled with FITC, and detected with fluorescent labeled anti-FITC antibody. These antibodies are readily available commercially. In another example, the probe is labeled with FITC, and detected with anti-FITC antibody primary antibody and a labeled anti-anti FITC secondary antibody.

Nucleic acids can be amplified prior to detection or can be detected directly during an amplification step (i.e., “real-time” methods, such as in TaqMan® and Scorpion™ methods). In some embodiments, the target sequence is amplified using a labeled primer such that the resulting amplicon is detectably labeled. In some embodiments, the primer is fluorescently labeled. In some embodiments, the target sequence is amplified and the resulting amplicon is detected by electrophoresis.

With regard to the exemplary primers and probes, those skilled in the art will readily recognize that nucleic acid molecules can be double-stranded molecules and that reference to a particular site on one strand refers, as well, to the corresponding site on a complementary strand. In defining a variant position, allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference can be made to either strand in order to refer to a particular variant position, allele, or nucleotide sequence. Probes and primers, can be designed to hybridize to either strand and detection methods disclosed herein can generally target either strand.

In some embodiments, the primers and probes comprise additional nucleotides corresponding to sequences of universal primers (e.g., T7, M13, SP6, T3) which add the additional sequence to the amplicon during amplification to permit further amplification and/or prime the amplicon for sequencing.

### Methods of Treatment

The disclosure further provides methods of treating a patient selected by any method of the above embodiments, or identified as likely to experience a more favorable clinical outcome by any of the above methods, following the therapy. In some embodiments, the methods entail administering to the patients such a therapy. The therapy can be any one of the group of: a first line, second line, third line, a fourth line, or a fifth line therapy.

In some embodiments, provided are methods for treating a colorectal cancer patient selected for treatment based on the presence of a favorable genotype identified in Table A or as disclosed herein, in a biological sample from the patient, comprising, or alternatively consisting essentially of, or yet further consisting of administering to the patient a therapy comprising, or alternatively consisting essentially of, or yet further consisting of a therapeutically effective amount of TAS102 or an equivalent thereof.

In some aspects, the patient is selected by a method comprising, or alternatively consisting essentially of, or yet further consisting of screening a tissue or cell sample isolated from the patient for a polymorphism identified in Table A or as disclosed herein. Exemplary methods for screening are described in the diagnostic methods provided above and throughout the present disclosure. Any such diagnostic methods disclosed for the detection of a genetic polymorphism can be combined with the treatment methods provided herein.

In some aspects, therapy comprising, or alternatively consisting essentially of, or yet further consisting of additional anticancer agents, non-limiting example of such include irinotecan and bevacizumab and may further comprises, or consists essentially of, or yet further consists of a therapeutically effective amount of folinic acid and/or a pyrimidine analog. In some aspects, the therapy additionally comprises, or consists essentially of, or yet further consists of FOLFIRI (leucovorin+Fluorouracil (5-FU)+irinotecan). In some aspects, the therapy further comprises, or consists essentially of, or yet further consists of a therapeutically effective amount of oxaliplatin. In some aspects, the therapy further comprises, or consists essentially of, or yet further consists of FOLFOXFIRI (leucovorin+Fluorouracil (5-FU)+oxaliplatin+irinotecan). Oxaliplatin include but are not limited to Oxaliplatin 75-85 mg/m² IV+leucovorin 200 mg/m² IV infused over 2 hr, then 5-FU 300-400 mg/m² IV bolus over 2-4 minutes, then 5-FU 500-600 mg/m² IV infusion in D5W (500 mL) over 22 hr on day 1 and then repeat on day 2 without oxaliplatin, and then repeat the 2-day regimen every 2 weeks. In some aspect, treatment is provided following tumor resection.

In some aspects, the patient suffers from non-metastatic colorectal cancer or metastatic colorectal cancer. In some aspects, the colorectal cancer is colon cancer. In some aspects, the colorectal cancer is rectal cancer.

Exemplary dosing schedules and routes of administration for the treatment of colorectal cancer with TAS-102 and other therapies are known in the art. Exemplary dosing schedules for the treatment of colorectal cancer with irinotecan as a monotherapy include but are not limited to 125 mg/m² IV infusion over 90 minutes on days 1, 8, 15, 22, then 2 weeks off, then repeat or 350 mg/m² IV infusion over 30-90 minutes once every 3 weeks. Exemplary dosing schedules for the treatment of colorectal cancer with irinotecan as combination therapy include but are not limited to 180 mg/m² IV infusion over 30-90 minutes once on days 1, 15, and 29 IV (infuse over 30-90 min), followed by infusion with leucovorin and 5-fluorouracil; next cycle begins on day 43 (6 week cycle) or 125 mg/m² on days 1, 8, 15, and 22 (infuse over 90 min), followed by bolus doses of leucovorin and 5-fluorouracil.

Also provided is a therapy or a medicament comprising, or alternatively consisting essentially of, or yet further consisting of an effective amount of a chemotherapeutic as described herein for treatment of a human cancer patient having the appropriate expression level of the gene of interest as identified in the experimental examples. Further provided is a therapy comprising, or alternatively consisting essentially of, or yet further consisting of a platinum drug, or alternatively a platinum drug therapy, for use in treating a human cancer patient having the appropriate expression level of the gene of interest as identified in the experimental examples.

The methods are useful in the assistance of an animal, a mammal or yet further a human patient. For the purpose of illustration only, a mammal includes but is not limited to a human, a simian, a murine, a bovine, an equine, a porcine or an ovine subject. Accordingly, a formulation comprising, or alternatively consisting essentially of, or yet further consisting of the necessary therapy or equivalent thereof is further provided herein. The formulation can further comprise one or more preservatives or stabilizers.

The agents or drugs can be administered as a composition. A “composition” typically intends a combination of the active agent and another carrier, e.g., compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates.

Various delivery systems are known and can be used to administer a chemotherapeutic agent of the disclosure, e.g., encapsulation in liposomes, microparticles, microcapsules, expression by recombinant cells, receptor-mediated endocytosis. See e.g., Wu and Wu (1987) J. Biol. Chem. 262:4429-4432 for construction of a therapeutic nucleic acid as part of a retroviral or other vector, etc. Methods of delivery include but are not limited to intra-arterial, intra-muscular, intravenous, intranasal and oral routes. In a specific embodiment, it can be desirable to administer the pharmaceutical compositions of the disclosure locally to the area in need of treatment; this can be achieved by, for example, and not by way of limitation, local infusion during surgery, by injection or by means of a catheter.

The agents identified herein as effective for their intended purpose can be administered to subjects or individuals identified by the methods herein as suitable for the therapy. Therapeutic amounts can be empirically determined and will vary with the pathology being treated, the subject being treated and the efficacy and toxicity of the agent.

Methods of administering pharmaceutical compositions are well known to those of ordinary skill in the art and include, but are not limited to, oral, microinjection, intravenous or parenteral administration. The compositions are intended for topical, oral, or local administration as well as intravenously, subcutaneously, or intramuscularly. Administration can be effected continuously or intermittently throughout the course of the treatment. Methods of determining the most effective means and dosage of administration are well known to those of skill in the art and will vary with the cancer being treated and the patient and the subject being treated. Single or multiple administrations can be carried out with the dose level and pattern being selected by the treating physician.

### Kits

Kits or panel for use in detecting the polymorphism of interest in patient biological samples are provided. In some embodiments, a kit comprises, or consists essentially of, or yet further consists of at least one reagent necessary to perform the assay. For example, the kit can comprise an enzyme, a buffer or any other necessary reagent (e.g. PCR reagents and buffers). For example, in some aspects, a kit contains, in an amount sufficient for at least one assay, any of the hybridization assay probes, amplification primers, and/or antibodies suitable for detection in a packaging material.

The various components of the kit can be provided in a variety of forms. For example, in some aspects, the required enzymes, the nucleotide triphosphates, the probes, primers, and/or antibodies are be provided as a lyophilized reagent. These lyophilized reagents can be pre-mixed before lyophilization so that when reconstituted they form a complete mixture with the proper ratio of each of the components ready for use in the assay. In addition, the kits can contain a reconstitution reagent for reconstituting the lyophilized reagents of the kit. In exemplary kits for amplifying target nucleic acid derived from a colorectal cancer patients, the enzymes, nucleotide triphosphates and required cofactors for the enzymes are provided as a single lyophilized reagent that, when reconstituted, forms a proper reagent for use in the present amplification methods.

In some aspects, the kit or panel is for determining the likely clinical outcome of a colorectal cancer patient receiving a therapy comprising, or alternatively consisting essentially of, or yet further consisting of TAS-102. In some aspects, the kit or panel is for determining the eligibility of a colorectal cancer patient for receiving a therapy comprising, or alternatively consisting essentially of, or yet further consisting of TAS-102.

Typically, the kits will also include instructions recorded in a tangible form (e.g., contained on paper or an electronic medium) for using the packaged probes, primers, and/or antibodies in a detection assay for determining the presence or amount of the polymorphism of interest in a test sample.

In some aspects, the kits further comprise a solid support for anchoring the nucleic acid of interest on the solid support. The target nucleic acid can be anchored to the solid support directly or indirectly through a capture probe anchored to the solid support and capable of hybridizing to the nucleic acid of interest. Examples of such solid support include but are not limited to beads, microparticles (for example, gold and other nano particles), microarray, microwells, multiwell plates. The solid surfaces can comprise a first member of a binding pair and the capture probe or the target nucleic acid can comprise a second member of the binding pair. Binding of the binding pair members will anchor the capture probe or the target nucleic acid to the solid surface. Examples of such binding pairs include but are not limited to biotin/streptavidin, hormone/receptor, ligand/receptor, and antigen/antibody.

In one aspect, the kit further comprises, or consists essentially of, or yet further consists of an effective amount of the therapy. In one aspect, the therapy comprises, or alternatively consists essentially of, or yet alternatively consisting of, administration of a therapeutically effective amount of TAS102. As noted above, the therapy can be combined with other known therapies as determined by the treating physician.

The kit can comprise at least one probe or primer which is capable of specifically hybridizing to the gene of interest and instructions for use. For example, in some aspects, the kits comprise at least one of the above described nucleic acids. Exemplary kits for amplifying at least a portion of the gene of interest comprise two primers. For example, in some embodiments, the kit comprises, or consists essentially of, or yet further consists of a forward primer and a reverse primer that flank the polymorphism. In some embodiments, the kit comprises, or consists essentially of, or yet further consists of a forward primer having the sequence of SEQ ID NO:8 and a reverse primer having the sequence of SEQ ID NO:9, a forward primer having the sequence of SEQ ID NO:10 and a reverse primer having the sequence of SEQ ID NO:11, a forward primer having the sequence of SEQ ID NO:49 and a reverse primer having the sequence of SEQ ID NO: 11, a forward primer having the sequence of SEQ ID NO:12 and a reverse primer having the sequence of SEQ ID NO:13, a forward primer having the sequence of SEQ ID NO:14 and a reverse primer having the sequence of SEQ ID NO:15, a forward primer having the sequence of SEQ ID NO:16 and a reverse primer having the sequence of SEQ ID NO:17, a forward primer having the sequence of SEQ ID NO:18 and a reverse primer having the sequence of SEQ ID NO:19, or a forward primer having the sequence of SEQ ID NO:20 and a reverse primer having the sequence of SEQ ID NO:21. In some embodiments, the kit further comprises, or consists essentially of, or yet further consists of a nucleic acid probe for the detection of the amplicon. In some embodiments, the nucleic acid probe has about 5, about 10, about 15, about 20, or about 25, or about 30, about 35, about 40 or more contiguous nucleotides of any of SEQ ID NO:1-7 and overlaps the polymorphic site.

In some embodiments, the kit further comprises, or consists essentially of, or yet further consists of a nucleic acid probe for the detection of the amplicon. In some embodiments, the nucleic acid probe has about 5, about 10, about 15, about 20, or about 25, or about 30, about 35, about 40 or more contiguous nucleotides. In some aspects, the nucleic acid primers and/or probes are lyophilized.

In some embodiments, at least one of the primers for amplification is capable of hybridizing to the allelic variant sequence. For example, in some embodiments, at least one of the primers for amplification has about 5, about 10, about 15, about 20, or about 25, or about 30, about 35, about 40 or more contiguous nucleotides of any of genetic region of interest of SEQ ID NO:1-7 and overlaps the polymorphic site. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the surface is silica or glass. In another embodiment, the surface is a metal electrode.

The test samples used in the diagnostic kits include cells, protein or membrane extracts of cells, or biological fluids such as sputum, blood, serum, plasma, or urine. The test samples can also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest or target region.

As amenable, these suggested kit components can be packaged in a manner customary for use by those of skill in the art. For example, these suggested kit components can be provided in solution or as a liquid dispersion or the like.

Typical packaging materials would include solid matrices such as glass, plastic, paper, foil, micro-particles and the like, capable of holding within fixed limits hybridization assay probes, and/or amplification primers. Thus, for example, the packaging materials can include glass vials used to contain sub-milligram (e.g., picogram or nanogram) quantities of a contemplated probe, primer, or antibodies or they can be microtiter plate wells to which probes, primers, or antibodies have been operatively affixed, i.e., linked so as to be capable of participating in an amplification and/or detection methods.

The instructions will typically indicate the reagents and/or concentrations of reagents and at least one assay method parameter which might be, for example, the relative amounts of reagents to use per amount of sample. In addition, such specifics as maintenance, time periods, temperature, and buffer conditions can also be included.

The diagnostic systems contemplate kits having any of the hybridization assay probes, amplification primers, or antibodies described herein, whether provided individually or in one of the combinations described above, for use in determining the presence or amount of a polymorphism of interest, e.g. noted in Table A or as identified herein.

The disclosure now being generally described, it will be more readily understood by reference to the following example which is included merely for purposes of illustration of certain aspects and embodiments of the present disclosure, and are not intended to limit the disclosure.

### EXAMPLES

### Example 1

This example shows that functional significant single nucleotide polymorphisms in genes involved in the colorectal cancer treated with TAS-102.

TAS-102 is an orally administered combination of thymidine-based nucleoside analogue, trifluridine (FTD), and thymidine phosphorylase inhibitor (TPI), tipiracil hydrochloride [1]. FTD is an active cytotoxic component of TAS-102, and incorporation of tri-phosphorylated FTD into DNA confers its anti-tumor effect. FTD is rapidly degraded to an inactive form, 5-trifluoromethyl-2,4(1H,3,H)-pyrimidinedione (FTY), by thymidine phospholyrase (TP), hence inhibition of TP by TPI plays a critical role to maintain increased FTD concentrations leading to enhance cytotoxicity of TAS-102 [2,3]. In the present study, the effect of transporters that are involved in pharmacokinetics and pharmacodynamics of FTD and TPI was investigated. Nucleoside transporters (NTs) include concentrative NTs (CNTs) and equilibrative NTs (ENTs). They are expressed on the epithelial cells in the intestine, and transport nucleoside analogues, such as FTD [4-6]. Previous in vivo studies revealed that FTD was absorbed via CNT1 in the intestinal lumens of rat indicating FTD as a substrate for CNT1 [5]. Once FTD is taken up into the cells, thymidine kinase 1 (TK1) subsequently converts FTD to FTD monophosphate (F3dTMP) and finally FTD triphosphate (F3dTTP) is only incorporated into DNA leading to DNA strand breaks as DNA dysfunction [2,7,8,9]. According to the critical role of TAS-102 pharmacokinetics, the NTs and TK1 are considered to be involved not only in anticancer role but also in the toxicity of FTD as a potential biomarker during treatment [1,10]. A recent report suggested that decreased expression of hENT1 and TK1 might represent a resistant mechanism to FTD leading to decreased nuclear intake of FTD and overall impaired activity [11].

TPI lacks of anti-tumor activity in TAS-102, albeit it serves as inhibitor of FTD degradation and potentially acts as an anti-angiogenic agent [2]. According to the phase I study and pharmacokinetics studies, most of FTD is metabolized and excreted in urine after converted into inactive form, FTY. By contrast, most of TPI is not metabolized and mainly excreted in urine with unchanged form [1,12]. In addition, TPI is known as a substrate of the organic cation transporter (OCT2, SLC22A2), which is collaborating with Human multidrug and toxin extrusion 1 (MATE1, SLC47A1) to facilitate tubular reabsorption and secretion of drugs as another renal drug elimination separated from glomerular filtration [13,14].

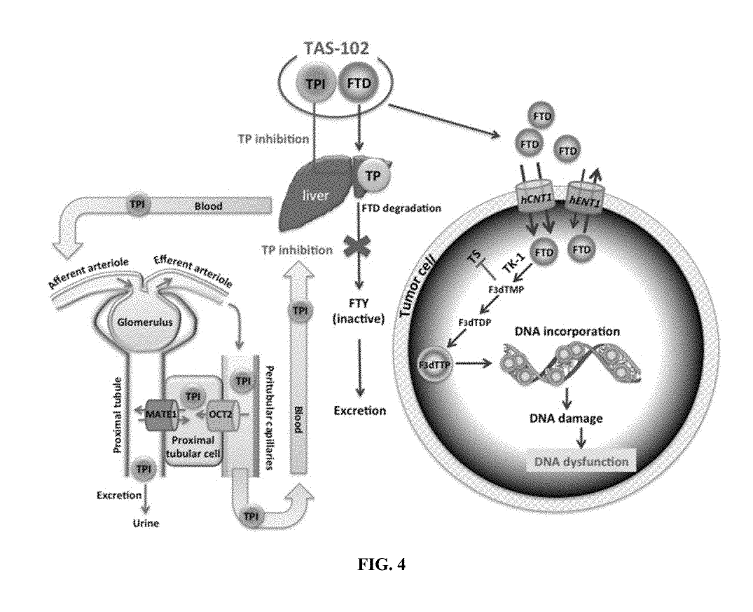

MATE1 and OCT2 play an important role in excretion of TPI, and renal clearance of which might be responsible for equilibration of TPI blood concentration (FIG. 4).

The hypothesis is that circulating unchanged TPI in blood might re-participate to inhibit TP in the liver leading to diminish FTD degradation by TP, and which might harmonize with NTs in producing synthetic ant-tumor effect of TAS-102 (FIG. 4). The gene polymorphisms involved in FTD and TPI pharmacokinetics were tested, especially FTD absorption by NTs (hCNT1, hENT1) and metabolism by TK1 and TPI excretion by OCT2 or MATE1 are associated with outcomes and toxicities in patients with refractory metastatic colorectal cancer (mCRC) treated with TAS-102.

### Materials and Methods

### Study Design and Patients

This was a retrospective exploratory study investigating three independents cohorts of patients with refractory mCRC; a training cohort of 52 patients receiving TAS-102 referred at Cancer Institute Hospital in Japan, a testing cohort of 129 patients receiving TAS-102 referred at Azienda Ospedaliero-Universitaria Pisana, Pisa, Italy, Istituto Oncologico Veneto, Padova, Italy, and Istituto Nazionale Tumori, Milano, Italy; and a control cohort of 52 patients treated with regorafenib referred at Azienda Ospedaliero-Universitaria Pisana. All patients underwent the treatment in salvage-line setting. Eligible patients had a histologically confirmed diagnosis of mCRC; history of previous standard chemotherapy consisting of 5-FU, L-OHP, CPT-11, bevacizumab, and cetuximab or panitumumab if KRAS wild-type; measurable disease according to Response Evaluation Criteria in Solid Tumors (RECIST) v1.1, and signed informed consent.

Patients assigned in the training cohort and in the control cohort received TAS-102 (Taiho Pharmaceutical Co., Ltd., Tokyo, Japan) 35 mg per square meter twice daily days 1-5 and 8-12, every 4 weeks. Patients in the control group received 160 mg regorafenib (Bayer, Leverkusen, Germany) once daily from day 1 to 21 every 4 weeks. Doses were adjusted based on haematological and non-haematological adverse events at the treating physician's discretion in accordance with the manufacturer's recommendations. The analyses were approved by the University of Southern California (USC) Institutional Review Board of Medical Sciences and conducted at the USC/Norris Comprehensive Cancer Center in accordance with the Declaration of Helsinki and Good Clinical Practice Guidelines.

### Selection of Single-Nucleotide Polymorphism

The selected ten candidates SNPs in genes involved in FTD metabolism and TPI excretion, TK1, hENT1, hCNT1, OCT2 and MATE1 according to either of the following criteria: i) SNPs with statistical significance reported in literatures, ii) tagging SNPs using the HapMap genotype data with r² threshold=0.8: http://snpinfo.niehs.nih.gov/snpinfo/snptag.htm, or iii) minor allele frequency with a cut-off of 10% or greater in both Caucasians and East Asians (in the Ensembl Genome Browser: http://uswest.ensembl.org/index.html). Functional significance was predicted using functional single-nucleotide polymorphism (F-SNP) database: http://compbio.cs.queensu.ca/F-SNP/. (Table 1).

TABLE 1
Analyzed genes polymorphisms in FTD and TPI metabolic pathwa

| Genes rs | Allele | Base | MAF† | Function of | |
|---|---|---|---|---|---|
| numbers | location | exchange | (CEU/JPN) | polymorphism | Forward/Reverse primer (5′-3′) |
| TK1 | 3′prime UTR | C > T | 0.32/0.15 | Transcriptional | F: GCAGACCAGTGGgTAGGAGA |
| rs1055759 | Chromosome | | | regulation | R: AGCCACTCCAGGAGGAAGTC |
| | 17:78174554 | | | | |
| hENT1 | Intron | A > G | 0.35/0.19 | Tag SNP | F: TGGGGGACACTCAGTAGAGG |
| rs750370 | Chromosome | | | | R: AACGTGTATGGTGGGGTTGT |
| | 6:44233216 | | | | |
| hENT1 | Intron | C > T | 0.30/0.27 | Tag SNP | F: CTGCCTCCTGTGCTCCAT |
| rs9394992 | Chromosome | | | | R: TGGGAAATGACTGAGCTGTG |
| | 5:4422255 | | | | |
| CNT1 | Non | G > A | 0.39/0.33 | Protein coding, | F: CTGGTCCTGTGGCTGTCTCT |
| rs2290272 | symonymous | | | splicing | R: GCCCCACAACTAGCACTCAC |
| | coding | | | regulation post | |
| | Chromosome | | | translation | |
| | 15:84904200 | | | | |
| MATE1 | Intron | G > A | 0.45/0.49 | Transcriptional | F: CCAGTTTGTGCTAAGCATCG |
| rs2289669 | Chromosome | | | regulation | R: ACACCTGGTGGGAAAACTTG |
| | 17:19560030 | | | | |
| MATE2 | Intron | A > C | 0.31/0.38 | Transcriptional | F: CAGGCATCACTAAGCTGCAA |
| rs15950380 | Chromosome | | | regulation | R: TTCTCTCCTTTCTGCATTAATTTG |
| | 17:13710258 | | | | |
| OCT2 | Non | C > A | 0.10/0.10 | Protein coding, | F: GAAGGCAGACTTCTTAGCAGAAT |
| rs315019 | symonymous | | | splicing | R: ATACAGTTGGGCTCCTGGTG |
| | coding | | | post | |
| | Chromosome | | | translation | |
| | 6:150249250 | | | | |
| OCT2 | Intron | A > G | 0.21/0.12 | Protein coding, | F: CCAGACCACTCAAGCTTTCTC |
| rs318000 | Chromosome | | | transcriptional | R: AGTCATGTTGAAAGCCAGCA |
| | 5:150223498 | | | regulation | |

MAF, Minor allele frequency; CEU, Caucasian; JPN, Japanese; F, forward primer; R, reverse primer; Tag SNP, tagging SNP.
†In Caucasian and Japanese from the Ensembl Genome Browser: http://uswest.ensembl.org/index.html.

### DNA Extraction and Genotyping

Genomic DNA was extracted from peripheral whole blood in patients of all cohorts using the QIAmp Kit (Qiagen, Valencia, Calif., USA) according to the manufacturer's protocol (www.qiagen.com). The candidate SNPs were genotyped using PCR-based direct DNA sequence analysis by ABI 3100A Capillary Genetic Analyzer and Sequencing Scanner v1.0 (Applied Biosystems). Amplification of extracted DNA was carried out using both forward and reverse primers for each gene polymorphism as shown in Table 1. For quality control purposes, 10% of the samples were randomly analyzed for each SNP by direct DNA sequencing, and resulted in the genotype concordance rate of 99% or more. The investigators analyzing SNPs were blinded to the clinical data of the study patients.

### Development of New Signature Based on Gene-Gene Interaction of OCT2 and MATE1 in TPI Excretion

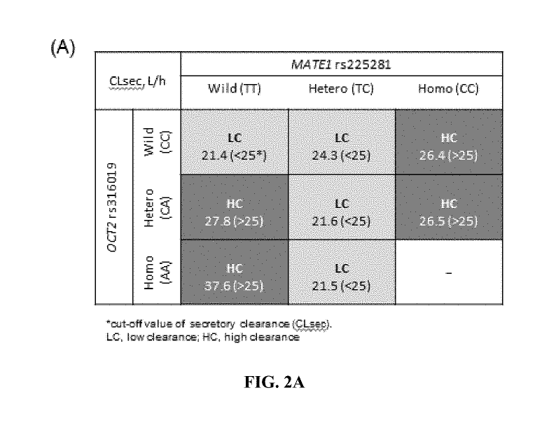

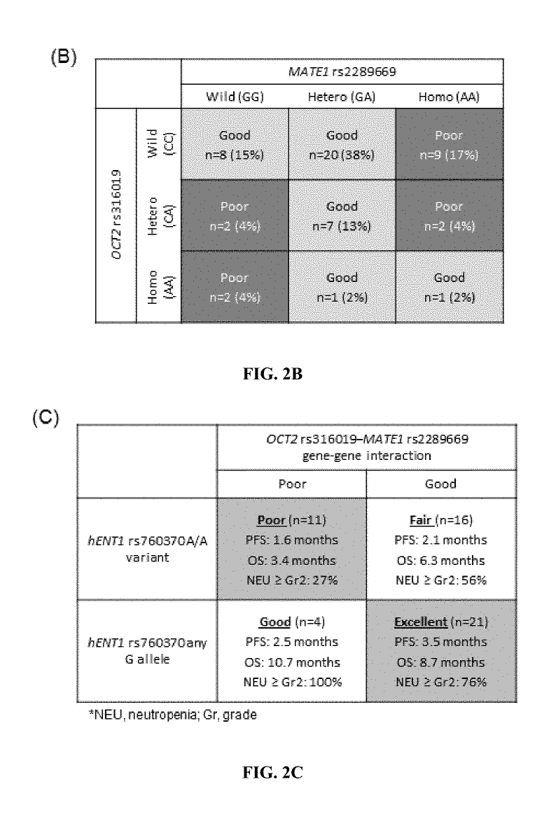

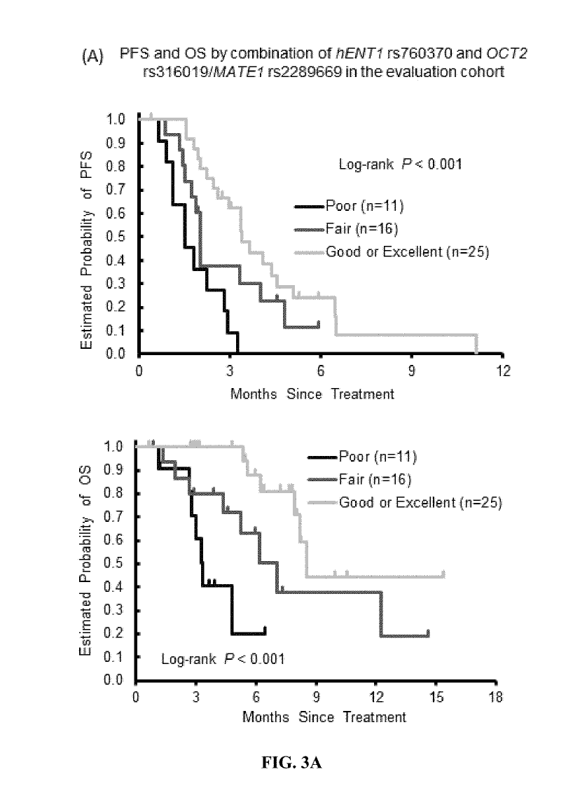

Christensen et al. investigated association between renal clearance (CLᵣₑₙₐₗ) of a biguanide metformin in healthy volunteers and gene polymorphism of OCT2, and gene-gene interaction of SNPs between OCT2 and MATE1 genes. As pharmacokinetic parameters, CLᵣₑₙₐₗ (amount of metformin in urine₀₋₂₄ₕ/AUC₀₋₂₄ₕ) and secretory clearance (CL_{sec}) (CLᵣₑₙₐₗ eGFRxBSA/1.73 m2) were calculated, and volunteers with OCT2 rs316019 heterozygote or homozygote along with MATE1 rs225281 heterozygote had significantly lower CLᵣₑₙₐₗ and CL_{sec} of metoformin. CLᵣₑₙₐₗ and CL_{sec} of other assemblies were shown in FIG. 2A, which demonstrated counteracting effects of these SNPs on the renal elimination of metformin [15].

Based upon the variation of the CLᵣₑₙₐₗ and the CL_{sec} of metformin in healthy volunteers in the study, first a cut-off with CL_{sec} of 25L/h was sept up. Second it was identified whether individual CL_{sec} the nine assemblies composed of OCT2 rs316019 (CC, CA, AA) and MATE1 rs225281 (TT, TC, CC) variants were above or below the cut-off. Finally, the nine assemblies were divided into two categories, ‘High clearance (HC)’ and ‘Low clearance (LC)’ by the cut-off.

There was no data of TT/CC assembly in the study (FIG. 2A). Grun et al. investigated genetic modulation by MATE1 and OCT2 gene polymorphism on metformin pharmacokinetics, stratifying volunteers into four strata according to genotypes: MATE1 rs2289669/OCT2 rs316019: 1, MATE1 wild type (WT) and OCT2 WT; 2, MATE1 homozygote (HM) and OCT2 WT; 3, MATE1 WT and OCT2 HM; and 4, MATE1 HM and OCT2 HT [16]. Although data of clearance were lacking in other five assemblies with small sample size in the study, CLᵣₑₙₐₗ of metformin was relatively higher in stratum 3 compared with those in stratum 1, which was consistent with the report by Christensen et al. [15]. In developing a signature, whether the categorization shown in FIG. 2A will be adapted to the SNPs in prediction of outcome of TAS-102 was tested.

### Statistical Analysis

The primary endpoint in this study was progression-free survival (PFS), and the secondary endpoints were overall survival (OS) and disease control rate (DCR). PFS was calculated from the date of starting treatment to the first observation of disease progression or death from any cause. If a patient had no disease progression or death, PFS was censored at the date of last follow up. OS was calculated from the date of starting treatment until the date of death from any cause or the date of the last follow-up, at which point OS was censored. The disease control rate (DCR) was defined as the proportion of patients who achieved a stable disease (SD) or progressed disease (PD) according to RECISTv1.1. Chi-square test was used to examine the difference in baseline patient characteristics among the three cohorts. Allelic distribution of all SNPs by ethnicity for deviation from Hardy-Weinberg equilibrium and the allelic frequencies of SNPs were tested using the Chi-square tests with one degree of freedom. The associations between candidate SNPs and disease control, and toxicities were examined using contingency tables and Fisher's exact test. The associations of SNPs with PFS and OS were analysed using Kaplan-Meier curves and the log-rank test. Because the true mode of inheritance of candidate SNPs was not established yet, a codominant or dominant genetic model if appropriate was considered. Multivariable analysis for PFS and OS was conducted using the Cox proportional hazards model, and the baseline characteristics that remained significantly associated with PFS and OS in multivariable models were included in the final model for each SNP.

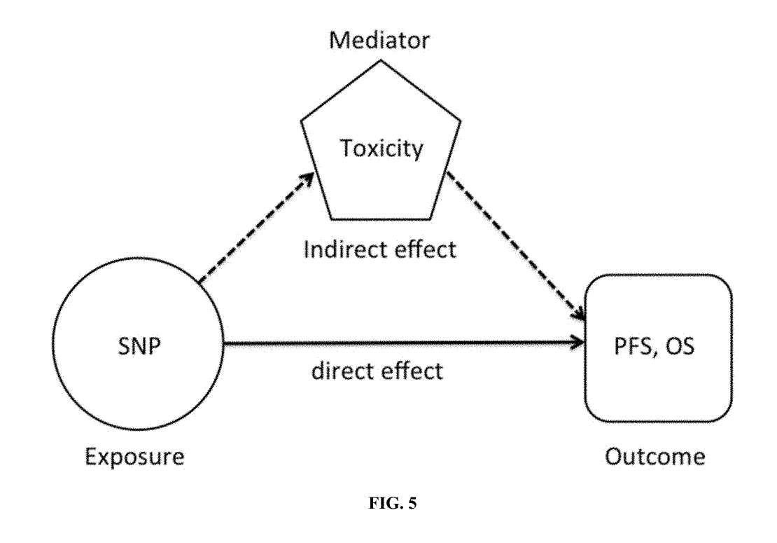

To evaluate that if identified SNPs predicted clinical outcome (PFS and OS) independently or mediated by toxicity, the mediation analysis using the counterfactual framework [17] was performed, by which the total effect of SNP on the outcome would be decomposed into a direct effect (SNP→outcome) and an indirect effect (SNP→toxicity→outcome) (FIG. 5). The following criteria need to be satisfied for toxicity to be considered as a mediator: 1) the SNP must predict the toxicity, 2) the toxicity must predict outcome, and 3) the SNP must predict outcome, which was expressed as total effect. Suppression effect might exist when the direct is larger than the total effect, under which circumstances, the third criteria might not hold, and the direct and indirect effects often have similar magnitudes and opposite signs. Proportion mediated is also a popular measure in mediation analysis, which can be defined as the ratio of the indirect effect to the total effect. The transformation of the effects would be used to calculate this proportion when it was on risk difference scale [18]. The SAS Macro % MEDIATION introduced by Valeri L and VanderWeele TJ [19] was applied to perform the mediation analysis. In this study, logistic regression was used to predict the effect between SNP and toxicity, and the Cox proportional hazard model was used to predict the outcome. The models were adjusted for the patient baseline characteristics that significantly predict PFS and OS in multivariable model. Bootstrap with 10,000 simulations was used to calculate the confidence interval for effects. In the training cohort, considering the dominant model and using a two-sided 0.05-level log-rank test, the minimum detectable hazard ratios with 80% power ranged from 2.42 to 3.23 for PFS (N=52, PFS events=43) across the minor allele frequency of 0.1 to 0.4. When applying the same model and test, the power was greater than 98% in the testing cohort. All analyses were carried out with SAS 9.4 (SAS Institute, Cary, N.C., USA). All tests were 2-sided at a significance level of 0.05.

### Baseline Patients and Tumor Characteristics

The baseline characteristics of the training, testing, and control cohorts are summarized in Table 2. Patients' characteristics were similar in the three cohorts except for a higher number of males and lower number of chemotherapy lines received before TAS-102 in the testing cohort; higher percentage of patients with ECOG performance status=0 and no history of adjuvant treatment in the control cohort, compared with the other cohorts, respectively. In addition, associations between baseline characteristics and clinical outcomes in each cohort were summarized in Table 4, 5 and 6. The median follow-up time, median PFS and OS were 6.4 months, 2.6 months and 8.0 months in the training cohort, 5.3 months, 2.0 months and 5.7 months in the testing cohort, and 5.3 months, 1.9 months and 5.3 months in the control cohort, respectively.

TABLE 2
Baseline patients and tumor characteristics

| | Training cohort | | Testing cohort | | Control cohort | | |
|---|---|---|---|---|---|---|---|
| | TAS-102 (N = 52) | | TAS-102 (N = 129) | | Regorafenib (N = 52) | | |
| Cohort | N | % | N | % | N | % | P value * |
| Sex | | | | | | | 0.026 |
| Male | 23 | 44.2 | 80 | 63.0 | 23 | 44.2 | |
| Female | 29 | 55.8 | 49 | 38.0 | 29 | 55.8 | |
| Age (year) | | | | | | | |
| Median (range) | 61 (34-83) | | 66 (35-83) | | 64 (42-81) | | 0.11 |
| <61 | 25 | 48.1 | 44 | 34.1 | 19 | 36.5 | 90.21 |
| ≥61 | 27 | 51.9 | 85 | 65.9 | 33 | 63.5 | |
| ECOG Performance status | | | | | | | 0.009 |
| ECOG 0 | 33 | 63.5 | 73 | 56.6 | 42 | 80.8 | |
| ECOG 1 | 19 | 36.5 | 56 | 43.4 | 10 | 19.2 | |
| Primary tumor site | | | | | | | 0.41 |
| Right | 19 | 37.3 | 40 | 32.0 | 22 | 42.3 | |
| Left | 32 | 62.7 | 85 | 65.0 | 30 | 57.7 | |
| Liver metastasis | | | | | | | 0.18 |
| Yes | 38 | 73.1 | 97 | 75.2 | 45 | 86.5 | |
| No | 14 | 26.9 | 32 | 24.8 | 7 | 13.5 | |
| Lung metastasis | | | | | | | 0.55 |
| Yes | 36 | 69.2 | 89 | 69.0 | 40 | 76.9 | |
| No | 16 | 30.5 | 40 | 31.0 | 12 | 23.1 | |
| Lymph node metastasis | | | | | | | 0.57 |
| Yes | 23 | 44.2 | 56 | 43.4 | 27 | 51.9 | |
| No | 29 | 55.8 | 73 | 56.5 | 25 | 48.1 | |
| Peritoneal metastasis | | | | | | | 0.78 |
| Yes | 12 | 23.1 | 32 | 24.5 | 15 | 28.9 | |
| No | 40 | 76.9 | 97 | 75.2 | 37 | 71.1 | |
| Number of metastases | | | | | | | 0.14 |
| <2 | 32 | 61.5 | 70 | 54.3 | 22 | 42 3 | |
| ≥2 | 20 | 38.5 | 59 | 45.7 | 30 | 57.7 | |
| Primary remain | | | | | | | 0.86 |
| Yes | 7 | 13.5 | 13 | 10.6 | 6 | 12 | |
| No | 45 | 86.5 | 110 | 85.4 | 46 | 88 | |
| Adjuvant history | | | | | | | 0.003 |
| Yes | 24 | 46.1 | 47 | 36.4 | 8 | 15.4 | |
| No | 28 | 53.9 | 82 | 63.6 | 44 | 84.6 | |
| Pre-chemotherapy numbers | | | | | | | <0.001 |
| <4 | 30 | 57.7 | 106 | 82.2 | 31 | 59.6 | |
| ≥4 | 22 | 42.3 | 23 | 17.8 | 21 | 40.4 | |
| KRAS exon2 status | | | | | | | 0.77 |
| Wild-type | 28 | 54.9 | — | — | 26 | 52.0 | |
| Mutant | 23 | 45.1 | — | — | 24 | 48.0 | |

* P value was based on Chi-square test, or the Kruskai-Wallis test when appropriate.

TABLE 4
Baseline characteristics and clinical outcome in the training cohort treated with TAS-102

| | | Progression-free Survival | | | Overall Survival | | |
|---|---|---|---|---|---|---|---|
| | | Median, months | | | Median, months | | |
| | | (95% Cl) | HR (95% Cl) | P value⁺ | (95% Cl) | HR (95% Cl) | P value⁺ |
| Age | | | | 0.48 | | | 0.55 |
| <61 | 25 | 2.2 (1.8, 3.4) | 1 (Reference) | | 8.1 (5.3, 12.4) | 1 (Reference) | |
| ≥61 | 27 | 3.0 (1.9, 4.1) | 0.81 (0.44, 1.49) | | 8.3 (4.9, 14.8+) | 1.30 (0.54, 3.09) | |
| Sex | | | | 0.087 | | | 0.064 |
| Male | 23 | 2.1 (1.6, 3.3) | 1 (Reference) | | 4.9 (3.3, 8.1+) | 1 (Reference) | |
| Remale | 29 | 3.4 (2.1, 4.2) | 0.60 (0.32, 1.12) | | 8.3 (6.3, 15.8+) | 0.47 (0.18, 1.20) | |
| Performance status | | | | 0.84 | | | 0.43 |
| ECOG0 | 33 | 2.9 (2.8, 3.7) | 1 (Reference) | | 8.7 (6.3, 14.8+) | 1 (Reference) | |
| ECOG1 | 19 | 2.7 (1.8, 3.4) | 1.06 (0.58, 2.00) | | 7.2 (4.9, 15.8+) | 1.42 (0.59, 3.37) | |
| Tumor site | | | | 0.42 | | | 0.95 |
| Right | 19 | 3.3 (1.6, 6.6+) | 1 (Reference) | | 8.7 (3.4, 14.8+) | 1 (Reference) | |
| Left | 32 | 2.9 (2.0, 3.4) | 1.30 (0.67, 2.53) | | 8.1 (5.3, 15.8+) | 0.97 (0.40, 2.38) | |
| Liver metastasis | | | | 0.010 | | | 0.27 |
| Yes | 38 | 2.3 (1.8, 3.4) | 1 (Reference) | | 7.2 (4.9, 14.8+) | 1 (Reference) | |
| No | 14 | 4.2 (1.8, 11.3+) | 0.43 (0.20, 0.91) | | 8.7 (5.4, 15.8+) | 0.57 (0.21, 1.58) | |
| Lung metastasis | | | | 0.58 | | | 0.42 |
| Yes | 36 | 2.9 (2.1, 4.1) | 1 (Reference) | | 8.3 (5.7, 14.8+) | 1 (Reference) | |
| No | 16 | 2.5 (1.4, 3.4) | 1.19 (0.61, 2.30) | | 6.3 (4.4, 15.8+) | 1.44 (0.59, 3.48) | |
| LN metastasis | | | | 0.43 | | | 0.90 |
| Yes | 23 | 3.0 (1.8, 4.4) | 1 (Reference) | | 8.3 (4.4, 15.8+) | 1 (Reference) | |
| No | 29 | 2.6 (1.6, 3.4) | 1.27 (0.69, 2.34) | | 7.2 (5.3, 14.8+) | 1.08 (0.46, 2.51) | |
| Peritoneal | | | | 0.25 | | | 0.71 |
| Yes | 12 | 3.1 (1.9, 11.3+) | 1 (Reference) | | 6.3 (4.4, 12.4) | 1 (Reference) | |
| No | 40 | 2.3 (1.8, 3.5) | 1.48 (0.71, 3.09) | | 8.3 (5.7, 14.8+) | 0.84 (0.33, 2.15) | |
| Number of metastases | | | | 0.51 | | | 0.26 |
| ≤2 | 32 | 2.7 (2.1, 3.7) | 1 (Reference) | | 8.3 (6.3, 15.8+) | 1 (Reference) | |
| >2 | 20 | 2.9 (1.8, 3.6) | 1.22 (0.64, 2.31) | | 8.1 (3.3, 12.4+) | 1.63 (0.68, 3.89) | |
| Pre-chemonumbers | | | | 0.062 | | | 0.032 |
| <4 | 30 | 3.0 (2.1, 4.4) | 1 (Reference) | | 8.7 (6.3, 15.6+) | 1 (Reference) | |
| ≥4 | 22 | 2.1 (1.8, 3.4) | 1.73 (0.92, 3.23) | | 6.3 (3.1, 12.4+) | 2.48 (1.04, 5.90) | |
| Adjuvant history | | | | 0.028 | | | 0.033 |
| Yes | 24 | 3.7 (2.1, 4.6) | 1 (Reference) | | 8.7 (6.3, 15.8+) | 1 (Reference) | |
| No | 28 | 2.1 (1.6, 2.9) | 1.90 (1.02, 3.63) | | 6.3 (4.9, 10.1+) | 2.44 (0.96, 8.17) | |
| Primary tumor | | | | 0.48 | | | 0.37 |
| resection | | | | | | | |
| Yes | 45 | 3.0 (2.1, 3.5) | 1 (Reference) | | 8.1 (5.7, 12.4) | 1 (Reference) | |
| No | 7 | 2.1 (0.9, 6.0+) | 1.39 (0.54, 3.58) | | 8.1+ (1.2, 8.1+) | 1.72 (0.49, 5.99) | |
| KRAS | | | | 0.018 | | | 0.060 |
| Wildtype | 28 | 2.1 (1.8, 3.0) | 1 (Reference) | | 7.2 (3.4, 8.7) | 1 (Reference) | |
| Mutant | 23 | 3.3 (1.8, 5.2) | 0.51 (0.26, 0.99) | | 8.3 (6.7, 15.8+) | 0.42 (0.16, 1.07) | |
| Histology | | | | 0.86 | | | 0.17 |
| well | 13 | 2.1 (1.5, 5.2) | 1 (Reference) | | 8.3 (2.0, 8.3+) | 1 (Reference) | |
| Mod-por | 36 | 3.0 (1.9, 3.5) | 0.94 (0.44, 2.00) | | 5.7 (5.7, 15.8+) | 0.52 (0.20, 1.40) | |
| Prior Bev | | | | 0.28 | | | 0.073 |
| Yes | 48 | 2.9 (2.1, 3.5) | 1 (Reference) | | 8.3 (6.3, 16.8+) | 1 (Reference) | |
| No | 4 | 2.5 (1.6, 3.0+) | 1.86 (0.55, 6.24) | | 4.1 (3.4, 4.9+) | 3.43 (0.71, 16.58) | |
| Prior EGFR | | | | 0.012 | | | 0.024 |
| Yes | 28 | 2.1 (1.8, 3.0) | 1 (Reference) | | 6.3 (3.4, 12.4+) | 1 (Reference) | |
| No | 24 | 3.7 (2.3, 5.2) | 0.49 (0.26, 0.85) | | 15.5+ (6.3, 15.8+) | 0.35 (0.14, 0.91) | |
| Prior REGORA | | | | 0.18 | | | 0.21 |
| Yes | 36 | 2.1 (1.8, 3.4) | 1 (Reference) | | 8.1 (4.9, 12.4) | 1 (Reference) | |
| No | 16 | 3.7 (2.1, 4.9) | 0.65 (0.33, 1.26) | | 14.8+ (5.3, 14.8+) | 0.50 (0.17, 1.60) | |

+g value was based on log-rank test in the univariate analysis.

TABLE 5
Baseline characteristics and clinical outcome in the training cohort treated with TAS-102

| | | Progression-free Survival | | | Overall Survival | | |
|---|---|---|---|---|---|---|---|
| | | Median, months | | | Median, months | | |
| | N | (95% Cl) | HR (95% Cl) | P value⁺ | (95% Cl) | HR (95% Cl) | P value⁺ |
| Age group | | | | 0.010 | | | 0.029 |
| <61 | 44 | 1.9 (1.7, 2.0) | 1 (Reference) | | 4.4 (3.7, 5.4) | 1 (Reference) | |
| ≥61 | 85 | 2.0 (1.8, 2.5) | 0.51 (0.41, 0.91) | | 9.0 (5.2, 9.0) | 0.56 (0.32, 0.96) | |
| Sex | | | | 0.93 | | | 0.46 |
| Male | 80 | 1.9 (1.9, 2.0) | 1 (Reference) | | 9.0 (4.6, 9.0) | 1 (Reference) | |
| Remale | 49 | 2.1 (1.9, 2.5) | 0.98 (0.66, 1.47) | | 5.3 (3.8, 7.3) | 1.22 (0.70, 2.12) | |
| Performance status | | | | 0.069 | | | 0.003 |
| ECOG 0 | 73 | 2.0 (1.9, 2.4) | 1 (Reference) | | 9.0 (5.3, 9.0) | 1 (Reference) | |
| ECOG 1+ | 58 | 1.9 (1.8, 2.1) | 1.43 (0.98, 2.13) | | 4.1 (3.1, 7.1) | 2.15 (1.25, 3.71) | |
| Tumor site | | | | 0.75 | | | 0.38 |
| Right | 40 | 1.9 (1.8, 2.3) | 1 (Reference) | | 5.1 (3.7, 8.7) | 1 (Reference) | |
| Left | 85 | 2.0 (1.9, 2.3) | 1.07 (0.70, 1.64) | | 9.0 (4.2, 9.0) | 0.78 (0.44, 1.37) | |
| Liver metastasis | | | | 0.006 | | | 0.28 |
| Yes | 97 | 1.9 (1.8, 2.0) | 1 (Reference) | | 5.4 (4.1, 9.0) | 1 (Reference) | |
| No | 32 | 2.4 (2.0, 6.5) | 0.52 (0.32, 0.88) | | 8.7+ (3.8, 8.7+) | 0.69 (0.36, 1.37) | |
| Lung metastasis | | | | 0.017 | | | 0.15 |
| Yes | 89 | 2.1 (1.9, 2.5) | 1 (Reference) | | 8.8+ (4.7, 8.8+) | 1 (Reference) | |
| No | 40 | 1.9 (1.7, 2.0) | 1.82 (1.06, 2.47) | | 4.1 (3.7, 9.0) | 1.46 (0.84, 2.55) | |
| LN metastasis | | | | 0.98 | | | 0.44 |
| Yes | 58 | 2.0 (1.9, 2.1) | 1 (Reference) | | 8.8+ (4.0, 8.8+) | 1 (Reference) | |
| No | 73 | 2.0 (1.9, 2.4) | 0.99 (0.67, 1.47) | | 5.4 (3.9, 9.0) | 1.24 (0.71, 2.14) | |
| Peritoneal | | | | 0.17 | | | 0.47 |
| Yes | 32 | 1.9 (1.7, 2.0) | 1 (Reference) | | 5.3 (3.7, 7.1) | 1 (Reference) | |
| No | 97 | 2.0 (1.9, 2.3) | 0.74 (0.47, 1.18) | | 9.0 (4.1, 9.0) | 0.80 (0.44, 1.46) | |
| Number of metastases | | | | 0.38 | | | 0.48 |
| ≤2 | 70 | 2.0 (1.9. 2.4) | 1 (Reference) | | 5.5 (3.8, 9.0) | 1 (Reference) | |
| >2 | 59 | 2.0 (1.9, 2.1) | 1.18 (0.80, 1.75) | | 8.8+ (4.4, 8.8+) | 0.83 (0.48, 1.42) | |
| Pre-chemonumbers | | | | 0.029 | | | 0.058 |
| <4 | 51 | 1.9 (1.5, 2.0) | 1 (Reference) | | 6.1 (3.8, 9.0+) | 1 (Reference) | |
| ≥4 | 48 | 2.1 (1.9, 2.8) | 0.66 (0.43, 0.99) | | 8.7+ (5.2, 8.7+) | 0.57 (0.31, 1.04) | |
| Adjuvant history | | | | 0.26 | | | 0.48 |
| Yes | 47 | 2.0 (1.9, 2.5) | 1 (Reference) | | 8.6+ (4.2, 8.5+) | 1 (Reference) | |
| No | 82 | 1.9 (1.9, 2.1) | 1.25 (0.83, 1.88) | | 5.5 (3.9, 9.0) | 1.23 (0.70, 2.18) | |
| Primary tumor resection | | | | 0.007 | | | 0.12 |
| Yes | 110 | 2.0 (1.9, 2.3) | 1 (Reference) | | 9.0 (5.1, 9.0) | 1 (Reference) | |
| No | 13 | 1.8 (1.8, 2.0) | 2.13 (1.18, 3.90) | | 3.9 (2.7, 8.7) | 1.87 (0.84, 416) | |
| RAS status | | | | 0.35 | | | 0.18 |
| Wildtype | 47 | 2.0 (1.8, 2.3) | 1 (Reference) | | 8.8+ (3.7, 8.8+) | 1 (Reference) | |
| Mutant | 69 | 1.9 (1.9. 2.1) | 1.21 (0.80, 1.83) | | 5.1 (3.7, 9.0) | 1.48 (0.82, 2.64) | |
| BRAF status | | | | 0.26 | | | 0.028 |
| Wildtype | 108 | 2.0 (1.9, 2.3) | 1 (Reference) | | 5.8 (4.6, 8.0) | 1 (Reference) | |
| Mutant | 9 | 1.8 (1.2, 2.0) | 1.40 (0.86, 2.99) | | 3.3 (1.2, 8.7) | 2.38 (1.07, 5.31) | |
| Prior REGORA | | | | 0.40 | | | 0.35 |
| Yes | 36 | 2.0 (1.8, 2.8) | 1 (Reference) | | 8.7+ (4.2, 8.7+) | 1 (Reference) | |
| No | 93 | 2.0 (1.9, 2.1) | 1.20 (0.77, 1.87) | | 5.4 (3.9, 9.0) | 1.36 (0.71, 2.58) | |
| Time to metastasis | | | | 0.14 | | | 0.064 |
| Synchronous | 78 | 1.9 (1.9, 2.1) | 1 (Reference) | | 5.2 (3.7, 9.0) | 1 (Reference) | |
| Metachronous | 53 | 2.0 (1.9, 2.6) | 0.75 (0.50, 1.12) | | 8.5+ (4.7, 8.5+) | 0.59 (0.34, 1.05) | |
| Adjuvant Oxa | | | | 0.79 | | | 0.61 |
| Yes | 37 | 2.0 (1.7, 2.5) | 1 (Reference) | | 8.5 (4.1, 8.5+) | 1 (Reference) | |
| No | 92 | 1.9 (1.9, 2.1) | 1.06 (0.68, 1.85) | | 5.8 (3.9, 9.0) | 1.17 (0.64, 2.16) | |
| Grading | | | | 0.57 | | | 0.33 |
| 1-2 | 81 | 2.1 (1.9, 2.3) | 1 (Reference) | | 9.0 (5.1, 9.0) | 1 (Reference) | |
| 3 | 29 | 1.9 (1.8, 2.6) | 1.15 (0.71, 1.85) | | 5.3 (3.8, 8.5) | 1.38 (0.72, 2.66) | |
| Mucinous histology | | | | 0.90 | | | 0.37 |
| Yes | 18 | 1.9 (1.7, 2.1) | 1 (Reference) | | 5.5 (2.8, 8.7) | 1 (Reference) | |
| No | 99 | 2.0 (1.9, 2.3) | 0.97 (0.53, 1.74) | | 9.0 (4.7, 9.0) | 0.72 (0.35, 1.49) | |
| Previous anti-VEGF | | | | 0.42 | | | 0.48 |
| Yes | 120 | 2.0 (1.9, 2.1) | 1 (Reference) | | 5.5 (4.4, 9.0) | 1 (Reference) | |
| No | 7 | 2.8 (0.4, 4.4) | 0.87 (0.25, 1.83) | | 8.8+ (3.8, 6.6+) | 0.80 (0.15, 2.47) | |
| Previous anti-EGFR | | | | 0.030 | | | 0.002 |
| Yes | 60 | 2.1 (1.9, 2.8) | 1 (Reference) | | 8.8+ | 1 (Reference) | |
| No | 78 | 1.9 (1.8, 2.0) | 1.53 (1.02, 2.29) | | 4.4 (3.7, 5.5) | 2.83 (1.38, 5.00) | |

+g value was based on log-rank test in the univariate analysis

TABLE 6
Baseline characteristics and clinical outcome in the control cohort treated with regorafenib

| | | Progression-free Survival | | | Overall Survival | | |
|---|---|---|---|---|---|---|---|
| | | Median, months | | | Median, months | | |
| | N | (95% Cl) | HR (95% Cl) | P value⁺ | (95% Cl) | HR (95% Cl) | P value⁺ |
| Age group | | | | 0.043 | | | 0.27 |
| <61 | 19 | 1.8 (1.4, 2.3) | 1 (Reference) | | 5.1 (2.2, 8.1) | 1 (Reference) | |
| ≥61 | 33 | 2.1 (1.8, 3.4) | 0.58 (0.31, 1.07) | | 5.8 (3.7, 8.0) | 0.74 (0.41, 1.32) | |
| Sex | | | | 0.94 | | | 0.55 |
| Male | 23 | 1.8 (1.7, 3.1) | 1 (Reference) | | 5.4 (2.6, 6.8) | 1 (Reference) | |
| Remale | 29 | 2.0 (1.8, 2.9) | 1.02 (0.58, 1.86) | | 5.5 (3.8, 8.0) | 1.18 (0.67, 2.06) | |
| Performance status | | | | 0.014 | | | 0.029 |
| ECOG 0 | 42 | 2.1 (1.8, 2.9) | 1 (Reference) | | 5.9 (4.5, 8.0) | 1 (Reference) | |
| ECOG 1 | 10 | 1.2 (0.0, 2.2) | 2.27 (1.10, 4.85) | | 2.4 (0.7, 6.0) | 2.09 (1.02, 4.29) | |
| Tumor site | | | | 0.41 | | | 0.45 |
| Right | 22 | 1.8 (1.4, 2.3) | 1 (Reference) | | 3.9 (2.0, 8.0) | 1 (Reference) | |
| Left | 30 | 2.2 (1.8, 3.4) | 0.80 (0.45, 1.40) | | 7.2 (4.5, 9.2) | 0.82 (0.46, 1.46) | |
| Liver metastasis | | | | 0.033 | | | 0.19 |
| Yes | 46 | 1.9 (1.8, 2.2) | 1 (Reference) | | 4.7 (3.8, 7.9) | 1 (Reference) | |
| No | 7 | 4.8 (0.9, 9.1) | 0.48 (0.20, 1.07) | | 8.6 (1.4, 12.8) | 0.51 (0.28, 1.41) | |
| Lung metastasis | | | | 0.52 | | | 0.50 |
| Yes | 40 | 1.9 (1.7, 2.8) | 1 (Reference) | | 6.9 (4.2, 8.0) | 1 (Reference) | |
| No | 12 | 2.0 (1.8, 3.2) | 1.17 (0.61, 2.25) | | 3.7 (1.8, 9.7) | 1.24 (0.66, 2.39) | |
| LN metastasis | | | | 0.011 | | | 0.072 |
| Yes | 27 | 1.8 (1.5, 2.0) | 1 (Reference) | | 3.8 (2.4, 6.0) | 1 (Reference) | |
| No | 25 | 2.8 (1.8, 4.8) | 0.52 (0.29, 0.92) | | 7.7 (4.7, 10.4) | 0.82 (0.35, 1.09) | |
| Peritoneal | | | | 0.64 | | | 0.51 |
| Yes | 15 | 1.9 (1.4, 3.1) | 1 (Reference) | | 5.1 (2.0, 8.1) | 1 (Reference) | |
| No | 37 | 2.0 (1.7, 2.8) | 0.87 (0.47, 1.80) | | 5.8 (3.7, 8.0) | 0.82 (0.44, 1.61) | |
| Number of metastases | | | | 0.018 | | | 0.088 |
| ≤2 | 22 | 2.8 (1.8, 4.8) | 1 (Reference) | | 5.3 (4.8, 10.4) | 1 (Reference) | |
| >2 | 30 | 1.9 (1.7, 2.2) | 1.86 (1.04, 3.32) | | 4.0 (2.6, 7.9) | 1.59 (0.90, 2.80) | |
| Pre-chemonumbers | | | | 0.85 | | | 0.69 |
| <4 | 31 | 2.0 (1.7, 2.9) | 1 (Reference) | | 5.0 (3.8, 8.1) | 1 (Reference) | |
| ≥4 | 21 | 1.8 (1.7, 4.3) | 0.96 (0.64, 1.87) | | 4.7 (2.6, 8.0) | 1.18 (0.88, 2.04) | |
| Adjuvant history | | | | 0.36 | | | 0.16 |
| Yes | 8 | 1.8 (0.8, 3.1) | 1 (Reference) | | 3.9 (0.6, 10.4) | 1 (Reference) | |
| No | 44 | 2.0 (1.8, 2.9) | 0.71 (0.33, 1.53) | | 5.9 (3.7, 8.0) | 0.59 (0.27, 1.26) | |
| Primary tumor resection | | | | 0.58 | | | 0.94 |
| Yes | 46 | 1.9 (1.8, 2.8) | 1 (Reference) | | 5.8 (3.6, 7.9) | 1 (Reference) | |
| No | 8 | 2.0 (0.9, 12.4+) | 0.79 (0.33, 1.87) | | 4.2 (1.4, 19.0+) | 1.04 (0.44, 2.44) | |
| KRAS | | | | 0.85 | | | 0.78 |
| Wildtype | 28 | 1.9 (1.7, 3.4) | 1 (Reference) | | 5.3 (3.7, 8.1) | 1 (Reference) | |
| Mutant | 24 | 2.0 (1.7, 2.8) | 1.05 (0.80, 1.87) | | 4.8 (2.4, 7.9) | 0.93 (0.62, 1.64) | |

+g value was based on log-rank test in the univariate analysis.

### Association of Clinical Outcomes and FTD Metabolism-Related Genetic Variants in Patients Receiving TAS-102

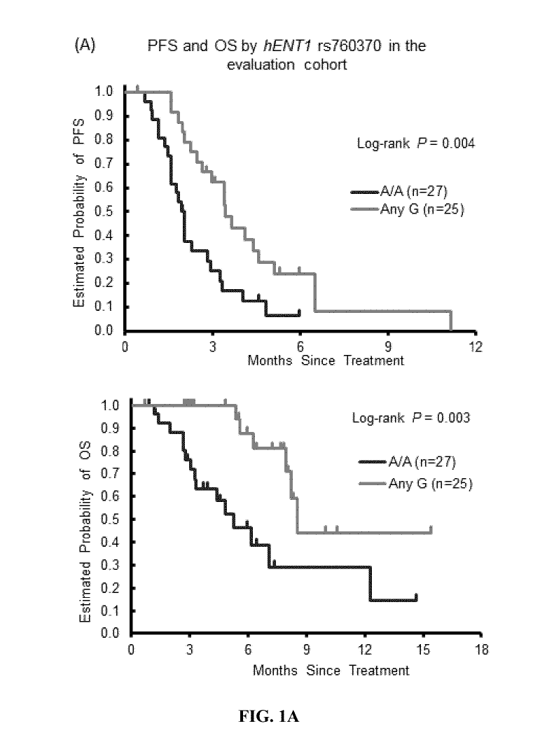

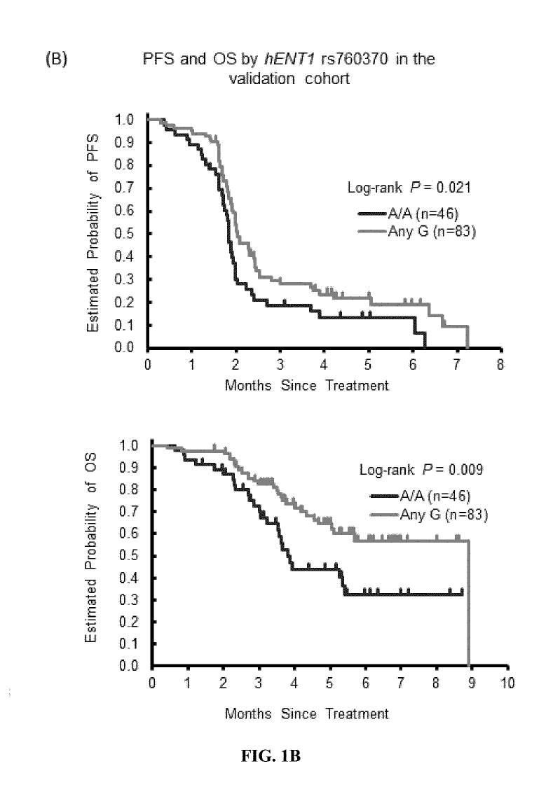

Allelic frequencies and genotype distribution for each SNP were close to those reported for the Caucasian population or Japanese population, and all of candidate SNPs were successfully genotyped in each cohort except for MATE1 rs2289669 in one patients from the training cohort due to low quality of extracted genomic DNA. In univariate analysis for the training cohort, patients carrying any G allele in hENT1 rs760370 had significant longer PFS [3.5 vs. 2.1 months, hazard ratio (HR) 0.44, 95% CI: 0.23-0.83, P=0.004] and OS (8.7 vs. 5.3 months, HR 0.27, 95% CI: 0.10-0.70, P=0.003) than those with the A/A variant (FIG. 1A). Patients with any T allele in hENT1 rs9394992 had significant longer PFS [3.4 vs. 1.9 months, hazard ratio (HR) 0.48, 95% CI: 0.25-0.91, P=0.011] and OS (8.7 vs. 4.4 months, HR 0.21, 95% CI: 0.08-0.51, P<0.001) than those with the C/C variant. In multivariate analysis adjusted for liver metastases and adjuvant history, both hENT1 rs760370 and hENT1 rs9394992 showed statistically significance in PFS (HR 0.44, P=0.023 and HR0.44, P=0.016) and OS (HR 0.27, P=0.003 and HR0.17, P=0.008), respectively. In univariate analysis for the testing cohort, patients carrying any G allele in hENT1 rs760370 had significant longer PFS [2.1 vs. 1.9 months, hazard ratio (HR) 0.64, 95% CI: 0.43-0.95, P=0.021] and OS (9.0 vs. 3.9 months, HR 0.50, 95% CI: 0.29-0.86, P=0.009) than those with the A/A variant (FIG. 1B). In multivariable analysis adjusted for adjusted for age group (≤60 vs. >60), liver metastasis, ECOG performance status and previous anti-EGFR, hENT1 rs760370 remained significantly associated with PFS (HR 0.65, 95% CI: 0.43-0.98, P=0.038) and OS (HR 0.54, 95% CI: 0.31-0.93, P=0.027), respectively (Table 3, Table 7).

TABLE 3
Association between gene polymorphism and clinical outcome

| | | Disease Control Rate | | | Progression-free Survival | | | | | Overall Survival | | | | |
|---|---|---|---|---|---|---|---|---|---|---|---|---|---|---|
| | | | | | Median, | | | | | Median, | | | | |
| | | | | P | months | HR | P | HR | P | months | HR | P | HR | P |
| | N | SD | PD | value* | (95% Cl) | (95% Cl)⁺ | value* | (95% Cl)^{‡} | value* | (95% Cl) | (95% Cl)⁺ | value* | (95% Cl)^{‡} | value* |
| Training cohort | | | | | | | | | | | | | | |
| hENT1 | | | | 0.051 | | | 0.004 | | 0.023 | | | 0.003 | | 0.008 |
| rs760370 | | | | | | | | | | | | | | |
| A/A | 27 | 4 (17%) | 19 (83%) | | 2.1 (1.6, 2.9) | 1 (Reference) | | 1 (Reference) | | 5.3 (3.1, 12.4) | 1 (Reference) | | 1 (Reference) | |
| A/G* | 22 | 9 (45%) | 11 (55%) | | 3.5 (2.5, 4.6) | 0.44 (0.23, 0.83) | | 0.44 (0.22, 0.89) | | 8.7 (8.1, 15.6+) | 0.27 (0.10, 0.70) | | 0.17 (0.05, 0.63) | |
| G/G* | 3 | 2 (67%) | 1 (33%) | | | | | | | | | | | |
| hENT1 | | | | 0.071 | | | 0.011 | | 0.016 | | | <0.001 | | 0.001 |
| rs9394992 | | | | | | | | | | | | | | |
| C/C | 21 | 3 (17%) | 15 (83%) | | 1.9 (1.6, 2.7) | 1 (Reference) | | 1 (Reference) | | 4.4 (2.7, 7.2) | 1 (Reference) | | 1 (Reference) | |
| C/T* | 25 | 8 (36%) | 14 (64%) | | 3.4 (2.3, 4.4) | 0.48 (0.25, 0.91) | | 0.44 (0.23, 0.86) | | 8.7 (8.1, 15.6+) | 0.21 (0.08, 0.51) | | 0.20 (0.08, 0.51) | |
| T/T* | 6 | 4 (67%) | 2 (33%) | | | | | | | | | | | |
| MATE1 | | | | 0.091 | | | 0.035 | | 0.046 | | | 0.095 | | 0.19 |
| rs2289669 | | | | | | | | | | | | | | |
| G/G | 12 | 1 (10%) | 9 (90%) | | 2.0 (1.4, 3.4) | 1 (Reference) | | 1 (Reference) | | 8.1 (5.4, 10.7+) | 1 (Reference) | | 1 (Reference) | |
| G/A | 28 | 12 (45%) | 14 (54%) | | 3.4 (2.1, 4.4) | 0.44 (0.20, 0.93) | | 0.41 (0.18, 0.94) | | 12.4 (5.7, 15.6+) | 0.75 (0.26, 2.28) | | 0.59 (0.23, 2.11) | |
| A/A | 12 | 2 (20%) | 8 (80%) | | 2.3 (1.2, 3.3) | 0.94 (0.40, 2.18) | | 0.92 (0.38, 2.24) | | 4.9 (2.8, 6.5+) | 2.40 (0.59, 8.37) | | 1.98 (0.56, 6.99) | |
| | | | | 0.13 | | | 0.055 | | 0.10 | | | 0.96 | | 0.88 |
| G/G | 12 | 1 (10%) | 9 (90%) | | 2.0 (1.4, 3.4) | 1 (Reference) | | 1 (Reference) | | 8.1 (5.4, 10.7+) | 1 (Reference) | | 1 (Reference) | |
| Any A | 40 | 14 (39%) | 22 (61%) | | 3.0 (2.1, 3.7) | 0.56 (0.28, 1.13) | | 0.53 (0.25, 1.13) | | 8.3 (5.3, 15.6+) | 1.03 (0.37, 2.83) | | 0.93 (0.33, 2.59) | |
| OCT2 | | | | 0.16 | | | 0.40 | | 0.50 | | | 0.93 | | 0.35 |
| rs316019 | | | | | | | | | | | | | | |
| C/C | 37 | 9 (27%) | 24 (73%) | | 2.5 (2.1, 3.4) | 1 (Reference) | | 1 (Reference) | | 8.1 (5.7, 12.4) | 1 (Reference) | | 1 (Reference) | |
| C/A* | 11 | 3 (33%) | 6 (67%) | | 3.0 (1.2, 6.0+) | 0.74 (0.35, 1.52) | | 1.32 (0.58, 3.02) | | 10.7+ (2.7, 10.7+) | 1.04 (0.38, 2.89) | | 1.74 (0.55, 5.51) | |
| A/A* | 4 | 3 (75%) | 1 (25%) | | | | | | | | | | | |
| MATE1_OCT019 | | | | 0.070 | | | 0.006 | | 0.020 | | | 0.026 | | 0.060 |
| poor | 15 | 1 (8%) | 11 (92%) | | 2.3 (1.2, 2.9) | 1 (Reference) | | 1 (Reference) | | 4.9 (2.8, 10.7) | 1 (Reference) | | 1 (Reference) | |
| good | 37 | 14 (41%) | 20 (59%) | | 3.4 (2.1, 4.1) | 0.44 (0.22, 0.85) | | 0.45 (0.23, 0.88) | | 8.3 (6.3, 15.6) | 0.39 (0.15, 0.98) | | 0.41 (0.16, 1.04) | |
| hENT1rs750370_19 | | | | 0.044 | | | <0.001 | | 0.016 | | | <0.001 | | 0.009 |
| Poor | 11 | 0 | 9 (100%) | | 1.6 (0.9, 2.9) | 1 (Reference) | | 1 (Reference) | | 3.4 (2.7, 6.5) | 1 (Reference) | | 1 (Reference) | |
| Faire | 16 | 4 (29%) | 10 (71%) | | 2.1 (1.6, 4.1) | 0.42 (0.17, 1.03) | | 0.48 (0.20, 1.16) | | 7.2 (2.7, 14.8) | 0.37 (0.12, 1.16) | | 0.37 (0.11, 1.23) | |
| Good* | 4 | 1 (33%) | 2 (67%) | | 3.5 (2.5, 4.6) | 0.26 (0.11, 0.59) | | 0.28 (0.11, 0.55) | | 8.7 (8.1, 15.6) | 0.14 (0.04, 0.48) | | 0.08 (0.02, 0.41) | |
| Excellent* | 21 | 10 (50%) | 10 (50%) | | | | | | | | | | | |
| Testing cohort | | | | | | | | | | | | | | |
| hENT1 | | | | 0.24 | | | 0.030 | | 0.029 | | | 0.032 | | 0.054 |
| rs750370 | | | | | | | | | | | | | | |
| A/A | 46 | 9 (20%) | 36 (80%) | | 1.9 (1.7, 2.0) | 1 (Reference) | | 1 (Reference) | | 3.9 (3.3, 5.5) | 1 (Reference) | | 1 (Reference) | |
| A/G | 64 | 22 (34%) | 42 (66%) | | 2.1 (1.9, 2.5) | 0.59 (0.38, 0.90) | | 0.58 (0.37, 0.89) | | 8.7+ (4.7, 8.7+) | 0.48 (0.27, 0.85) | | 0.50 (0.28, 0.89) | |
| G/G | 19 | 6 (32%) | 13 (68%) | | 2.0 (1.6, 2.5) | 0.86 (0.48, 1.55) | | 1.00 (0.55, 1.80) | | 9.0 (2.9, 9.0+) | 0.58 (0.24, 1.41) | | 0.78 (0.31, 1.99) | |
| | | | | 0.15 | | | 0.021 | | 0.038 | | | 0.009 | | 0.027 |
| A/A | 46 | 9 (20%) | 36 (80%) | | 1.9 (1.7, 2.0) | 1 (Reference) | | 1 (Reference) | | 3.9 (3.3, 5.5) | 1 (Reference) | | 1 (Reference) | |
| Any G | 83 | 28 (34%) | 55 (66%) | | 2.1 (1.9, 2.4) | 0.64 (0.43, 0.95) | | 0.65 (0.43, 0.98) | | 9.0 (5.1, 9.0+) | 0.50 (0.29, 0.86) | | 0.54 (0.31, 0.93) | |
| hENT1 | | | | 1.00 | | | 0.90 | | 0.56 | | | 0.51 | | 0.76 |
| rs9394992 | | | | | | | | | | | | | | |
| C/C | 58 | 17 (29%) | 41 (71%) | | 2.0 (1.9, 2.1) | 1 (Reference) | | 1 (Reference) | | 5.8 (4.7, 8.8+) | 1 (Reference) | | 1 (Reference) | |
| C/T | 59 | 17 (29%) | 41 (71%) | | 2.0 (1.8, 2.3) | 0.92 (0.61, 1.38) | | 0.81 (0.54, 1.22) | | 4.4 (3.6, 9.0+) | 1.38 (0.78, 2.43) | | 1.23 (0.69, 2.18) | |
| T/T | 12 | 3 (25%) | 9 (75%) | | 2.5 (0.4, 3.9) | 0.90 (0.45, 1.78) | | 0.79 (0.39, 1.58) | | 7.3+ (2.5, 7.3+) | 1.06 (0.40, 2.79) | | 1.02 (0.38, 2.71) | |
| | | | | 1.00 | | | 0.65 | | 0.25 | | | 0.32 | | 0.54 |
| C/C | 58 | 17 (29%) | 41 (71%) | | 2.0 (1.9, 2.1) | 1 (Reference) | | 1 (Reference) | | 5.8 (4.7, 8.8+) | 1 (Reference) | | 1 (Reference) | |
| Any T | 71 | 20 (29%) | 50 (71%) | | 2.0 (1.9, 2.4) | 0.92 (0.62, 1.35) | | 0.81 (0.54, 1.19) | | 5.2 (3.7, 9.0+) | 1.31 (0.76, 2.27) | | 1.19 (0.69, 2.07) | |
| MATE1 | | | | 0.90 | | | 0.98 | | 0.53 | | | 0.34 | | 0.32 |
| rs2289659 | | | | | | | | | | | | | | |
| G/G | 36 | 10 (27%) | 27 (73%) | | 1.9 (1.9, 2.4) | 1 (Reference) | | 1 (Reference) | | 9.0 (4.4, 9.0+) | 1 (Reference) | | 1 (Reference) | |
| G/A | 54 | 20 (31%) | 44 (69%) | | 2.0 (1.8, 2.3) | 1.04 (0.66, 1.64) | | 1.15 (0.73, 1.53) | | 4.6 (3.7, 8.8+) | 1.55 (0.81, 2.96) | | 1.67 (0.56, 3.25) | |
| A/A | 27 | 7 (26%) | 20 (74%) | | 2.0 (1.7, 2.3) | 1.01 (0.58, 1.74) | | 1.11 (0.63, 1.96) | | 5.5+ (3.7, 8.5+) | 1.13 (0.50, 2.58) | | 1.42 (0.61, 3.32) | |
| | | | | 0.83 | | | 0.87 | | 0.55 | | | 0.25 | | 0.15 |
| G/G | 38 | 10 (27%) | 27 (73%) | | 1.9 (1.9, 2.4) | 1 (Reference) | | 1 (Reference) | | 9.0 (4.4, 9.0+) | 1 (Reference) | | 1 (Reference) | |
| Any A | 91 | 27 (30%) | 64 (70%) | | 2.0 (1.9, 2.1) | 1.03 (0.68, 1.58) | | 1.14 (0.74, 1.75) | | 5.3 (3.9, 8.8+) | 1.42 (0.76, 2.64) | | 1.60 (0.85, 3.04) | |
| OCT2 | | | | 0.65 | | | 0.60 | | 0.31 | | | 0.57 | | 1.00 |
| rs316019 | | | | | | | | | | | | | | |
| C/C | 98 | 27 (28%) | 70 (72%) | | 2.0 (1.9, 2.3) | 1 (Reference) | | 1 (Reference) | | 5.5 (4.7, 9.0+) | 1 (Reference) | | 1 (Reference) | |
| C/A | 31 | 10 (32%) | 21 (68%) | | 1.9 (1.7, 2.4) | 1.12 (0.72, 1.76) | | 1.28 (0.79, 2.07) | | 4.6 (3.6, 7.1+) | 1.19 (0.64, 2.19) | | 1.00 (0.53, 1.89) | |
| MATE1_OCT019 | | | | 0.83 | | | 0.77 | | 0.68 | | | 0.59 | | 0.73 |
| Poor | 36 | 10 (26%) | 28 (74%) | | 2.0 (1.8, 2.1) | 1 (Reference) | | 1 (Reference) | | 8.5+ (3.6, 8.5+) | 1 (Reference) | | 1 (Reference) | |
| Good | 91 | 27 (30%) | 63 (70%) | | 2.0 (1.9, 2.3) | 0.94 (0.62, 1.43) | | 0.91 (0.59, 1.42) | | 5.4 (4.1, 9.0+) | 1.18 (0.64, 2.17) | | 1.12 (0.59, 2.10) | |
| hENT1370_OCT019 | | | | 0.41 | | | 0.53 | | 0.11 | | | 0.025 | | 0.032 |
| Poor | 14 | 3 (21%) | 11 (79%) | | 2.0 (1.7, 2.4) | 1 (Reference) | | 1 (Reference) | | 3.6 (2.3, 8.5) | 1 (Reference) | | 1 (Reference) | |
| Fair | 32 | 6 (19%) | 25 (81%) | | 1.9 (1.6, 2.0) | 1.24 (0.64, 2.40) | | 1.13 (0.57, 2.24) | | 4.0 (3.1, 8.8) | 0.77 (0.34, 1.75) | | 0.58 (0.25, 1.37) | |
| Good* | 24 | 7 (29%) | 17 (71%) | | 2.1 (1.9, 2.4) | 0.73 (0.40, 1.34) | | 0.71 (0.38, 1.30) | | 9.0 (5.1, 9.0) | 0.42 (0.20, 0.90) | | 0.37 (0.17, 0.80) | |
| Excellent* | 59 | 21 (36%) | 38 (64%) | | | | | | | | | | | |
| Control cohort | | | | | | | | | | | | | | |
| hENT1 | | | | 0.93 | | | 0.71 | | 0.68 | | | 0.77 | | 0.95 |
| rs760370 | | | | | | | | | | | | | | |
| A/A | 21 | 7 (35%) | 15 (65%) | | 2.1 (1.8, 3.1) | 1 (Reference) | | 1 (Reference) | | 5.9 (4.5, 8.0) | 1 (Reference) | | 1 (Reference) | |
| A/G | 19 | 7 (39%) | 11 (61%) | | 1.9 (1.6, 3.9) | 1.26 (0.67, 2.37) | | 1.08 (0.57, 2.08) | | 5.7 (2.0, 8.9) | 1.25 (0.67, 2.35) | | 0.95 (0.50, 1.90) | |
| G/G | 12 | 3 (27%) | 8 (73%) | | 1.4 (0.4, 4.3) | 1.24 (0.59, 2.59) | | 1.57 (0.57, 4.35) | | 2.4 (1.2, 9.1) | 1.17 (0.55, 2.46) | | 1.11 (0.48, 2.56) | |
| | | | | 1.00 | | | 0.41 | | 0.67 | | | 0.48 | | 0.97 |
| A/A | 21 | 7 (35%) | 13 (65%) | | 2.1 (1.8, 3.1) | 1 (Reference) | | 1 (Reference) | | 5.9 (4.5, 5.0) | 1 (Reference) | | 1 (Reference) | |
| Any G | 31 | 10 (34%) | 19 (66%) | | 1.8 (1.5, 2.2) | 1.25 (0.71, 2.20) | | 1.15 (0.61, 2.15) | | 4.1 (2.2, 7.8 ) | 1.22 (0.70, 2.13) | | 1.01 (0.55, 1.88) | |
| hENT | | | | 0.30 | | | 0.29 | | 0.62 | | | 0.039 | | 0.22 |
| rs9394992 | | | | | | | | | | | | | | |
| C/C | 20 | 6 (30%) | 14 (70%) | | 2.0 (1.2, 2.7) | 1 (Reference) | | 1 (Reference) | | 4.4 (2.6, 6.0) | 1 (Reference) | | 1 (Reference) | |
| C/T* | 25 | 7 (30%) | 16 (70%) | | 1.9 (1.7, 3.9) | 0.75 (0.42, 1.32) | | 0.85 (0.46, 1.59) | | 7.7 (3.7, 8.9) | 0.57 (0.31, 1.05) | | 0.64 (0.32, 1.30) | |
| T/T* | 7 | 4 (67%) | 2 (33%) | | | | 0.56 | | 0.66 | | | 0.65 | | 0.95 |
| MATE1 | | | | 0.44 | | | | | | | | | | |
| rs2289669 | | | | | | | | | | | | | | |
| G/G | 19 | 7 (41%) | 10 (59%) | | 2.1 (1.5, 3.1) | 1 (Reference) | | 1 (Reference) | | 5.7 (2.2, 7.8) | 1 (Reference) | | 1 (Reference) | |
| G/A* | 27 | 7 (27%) | 19 (73%) | | 1.8 (1.7, 2.8) | 0.65 (0.48, 1.52) | | 0.86 (0.43, 1.70) | | 5.3 (3.7, 8.0) | 0.85 (0.50, 1.57) | | 0.99 (0.52, 1.91) | |
| A/A* | 6 | 3 (50%) | 5 (50%) | | | | 0.99 | | 0.36 | | | 0.51 | | 0.81 |
| OCT2 | | | | 0.49 | | | | | | | | | | |
| rs316019 | | | | | | | | | | | | | | |
| C/C | 44 | 16 (39%) | 25 (61%) | | 2.1 (1.7, 3.2) | 1 (Reference) | | 1 (Reference) | | 3.3 (2.6, 7.9) | 1 (Reference) | | 1 (Reference) | |
| C/A* | 7 | 1 (14%) | 6 (86%) | | 1.8 (1.5, 2.0) | 1.00 (0.44, 2.25) | | 1.50 (0.62, 3.61) | | 5.3 (3.6, 9.6) | 0.79 (0.35, 1.75) | | 0.90 (0.39, 2.09) | |
| A/A* | 1 | 0 | 1 (100%) | | | | | | | | | | | |

*P value was based on the Fisher's exact test for tumor response, log-rank test in the univariate analysis (+) and Wald test in the multivariate analysis within Cox regression model adjusted for liver metastasis and adjuvant history in training cohort; age group (<61 vs ≥53), liver metastasis, ECOG performance status, previous anti-EGFR ( [symbol] ) in testing cohort; ECOG performance.
+Estimates not reached yet. [symbol]
Combined for estimates of HR. [symbol]
indicates data missing or illegible when filed

TABLE 7
Gene polymorphism and clinical outcome in the training, testing and control cohorts

| | | Disease control | | | Profression-free Survival | | | | |
|---|---|---|---|---|---|---|---|---|---|
| | | | | | Median, | | | | |
| | | | | P | months | HR | P | HR | P |
| | N | SD | PD | value* | (95% Cl) | (95% Cl) + | value* | (95% Cl) ‡ | value* |
| Training | | | | | | | | | |
| cohort | | | | | | | | | |
| TK1 | | | | 0.53 | | | 0.26 | | 0.72 |
| rs1065769 | | | | | | | | | |
| C/C | 29 | 10 (37%) | 17 (63%) | | 3.2 (2.1, 3.7) | 1 (Reference) | | 1 (Reference) | |
| C/T^{a} | 22 | 5 (26%) | 14 (74%) | | 2.3 (1.6, 3.5) | 1.39 (0.75, 2.58) | | 1.13 (0.58, 2.18) | |
| T/T^{a} | 1 | | | | | | | | |
| CNT1 | | | | 0.17 | | | 0.69 | | 0.39 |
| rs2290272 | | | | | | | | | |
| G/G | 15 | 4 (33%) | 8 (67%) | | 2.3 (1.2, 4.6) | 1 (Reference) | | 1 (Reference) | |
| G/A | 28 | 11 (41%) | 16 (59%) | | 3.4 (1.8, 3.7) | 0.89 (0.42, 1.89) | | 0.64 (0.29, 1.39) | |
| A/A | 8 | 0 | 6 (100%) | | 2.8 (2.1, 3.3) | 1.25 (0.48, 3.28) | | 1.01 (0.38, 2.68) | |
| | | | | 1.00 | | | 0.91 | | 0.37 |
| G/G | 15 | 4 (33%) | 8 (67%) | | 2.3 (1.2. 4.6) | 1 (Reference) | | 1 (Reference) | |
| any A | 36 | 11 (33%) | 22 (67%) | | 2.9 (2.1, 3.4) | 0.96 (0.47, 1.98) | | 0.71 (0.33, 1.50) | |
| MATE2- | | | | 0.12 | | | 0.59 | | 0.85 |
| rs16960360 | | | | | | | | | |
| A/A | 23 | 7 (35%) | 13 (65%) | | 3.0 (1.8, 3.5) | 1 (Reference) | | 1 (Reference) | |
| A/C^{b} | 25 | 5 (23%) | 17 (77%) | | 2.5 (1.8, 4.4) | 0.85 (0.46, 1.58) | | 1.07 (0.55, 2.07) | |
| C/C^{b} | 4 | 3 (75%) | 1 (25%) | | | | | | |
| OCT2 | | | | 0.23 | | | 0.30 | | 0.80 |
| rs316000 | | | | | | | | | |
| A/A | 38 | 9 (26%) | 25 (74%) | | 2.3 (2.0, 3.4) | 1 (Reference) | | 1 (Reference) | |
| A/G^{c} | 8 | 2 (33%) | 4 (67%) | | 3.0 (1.2, 6.0+) | 0.69 (0.33, 1.45) | | 1.11 (0.48, 2.56) | |
| G/G^{c} | 6 | 4 (67%) | 2 (33%) | | | | | | |
| Testing | | | | | | | | | |
| cohort | | | | | | | | | |
| OCT2 | | | | 0.18 | | | 0.33 | | 0.33 |
| rs316000 | | | | | | | | | |
| A/A | 84 | 21 (25%) | 62 (75%) | | 2.0 (1.9, 2.1) | 1 (Reference) | | 1 (Reference) | |
| A/G | 40 | 13 (33%) | 27 (68%) | | 1.9 (1.7, 2.4) | 0.94 (0.62, 1.44) | | 1.05 (0.66, 1.67) | |
| G/G | 5 | 3 (60%) | 2 (40%) | | 3.7 (1.7, 6.8+) | 0.43 (0.14, 1.38) | | 0.41 (0.12, 1.38) | |
| | | | | 0.23 | | | 0.45 | | 0.73 |
| A/A | 84 | 21 (25%) | 62 (75%) | | 2.0 (1.9, 2.1) | 1 (Reference) | | 1 (Reference) | |
| Any G | 45 | 16 (36%) | 29 (64%) | | 1.9 (1.8, 2.6) | 0.85 (0.5, 1.29) | | 0.93 (0.59, 1.45) | |
| Control | | | | | | | | | |
| cohort | | | | | | | | | |
| OCT2 | | | | 0.67 | | | | | |
| rs316000 | | | | | | | | | |
| A/A | 39 | 14 (39%) | 22 (61%) | | | | | | |
| A/G | 12 | 3 (25%) | 9 (75%) | | | | | | |
| G/G | 1 | 0 | 1 (100%) | | | | | | |
| | | | | 0.50 | | | 0.88 | | 0.70 |
| G/G | 39 | 14 (39%) | 22 (61%) | | 2.1 (1.7, 3.1) | 1 (Reference) | | 1 (Reference) | |
| Any A | 13 | 3 (23%) | 10 (77%) | | 1.8 (1.6, 2.8) | 0.95 (0.50, 1.83) | | 0.88 (0.45, 1.70) | |
| | | | | | Overall Survival | | | | |
| | | | | | Median, | | | | |
| | | | | | months | HR | P | HR | P |
| | | | | | (95% Cl) | (95% Cl) + | value* | (95% Cl) ‡ | value* |
| | | | | Training | | | | | |
| | | | | cohort | | | | | |
| | | | | TK1 | | | 0.39 | | 0.20 |
| | | | | rs1065769 | | | | | |
| | | | | C/C | 7.2 (5.3, 15.6) | 1 (Reference) | | 1 (Reference) | |
| | | | | C/T^{a} | 12.4 (4.9, 12.4) | 0.68 (0.27, 1.69) | | 0.54 (0.21, 1.38) | |
| | | | | T/T^{a} | | | | | |
| | | | | CNT1 | | | 0.61 | | 0.77 |
| | | | | rs2290272 | | | | | |
| | | | | G/G | 15.6+ (2.8, 15.6+) | 1 (Reference) | | 1 (Reference) | |
| | | | | G/A | 8.1 (5.3, 8.7) | 1.57 (0.50, 4.97) | | 1.34 (0.42, 4.28) | |
| | | | | A/A | 7.2 (3.3, 12.4+) | 1.89 (0.50, 7.07) | | 1.65 (0.43, 6.34) | |
| | | | | | | | 0.35 | | 0.54 |
| | | | | G/G | 15.6+ (2.8, 15.6+) | 1 (Reference) | | 1 (Reference) | |
| | | | | any A | 8.1 (5.3, 8.7) | 1.65 (0.55, 4.96) | | 1.42 (0.46, 4.34) | |
| | | | | MATE2- | | | 0.93 | | 0.80 |
| | | | | rs16960360 | | | | | |
| | | | | A/A | 8.7 (5.4, 14.8+) | 1 (Reference) | | 1 (Reference) | |
| | | | | A/C^{b} | 8.1 (4.9, 15.6+) | 1.04 (0.44, 2.48) | | 1.12 (0.47, 2.68) | |
| | | | | C/C^{b} | | | | | |
| | | | | OCT2 | | | 0.65 | | 1.00 |
| | | | | rs316000 | | | | | |
| | | | | A/A | 8.1 (5.3, 12.4) | 1 (Reference) | | 1 (Reference) | |
| | | | | A/G^{c} | 10.7+ (2.7, 10.7+) | 0.78 (0.26, 2.34) | | 0.99 (0.31, 3.17) | |
| | | | | G/G^{c} | | | | | |
| | | | | Testing | | | | | |
| | | | | cohort | | | | | |
| | | | | OCT2 | | | 0.67 | | 0.41 |
| | | | | rs316000 | | | | | |
| | | | | A/A | 5.5 (3.9, 9.0+) | 1 (Reference) | | 1 (Reference) | |
| | | | | A/G | 5.8 (4.2, 7.1+) | 0.87 (0.48, 1.57) | | 0.66 (0.36, 1.23) | |
| | | | | G/G | 7.0+ (3.6, 7.0+) | 0.45 (0.06, 3.28) | | 0.64 (0.08, 4.91) | |
| | | | | | | | 0.51 | | 0.18 |
| | | | | A/A | 5.5 (3.9, 9.0+) | 1 (Reference) | | 1 (Reference) | |
| | | | | Any G | 5.8 (4.2, 7.1+) | 0.83 (0.46, 1.47) | | 0.66 (0.36, 1.21) | |
| | | | | Control | | | | | |
| | | | | cohort | | | | | |
| | | | | OCT2 | | | | | |
| | | | | rs316000 | | | | | |
| | | | | A/A | | | | | |
| | | | | A/G | | | | | |
| | | | | G/G | | | | | |
| | | | | | | | 0.88 | | 0.54 |
| | | | | G/G | 5.7 (2.6, 8.0) | 1 (Reference) | | 1 (Reference) | |
| | | | | Any A | 4.7 (3.6, 7.6) | 0.95 (0.50, 1.83) | | 0.80 (0.40, 1.61) | |

*P value was based on the Fisher's exact test for tumor response, the log-rank test in the univariate analysis and Wald test in the multivariate analysis within Cox regression model.
‡ Adjusted for liver metastasis and adjuvant history in training cohort; age group (<61 vs ≥61), liver metastasis, ECOG performance status, previous anti-EGFR in testing cohort; ECOG performance status and number of metastases in control cohort.
^{a, b, c}Combined for estimates of HR.

### Association of Clinical Outcomes and Genetic Variants of TPI Excretion-Related Transporters in Patients Receiving TAS-102

Allelic frequencies and genotype distribution in each SNPs were close to those reported for the Caucasian population, and the all candidate SNPs was successfully genotyped in each cohort. In univariate analysis, patients carrying the G/A allele in MATE1 rs2289669 had significant longer PFS (3.4 vs. 2.0 months, HR 0.44, 95% CI: 0.20-0.93, P=0.035) and marginal significant longer OS (12.4 vs. 8.1 months, HR 0.76, 95% CI: 0.26-2.28, P=0.096) than those with the G/G variant. In multivariate analysis adjusted for liver metastases and adjuvant history, MATE1 rs2289669 G/A variant was remained significant in PFS (HR 0.41, 95% CI: 0.18-0.94, P=0.046). No significant difference was observed in OCT2 rs316019 and OCT2 rs316000. In the testing cohort, there was no evidence of significance in both MATE1 and OCT2 gene polymorphisms (Table 3).

### A Signature Based on Gene-Gene Interaction of OCT2 and MATE1 in TPI Excretion

In the absence of enough evidence that MATE1 and OCT2 genes impacted on clinical outcome, that there might be a gene-gene interaction between MATE1 and OCT2 genes influencing on altered pharmacokinetic and pharmacodynamic response to TPI, as reported in a recent study for metformin [15,16] was hypothesized. In developing a signature, if the categorization of OCT2 rs316019/MATE1 rs225281 shown in FIG. 2A could be adapted to TPI was considered. In the results, patients carrying the G/A allele in MATE1 rs2289669 had significant longer PFS compared to those carrying the G/G or A/A variants. Should patients with the G/A variant in MATE1 rs2289669 carry low CL_{sec}, the results are reasonable based on the concept that high CL_{sec} parallels drug excretion. In addition, no significant difference between C/C wild type and C/A heterozygote in OCT2 rs316019 also sustains existence of gene-gene interaction with MATE1 rs2289669. Taken together with the previous reports, it is reasonable to follow the classification based on gene-gene interaction between OCT2 rs316019 and MATE1 rs225281 to assess the clinical outcome in patients treated with TAS-102. The patients were divided in the training cohorts into two categories as described above in the previous study, and decided to use good clinical outcome (Good: longer PFS or OS) and poor clinical outcome (Poor: shorter PFS or OS) instead of ‘LC’ and ‘HC’ that represents more clearly clinical value of the categories. In addition, The assigned AA/AA variant in OCT2 rs316019/MATE1 rs2289669 into the Good category based on the outcome of a patient with the variant in training cohort (FIG. 2B) was decided.

In univariate analysis for the training cohort, combination of MATE1 rs2289669 and OCT2 rs316019 gene variants showed that patients in Good category had significant longer PFS [3.4 vs. 2.3 months, hazard ratio (HR) 0.44, 95% CI: 0.22-0.85, P=0.006] and OS (8.3 vs. 4.9 months, HR 0.39, 95% CI: 0.15-0.98, P=0.026) than those in Poor category. In multivariable analysis adjusted for liver metastases and adjuvant history, the combination of MATE1 rs2289669 and OCT2 rs316019 showed statistically significance in PFS (HR 0.45, 95% CI: 0.23-0.88, P=0.020) and marginal significance in OS (HR 0.41, 95% CI: 0.16-1.04, P=0.060). In the testing cohort, the combination of MATE1 rs2289669 and OCT2 rs316019 did not show significance in PFS and OS (Table 3).

A Novel Classification Composed of hENT1 and OCT2/MATE1 Gene Polymorphisms with Clinical Outcome

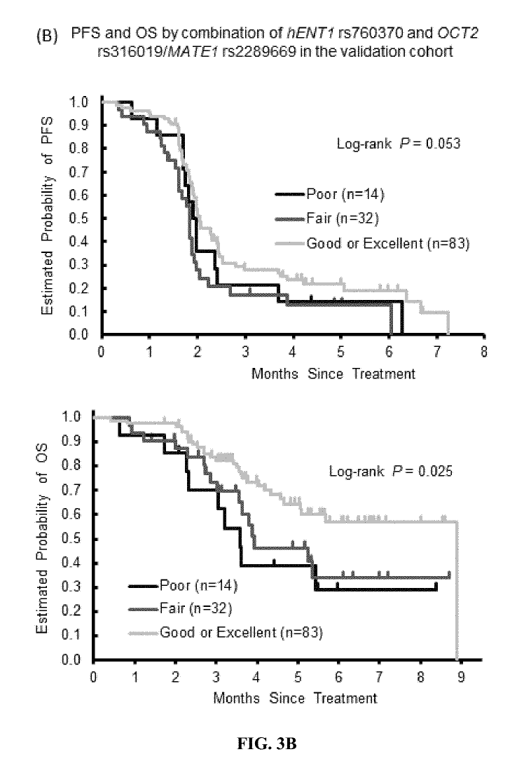

Based upon the results obtained regarding the two different types of transporter involved in metabolism of FTD or TPI, a combination of hENT1 rs760370, OCT2 rs316019 and MATE1 rs2289669 variants might predict overall efficacy of TAS-102 was tested. Based on the individual results of hENT1 rs760370 and combination of MATE1 rs2289669 and OCT2 rs316019 for outcome, the newly defined four categories combining these genes: hENT1 rs760370/OCT2 rs316019 and MATE1 rs2289669 combination: Excellent (n=21), any G allele/Good; Good (n=4), any G allele/Poor; Fair (n=16), A/A variant/Good; and Poor (n=11), A/A variant/Poor. Furthermore, the integrated categories Good and Excellent into one category for analysis to increase the number of sample size (FIG. 2C). PFS, OS and frequency of grade 2≤neutropenia were mentioned in each category. In univariate analysis for the training cohort, this novel classification could clearly recognize the benefit on PFS and OS among the three categories (Poor vs. Fair vs. Good or Excellent: PFS, 1.6 vs. 2.1 vs. 3.5 months, P<0.001; OS, 8.7 vs. 7.2 vs. 3.4, P<0.001) (Table 3, FIG. 3A). This significance remained in multivariate analysis for PFS (P=0.016) and OS (P=0.009). In the testing cohort, these findings were confirmed in OS (3.6 vs. 4.0 vs. 9.0 months, P=0.025) and PFS (2.0 vs. 1.9 vs. 2.1 months, P=0.053). In multivariate analysis, the significance remained in OS (P=0.032) (Table 3, FIG. 3B).

### Gene Polymorphism in FTD and TPI Related Pathway and Clinical Outcomes in the Control Cohort

In the control cohort receiving regorafenib without previous TAS-102 treatment, genotyping for the all candidate SNPs was valid for analysis in all patients. Uni- and multivariate analyses showed no significant differences among the SNPs in PFS or OS (Table 3, Table 7).

SNPs and Toxicity with Clinical Outcomes

As TAS-102-related toxicities, neutropenia, anemia, anorexia, nausea and diarrhea were analyzed for association with clinical outcomes. In univariate analysis for the training cohort, grade 3≤neutropenia (n=20) was marginally associated with longer PFS and OS compared to that less than grade 3 (n=32) (PFS, 3.4 vs. 2.1 months, HR 0.68, 95% CI: 0.37-1.28, P=0.213; OS, 8.3 vs. 6.3 months, HR 0.59, 95% CI: 0.24-1.43, P=0.222). These findings were confirmed in the testing cohort showing statistically significance in PFS (2.3 vs. 1.9 months, HR 0.50, 95% CI: 0.32-0.77, P<0.001) and OS (8.8 vs. 3.7 months, HR 0.21, 95% CI: 0.10-0.44, P<0.001). There was no significant association between SNPs and grade 3≤neutropenia. The correlation between SNPs and grade 2<neutropenia (n=32) in the training cohort and found that grade 2<neutropenia (n=20) was significantly associated with longer OS compared to grade 2>neutropenia (8.3 vs. 4.4 months, HR 0.41, 95% CI: 0.17-0.98, P=0.028) has been analyzed. Gr2<neutropenia was more frequent in patients with the any G allele (n=20/25, 80%) in hENT1 rs760370 compared to those with the A/A variant (n=12/27, 44%).

### Mediation Analysis Between SNPs, Toxicity, and Outcome

In the training cohort, both hENT1 rs760370 and hENT1 rs9394992 showed significant association with clinical outcome, as well as Gr2≤neutropenia; additionally, Gr2<neutropenia was significantly associated with OS. Therefore, mediation analysis was constructed to examine if Gr2<neutropenia served as a mediator in the pathway of hENT1 SNPs predicting OS. The results were summarized in Table 8. In hENT1 rs760370 model, the total effect of 0.16 (95% CI: 0.04-0.57) was decomposed into direct effect of 0.22 (95% CI: 0.06-0.77) and indirect effect of 0.73 (0.46-1.14), and the proportional mediated by Gr2<neutropenia was 7.1%. In hENT1 rs9394992 model, total effect was 0.17 (95% CI: 0.06-0.46), which was decomposed into direct effect of 0.23 (95% CI: 0.09-0.60) and indirect of 0.73 (95% CI: 0.48-0.13), and the proportional mediated by Gr2<neutropenia was 7.4%.

TABLE 8
Mediation analysis: total, direct, and indirect effects

| | | | |
|---|---|---|---|
| Effect | Estimate | 95% Confidence Interval | P value |

hENT1 rs760370 model

| | | | |
|---|---|---|---|
| Total effect | 0.16 | 0.04-0.57 | 0.005 |
| Direct effect | 0.22 | 0.66-0.77 | 0.018 |
| Indirect effect | 0.73 | 0.46-1.14 | 0.17 |
| Proportion mediated | 0.071 | | |

hENT1 rs9394992 model

| | | | |
|---|---|---|---|
| Total effect | 0.17 | 0.06-0.46 | <0.001 |
| Direct effect | 0.23 | 0.09-0.60 | 0.003 |
| Indirect effect | 0.73 | 0.48-1.13 | 0.16 |
| Proportion mediated | 0.074 | | |

### Discussion

The results present the first evidence that the NTs gene polymorphisms, hENT1 rs760370 involved in cellular uptake of FTD confer clinical outcomes in refractory mCRC patients treated with TAS-102. The most important finding is that TPI also participates in enhancement of FTD anti-tumor activity, because TPI itself has been known to be ineffective in terms of efficacy or toxicity. Furthermore, MATE1 rs2289669 and OCT2 rs316019 gene-gene interaction might confer equilibration of TPI blood concentration. Finally, a significant predictive marker according to FTD and/or TPI-derived anti-tumor manners in TAS-102 treatment has been presented.

NTs are located in the cell membrane that mediates the cellular uptake and release of physiologic nucleosides and nucleoside analogues. NTs proteins are mainly classified into hENTs (hENT1 and hENT2) and hCNTs (hCNT1, 2 and 3) [6,20]. While hENTs have a bi-directional manner depending on the nucleoside concentration gradient between the inside and outside of the cell membrane, hCNTs transport purine nucleosides inward of the cell membrane against the concentration gradient of the substrate [21].

Higher levels of hENT1 mRNA were reported in various types of cancer including colorectal, breast, lung and stomach compared to normal tissues [22]. Association of hENT1 protein expression and a nucleoside analogue with gemcitabine efficacy was first investigated by Spratlin et al. showing that tumor cells with high expression of hENT1 were associated with longer survival compared to those with the absence of staining in patients with advanced pancreatic adenocarcinoma [23]. Thereby, clinical evaluation of hENT1 protein expression has been considered as predictive marker for gemcitabine.

Recently, an in vitro study identified the down regulation of hENT mRNA levels as a possible mechanism of FTD resistance in human colorectal cancer cell [11]. Interestingly, low levels of hENT RNA were associated with low intracellular levels of TFT in resistant cells compared to FTD sensitive cells. Unlike hENT, no specific correlation of hCNT mRNA expression and TFT accumulation was observed, thus leading us to hypothesize a critical role in FTD uptake and sensitivity for hENT1 but not for hCNT. Nevertheless, a previous in vivo study demonstrated that FTD was absorbed via rCNT1 in the intestinal lumen rats [5]. However, it should be noted that, in contrast to ubiquitous distribution of hENT1 expression in tissue, hCNT1 mRNA expression in tumors was more limited in tumors like kidney, uterine, lung and small intestine [22].

A previous study investigated whether SNP of NTs were associated with toxicity and survival of pancreatic cancer patients, and reported that hENT1 rs760370, rs9394992 and rs324148 ACT haplotype was significantly associated with longer OS compared with the most common haplotype of each gene respectively [24]. No significant association of the hENT1 SNPs and neutropenia was observed. Tanaka et al. demonstrated that the hENT1 rs760370 G/G variant was associated with poor tumor response and hENT1 rs9394992 with any T allele was identified with increased neutropenia, which was opposite to the results [25]. However, the opposite results can be logically explained by the following reasons: 1) gemcitabine and FTD intracellular uptake is mainly mediated by hENT1 and hCNT1, respectively, 2) hENT1 and hCNT1 act as bi-directional transporter and inward transporter, respectively. This suggests the minor alleles might serve as a predictor of less function of the NTs, leading to intracellular accumulation of FTD due to extracellular transport dysfunction, along with diminished gemcitabine concentration in cells due to dysfunction of intracellular uptake.

Taken together with the study results, hENT1 polymorphisms confer the efficacy independently of neutropenia has been assured because of FTD accumulation in cells by extracellular transport dysfunction of hENT1, while both hENT1 and hCNT1 are involved in FTD transport in tumor cells. However the hENT1 gene alleles show the opposite trend in efficacy between gemcitabine and FTD, which demonstrates a similar trend in neutropenia, suggesting a potent cause of neutropenia is mainly hENT1 dysfunction in the extracellular transport. In addition, the minor allele of hENT1 rs760370 is probably associated with high hENT1 mRNA expression leading to increased FTD uptake into cells leading to longer survival and FTD-induced neutropenia.

TPI has just been regarded as an assistant component of TAS-102 that maintains FTD blood concentration by inhibiting TP. TPI is mainly excreted by OCT2 and MATE1 in the proximal tubular cell membrane as their substrate, while the role of glomerular filtration on renal TPI elimination is negligible [13]. OCT2 is localized to the basal membrane of renal proximal tubular cells and in neurons, while MATE1 is localized in the luminal membrane of renal tubules and in the canalicular membrane of hepatocytes [26,27]. Several substrates such as N,N-Dimethylimidodicarbonimidic diamide (metformin) overlap among these transporters suggesting a well-organized coordination in renal tubular secretion of drugs by OCT2 mediated uptake from blood into tubular cells and MATE1 mediated export into the urine [14].

In addition, a gene-gene interaction between OCT2 and MATE1 polymorphisms has been reported that determined the association of renal clearance of metformin and SNPs of OCT2 and MATE1 [16,27,28,29]. As described in the results, the distribution of the secretory clearance (CLsec) of metformin for categorizing expected those of TPI has been referred, because neither of the drugs confers nephrotoxicity as their similarity [12]. Being the first to provide the new categorization classified based on estimated differences in CLsec by gene-gene interaction of the transporter genes as regulator of TPI excretion. TPI was associated with PFS and OS between the categories based on OCT2 rs316019 and MATE1 rs2289669 gene-gene interaction for efficacy according to expected CLsec, consistent with the hypothesis. In addition, the combination of polymorphisms significantly identified survival benefit from TPI compared with any single polymorphism, indicating utility of the unique classification by gene-gene interaction not only in metformin but also TPI. Thus, non-excreted, unchanged circulating TPI might re-induce inhibition of FTD degradation by TP at the liver, maintaining the anti-tumor effect of FTD [30]. In the additional analysis, a further categorization of hENT1 rs360370 polymorphism with the former category derived from OCT2 rs316019 and MATE1 rs2289669 gene-gene interaction demonstrated the survival benefit more clear than those in the individual. The results suggest that the NTs for FTD uptake and transporters in the renal tubule for TPI excretion might harmonize well with each other when dosing TAS-102 in patients. However, further study to confirm the presence of gene-gene interaction in association with renal clearance for TPI excretion and harmonization of these transporters is warranted to build a tailor-made strategy for TAS-102.

Drug-related toxicities are often focused on their potential impact on clinical outcomes in chemotherapy [23,24,31]. It has been found that neutropenia mainly caused treatment delay of TAS-102 in the training cohort (22/24, 91.8%) suggesting the need for careful monitoring during treatment. Although no statistical correlation between grade 3≤neutropenia and SNPs was observed, finding that grade 2≤neutropenia is a predictor of outcomes in early phase after treatment start of TAS-102 due to its high incidence compared to those of grade 3≤neutropenia (61.5% vs. 38.5%). Intriguingly, no association of OCT2 and/or MATE1 polymorphisms and toxicities were observed, suggesting FTD uptake along with hENT1 gene variants mainly confers the TAS-102 toxicities.

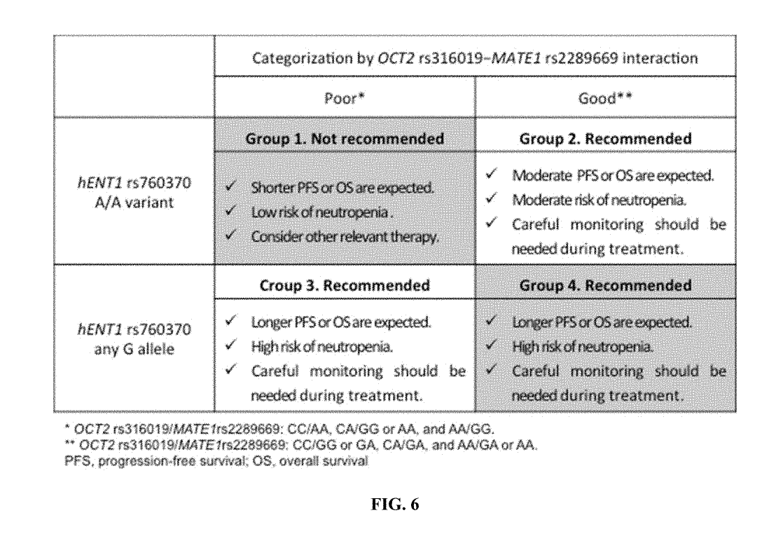

The study is limited by retrospective study design with no correction for multiple testing, and the lack of in vitro/in vivo evidences regarding the role of the SNPs, including secretory clearance of TPI categorized by gene-gene interaction between the OCT2 and MATE1 polymorphism. However, the following characteristics represent the strength of the study: the presence of a control group of patients with comparable clinical characteristics and disease stage; the presence of a larger testing cohort of patients with comparable clinical characteristics, receiving the same treatment. Furthermore, the role of specific transporters involved in pharmacokinetics or pharmacodynamics of FTD and TPI has been clarified, suggesting the new predictive concept based on gene-gene interaction in TAS-102 treatment. To summarize the findings, a treatment algorithm for TAS-102 in patients with mCRC in terms of efficacy and toxicity has been suggested. Patients in Group 3 and 4 are eligible to TAS-102 treatment, however under careful monitoring of onset of neutropenia. TAS-102 is not recommended for patients in Group 1 regarded as less sensitive to TAS-102 despite low risk of neutropenia. In group 2 patients, TAS-102 is recommended, but patients should be considered and monitored (FIG. 6). The results and suggestions of this study will provide guideline to support decision-making in salvage-line treatment in patients with refractory mCRC.

The study demonstrates the first evidence that genetic variants in the TAS-102 pharmacokinetic pathway, hENT1 germline polymorphisms and combination variants of OCT1 and MATE1 polymorphisms may serve as predictive and prognostic markers in refractory mCRC patients receiving TAS-102.

### Example 2: ATM and XRCC3 Variants

TAS-102 is an orally administered drug combining the thymidine-based nucleoside analogue, trifluridine (FTD), and tipiracil hydrochloride, a thymidine phosphorylase inhibitor (TPI) (Hong et al. [1]). Based on results from the phase III RECOURSE trial, the FDA approved TAS-102 in 2015 for patients with refractory metastatic colorectal cancer (mCRC) who have previously received fluoropyrimidine, oxaliplatin, and irinotecan-based chemotherapy (Mayer et al. [32]). FTD is the active anti-tumor component of TAS-102. Incorporation of tri-phosphorylated FTD into DNA acts as the drug's main anti-tumor mechanism of action. The TPI ensures sufficient blood concentration of FTD by preventing its rapid degradation (Temmink et al., Bijnsdorp et al. [3,4]). However, the mechanism of DNA repair following FTD incorporation is still unclear. Previous preclinical reports demonstrated that FTD incorporation into DNA induces single-strand breaks followed by double strand breaks (DSBs) during the G2/M-phase of the cell cycle. In contrast, 5-fluorouracil (5-FU) has been shown to arrest the cell cycle at the G1/S-phase in cancer cell-lines (Suzuki et al. [8]). Without being bound by theory, these results support the conclusion that the antitumor activity of FTD primarily originates from DSBs that confer potent efficacy for 5-FU refractory mCRC.

Homologous recombination (HR) is the primary mechanism of DNA repair triggered by DSBs. The initial cellular DDR to DSBs is mediated by the upstream DDR kinase, ataxia telangiectasia mutated (ATM). ATM subsequently phosphorylates downstream HR-related key genes (Branzei et al., Liu et al., Krajewska et al., Jasin et al. [33,34,35,36]). Cell cycle checkpoints also play an important role in DNA damage and mediate cell cycle arrest to promote DNA repair. ATM and Ataxia Telangiectasia and Rad3-Related Protein (ATR) are known as upstream proteins of checkpoints, responding to different types of DNA damage. Cell cycle arrest mediated by phosphorylation of ATR-checkpoint kinase 1 (Chk1 or CHEK1) and the ATM-checkpoint kinase 2 (Chk2 or CHEK2) by ATR and ATM as DDR suppresses the activity of cyclin-dependent kinase (CDK), leading to cell cycle arrest and DNA repair (Branzei et al. [33]).

This study examined the relationship between homologous recombination (HR) of DNA doublestrand breaks (DSB) and TAS-102 efficacy. Applicant therefore tested whether genetic polymorphisms in the HR pathway are associated with clinical outcomes in patients with refractory metastatic colorectal cancer (mCRC) treated with TAS-102.

Applicant analyzed genomic DNA extracted from 233 blood samples of three different cohorts: an evaluation set of 52 patients receiving TAS-102 (median age 61 years, male 44%, median followup time 6.4 months); a validation cohort of 129 patients receiving TAS-102, and a control cohort of 52 patients receiving regorafenib without history of TAS-102 treatment (median age 64 years, male 44%, all patients deceased). Single nucleotide polymorphisms (SNPs) of genes involved in HR (ATM, BRCA1, BRCA2, XRCC3, FANCD2, H2AX, RAD51) and cell cycle checkpoints (ATR, CHEK1, CHEK2, CDKN1A, TP53, CHE1, PIN1, PCNA) were analyzed by PCR-based direct sequencing for association with progression-free survival (PFS) and overall survival (OS). Candidate SNPs were selected by their frequency and potential function.

### Study Design and Patients

This study was a retrospective exploratory study that investigated three independents cohorts consisted of patients with refractory mCRC patients; an evaluation cohort of 52 patients receiving TAS-102 referred at Cancer Institute Hospital in Japan, a control cohort of 52 patients treated with regorafenib referred at Azienda Ospedaliero-Universitaria Pisana and a validation cohort of 129 patients receiving TAS-102 referred at Azienda Ospedaliero-Universitaria Pisana, Pisa, Italy, Istituto Oncologico Veneto, Padova, Italy, Istituto Nazionale Tumori, Milano in Italy. All patients underwent the treatment in salvage-line setting. Eligible patients had a histologically confirmed diagnosis of mCRC; history of previous standard chemotherapy consisting of 5-FU, L-OHP, CPT-11, bevacizumab, and cetuximab or panitumumab if KRAS wild-type; measurable or evaluable disease according to Response Evaluation Criteria in Solid Tumors (RECIST) v1.1, and signed informed consent. In all patients, adverse events were graded according to the Common Terminology Criteria for Adverse Events, version 4.0.

Patients treated in the evaluation cohort and in the control cohort received TAS-102 (Taiho Pharmaceutical Co., Ltd., Tokyo, Japan) 35 mg per square meter twice daily days 1-5 and 8-12, every 4 weeks. Patients in the control group received 160 mg regorafenib (Bayer, Leverkusen, Germany) once daily from day 1 to 21 every 4 weeks. Doses were adjusted based on haematological and non-haematological adverse events at treating physician's discretion in accordance with the manufacturer's recommendations. The analyses were approved by the University of Southern California (USC) Institutional Review Board of Medical Sciences and conducted at the USC/Norris Comprehensive Cancer Center in accordance with the Declaration of Helsinki and Good Clinical Practice Guidelines.

### Selection of Candidate Single Nucleotide Polymorphism and Methods for Detection

The candidate 22 SNPs in the HR pathway and cell cycle checkpoint used in this study mapped to the following genes: ATM (NM_000051, NM_138292, NM_138293), BRCA1 (NM_007294, NM_007295, NM_007296, NM_007297, NM_007298), BRCA2 (NM_000059), XRCC3 (NM_001100118, NM_001100119, NM_005432), FANCD2 (NM_001018115, NM_033084, NM_001319984), H2AX, RAD51 (NM_001164269, NM_001164270, NM_002875, NM_133487), ATR (NM_001184), CHEK1 (NM_001114121, NM_001114122, NM_001244846, NM_001274, NM_001330427), CHEK2 (NM_001005735, NM_001257387, NM_007194, NM_145862), TP53 (NM_001276761, NM_000546, NM_001126112, NM_001126113, NM_001126114), CHE1 (a.k.a. AATF, NM_012138), PIN1 (NM_006221), CDKN1A (a.k.a. p21, NM_078467, NM_000389, NM_001220777, NM_001220778, NM_001291549) and PCNA (NM_182649, NM_002592). The SNPs were selected using one or more of the following criteria: i) SNP with biological significance according to published literature review, ii) tagging SNPs selected by the HapMap genotype data with r2 threshold=0.8: http://snpinfo.niehs.nih.gov/snpinfo/snptag.php, or iii) minor allele frequency ≥10% in both Caucasians and East Asian (in the Ensembl Genome Browser:

http://uswest.ensembl.org/index.html). Functional significance was predicted based on functional single-nucleotide polymorphism (F-SNP) database:
http://compbio.cs.queensu.ca/F-SNP/ (Table B).

Genomic DNA was extracted from peripheral whole blood in patients of all cohorts using the QIAmp Kit (Qiagen, Valencia, Calif., USA) according to the manufacturer's protocol (www.qiagen.com). The candidate SNPs were tested using PCR-based direct DNA sequence analysis by ABI 3100A Capillary Genetic Analyzer and Sequencing Scanner v1.0 (Applied Biosystems). Both forward and reverse primers used for amplification of extracted DNA are listed in Table B. For quality control purposes, a randomly selected 10% of the samples was analyzed by direct DNA sequencing for each SNP, and the genotype concordance rate was of 99% or more. The investigators analyzing SNPs were blinded to the clinical data.

TABLE B
Analyzed genes polymorphisms in DNA repair and cell cycle checkpoint pathway

| | | | Minor | | |
|---|---|---|---|---|---|
| | | | Allele | | |
| Gene & | Allele | | Freq. | Function of | Forward/Reverse primer |
| rs number | location | Base | CEU/JPN | polymorphism | (5′-3′) |
| ATM | Intron | C > G | 0.45/ | Transcriptional | F: (SEQ ID NO: 8) |
| | | | 0.56 | regulation, | GCCTTGGGCAAGGGT |
| | | | | conserved | ACTTA |
| rs609429 | Chromosome 11: | | | | R: (SEQ ID NO: 9) |
| | 108325782 | | | | CCCACGTCTGCCTAT |
| | | | | | CTGTC |
| BRCA1 | Non- | G > A | 0.36/ | Protein coding, | F: (SEQ ID NO: 43) |
| | synonymous | | 0.26 | splicing | CCAGAGTGGGCAGA |
| | | | | regulation, post | GAATGT |
| rs799917 | Chromosome 17: | | | translation, | R: (SEQ ID NO: 44) |
| | 43092919 | | | conserved | AACCACAGTCGGGAA |
| | | | | | ACAAG |
| BRCA1 | Non- | T > C | 0.36/ | Protein coding, | F: (SEQ ID NO: 45) |
| | synonymous | | 0.26 | splicing | GCTTGAATGTTTTCA |
| | | | | regulation, post | TCACTGG |
| rs16941 | Chromosome 17: | | | translation | R: (SEQ ID NO: 46) |
| | 43092418 | | | | CAGTGAGCACAATTA |
| | | | | | GCCGTA |
| BRCA2 | Exon | A > C | 0.33/ | Protein coding, | F: (SEQ ID NO: 47) |
| | | | 0.25 | splicing | GGAACCAAATGATAC |
| | | | | regulation, post | TGATCCA |
| rs144848 | Chromosome 13: | | | translation | R: (SEQ ID NO: 48) |
| | 32332592 | | | | ACCATTCACAGGCCA |
| | | | | | AAGAC |
| XRCC3 | Exon | G > A | 0.42/ | Protein coding, | F: (SEQ ID NO: 49) |
| | | | 0.12 | splicing | CTCACCTGGTTGATG |
| | | | | regulation, post | CACAG |
| | | | | translation | Or |
| | | | | | F: (SEQ ID NO: 10) |
| | | | | | CCGCATCCTGGCTAA |
| | | | | | AAATA |
| rs861539 | Chromosome 14: | | | | R: (SEQ ID NO: 11) |
| | 103699416 | | | | CCATTCCGCTGTGAA |
| | | | | | TTTG |
| FANCD2 | Synonymous | T > G | 0.17/ | Protein coding, | F: (SEQ ID NO: 50) |
| | | | 0.07 | splicing | CATGTGCCTCTGCTC |
| | | | | regulation, | AAAAA |
| rs2272125 | Chromosome 3: | | | transcriptional | R: (SEQ ID NO: 51) |
| | 10096385 | | | regulation, | GATCCAAGGCTTACC |
| | | | | conserved | TGCAA |
| FANCD2 | Intronic | G > A | 0.48/ | Transcriptional | F: (SEQ ID NO: 52) |
| | | | 0.75 | regulation | CACTGCCATACCACC |
| | | | | | ACTTG |
| rs6792811 | Chromosome 3: | | | | R: (SEQ ID NO: 53) |
| | 10100768 | | | | GATTACAGGCGTGAG |
| | | | | | CCATT |
| H2AX | Non- | T > C | 0.44/ | Protein coding, | F: (SEQ ID NO: 54) |
| | synonymous | | 0.64 | splicing | TCTGGGACCAGAGAG |
| | | | | regulation, post | AGAGG |
| rs643788 | Chromosome 11: | | | translation, | R: (SEQ ID NO: 55) |
| | 119097048 | | | conserved | TCCAGTCCATTTCTCC |
| | | | | | TTGC |
| H2AX | 3′ prime near | C > T | 0.44/ | Transcriptional | F: (SEQ ID NO: 56) |
| | | | 0.62 | regulation | AGACCAAAGGGGTG |
| | | | | | GAGTCT |
| rs2509049 | Chromosome 11: | | | | R: (SEQ ID NO: 57) |
| | 119095811 | | | | TCCATTCTCCCTTTGT |
| | | | | | CAGG |
| RAD51 | 5′ prime UTR | G > C | 0.07/ | Transcriptional | F: (SEQ ID NO: 58) |
| | | | 0.14 | regulation | AGCTGGGAACTGCAA |
| | | | | | CTCAT |
| rs1801320 | Chromosome 15: | | | | R: (SEQ ID NO: 59) |
| | 40695330 | | | | CGCCTCACACACTCA |
| | | | | | CCTC |
| ATR | Non- | G > A | 0.42/ | Protein coding, | F: (SEQ ID NO: 60) |
| | synonymous | | 0.58 | splicing | AGCAGAACACAACCT |
| | | | | regulation, | ATCTGC |
| rs2227928 | Chromosome 3: | | | transcriptional | R: (SEQ ID NO: 61) |
| | 142562770 | | | regulation, post | CAGCTCCTTTGCAGT |
| | | | | translation, | TGATG |
| | | | | conserved | |
| CHEK1 | Intronic | G > A | 0.50/ | Transcriptional | F: (SEQ ID NO: 62) |
| | | | 0.41 | regulation, | TGGGCTATCAATGGA |
| | | | | conserved | AGAAAA |
| rs521102 | Chromosome 11: | | | | R: (SEQ ID NO: 63) |
| | 125644678 | | | | AGGTGTGAGCCACAG |
| | | | | | CCTAT |
| CHEK2 | Intronic | T > C | 0.45/ | Transcriptional | F: (SEQ ID NO: 64) |
| rs2267130 | | | 0.34 | regulation, | GGCTTGGAAGTTCAA |
| | | | | conserved | TCAGG |
| | Chromosome 22: | | | | R: (SEQ ID NO: 65) |
| | 28703766 | | | | TTCATTCCCAGGTAG |
| | | | | | CATCC |
| CHEK2 | 5′ prime UTR | C > G | 0.25/ | Transcriptional | F: (SEQ ID NO: 66) |
| rs2236142 | | | 0.66 | regulation, | ACCAATGAGGAGCA |
| | | | | conserved | GCAGAT |
| | Chromosome 22: | | | | R: (SEQ ID NO: 67) |
| | 28741956 | | | | CTGATTGGCTGGGGA |
| | | | | | GTC |
| TP53 | Non- | C > G | 0.23/ | Protein coding, | F: (SEQ ID NO: 68) |
| | synonymous | | 0.41 | splicing | TTCTGGGAAGGGACA |
| | | | | regulation, post | GAAGA |
| rs1042522 | Chromosome 17: | | | translation | R: (SEQ ID NO: 69) |
| | 7676154 | | | | GAAGACCCAGGTCCA |
| | | | | | GATGA |
| CHE1 | Synonymous | T > C | 0.35/ | Protein coding, | F: (SEQ ID NO: 70) |
| (AATF) | | | 0.07 | splicing | GCCTTTGAACGCTCA |
| | | | | regulation, | ATCTT |
| rs1045056 | Chromosome 17: | | | conserved. | R: (SEQ ID NO: 71) |
| | 36989342 | | | | AACTCTCTGGGACAG |
| | | | | | GCTGA |
| CHE1 | Intronic | A > G | 0.36/ | Tag SNP | F: (SEQ ID NO: 72) |
| (AATF) | | | 0.08 | | AGCTGACCCCCTGAA |
| | | | | | AGTCT |
| rs1564796 | Chromosome 17: | | | | R: (SEQ ID NO: 73) |
| | 36973537 | | | | AGCAATGAAGGCAG |
| | | | | | GAGAAA |
| PIN1 | Promoter | G > C | 0.13/ | Unknown | F: (SEQ ID NO: 74) |
| | region | | 0.05 | | ACTCTGGGTCCCCAA |
| | | | | | ATACC |
| rs2233678 | Chromosome 19: | | | | R: (SEQ ID NO: 75) |
| | 9834503 | | | | GTGCCGACATTGACA |
| | | | | | TTCAT |
| PIN1 | Promoter | T > C | 0.34/ | Unknown | F: (SEQ ID NO: 76) |
| | region | | 0.61 | | CCAGACTGCGAGGGA |
| | | | | | ATAAA |
| rs2233679 | Chromosome 19: | | | | R: (SEQ ID NO: 77) |
| | 9834678 | | | | TGTTTCCCACAGATG |
| | | | | | TCCAA |
| CDKNA1 | Non- | C > A | 0.04/ | Protein coding, | F: (SEQ ID NO: 78) |
| | synonymous | | 0.39 | splicing | GTCCGTCAGAACCCA |
| | | | | regulation, post | TGC |
| rs1801270 | Chromosome 6: | | | translation, | R: (SEQ ID NO: 79) |
| | 36684194 | | | transcription | GTGTCTCGGTGACAA |
| | | | | regulation, | AGTCG |
| | | | | conserved | |
| CDKNA1 | 3′ prime UTR | C > T | 0.04/ | Transcriptional | F: (SEQ ID NO: 80) |
| | | | 0.39 | regulation | TGGCTGACTTCTGCT |
| | | | | | GTCTC |
| rs1059234 | Chromosome 6: | | | | R: (SEQ ID NO: 81) |
| | 36685820 | | | | AAGATGTAGAGCGG |
| | | | | | GCCTTT |
| PCNA | Intronic | C > T | 0.40/ | Tag SNP | F: (SEQ ID NO: 82) |
| | | | 0.37 | | GAAGGGCTGTATTTC |
| | | | | | GAACG |
| rs25406 | Chromosome 20: | | | | R: (SEQ ID NO: 83) |
| | 5118990 | | | | ATAATGGCATCCTCC |
| | | | | | AGCAG |

CEU refers to the frequency in the Caucasian population. JPN refers to the frequency in the Japanese population. UTR refers to either the 5′ or 3′ untranslated region of the gene or mRNA.

The amplicon generated comprising, or alternatively consisting essentially of, or yet further consisting of ATM rs609429 using primers SEQ ID NO:8 and SEQ ID NO:9 has the following sequence (S=C or G): 5′-GCCTTGGGCAAGGGTACTTAATCTT TTCTCAACCTCAATTTCCTGGTTATAAAATGAGAAGATAC(S)TAACTTACTATATT GATAACAATTCAGTGATTTTATATACTGTGTGTATGTACACACAGATACACATACA TACATATAGAGAGAGACAGACAGACAGACAGATAGGCAGACGTGGG-3′ (SEQ ID NO:22). Thus, the sequence of the amplicon from the (C) allele is 5′-GCCTTGGGCAAGGG TACTTAATCTTTTCTCAACCTCAATTTCCTGGTTATAAAATGAGAAGATACCTAACTT ACTATATTGATAACAATTCAGTGATTTTATATACTGTGTGTATGTACACACAGATAC ACATACATACATATAGAGAGAGACAGACAGACAGACAGATAGGCAGACGTGGG-3′ (SEQ ID NO:84) and the sequence of the amplicon from the (G) allele is 5′-GCCTTGGGCAA GGGTACTTAATCTTTTCTCAACCTCAATTTCCTGGTTATAAAATGAGAAGATACGTA ACTTACTATATTGATAACAATTCAGTGATTTTATATACTGTGTGTATGTACACACAGA TACACATACATACATATAGAGAGAGACAGACAGACAGACAGATAGGCAGACGTGG G-3′ (SEQ ID NO:85).

The amplicon generated comprising, or alternatively consisting essentially of, or yet further consisting of XRCC3 rs861539 using primers comprising, or alternatively consisting essentially of, or yet further consisting of the sequence SEQ ID NO:10 and SEQ ID NO:11 has the following sequence (R=A or G): 5′-CCGCATCCTGGCTAAAAAT ACGAGCTCAGGGGTGCAACCCTGCCTTGGTGCTCACCTGGTTGATGCACAGCACAG GGCTCTGGAAGGCACTGCTCAGCTCACGCAGC(R)TGGCCCCCAGGGACTGCAGATG CCTGGCCCTGGGGGCGGAGGCCTGGCTGTCAAATTCACAGCGGAATGG-3′ (SEQ ID NO:23). Thus, the sequence of the amplicon from the (A) allele is 5′-CCGCATCCTGGCTA AAAATACGAGCTCAGGGGTGCAACCCTGCCTTGGTGCTCACCTGGTTGATGCACAG CACAGGGCTCTGGAAGGCACTGCTCAGCTCACGCAGCATGGCCCCCAGGGACTGCA GATGCCTGGCCCTGGGGGCGGAGGCCTGGCTGTCAAATTCACAGCGGAATGG-3′ (SEQ ID NO:96), and the sequence of the amplicon from the (G) allele is 5′-CCGCATCCTGG CTAAAAATACGAGCTCAGGGGTGCAACCCTGCCTTGGTGCTCACCTGGTTGATGCAC AGCACAGGGCTCTGGAAGGCACTGCTCAGCTCACGCAGCGTGGCCCCCAGGGACTG CAGATGCCTGGCCCTGGGGGCGGAGGCCTGGCTGTCAAATTCACAGCGGAATGG-3′ (SEQ ID NO:97).

The amplicon generated comprising, or alternatively consisting essentially of, or yet further consisting of XRCC3 rs861539 using primers comprising, or alternatively consisting essentially of, or yet further consisting of the sequences SEQ ID NO:49 and SEQ ID NO:11 has the following sequence (R=A or G): 5′-CTCACCTGGTTGATGCACAGCACAGGG CTCTGGAAGGCACTGCTCAGCTCACGCAGC(R)TGGCCCCCAGGGACTGCAGATGCCT GGCCCTGGGGGCGGAGGCCTGGCTGTCAAATTCACAGCGGAATGG-3′ (SEQ ID NO:98). Thus, the sequence of the amplicon from the (A) allele is 5′-CTCACCTGGTTGATG CACAGCACAGGGCTCTGGAAGGCACTGCTCAGCTCACGCAGCATGGCCCCCAG GGACTGCAGATGCCTGGCCCTGGGGGCGGAGGCCTGGCTGTCAAATTCACAGCG GAATGG-3′ (SEQ ID NO:99), and the sequence of the amplicon from the (G) allele is 5′-CTCA CCTGGTTGATGCACAGCACAGGGCTCTGGAAGGCACTGCTCAGCTCACGCAGCGTG GCC CCCAGGGACTGCAGATGCCTGGCCCTGGGGGCGGAGGCCTGGCTGTCAAATTC ACAGCGGAATGG-3′ (SEQ ID NO:100).

### Statistical Analysis

The primary endpoint in this study was progression-free survival (PFS), and the secondary endpoints were overall survival (OS) and disease control rate (DCR). PFS was defined as the interval between the date of starting treatment and the date of confirmed disease progression or death. Data of patients without disease progression or death were censored at the date of last follow up. OS was calculated from the date of starting treatment until the date of death from any cause. In patients who were lost to follow-up, data were censored at the date of last follow up. The disease control rate (DCR) was defined as the proportion of patients who achieved a stable disease (SD) or progressed disease (PD) according to RECIST v1.1. Chi-square tests were used to examine the difference in baseline patient characteristics among three cohorts. Allelic distribution of all SNPs by ethnicity for deviation from Hardy-Weinberg equilibrium and the allelic frequencies of SNPs were tested using the Chi-square tests. Fisher's exact test was applied to examine the associations between SNPs and disease control, and between SNPs and toxicity. PFS and OS were estimated by the Kaplan-Meier method and were compared using the log-rank test, with predictive or prognostic clinical factors and candidate SNPs that was identified by univariate analysis using codominant or dominant genetic model if appropriate. Multivariable analysis using the Cox proportional hazards model was conducted to identify factors influencing PFS and OS. The baseline demographic and clinical characteristics that remained statistically significantly associated with PFS and OS in multivariable analyses were included in the final models. The minimum detectable hazard ratios ranged from 2.42 to 3.23 corresponding to the minor allele frequency of 0.1 to 0.4 in the association between a SNP and PFS among the evaluation cohort (N=52, PFS events=43) considering a dominant model and using a two-sided 0.05-level log-rank test with 80% power. The power was greater than 98% when applying the same model and test to the validation cohort. All analyses were carried out with SAS 9.4 (SAS Institute, Cary, N.C., USA). All tests were 2-sided at a significance level of 0.050. P values were not adjusted for multiple testing.

### Baseline Characteristics

The median follow-up time, median PFS and OS were 6.4 months, 2.6 months and 8.0 months in the evaluation cohort, 5.3 months, 2.0 months and 5.7 months in the validation cohort, and 5.3 months, 1.9 months and 5.3 months in the control cohort, respectively. All patients deceased at the last follow-up time in the control cohort. The baseline characteristics of the evaluation, validation, and control cohorts are summarized in Table C. Patient's gender, performance status, and adjuvant treatment history were differently distributed. The associations between baseline characteristics and clinical outcome were summarized in Tables D-F for evaluation, validation, and control cohorts, respectively. In detail, liver metastasis, KRAS wild type and prior exposure to anti-EGFR anti-body was significantly associated with shorter PFS; history of adjuvant treatment was associated with longer PFS in evaluation cohort. In addition, the number of prior chemotherapy, adjuvant treatment history and anti-EGFR anti-body were correlated with OS. In the validation cohort, age <61 and previous exposure of anti-EGFR antibodies are significantly associated with shorter PFS and OS; liver metastasis and ECOG performance status ≥1 significantly correlated with shorter PFS and OS, respectively. In the control cohort, patients with poor ECOG performance status and multiple metastatic sites had shorter PFS and OS; age <61 and liver metastasis also showed significantly shorter PFS.

TABLE C
Summary of Baseline Characteristics

| | Evaluation | | | | | | |
|---|---|---|---|---|---|---|---|
| | cohort | | Validation cohort | | Control cohort | | |
| | TAS-102 | | TAS-102 | | Regorafenib | | |
| | (N = 52) | | (N = 129) | | (N = 52) | | P value |
| Cohort | N | % | N | % | N | % | * |
| Sex | | | | | | | 0.026 |
| Male | 23 | 44.2 | 80 | 62 | 23 | 44.2 | |
| Female | 29 | 55.8 | 49 | 38 | 29 | 55.8 | |
| Age (year) | | | | | | | |
| Median (range) | 61 (34-83) | | 66 (35-83) | | 64 (42-81) | | 0.11 |
| <61 | 25 | 48.1 | 44 | 34.1 | 19 | 36.5 | 0.21 |
| ≥61 | 27 | 51.9 | 85 | 65.9 | 33 | 63.5 | |
| ECOG Performance status | | | | | | | 0.009 |
| ECOG 0 | 33 | 63.5 | 73 | 56.6 | 42 | 80.8 | |
| ECOG 1 | 19 | 36.5 | 56 | 43.4 | 10 | 19.2 | |
| Primary tumor site | | | | | | | 0.41 |
| Right | 19 | 37.3 | 40 | 32 | 22 | 42.3 | |
| Left | 32 | 62.7 | 85 | 68 | 30 | 57.7 | |
| Liver metastasis | | | | | | | 0.18 |
| Yes | 38 | 73.1 | 97 | 75.2 | 45 | 86.5 | |
| No | 14 | 26.9 | 32 | 24.8 | 7 | 13.5 | |
| Lung metastasis | | | | | | | 0.55 |
| Yes | 36 | 69.2 | 89 | 69 | 40 | 76.9 | |
| No | 16 | 30.8 | 40 | 31 | 12 | 23.1 | |
| Lymph node metastasis | | | | | | | 0.57 |
| Yes | 23 | 44.2 | 56 | 43.4 | 27 | 51.9 | |
| No | 29 | 55.8 | 73 | 56.6 | 25 | 48.1 | |
| Peritoneal metastasis | | | | | | | 0.78 |
| Yes | 12 | 23.1 | 32 | 24.8 | 15 | 28.9 | |
| No | 40 | 76.9 | 97 | 75.2 | 37 | 71.1 | |
| Number of metastases | | | | | | | 0.14 |
| <2 | 32 | 61.5 | 70 | 54.3 | 22 | 42.3 | |
| ≥2 | 20 | 38.5 | 59 | 45.7 | 30 | 57.7 | |
| Primary remain | | | | | | | 0.86 |
| Yes | 7 | 13.5 | 13 | 10.6 | 6 | 12 | |
| No | 45 | 86.5 | 110 | 89.4 | 46 | 88 | |
| Adjuvant history | | | | | | | 0.003 |
| Yes | 24 | 46.1 | 47 | 36.4 | 8 | 15.4 | |
| No | 28 | 53.9 | 82 | 63.6 | 44 | 84.6 | |
| Pre-chemotherapy numbers | | | | | | | <0.001 |
| <4 | 30 | 57.7 | 106 | 82.2 | 31 | 59.6 | |
| ≥4 | 22 | 42.3 | 23 | 17.8 | 21 | 40.4 | |
| KRAS exon2 status | | | | | | | 0.77 |
| Wild-type | 28 | 54.9 | NA | NA | 26 | 52 | |
| Mutant | 23 | 45.1 | NA | NA | 24 | 48 | |

* P value was based on Chi-square test, or the Kruskal-Wallis test when appropriate.

TABLE D
Baseline characteristics and clinical outcome in the evaluation cohort treated with TAS-102.

| | | Progression-Free Survival | | | Overall Survival | | |
|---|---|---|---|---|---|---|---|
| | | Median, | | | Median, | | |
| | | months | HR | | months | HR | |
| | N | (95% Cl) | (95% Cl) | P‡ | (95% Cl) | (95% Cl) | P‡ |
| Age | | | | 0.48 | | | 0.55 |
| <61 | 25 | 2.2 | 1 (Ref.) | | 8.1 | 1 (Ref.) | |
| | | (1.8, 3.4) | | | (5.3, 12.4) | | |
| ≥61 | 27 | 3.0 | 0.81 | | 6.3 | 1.30 | |
| | | (1.9, 4.1) | (0.44, 1.49) | | (4.9, 14.8+) | (0.54, 3.09) | |
| Sex | | | | 0.087 | | | 0.064 |
| Male | 23 | 2.1 | 1 (Ref.) | | 4.9 | 1 (Ref.) | |
| | | (1.6, 3.3) | | | (3.3, 8.1+) | | |
| Female | 29 | 3.4 | 0.60 | | 8.3 | 0.47 | |
| | | (2.1, 4.2) | (0.32, 1.12) | | (6.3, 15.6+) | (0.18, 1.20) | |
| Performance status | | | | 0.84 | | | 0.43 |
| ECOG 0 | 33 | 2.9 | 1 (Ref.) | | 8.7 | 1 (Ref.) | |
| | | (2.0, 3.7) | | | (6.3, 14.8+) | | |
| ECOG 1 | 19 | 2.7 | 1.06 | | 7.2 | 1.42 | |
| | | (1.6, 3.4) | (0.56, 2.00) | | (4.9, 15.6+) | (0.59, 3.37) | |
| Tumor site | | | | 0.42 | | | 0.95 |
| Right | 19 | 3.3 | 1 (Ref.) | | 8.7 | 1 (Ref.) | |
| | | (1.6, 6.6+) | | | (3.4, 14.8+) | | |
| Left | 32 | 2.9 | 1.30 | | 8.1 | 0.97 | |
| | | (2.0, 3.4) | (0.67, 2.53) | | (5.3, 15.6+) | (0.40, 2.38) | |
| Liver metastasis | | | | 0.010 | | | 0.27 |
| Yes | 38 | 2.3 | 1 (Ref.) | | 7.2 | 1 (Ref.) | |
| | | (1.8, 3.4) | | | (4.9, 14.8+) | | |
| No | 14 | 4.2 | 0.43 | | 8.7 | 0.57 | |
| | | (1.8, 11.3+) | (0.20, 0.91) | | (5.4, 15.6+) | (0.21, 1.58) | |
| Lung metastasis | | | | 0.58 | | | 0.42 |
| Yes | 36 | 2.9 | 1 (Ref.) | | 8.3 | 1 (Ref.) | |
| | | (2.1, 4.1) | | | (5.7, 14.8+) | | |
| No | 16 | 2.5 | 1.19 | | 6.3 | 1.44 | |
| | | (1.4, 3.4) | (0.61, 2.30) | | (4.4, 15.6+) | (0.59, 3.48) | |
| LN metastasis | | | | 0.43 | | | 0.90 |
| Yes | 23 | 3.0 | 1 (Ref.) | | 8.3 | 1 (Ref.) | |
| | | (1.8, 4.4) | | | (4.4, 15.6+) | | |
| No | 29 | 2.5 | 1.27 | | 7.2 | 1.06 | |
| | | (1.6, 3.4) | (0.69, 2.34) | | (5.3, 14.8+) | (0.45, 2.51) | |
| Peritoneal | | | | 0.25 | | | 0.71 |
| Yes | 12 | 3.1 | 1 (Ref.) | | 6.3 | 1 (Ref.) | |
| | | (1.9, 11.3+) | | | (4.4, 12.4) | | |
| No | 40 | 2.3 | 1.48 | | 8.3 | 0.84 | |
| | | (1.8, 3.5) | (0.71, 3.09) | | (5.7, 14.8+) | (0.33, 2.15) | |
| Number of | | | | 0.51 | | | 0.26 |
| metastases | | | | | | | |
| ≤2 | 32 | 2.7 | 1 (Ref.) | | 8.3 | 1 (Ref.) | |
| | | (2.1, 3.7) | | | (6.3, 15.6+) | | |
| >2 | 20 | 2.9 | 1.22 | | 8.1 | 1.63 | |
| | | (1.6, 3.5) | (0.64, 2.31) | | (3.3, 12.4+) | (0.68, 3.89) | |
| Pre-chemo | | | | 0.062 | | | 0.032 |
| numbers | | | | | | | |
| <4 | 30 | 3.0 | 1 (Ref.) | | 8.7 | 1 (Ref.) | |
| | | (2.1, 4.4) | | | (6.3, 15.6+) | | |
| ≥4 | 22 | 2.1 | 1.73 | | 6.3 | 2.48 | |
| | | (1.8, 3.4) | (0.92, 3.23) | | (3.1, 12.4+) | (1.04, 5.90) | |
| Adjuvant history | | | | 0.028 | | | 0.033 |
| Yes | 24 | 3.7 | 1 (Ref.) | | 8.7 | 1 (Ref.) | |
| | | (2.1, 4.6) | | | (6.3, 15.6+) | | |
| No | 28 | 2.1 | 1.90 | | 6.3 | 2.44 | |
| | | (1.6, 2.9) | (1.02, 3.53) | | (4.9, 10.1+) | (0.96, 6.17) | |
| Primary tumor | | | | 0.48 | | | 0.37 |
| resection | | | | | | | |
| Yes | 45 | 3.0 | 1 (Ref.) | | 8.1 | 1 (Ref.) | |
| | | (2.1, 3.5) | | | (5.7, 12.4) | | |
| No | 7 | 2.1 | 1.39 | | 8.1+ | 1.72 | |
| | | (0.9, 6.0+) | (0.54, 3.58) | | (1.2, 8.1+) | (0.49, 5.99) | |
| KRAS | | | | 0.018 | | | 0.060 |
| Wild type | 28 | 2.1 | 1 (Ref.) | | 7.2 | 1 (Ref.) | |
| | | (1.8, 3.0) | | | (3.4, 8.7) | | |
| Mutant | 23 | 3.3 | 0.51 | | 8.3 | 0.42 | |
| | | (1.8, 5.2) | (0.26, 0.99) | | (5.7, 15.6+) | (0.16, 1.07) | |
| Histology | | | | 0.86 | | | 0.17 |
| well | 13 | 2.1 | 1 (Ref.) | | 6.3 | 1 (Ref.) | |
| | | (1.6, 5.2) | | | (2.0, 8.3+) | | |
| Mod-por | 36 | 3.0 | 0.94 | | 8.7 | 0.52 | |
| | | (1.9, 3.5) | (0.44, 2.00) | | (5.7, 15.6+) | (0.20, 1.40) | |
| Prior bev | | | | 0.28 | | | 0.073 |
| Yes | 48 | 2.9 | 1 (Ref.) | | 8.3 | 1 (Ref.) | |
| | | (2.1, 3.5) | | | (6.3, 15.6+) | | |
| No | 4 | 2.5 | 1.86 | | 4.1 | 3.43 | |
| | | (1.6, 3.0+) | (0.55, 6.24) | | (3.4, 4.9+) | (0.71, 16.58) | |
| Prior EGFR | | | | 0.012 | | | 0.024 |
| Yes | 28 | 2.1 | 1 (Ref.) | | 6.3 | 1 (Ref.) | |
| | | (1.8, 3.0) | | | (3.4, 12.4+) | | |
| No | 24 | 3.7 | 0.49 | | 15.6+ | 0.35 | |
| | | (2.3, 5.2) | (0.26, 0.95) | | (6.3, 15.6+) | (0.14, 0.91) | |
| Prior REGORA | | | | 0.18 | | | 0.21 |
| Yes | 36 | 2.1 | 1 (Ref.) | | 8.1 | 1 (Ref.) | |
| | | (1.8, 3.4) | | | (4.9, 12.4) | | |
| No | 16 | 3.7 | 0.65 | | 14.8+ | 0.50 | |
| | | (2.1, 4.9) | (0.33, 1.26) | | (5.3, 14.8+) | (0.17, 1.50) | |

(Ref.) is an abbreviation of Reference.
‡P value was based on log-rank test in the univariate analysis.
Bold P values are <0.05.

TABLE E
Baseline characteristics and clinical outcomes in the validation
cohort treated with TAS-102

| | | Progression-Free Survival | | | Overall Survival | | |
|---|---|---|---|---|---|---|---|
| | | Median, | | | Median, | | |
| | | months | HR | | months | HR | |
| | N | (95% CI) | (95% CI) | P ‡ | (95% CI) | (95% CI) | P ‡ |
| Age group | | | | 0.010 | | | 0.029 |
| <61 | 44 | 1.9 | 1 (Ref.) | | 4.4 | 1 (Ref.) | |
| | | (1.7, 2.0) | | | (3.7, 5.4) | | |
| ≥61 | 85 | 2.0 | 0.61 | | 9.0 | 0.56 | |
| | | (1.9, 2.5) | (0.41, 0.91) | | (5.2, 9.0) | (0.32, 0.96) | |
| Sex | | | | 0.93 | | | 0.46 |
| Male | 80 | 1.9 | 1 (Ref.) | | 9.0 | 1 (Ref.) | |
| | | (1.9, 2.0) | | | (4.6, 9.0) | | |
| Female | 49 | 2.1 | 0.98 | | 5.3 | 1.22 | |
| | | (1.9, 2.5) | (0.66, 1.47) | | (3.8, 7.3) | (0.70, 2.12) | |
| Performance status | | | | 0.059 | | | 0.003 |
| ECOG 0 | 73 | 2.0 | 1 (Ref.) | | 9.0 | 1 (Ref.) | |
| | | (1.9, 2.4) | | | (5.3, 9.0) | | |
| ECOG 1+ | 56 | 1.9 | 1.43 | | 4.1 | 2.15 | |
| | | (1.8, 2.1) | (0.96, 2.13) | | (3.1, 7.1) | (1.25, 3.71) | |
| Tumor site | | | | 0.75 | | | 0.38 |
| Right | 40 | 1.9 | 1 (Ref.) | | 5.1 | 1 (Ref.) | |
| | | (1.8, 2.3) | | | (3.7, 8.7) | | |
| Left | 85 | 2.0 | 1.07 | | 9.0 | 0.78 | |
| | | (1.9, 2.3) | (0.70, 1.64) | | (4.2, 9.0) | (0.44, 1.37) | |
| Liver metastasis | | | | 0.006 | | | 0.28 |
| Yes | 97 | 1.9 | 1 (Ref.) | | 5.4 | 1 (Ref.) | |
| | | (1.8, 2.0) | | | (4.1, 9.0) | | |
| No | 32 | 2.4 | 0.52 | | 8.7+ | 0.69 | |
| | | (2.0, 6.5) | (0.32, 0.86) | | (3.8, 8.7+) | (0.35, 1.37) | |
| Lung metastasis | | | | 0.017 | | | 0.16 |
| Yes | 89 | 2.1 | 1 (Ref.) | | 8.8+ | 1 (Ref.) | |
| | | (1.9, 2.5) | | | (4.7, 8.8+) | | |
| No | 40 | 1.9 | 1.62 | | 4.1 | 1.46 | |
| | | (1.7, 2.0) | (1.06, 2.47) | | (3.7, 9.0) | (0.84, 2.55) | |
| LN metastasis | | | | 0.98 | | | 0.44 |
| Yes | 56 | 2.0 | 1 (Ref.) | | 8.8+ | 1 (Ref.) | |
| | | (1.9, 2.1) | | | (4.0, 8.8+) | | |
| No | 73 | 2.0 | 0.99 | | 5.4 | 1.24 | |
| | | (1.9, 2.4) | (0.67, 1.47) | | (3.9, 9.0) | (0.71, 2.14) | |
| Peritoneal | | | | 0.17 | | | 0.47 |
| Yes | 32 | 1.9 | 1 (Ref.) | | 5.3 | 1 (Ref.) | |
| | | (1.7, 2.0) | | | (3.7, 7.1) | | |
| No | 97 | 2.0 | 0.74 | | 9.0 | 0.80 | |
| | | (1.9, 2.3) | (0.47, 1.16) | | (4.1, 9.0) | (0.44, 1.46) | |
| Number of metastases | | | | 0.38 | | | 0.48 |
| ≤2 | 70 | 2.0 | 1 (Ref.) | | 5.5 | 1 (Ref.) | |
| | | (1.9, 2.4) | | | (3.8, 9.0) | | |
| >2 | 59 | 2.0 | 1.18 | | 8.8+ | 0.83 | |
| | | (1.9, 2.1) | (0.80, 1.75) | | (4.4, 8.8+) | (0.48, 1.42) | |
| Pre-chemo numbers | | | | 0.029 | | | 0.059 |
| <4 | 81 | 1.9 | 1 (Ref.) | | 5.1 | 1 (Ref.) | |
| | | (1.8, 2.0) | | | (3.8, 9.0+) | | |
| ≥4 | 48 | 2.1 | 0.66 | | 8.7+ | 0.57 | |
| | | (1.9, 2.8) | (0.43, 0.99) | | (5.2, 8.7+) | (0.31, 1.04) | |
| Adjuvant history | | | | 0.26 | | | 0.46 |
| Yes | 47 | 2.0 | 1 (Ref.) | | 8.5+ | 1 (Ref.) | |
| | | (1.9, 2.5) | | | (4.2, 8.5+) | | |
| No | 82 | 1.9 | 1.25 | | 5.5 | 1.23 | |
| | | (1.9, 2.1) | (0.83, 1.89) | | (3.9, 9.0) | (0.70, 2.18) | |
| Primary tumor resection | | | | 0.007 | | | 0.12 |
| Yes | 110 | 2.0 | 1 (Ref.) | | 9.0 | 1 (Ref.) | |
| | | (1.9, 2.3) | | | (5.1, 9.0) | | |
| No | 13 | 1.8 | 2.13 | | 3.9 | 1.87 | |
| | | (1.6, 2.0) | (1.16, 3.90) | | (2.7, 8.7) | (0.84, 4.16) | |
| RAS status | | | | 0.35 | | | 0.18 |
| Wild type | 47 | 2.0 | 1 (Ref.) | | 8.8+ | 1 (Ref.) | |
| | | (1.8, 2.3) | | | (3.7, 8.8+) | | |
| Mutant | 69 | 1.9 | 1.21 | | 5.1 | 1.48 | |
| | | (1.9, 2.1) | (0.80, 1.83) | | (3.7, 9.0) | (0.82, 2.64) | |
| BRAF status | | | | 0.28 | | | 0.028 |
| Wild type | 106 | 2.0 | 1 (Ref.) | | 5.8 | 1 (Ref.) | |
| | | (1.9, 2.3) | | | (4.6, 9.0) | | |
| Mutant | 9 | 1.8 | 1.40 | | 3.3 | 2.38 | |
| | | (1.2, 2.0) | (0.66, 2.99) | | (1.2, 8.7) | (1.07, 5.31) | |
| Prior REGORA | | | | 0.40 | | | 0.35 |
| Yes | 36 | 2.0 | 1 (Ref.) | | 8.7+ | 1 (Ref.) | |
| | | (1.8, 2.8) | | | (4.2, 8.7+) | | |
| No | 93 | 2.0 | 1.20 | | 5.4 | 1.36 | |
| | | (1.9, 2.1) | (0.77, 1.87) | | (3.9, 9.0) | (0.71, 2.58) | |
| Time to metastasis | | | | 0.14 | | | 0.064 |
| Synchronous | 76 | 1.9 | 1 (Ref.) | | 5.2 | 1 (Ref.) | |
| | | (1.9, 2.1) | | | (3.7, 9.0) | | |
| Metachronous | 53 | 2.0 | 0.75 | | 8.5+ | 0.59 | |
| | | (1.9, 2.6) | (0.50, 1.12) | | (4.7, 8.5+) | (0.34, 1.05) | |
| Adjuvant Oxa | | | | 0.79 | | | 0.61 |
| Yes | 37 | 2.0 | 1 (Ref.) | | 8.5+ | 1 (Ref.) | |
| | | (1.7, 2.5) | | | (4.1, 8.5+) | | |
| No | 92 | 1.9 | 1.06 | | 5.8 | 1.17 | |
| | | (1.9, 2.1) | (0.68, 1.65) | | (3.9, 9.0) | (0.64, 2.16) | |
| Grading | | | | 0.57 | | | 0.33 |
| 1-2 | 81 | 2.1 | 1 (Ref.) | | 9.0 | 1 (Ref.) | |
| | | (1.9, 2.3) | | | (5.1, 9.0) | | |
| 3 | 29 | 1.9 | 1.15 | | 5.3 | 1.38 | |
| | | (1.8, 2.5) | (0.71, 1.85) | | (3.8, 8.5) | (0.72, 2.66) | |
| Mucinous histology | | | | 0.90 | | | 0.37 |
| Yes | 18 | 1.9 | 1 (Ref.) | | 5.5 | 1 (Ref.) | |
| | | (1.7, 2.1) | | | (2.8, 8.7) | | |
| No | 99 | 2.0 | 0.97 | | 9.0 | 0.72 | |
| | | (1.9, 2.3) | (0.53, 1.74) | | (4.7, 9.0) | (0.35, 1.49) | |
| Previous anti-VEGF | | | | 0.42 | | | 0.48 |
| Yes | 120 | 2.0 | 1 (Ref.) | | 5.5 | 1 (Ref.) | |
| | | (1.9, 2.1) | | | (4.4, 9.0) | | |
| No | 7 | 2.8 | 0.67 | | 6.6+ | 0.60 | |
| | | (0.4, 4.4) | (0.25, 1.83) | | (3.6, 6.6+) | (0.15, 2.47) | |
| Previous anti-EGFR | | | | 0.030 | | | 0.002 |
| Yes | 50 | 2.1 | 1 (Ref.) | | 8.8+ | 1 (Ref.) | |
| | | (1.9, 2.8) | | | | | |
| No | 78 | 1.9 | 1.53 | | 4.4 | 2.63 | |
| | | (1.8, 2.0) | (1.02, 2.29) | | (3.7, 5.5) | (1.38, 5.00) | |

(Ref.) is an abbreviation of Reference. ‡ P value was based on log-rank test in the univariate analysis. Bold P values are <0.05.

TABLE F
Baseline characteristics and clinical outcomes in the
control cohort treated with regorafenib

| | | Progression-Free Survival | | | Overall Survival | | |
|---|---|---|---|---|---|---|---|
| | | Median, | | | Median, | | |
| | | months | HR | | months | HR | |
| | N | (95% CI) | (95% CI) | P ‡ | (95% CI) | (95% CI) | P ‡ |
| Age | | | | 0.043 | | | 0.27 |
| <61 | 19 | 1.8 | 1 (Ref.) | | 5.1 | 1 (Ref.) | |
| | | (1.4, 2.3) | | | (2.2, 8.1) | | |
| ≥61 | 33 | 2.1 | 0.58 | | 5.8 | 0.74 | |
| | | (1.8, 3.4) | (0.31, 1.07) | | (3.7, 8.0) | (0.41, 1.32) | |
| Sex | | | | 0.94 | | | 0.55 |
| Male | 23 | 1.8 | 1 (Ref.) | | 5.4 | 1 (Ref.) | |
| | | (1.7, 3.1) | | | (2.6, 8.8) | | |
| Female | 29 | 2.0 | 1.02 | | 5.5 | 1.18 | |
| | | (1.8, 2.9) | (0.58, 1.80) | | (3.6, 8.0) | (0.67, 2.06) | |
| Performance status | | | | 0.014 | | | 0.029 |
| ECOG 0 | 42 | 2.1 | 1 (Ref.) | | 5.9 | 1 (Ref.) | |
| | | (1.8, 2.9) | | | (4.5, 8.0) | | |
| ECOG 1 | 10 | 1.2 | 2.27 | | 2.4 | 2.09 | |
| | | (0.0, 2.2) | (1.10, 4.65) | | (0.7, 6.0) | (1.02, 4.29) | |
| Tumor site | | | | 0.41 | | | 0.45 |
| Right | 22 | 1.8 | 1 (Ref.) | | 3.9 | 1 (Ref.) | |
| | | (1.4, 2.3) | | | (2.0, 6.0) | | |
| Left | 30 | 2.2 | 0.80 | | 7.2 | 0.82 | |
| | | (1.8, 3.4) | (0.45, 1.40) | | (4.5, 9.2) | (0.46, 1.46) | |
| Liver metastasis | | | | 0.033 | | | 0.19 |
| Yes | 45 | 1.9 | 1 (Ref.) | | 4.7 | 1 (Ref.) | |
| | | (1.8, 2.2) | | | (3.6, 7.9) | | |
| No | 7 | 4.8 | 0.46 | | 6.6 | 0.61 | |
| | | (0.9, 9.1) | (0.20, 1.07) | | (1.4, 12.6) | (0.26, 1.41) | |
| Lung metastasis | | | | 0.62 | | | 0.50 |
| Yes | 40 | 1.9 | 1 (Ref.) | | 5.9 | 1 (Ref.) | |
| | | (1.7, 2.8) | | | (4.2, 8.0) | | |
| No | 12 | 2.0 | 1.17 | | 3.7 | 1.24 | |
| | | (1.6, 3.2) | (0.61, 2.26) | | (1.8, 9.7) | (0.65, 2.39) | |
| LN metastasis | | | | 0.011 | | | 0.072 |
| Yes | 27 | 1.8 | 1 (Ref.) | | 3.8 | 1 (Ref.) | |
| | | (1.5, 2.0) | | | (2.4, 6.0) | | |
| No | 25 | 2.8 | 0.52 | | 7.7 | 0.62 | |
| | | (1.8, 4.6) | (0.29, 0.92) | | (4.7, 10.4) | (0.35, 1.09) | |
| Peritoneal | | | | 0.64 | | | 0.51 |
| Yes | 15 | 1.9 | 1 (Ref.) | | 5.1 | 1 (Ref.) | |
| | | (1.4, 3.1) | | | (2.0, 8.1) | | |
| No | 37 | 2.0 | 0.87 | | 5.5 | 0.82 | |
| | | (1.7, 2.9) | (0.47, 1.60) | | (3.7, 8.0) | (0.44, 1.51) | |
| Number of metastases | | | | 0.018 | | | 0.088 |
| ≤2 | 22 | 2.8 | 1 (Ref.) | | 6.3 | 1 (Ref.) | |
| | | (1.8, 4.6) | | | (4.6, 10.4) | | |
| >2 | 30 | 1.9 | 1.86 | | 4.0 | 1.59 | |
| | | (1.7, 2.2) | (1.04, 3.32) | | (2.6, 7.9) | (0.90, 2.80) | |
| Pre-chemo numbers | | | | 0.85 | | | 0.59 |
| <4 | 31 | 2.0 | 1 (Ref.) | | 6.0 | 1 (Ref.) | |
| | | (1.7, 2.9) | | | (3.6, 8.1) | | |
| ≥4 | 21 | 1.8 | 0.95 | | 4.7 | 1.16 | |
| | | (1.7, 4.3) | (0.54, 1.67) | | (2.6, 8.0) | (0.66, 2.04) | |
| Adjuvant history | | | | 0.36 | | | 0.15 |
| Yes | 8 | 1.8 | 1 (Ref.) | | 3.9 | 1 (Ref.) | |
| | | (0.8, 3.1) | | | (0.8, 10.4) | | |
| No | 44 | 2.0 | 0.71 | | 5.9 | 0.59 | |
| | | (1.8, 2.9) | (0.33, 1.53) | | (3.7, 8.0) | (0.27, 1.28) | |
| Primary tumor resection | | | | 0.58 | | | 0.94 |
| Yes | 46 | 1.9 | 1 (Ref.) | | 5.6 | 1 (Ref.) | |
| | | (1.8, 2.8) | | | (3.6, 7.9) | | |
| No | 6 | 2.0 | 0.79 | | 4.2 | 1.04 | |
| | | (0.9, 12.4+) | (0.33, 1.87) | | (1.4, 19.0+) | (0.44, 2.44) | |
| KRAS | | | | 0.85 | | | 0.78 |
| Wild type | 26 | 1.9 | 1 (Ref.) | | 6.3 | 1 (Ref.) | |
| | | (1.7, 3.4) | | | (3.7, 8.1) | | |
| Mutant | 24 | 2.0 | 1.05 | | 4.8 | 0.93 | |
| | | (1.7, 2.8) | (0.60, 1.87) | | (2.4, 7.9) | (0.52, 1.64) | |

(Ref.) is an abbreviation of Reference. ‡ P value was based on log-rank test in the univariate analysis. Bold P values are <0.05.

Association of Clinical Outcome and DNA Repair-Related Genetic Variants in the Evaluation and Validation Cohort Treated with TAS-102

The allelic frequencies and genotype distribution in each SNP were close to those reported for the Caucasian population or Japanese population. All candidate SNPs were successfully genotyped in each cohort, except for three patients with PCNA rs25406 in the evaluation cohort and four patients with XRCC3 rs861539 in the validation cohort because of insufficient quality of extracted genomic DNA.

In univariate analysis for the evaluation cohort, patients with any G allele in ATM rs609429 had a longer OS compared to those with the C/C variant [8.7 vs. 4.4 months, hazard ratio (HR) 0.37, 95% CI: 0.14-0.99, P=0.022] (FIG. 1A). Patients carrying the G/A variant in XRCC3 rs861539 had significant longer PFS (3.8 vs. 2.3 months, HR 0.44, 95% CI: 0.21-0.92, P=0.024) and OS (15.6 vs. 6.3 months, HR 0.25, 95% CI: 0.08-0.79, P=0.012) than those with the G/G variant (FIG. 2). Patients with any C allele in CHEK2 rs2267130 had significant longer PFS (3.4 vs. 2.1 months, HR 0.48, 95% CI: 0.25-0.90, P=0.010) compared to those with the T/T variant. In multivariable analysis adjusted for liver metastases and adjuvant history, ATM rs609429 remained significant for OS (HR 0.24, P=0.020), and XRCC3 rs861539 showed marginal significance in PFS (HR 0.52, P=0.091) and OS (HR 0.31, P=0.056). CHEK2 rs2267130 did not remain as an independent factor. PIN1 rs2233678 showed significant association with PFS and OS in both uni- and multivariable analysis. However, the number of patients with G/C variants was only two.

In univariate analysis for the validation cohort, patients carrying any G allele in ATM rs609429 showed longer PFS (2.0 vs. 1.9 months, HR 0.78, 95% CI: 0.52-1.16, P=0.20) and OS (not reached vs. 4.4 months, HR 0.73, 95% CI: 0.43-1.25, P=0.24) than those with the C/C variant; however, the effects were not statistically significant (FIGS. 1C and 1D). XRCC3 rs861539 showed a different distribution of variants compared with those of the evaluation cohort having no A/A variant. However, patients with the G/A variant showed a trend of longer OS compared with the C/C variant (9.0 vs. 5.5 months, HR 0.75), which is consistent with the results in the evaluation cohort. None of the candidate SNPs remained significant in a multivariable model.

Association of clinical outcome and DNA repair-related genetic variants in the control cohort treated with regorafenib: In the control cohort receiving regorafenib without previous treatment with TAS-102, genotyping for the all candidate SNPs was available in all patients. Uni- and multivariate analyses showed no significant association between ATM rs609429, XRCC3 rs861539 and CHEK2 rs2267130 genotypes with PFS or OS.

Detailed results of these analyses are presented in Tables G-M below.

TABLE G
Association between gene polymorphism and disease control

| | | Disease Control | | |
|---|---|---|---|---|
| | N | CR/PR/SD | PD | P value* |
| Evaluation Cohort | | | | |
| ATM rs609429 | | | | 0.49 |
| C/C | 14 | 5 (45%) | 6 (55%) | |
| G/C | 27 | 8 (32%) | 17 (68%) | |
| G/G | 11 | 2 (20%) | 8 (80%) | |
| | | | | 0.46 |
| C/C | 14 | 5 (45%) | 6 (55%) | |
| Any G | 38 | 10 (29%) | 25 (71%) | |
| XRCC3 rs861539 | | | | 0.33 |
| G/G | 35 | 8 (27%) | 22 (73%) | |
| G/A | 17 | 7 (44%) | 9 (56%) | |
| CHEK2 rs2267130 | | | | 0.075 |
| T/T | 22 | 3 (15%) | 17 (85%) | |
| T/C | 22 | 9 (47%) | 10 (53%) | |
| C/C | 8 | 3 (43%) | 4 (57%) | |
| | | | | 0.031 |
| T/T | 22 | 3 (15%) | 17 (85%) | |
| Any C | 30 | 12 (46%) | 14 (54%) | |
| Validation Cohort | | | | |
| ATM rs609429 | | | | 0.29 |
| C/C | 51 | 11 (22%) | 39 (78%) | |
| C/G | 63 | 20 (32%) | 43 (68%) | |
| G/G | 15 | 6 (40%) | 9 (60%) | |
| | | | | 0.23 |
| C/C | 51 | 11 (22%) | 39 (78%) | |
| Any G | 78 | 26 (33%) | 52 (67%) | |
| XRCC3 rs861539 | | | | 0.13 |
| G/G | 50 | 16 (32%) | 34 (68%) | |
| G/A | 50 | 16 (33%) | 33 (67%) | |
| A/A | 25 | 3 (12%) | 22 (88%) | |
| | | | | 0.54 |
| G/G | 50 | 16 (32%) | 34 (68%) | |
| Any A | 75 | 19 (26%) | 55 (74%) | |
| | | | | 0.049 |
| Any G | 100 | 32 (32%) | 67 (68%) | |
| A/A | 25 | 3 (12%) | 22 (88%) | |
| CHEK2 rs2267130 | | | | 0.76 |
| T/T | 35 | 8 (24%) | 26 (76%) | |
| T/C | 63 | 20 (32%) | 43 (68%) | |
| C/C | 31 | 9 (29%) | 22 (71%) | |
| | | | | 0.51 |
| T/T | 35 | 8 (24%) | 26 (76%) | |
| Any C | 94 | 29 (31%) | 65 (69%) | |
| Control Cohort | | | | |
| ATM rs609429 | | | | 0.89 |
| C/C | 28 | 8 (32%) | 17 (68%) | |
| C/G | 21 | 8 (38%) | 13 (62%) | |
| G/G | 3 | 1 (33%) | 2 (67%) | |
| | | | | 0.77 |
| C/C | 28 | 8 (32%) | 17 (68%) | |
| Any G | 24 | 9 (38%) | 15 (63%) | |
| XRCC3 rs861539 | | | | 0.77 |
| G/G | 15 | 4 (27%) | 11 (73%) | |
| G/A | 30 | 10 (37%) | 17 (63%) | |
| A/A | 7 | 3 (43%) | 4 (57%) | |
| | | | | 0.53 |
| G/G | 15 | 4 (27%) | 11 (73%) | |
| Any A | 37 | 13 (38%) | 21 (62%) | |
| CHEK2 rs2267130 | | | | 0.79 |
| T/T | 15 | 4 (31%) | 9 (69%) | |
| T/C | 26 | 10 (40%) | 15 (60%) | |
| C/C | 11 | 3 (27%) | 8 (73%) | |
| | | | | 1.00 |
| T/T | 15 | 4 (31%) | 9 (69%) | |
| Any C | 37 | 13 (36%) | 23 (64%) | |

CR, complete response;
PR, partial response;
SD, stable disease;
PD, progressive disease.
*P value was based on the Fisher's exact test for response, log-rank test in the univariate analysis (†) and Wald test in the multivariable analysis within Cox regression model adjusted for liver metastasis and adjuvant history in the evaluation cohort; age group (<61 vs. ≥61), liver metastasis, ECOG performance status, previous anti-EGFR (‡) in the validation cohort; and ECOG performance status and number of metastases in the control cohort.
+ Estimates not reached yet.

TABLE H
Association between gene polymorphism and PFS

| | | Progression-Free Survival | | | | |
|---|---|---|---|---|---|---|
| | | Median, | | | | |
| | | months | HR | | HR | |
| | N | (95% CI) | (95% CI) † | P * | (95% CI) ‡ | P * |
| Evaluation Cohort | | | | | | |
| ATM rs609429 | | | | 0.52 | | 0.70 |
| C/C | 14 | 3.3 (1.6, 6.0+) | 1 (Ref.) | | 1 (Ref.) | |
| G/C | 27 | 2.9 (1.8, 3.4) | 1.53 (0.68, 3.43) | | 1.30 (0.53, 3.19) | |
| G/G | 11 | 2.6 (1.2, 4.1) | 1.55 (0.59, 4.04) | | 1.56 (0.56, 4.37) | |
| | | | | 0.25 | | 0.48 |
| C/C | 14 | 3.3 (1.6, 6.0+) | 1 (Ref.) | | 1 (Ref.) | |
| Any G | 38 | 2.7 (2.1, 3.4) | 1.53 (0.70, 3.35) | | 1.37 (0.57, 3.26) | |
| XRCC3 rs861539 | | | | 0.024 | | 0.091 |
| G/G | 35 | 2.3 (1.9, 3.3) | 1 (Ref.) | | 1 (Ref.) | |
| G/A | 17 | 3.8 (1.6, 6.6) | 0.44 (0.21, 0.92) | | 0.52 (0.24, 1.11) | |
| CHEK2 rs2267130 | | | | 0.028 | | 0.089 |
| T/T | 22 | 2.1 (1.6, 2.9) | 1 (Ref.) | | 1 (Ref.) | |
| T/C | 22 | 3.7 (2.1, 5.2) | 0.44 (0.22, 0.89) | | 0.46 (0.22, 0.94) | |
| C/C | 8 | 2.7 (0.9, 4.6+) | 0.62 (0.25, 1.58) | | 0.59 (0.23, 1.51) | |
| | | | | 0.010 | | 0.031 |
| T/T | 22 | 2.1 (1.6, 2.9) | 1 (Ref.) | | 1 (Ref.) | |
| Any C | 30 | 3.4 (2.1, 4.6) | 0.48 (0.25, 0.90) | | 0.49 (0.26, 0.94) | |
| Validation Cohort | | | | | | |
| ATM rs609429 | | | | 0.41 | | 0.23 |
| C/C | 51 | 1.9 (1.8, 2.3) | 1 (Ref.) | | 1 (Ref.) | |
| C/G | 63 | 2.0 (1.9, 2.3) | 0.76 (0.50, 1.16) | | 0.77 (0.50, 1.20) | |
| G/G | 15 | 2.0 (1.2, 6.1) | 0.85 (0.45, 1.63) | | 1.32 (0.67, 2.59) | |
| | | | | 0.20 | | 0.44 |
| C/C | 51 | 1.9 (1.8, 2.3) | 1 (Ref.) | | 1 (Ref.) | |
| Any G | 78 | 2.0 (1.9, 2.3) | 0.78 (0.52, 1.16) | | 0.85 (0.56, 1.29) | |
| XRCC3 rs861539 | | | | 0.40 | | 0.54 |
| G/G | 50 | 2.1 (1.7, 2.5) | 1 (Ref.) | | 1 (Ref.) | |
| G/A | 50 | 2.0 (1.9, 2.3) | 0.96 (0.62, 1.50) | | 0.95 (0.59, 1.51) | |
| A/A | 25 | 1.9 (1.8, 2.0) | 1.34 (0.80, 2.25) | | 1.26 (0.74, 2.14) | |
| | | | | 0.72 | | 0.82 |
| G/G | 50 | 2.1 (1.7, 2.5) | 1 (Ref.) | | 1 (Ref.) | |
| Any A | 75 | 2.0 (1.9, 2.1) | 1.07 (0.72, 1.61) | | 1.05 (0.69, 1.60) | |
| | | | | 0.18 | | 0.28 |
| Any G | 100 | 2.0 (1.9, 2.3) | 1 (Ref.) | | 1 (Ref.) | |
| A/A | 25 | 1.9 (1.8, 2.0) | 1.37 (0.86, 2.18) | | 1.30 (0.81, 2.09) | |
| CHEK2 rs2267130 | | | | 0.88 | | 0.50 |
| T/T | 35 | 1.9 (1.9, 2.0) | 1 (Ref.) | | 1 (Ref.) | |
| T/C | 63 | 2.0 (1.7, 2.3) | 1.12 (0.69, 1.79) | | 1.17 (0.72, 1.91) | |
| C/C | 31 | 2.0 (1.8, 2.4) | 1.08 (0.62, 1.86) | | 0.87 (0.49, 1.54) | |
| | | | | 0.64 | | 0.83 |
| T/T | 35 | 1.9 (1.9, 2.0) | 1 (Ref.) | | 1 (Ref.) | |
| Any C | 94 | 2.0 (1.8, 2.3) | 1.10 (0.71, 1.72) | | 1.05 (0.66, 1.67) | |
| Control Cohort | | | | | | |
| ATM rs609429 | | | | | | |
| C/C | 28 | | | | | |
| C/G | 21 | | | | | |
| G/G | 3 | | | | | |
| | | | | 0.86 | | 0.84 |
| C/C | 28 | 1.9 (1.6, 2.8) | 1 (Ref.) | | 1 (Ref.) | |
| Any G | 24 | 2.0 (1.7, 3.1) | 0.95 (0.55, 1.66) | | 1.06 (0.60, 1.87) | |
| XRCC3 rs861539 | | | | 0.83 | | 0.61 |
| G/G | 15 | 1.7 (1.0, 2.2) | 1 (Ref.) | | 1 (Ref.) | |
| G/A | 30 | 1.9 (1.8, 3.1) | 0.83 (0.44, 1.56) | | 0.92 (0.47, 1.79) | |
| A/A | 7 | 2.7 (1.7, 4.3) | 0.83 (0.34, 2.06) | | 0.62 (0.23, 1.65) | |
| | | | | 0.54 | | 0.63 |
| G/G | 15 | 1.7 (1.0, 2.2) | 1 (Ref.) | | 1 (Ref.) | |
| Any A | 37 | 2.0 (1.8, 3.1) | 0.83 (0.45, 1.53) | | 0.85 (0.44, 1.63) | |
| CHEK2 rs2267130 | | | | 0.90 | | 0.76 |
| T/T | 15 | 1.9 (1.4, 3.4) | 1 (Ref.) | | 1 (Ref.) | |
| T/C | 26 | 1.8 (1.6, 3.1) | 1.02 (0.54, 1.95) | | 1.15 (0.58, 2.27) | |
| C/C | 11 | 2.0 (1.5, 2.7) | 1.18 (0.54, 2.59) | | 1.35 (0.61, 2.99) | |
| | | | | 0.83 | | 0.54 |
| T/T | 15 | 1.9 (1.4, 3.4) | 1 (Ref.) | | 1 (Ref.) | |
| Any C | 37 | 1.8 (1.7, 2.8) | 1.07 (0.58, 1.95) | | 1.22 (0.65, 2.28) | |

* P value was based on the Fisher's exact test for response, log-rank test in the univariate analysis (†) and Wald test in the multivariable analysis within Cox regression model adjusted for liver metastasis and adjuvant history in the evaluation cohort; age group (<61 vs. ≥61), liver metastasis, ECOG performance status, previous anti-EGFR (‡) in the validation cohort; and ECOG performance status and number of metastases in the control cohort. + Estimates not reached yet.

TABLE I
Association between gene polymorphism and OS

| | | Overall Survival | | | | |
|---|---|---|---|---|---|---|
| | | Median, | | | | |
| | | months | HR | | HR | |
| | N | (95% CI) | (95% CI) † | P * | (95% CI) ‡ | P * |
| Evaluation Cohort | | | | 0.055 | | 0.036 |
| ATM rs609429 | | 4.4 (3.1, 8.3+) | 1 (Ref.) | | 1 (Ref.) | |
| C/C | 14 | 12.4 (6.3, 15.6+) | 0.32 (0.11, 0.91) | | 0.19 (0.05, 0.68) | |
| G/C | 27 | 8.7 (1.2, 14.8+) | 0.52 (0.16, 1.73) | | 0.39 (0.10, 1.54) | |
| G/G | 11 | | | 0.022 | | 0.020 |
| | | 4.4 (3.1, 8.3+) | 1 (Ref.) | | 1 (Ref.) | |
| C/C | 14 | 8.7 (6.3, 15.6+) | 0.37 (0.14, 0.99) | | 0.24 (0.07, 0.80) | |
| Any G | 38 | | | 0.012 | | 0.056 |
| XRCC3 rs861539 | | 6.3 (4.9, 12.4+) | 1 (Ref.) | | 1 (Ref.) | |
| G/G | 35 | 15.6+ (8.1, 15.6+) | 0.25 (0.08, 0.79) | | 0.31 (0.09, 1.03) | |
| G/A | 17 | | | 0.38 | | 0.36 |
| CHEK2 rs2267130 | | 8.1 (4.4, 8.7+) | 1 (Ref.) | | 1 (Ref.) | |
| T/T | 22 | 12.4 (6.3, 15.6+) | 0.59 (0.22, 1.60) | | 0.56 (0.20, 1.55) | |
| T/C | 22 | 5.7 (1.4, 10.7+) | 1.21 (0.37, 3.99) | | 1.31 (0.39, 4.42) | |
| C/C | 8 | | | 0.43 | | 0.47 |
| | | 8.1 (4.4, 8.7+) | 1 (Ref.) | | 1 (Ref.) | |
| T/T | 22 | 8.3 (5.7, 15.6+) | 0.71 (0.29, 1.74) | | 0.71 (0.29, 1.77) | |
| Any C | 30 | | | | | |
| Validation Cohort | | | | 0.48 | | 0.27 |
| ATM rs609429 | | 4.4 (3.7, 9.0+) | 1 (Ref.) | | 1 (Ref.) | |
| C/C | 51 | 8.7+ (4.7, 8.7+) | 0.71 (0.40, 1.26) | | 0.71 (0.39, 1.30) | |
| C/G | 63 | 5.8 (2.7, 7.1+) | 0.82 (0.34, 2.00) | | 1.43 (0.56, 3.65) | |
| G/G | 15 | | | 0.24 | | 0.44 |
| | | 4.4 (3.7, 9.0+) | 1 (Ref.) | | 1 (Ref.) | |
| C/C | 51 | 8.7+ (5.1, 8.7+) | 0.73 (0.43, 1.25) | | 0.80 (0.45, 1.41) | |
| Any G | 78 | | | 0.48 | | 0.76 |
| XRCC3 rs861539 | | 5.5 (3.6, 8.7+) | 1 (Ref.) | | 1 (Ref.) | |
| G/G | 50 | 9.0 (4.6, 9.0+) | 0.75 (0.40, 1.43) | | 0.79 (0.41, 1.51) | |
| G/A | 50 | 4.2 (3.6, 8.8+) | 1.13 (0.57, 2.22) | | 0.93 (0.46, 1.88) | |
| A/A | 25 | | | 0.65 | | 0.55 |
| | | 5.5 (3.6, 8.7+) | 1 (Ref.) | | 1 (Ref.) | |
| G/G | 50 | 5.8 (4.1, 9.0+) | 0.88 (0.51, 1.54) | | 0.84 (0.47, 1.49) | |
| Any A | 75 | | | 0.40 | | 0.89 |
| | | 5.8 (4.7, 9.0+) | 1 (Ref.) | | 1 (Ref.) | |
| Any G | 100 | 4.2 (3.6, 8.8+) | 1.29 (0.70, 2.39) | | 1.05 (0.56, 1.97) | |
| A/A | 25 | | | 0.97 | | 0.50 |
| CHEK2 rs2267130 | | 5.8 (3.7, 9.0+) | 1 (Ref.) | | 1 (Ref.) | |
| T/T | 35 | 8.7+ (3.8, 8.7+) | 0.93 (0.50, 1.75) | | 0.83 (0.44, 1.58) | |
| T/C | 63 | 5.4 (3.9, 7.1+) | 0.95 (0.45, 2.00) | | 0.48 (0.21, 1.09) | |
| C/C | 31 | | | 0.82 | | 0.26 |
| | | 5.8 (3.7, 9.0+) | 1 (Ref.) | | 1 (Ref.) | |
| T/T | 35 | 5.5 (4.4, 8.7+) | 0.94 (0.52, 1.69) | | 0.70 (0.38, 1.30) | |
| Any C | 94 | | | | | |
| Control Cohort | | | | | | |
| ATM rs609429 | | | | | | |
| C/C | 28 | | | | | |
| C/G | 21 | | | | | |
| G/G | 3 | | | | | |
| | | | | 0.45 | | 0.54 |
| C/C | 28 | 4.6 (2.6, 7.6) | 1 (Ref.) | | 1 (Ref.) | |
| Any G | 24 | 5.9 (3.5, 10.2) | 1.91 (0.44, 8.31) | | 0.84 (0.47, 1.48) | |
| XRCC3 rs861539 | | | | 0.56 | | 0.43 |
| G/G | 15 | 4.1 (1.7, 6.0) | 1 (Ref.) | | 1 (Ref.) | |
| G/A | 30 | 6.8 (3.6, 8.7) | 0.86 (0.25, 2.96) | | 0.76 (0.40, 1.44) | |
| A/A | 7 | 5.4 (2.3, 9.1) | 0.34 (0.04, 2.95) | | 0.54 (0.21, 1.40) | |
| | | | | 0.29 | | 0.27 |
| G/G | 15 | 4.1 (1.7, 6.0) | 1 (Ref.) | | 1 (Ref.) | |
| Any A | 37 | 5.9 (3.8, 8.0) | 0.72 (0.22, 2.39) | | 0.71 (0.38, 1.31) | |
| CHEK2 rs2267130 | | | | 0.70 | | 0.73 |
| T/T | 15 | 3.8 (2.2, 8.0) | 1 (Ref.) | | 1 (Ref.) | |
| T/C | 26 | 6.2 (2.4, 8.9) | 0.77 (0.40, 1.48) | | 0.76 (0.38, 1.53) | |
| C/C | 11 | 5.7 (2.0, 7.8) | 0.89 (0.41, 1.97) | | 0.90 (0.41, 2.00) | |
| | | | | 0.46 | | 0.52 |
| T/T | 15 | 3.8 (2.2, 8.0) | 1 (Ref.) | | 1 (Ref.) | |
| Any C | 37 | 5.9 (4.4, 7.9) | 0.80 (0.44, 1.48) | | 0.81 (0.42, 1.54) | |

* P value was based on the Fisher's exact test for response, log-rank test in the univariate analysis (†) and Wald test in the multivariable analysis within Cox regression model adjusted for liver metastasis and adjuvant history in the evaluation cohort; age group (<61 vs. ≥61), liver metastasis, ECOG performance status, previous anti-EGFR (‡) in the validation cohort; and ECOG performance status and number of metastases in the control cohort. + Estimates not reached yet.

TABLE J
Gene polymorphism and disease control rate in the evaluation
cohort treated with TAS-102 (additional genes)

| | | Disease Control Rate | | |
|---|---|---|---|---|
| | N | SD | PD | P value* |
| BRCA1 rs799917 | | | | 0.83 |
| G/G | 23 | 7 (37%) | 12 (63%) | |
| G/A | 23 | 7 (32%) | 15 (68%) | |
| A/A | 6 | 1 (20%) | 4 (80%) | |
| | | | | 0.75 |
| G/G | 23 | 7 (37%) | 12 (63%) | |
| Any A | 29 | 8 (30%) | 19 (70%) | |
| BRCA1 rs16941 | | | | 0.83 |
| T/T | 23 | 7 (37%) | 12 (63%) | |
| T/C | 23 | 7 (32%) | 15 (68%) | |
| C/C | 6 | 1 (20%) | 4 (80%) | |
| | | | | 0.75 |
| T/T | 23 | 7 (37%) | 12 (63%) | |
| Any C | 29 | 8 (30%) | 19 (70%) | |
| BRCA2 rs144848 | | | | 0.51 |
| A/A | 32 | 8 (29%) | 20 (71%) | |
| A/C | 14 | 6 (46%) | 7 (54%) | |
| C/C | 6 | 1 (20%) | 4 (80%) | |
| | | | | 0.53 |
| A/A | 32 | 8 (29%) | 20 (71%) | |
| Any C | 20 | 7 (39%) | 11 (61%) | |
| RAD51 rs1801320 | | | | 0.67 |
| G/G | 44 | 12 (31%) | 27 (69%) | |
| G/C | 8 | 3 (43%) | 4 (57%) | |
| FANCD2 | | | | 0.38 |
| rs2272125 | | | | |
| T/T | 46 | 12 (30%) | 28 (70%) | |
| T/G | 6 | 3 (50%) | 3 (50%) | |
| FANCD2 | | | | 1.00 |
| rs6792811 | | | | |
| A/A | 35 | 10 (33%) | 20 (67%) | |
| G/A^{a} | 15 | 5 (29%) | 11 (71%) | |
| G/G^{a} | 2 | | | |
| H2AX rs2509049 | | | | 0.76 |
| T/T | 21 | 7 (35%) | 13 (65%) | |
| T/C | 23 | 5 (26%) | 14 (74%) | |
| C/C | 8 | 3 (43%) | 4 (57%) | |
| | | | | 1.00 |
| T/T | 21 | 7 (35%) | 13 (65%) | |
| Any C | 31 | 8 (31%) | 18 (69%) | |
| H2AX rs643788 | | | | 0.70 |
| C/C | 22 | 7 (33%) | 14 (67%) | |
| C/T | 22 | 5 (28%) | 13 (72%) | |
| T/T | 8 | 3 (43%) | 4 (57%) | |
| | | | | 1.00 |
| C/C | 22 | 7 (33%) | 14 (67%) | |
| Any T | 30 | 8 (32%) | 17 (68%) | |
| ATR rs2227928 | | | | 0.19 |
| A/A | 16 | 3 (20%) | 12 (80%) | |
| A/G | 24 | 7 (32%) | 15 (68%) | |
| G/G | 12 | 5 (56%) | 4 (44%) | |
| | | | | 0.32 |
| A/A | 16 | 3 (20%) | 12 (80%) | |
| Any G | 36 | 12 (39%) | 19 (61%) | |
| Che1 rs1045056 | | | | 0.70 |
| T/T | 41 | 13 (35%) | 24 (65%) | |
| T/C | 11 | 2 (22%) | 7 (78%) | |
| Che1 rs1564796 | | | | 1.00 |
| T/T | 40 | 12 (34%) | 23 (66%) | |
| T/C^{a} | 11 | 3 (25%) | 8 (67%) | |
| C/C^{a} | 1 | | | |
| p53 rs1042522 | | | | 1.00 |
| C/C | 21 | 6 (30%) | 14 (70%) | |
| C/G | 24 | 7 (35%) | 13 (65%) | |
| G/G | 7 | 2 (33%) | 4 (67%) | |
| | | | | 1.00 |
| C/C | 21 | 6 (30%) | 14 (70%) | |
| Any G | 31 | 9 (35%) | 17 (65%) | |
| Pin1 rs2233678 | | | | 1.00 |
| G/G | 50 | 15 (34%) | 29 (66%) | |
| G/C | 2 | 0 | 2 (100%) | |
| Pin1 rs2233679 | | | | 0.58 |
| C/C | 19 | 4 (24%) | 13 (76%) | |
| C/T | 24 | 8 (36%) | 14 (64%) | |
| T/T | 9 | 3 (43%) | 4 (57%) | |
| | | | | 0.35 |
| C/C | 19 | 4 (24%) | 13 (76%) | |
| Any T | 33 | 11 (38%) | 18 (62%) | |
| CHEK2 rs22236142 | | | | 0.70 |
| G/G | 22 | 8 (40%) | 12 (60%) | |
| G/C | 21 | 5 (28%) | 13 (72%) | |
| C/C | 9 | 2 (25%) | 6 (75%) | |
| | | | | 0.53 |
| G/G | 22 | 8 (40%) | 12 (60%) | |
| Any C | 30 | 7 (27%) | 19 (73%) | |
| PCNA rs25406 | | | | 0.19 |
| G/G | 19 | 3 (19%) | 13 (81%) | |
| G/A | 19 | 8 (50%) | 8 (50%) | |
| A/A | 11 | 4 (36%) | 7 (64%) | |
| | | | | 0.11 |
| G/G | 19 | 3 (19%) | 13 (81%) | |
| Any A | 30 | 12 (44%) | 15 (56%) | |
| p21 rs1801270 | | | | 0.73 |
| A/A | 15 | 4 (31%) | 9 (69%) | |
| A/C | 23 | 6 (29%) | 15 (71%) | |
| C/C | 14 | 5 (42%) | 7 (58%) | |
| | | | | 1.00 |
| A/A | 15 | 4 (31%) | 9 (69%) | |
| Any C | 37 | 11 (33%) | 22 (67%) | |
| p21 rs1059234 | | | | 0.66 |
| T/T | 15 | 5 (45%) | 6 (55%) | |
| T/C | 24 | 6 (27%) | 16 (73%) | |
| C/C | 13 | 4 (31%) | 9 (69%) | |
| | | | | 0.46 |
| T/T | 15 | 5 (45%) | 6 (55%) | |
| Any C | 37 | 10 (29%) | 25 (71%) | |

†*P value was based on the log-rank test in the univariate analysis and Wald test in the multivariable analysis within Cox regression model (‡) adjusted for liver metastasis and adjuvant history in the evaluation cohort; age group.
^{a}Grouped together for analysis.

TABLE K
Gene polymorphism and PFS in the evaluation cohort treated with TAS-102
(additional genes)

| | | Progression-Free Survival | | | | |
|---|---|---|---|---|---|---|
| | | Median, | | | | |
| | | months | HR | | HR | |
| | N | (95% CI) | (95% CI) † | P * | (95% CI) ‡ | P * |
| BRCA1 rs799917 | | | | 0.80 | | 0.56 |
| G/G | 23 | 2.3 (1.6, 3.4) | 1 (Ref.) | | 1 (Ref.) | |
| G/A | 23 | 3.2 (1.8, 4.6) | 0.84 (0.43, 1.62) | | 0.94 (0.47, 1.88) | |
| A/A | 6 | 2.7 (2.0, 4.2) | 1.06 (0.38, 2.90) | | 1.66 (0.58, 4.74) | |
| | | | | 0.64 | | 0.91 |
| G/G | 23 | 2.3 (1.6, 3.4) | 1 (Ref.) | | 1 (Ref.) | |
| Any A | 29 | 3.0 (2.0, 4.2) | 0.87 (0.47, 1.64) | | 1.04 (0.54, 2.00) | |
| BRCA1 rs16941 | | | | 0.80 | | 0.56 |
| T/T | 23 | 2.3 (1.6, 3.4) | 1 (Ref.) | | 1 (Ref.) | |
| T/C | 23 | 3.2 (1.8, 4.6) | 0.84 (0.43, 1.62) | | 0.94 (0.47, 1.88) | |
| C/C | 6 | 2.7 (2.0, 4.2) | 1.06 (0.38, 2.90) | | 1.66 (0.58, 4.74) | |
| | | | | 0.64 | | 0.91 |
| T/T | 23 | 2.3 (1.6, 3.4) | 1 (Ref.) | | 1 (Ref.) | |
| Any C | 29 | 3.0 (2.0, 4.2) | 0.87 (0.47, 1.64) | | 1.04 (0.54, 2.00) | |
| BRCA2 rs144848 | | | | 0.71 | | 0.72 |
| A/A | 32 | 2.7 (2.0, 3.4) | 1 (Ref.) | | 1 (Ref.) | |
| A/C | 14 | 3.5 (1.5, 4.9) | 0.76 (0.37, 1.56) | | 0.82 (0.39, 1.72) | |
| C/C | 6 | 2.7 (0.9, 6.0+) | 0.99 (0.38, 2.59) | | 1.28 (0.47, 3.50) | |
| | | | | 0.51 | | 0.81 |
| A/A | 32 | 2.7 (2.0, 3.4) | 1 (Ref.) | | 1 (Ref.) | |
| Any C | 20 | 3.3 (1.6, 4.6) | 0.82 (0.43, 1.54) | | 0.92 (0.48, 1.78) | |
| RAD51 rs1801320 | | | | 0.23 | | 0.48 |
| G/G | 44 | 2.5 (2.0, 4.1) | 1 (Ref.) | | 1 (Ref.) | |
| G/C | 8 | 3.2 (0.9, 3.4) | 1.57 (0.70, 3.50) | | 1.34 (0.60, 3.00) | |
| FANCD2 rs2272125 | | | | 0.96 | | 0.84 |
| T/T | 46 | 2.7 (2.1, 3.4) | 1 (Ref.) | | 1 (Ref.) | |
| T/G | 6 | 2.8 (1.6, 6.6+) | 0.98 (0.38, 2.51) | | 0.91 (0.35, 2.35) | |
| FANCD2 rs6792811 | | | | 0.39 | | 0.28 |
| A/A | 35 | 2.9 (2.1, 4.2) | 1 (Ref.) | | 1 (Ref.) | |
| G/A ^{a} | 15 | 2.1 (1.5, 3.4) | 1.29 (0.68, 2.47) | | 1.43 (0.74, 2.74) | |
| G/G ^{a} | 2 | | | | | |
| H2AX rs2509049 | | | | 0.41 | | 0.14 |
| T/T | 21 | 2.3 (1.6, 3.4) | 1 (Ref.) | | 1 (Ref.) | |
| T/C | 23 | 2.7 (1.8, 3.7) | 1.03 (0.53, 2.02) | | 1.07 (0.55, 2.11) | |
| C/C | 8 | 3.5 (1.4, 6.6) | 0.63 (0.23, 1.68) | | 0.37 (0.13, 1.10) | |
| | | | | 0.69 | | 0.54 |
| T/T | 21 | 2.3 (1.6, 3.4) | 1 (Ref.) | | 1 (Ref.) | |
| Any C | 31 | 2.9 (2.0, 3.7) | 0.88 (0.47, 1.67) | | 0.82 (0.43, 1.56) | |
| H2AX rs643788 | | | | 0.39 | | 0.13 |
| C/C | 22 | 2.6 (1.6, 3.4) | 1 (Ref.) | | 1 (Ref.) | |
| C/T | 22 | 2.7 (1.8, 3.7) | 1.10 (0.57, 2.14) | | 1.19 (0.60, 2.36) | |
| T/T | 8 | 3.5 (1.4, 6.6) | 0.65 (0.24, 1.72) | | 0.39 (0.13, 1.15) | |
| | | | | 0.80 | | 0.65 |
| C/C | 22 | 2.6 (1.6, 3.4) | 1 (Ref.) | | 1 (Ref.) | |
| Any T | 30 | 2.7 (2.0, 3.7) | 0.93 (0.49, 1.74) | | 0.86 (0.45, 1.65) | |
| ATR rs2227928 | | | | 0.26 | | 0.72 |
| A/A | 16 | 2.1 (1.6, 3.4) | 1 (Ref.) | | 1 (Ref.) | |
| A/G | 24 | 2.9 (1.6, 3.4) | 0.75 (0.38, 1.51) | | 0.82 (0.41, 1.63) | |
| G/G | 12 | 3.7 (2.1, 6.6) | 0.46 (0.19, 1.10) | | 0.43 (0.17, 1.08) | |
| | | | | 0.15 | | 0.23 |
| A/A | 16 | 2.1 (1.6, 3.4) | 1 (Ref.) | | 1 (Ref.) | |
| Any G | 36 | 3.0 (2.1, 3.7) | 0.64 (0.33, 1.23) | | 0.67 (0.35, 1.29) | |
| Che1 rs1045056 | | | | 0.52 | | 0.17 |
| T/T | 41 | 2.9 (1.9, 3.5) | 1 (Ref.) | | 1 (Ref.) | |
| T/C | 11 | 2.1 (1.2, 4.2) | 1.25 (0.61, 2.57) | | 1.70 (0.80, 3.64) | |
| Che1 rs1564796 | | | | 0.65 | | 0.38 |
| T/T | 40 | 2.9 (2.0, 3.5) | 1 (Ref.) | | 1 (Ref.) | |
| T/C ^{a} | 11 | 2.1 (1.2, 4.6) | 1.17 (0.57, 2.40) | | 1.41 (0.65, 3.07) | |
| C/C ^{a} | 1 | | | | | |
| p53 rs1042522 | | | | 0.88 | | 0.95 |
| C/C | 21 | 2.5 (1.6, 4.4) | 1 (Ref.) | | 1 (Ref.) | |
| C/G | 24 | 3.0 (1.9, 3.5) | 0.86 (0.45, 1.65) | | 1.12 (0.57, 2.21) | |
| G/G | 7 | 2.3 (0.7, 4.6+) | 1.02 (0.37, 2.77) | | 1.04 (0.38, 2.86) | |
| | | | | 0.70 | | 0.77 |
| C/C | 21 | 2.5 (1.6, 4.4) | 1 (Ref.) | | 1 (Ref.) | |
| Any G | 31 | 3.0 (2.1, 3.4) | 0.89 (0.48, 1.65) | | 1.10 (0.58, 2.09) | |
| Pin1 rs2233678 | | | | 0.049 | | 0.036 |
| G/G | 50 | 2.9 (2.1, 3.4) | 1 (Ref.) | | 1 (Ref.) | |
| G/C | 2 | 1.7 (1.6, 1.8+) | 3.66 (0.79, 16.99) | | 5.08 (1.11, 23.23) | |
| Pin1 rs2233679 | | | | 0.73 | | 0.99 |
| C/C | 19 | 2.3 (1.8, 3.4) | 1 (Ref.) | | 1 (Ref.) | |
| C/T | 24 | 2.9 (1.8, 3.7) | 0.90 (0.46, 1.75) | | 1.03 (0.52, 2.03) | |
| T/T | 9 | 3.4 (1.6, 4.6) | 0.70 (0.28, 1.79) | | 0.95 (0.34, 2.67) | |
| | | | | 0.55 | | 0.98 |
| C/C | 19 | 2.3 (1.8, 3.4) | 1 (Ref.) | | 1 (Ref.) | |
| Any T | 33 | 3.3 (1.8, 3.7) | 0.84 (0.45, 1.57) | | 1.01 (0.53, 1.94) | |
| CHEK2 rs22236142 | | | | 0.55 | | 0.92 |
| G/G | 22 | 2.9 (1.6, 4.9) | 1 (Ref.) | | 1 (Ref.) | |
| G/C | 21 | 2.5 (1.8, 3.5) | 1.43 (0.72, 2.84) | | 1.13 (0.55, 2.34) | |
| C/C | 9 | 2.7 (1.6, 4.4) | 1.15 (0.49, 2.74) | | 1.18 (0.50, 2.78) | |
| | | | | 0.35 | | 0.68 |
| G/G | 22 | 2.9 (1.6, 4.9) | 1 (Ref.) | | 1 (Ref.) | |
| Any C | 30 | 2.6 (2.1, 3.4) | 1.33 (0.70, 2.50) | | 1.15 (0.59, 2.22) | |
| PCNA rs25406 | | | | 0.51 | | 0.16 |
| G/G | 19 | 2.1 (1.6, 4.2) | 1 (Ref.) | | 1 (Ref.) | |
| G/A | 19 | 3.4 (1.9, 3.7) | 0.68 (0.32, 1.41) | | 0.46 (0.21, 1.02) | |
| A/A | 11 | 2.5 (1.4, 4.6) | 0.89 (0.39, 2.03) | | 0.65 (0.28, 1.51) | |
| | | | | 0.34 | | 0.072 |
| G/G | 19 | 2.1 (1.6, 4.2) | 1 (Ref.) | | 1 (Ref.) | |
| Any A | 30 | 3.3 (2.1, 3.5) | 0.74 (0.39, 1.42) | | 0.53 (0.27, 1.06) | |
| p21 rs1801270 | | | | 0.84 | | 0.70 |
| A/A | 15 | 3.3 (1.6, 4.1) | 1 (Ref.) | | 1 (Ref.) | |
| A/C | 23 | 2.3 (1.8, 3.5) | 0.82 (0.39, 1.74) | | 0.72 (0.34, 1.55) | |
| C/C | 14 | 2.7 (1.6, 4.6) | 0.97 (0.43, 2.17) | | 0.77 (0.33, 1.79) | |
| | | | | 0.70 | 0.40 | |
| A/A | 15 | 3.3 (1.6, 4.1) | 1 (Ref.) | | 1 (Ref.) | |
| Any C | 37 | 2.7 (2.1, 3.5) | 0.88 (0.44, 1.74) | | 0.74 (0.37, 1.49) | |
| p21 rs1059234 | | | | | 0.88 | 0.70 |
| T/T | 15 | 3.3 (1.6, 4.1) | 1 (Ref.) | | 1 (Ref.) | |
| T/C | 24 | 2.7 (2.1, 3.5) | 0.84 (0.40, 1.76) | | 0.75 (0.35, 1.60) | |
| C/C | 13 | 2.9 (1.6, 4.9) | 0.95 (0.42, 2.16) | | 0.73 (0.30, 1.74) | |
| | | | | 0.70 | | 0.40 |
| T/T | 15 | 3.3 (1.6, 4.1) | 1 (Ref.) | | 1 (Ref.) | |
| Any C | 37 | 2.7 (2.1, 3.5) | 0.88 (0.44, 1.74) | | 0.74 (0.37, 1.49) | |

†* P value was based on the log-rank test in the univariate analysis and Wald test in the multivariable analysis within Cox regression model (‡) adjusted for liver metastasis and adjuvant history in the evaluation cohort; age group. ^{a} Grouped together for analysis.

TABLE L
Gene polymorphism and overall survival in the evaluation cohort treated with
TAS-102 (additional genes)

| | Overall Survival | | | | |
|---|---|---|---|---|---|
| | Median, months | | | | |
| | (95% CI) | HR (95% CI)† | P* | HR (95% CI)‡ | P* |
| BRCA1 rs799917 | | | 0.35 | | 0.42 |
| G/G | 6.3 (4.4, 15.6+) | 1 (Ref.) | | 1 (Ref.) | |
| G/A | 8.1 (3.4, 14.8+) | 1.13 (0.47, 2.74) | | 1.36 (0.55, 3.40) | |
| A/A | 12.4+ | 0.28 (0.04, 2.20) | | 0.37 (0.05, 3.07) | |
| | | | 0.82 | | 0.78 |
| G/G | 6.3 (4.4, 15.6+) | 1 (Ref.) | | 1 (Ref.) | |
| Any A | 8.3 (5.3, 14.8+) | 0.90 (0.38, 2.15) | | 1.14 (0.46, 2.82) | |
| BRCA1 rs16941 | | | 0.35 | | 0.42 |
| T/T | 6.3 (4.4, 15.6+) | 1 (Ref.) | | 1 (Ref.) | |
| T/C | 8.1 (3.4, 14.8+) | 1.13 (0.47, 2.74) | | 1.36 (0.55, 3.40) | |
| C/C | 12.4+ | 0.28 (0.04, 2.20) | | 0.37 (0.05, 3.07) | |
| | | | 0.82 | | 0.78 |
| T/T | 6.3 (4.4, 15.6+) | 1 (Ref.) | | 1 (Ref.) | |
| Any C | 8.3 (5.3, 14.8+) | 0.90 (0.38, 2.15) | | 1.14 (0.46, 2.82) | |
| BRCA2 rs144848 | | | 0.78 | | 0.75 |
| A/A | 8.3 (5.4, 12.4) | 1 (Ref.) | | 1 (Ref.) | |
| A/C | 15.6+ (2.7, 15.6+) | 0.91 (0.33, 2.57) | | 0.97 (0.34, 2.76) | |
| C/C | 8.1 (2.7, 8.1+) | 1.48 (0.41, 5.34) | | 1.62 (0.44, 6.02) | |
| | | | 0.88 | | 0.78 |
| A/A | 8.3 (5.4, 12.4) | 1 (Ref.) | | 1 (Ref.) | |
| Any C | 8.1 (2.8, 15.6+) | 1.07 (0.44, 2.58) | | 1.14 (0.46, 2.79) | |
| RAD51 | | | 0.11 | | 0.12 |
| rs1801320 | | | | | |
| G/G | 8.3 (5.7, 15.6+) | 1 (Ref.) | | 1 (Ref.) | |
| G/C | 6.3 (1.4, 8.4+) | 2.19 (0.78, 6.14) | | 2.28 (0.81, 6.48) | |
| FANCD2 | | | 0.40 | | 0.61 |
| rs2272125 | | | | | |
| T/T | 8.3 (5.7, 15.6+) | 1 (Ref.) | | 1 (Ref.) | |
| T/G | 7.2 (2.7, 7.8+) | 1.66 (0.47, 5.83) | | 1.38 (0.39, 4.90) | |
| FANCD2 | | | 0.59 | | 0.73 |
| rs6792811 | | | | | |
| A/A | 8.7 (5.4, 14.8+) | 1 (Ref.) | | 1 (Ref.) | |
| G/A^{a} | 7.2 (3.3, 15.6+) | 1.27 (0.52, 3.08) | | 1.17 (0.48, 2.87) | |
| G/G^{a} | | | | | |
| H2AX rs2509049 | | | 0.16 | | 0.12 |
| T/T | 8.7 (3.3, 10.7+) | 1 (Ref.) | | 1 (Ref.) | |
| T/C | 8.1 (5.4, 12.4+) | 0.99 (0.40, 2.48) | | 0.90 (0.36, 2.27) | |
| C/C | 15.6+ (7.2, 15.6+) | 0.18 (0.02, 1.43) | | 0.11 (0.01, 0.92) | |
| | | | 0.48 | | 0.28 |
| T/T | 8.7 (3.3, 10.7+) | 1 (Ref.) | | 1 (Ref.) | |
| Any C | 8.1 (6.3, 15.6+) | 0.74 (0.30, 1.82) | | 0.61 (0.24, 1.52) | |
| H2AX rs643788 | | | 0.16 | | 0.12 |
| C/C | 8.7 (3.3, 10.7+) | 1 (Ref.) | | 1 (Ref.) | |
| C/T | 6.3 (4.4, 12.4) | 1.15 (0.46, 2.86) | | 1.00 (0.39, 2.52) | |
| T/T | 15.6+ (7.2, 15.6+) | 0.20 (0.02, 1.56) | | 0.12 (0.01, 0.98) | |
| | | | 0.68 | | 0.38 |
| C/C | 8.7 (3.3, 10.7+) | 1 (Ref.) | | 1 (Ref.) | |
| Any T | 8.1 (5.7, 15.6+) | 0.83 (0.34, 2.05) | | 0.66 (0.26, 1.67) | |
| ATR rs2227928 | | | 0.39 | | 0.20 |
| A/A | 5.7 (2.7, 12.4) | 1 (Ref.) | | 1 (Ref.) | |
| A/G | 8.1 (4.9, 15.6) | 0.71 (0.28, 1.78) | | 0.68 (0.26, 1.74) | |
| G/G | 10.1+ (3.3, 10.1) | 0.28 (0.06, 1.31) | | 0.18 (0.04, 0.93) | |
| | | | 0.21 | | 0.14 |
| A/A | 5.7 (2.7, 12.4) | 1 (Ref.) | | 1 (Ref.) | |
| Any G | 8.3 (6.3, 15.6) | 0.57 (0.24, 1.40) | | 0.50 (0.20, 1.27) | |
| Che1 rs1045056 | | | 0.26 | | 0.37 |
| T/T | 7.2 (5.4, 15.6+) | 1 (Ref.) | | 1 (Ref.) | |
| T/C | 12.4 (5.3, 12.4+) | 0.55 (0.18, 1.63) | | 0.60 (0.19, 1.85) | |
| Che1 rs1564796 | | | 0.29 | | 0.38 |
| T/T | 7.2 (5.4, 15.6+) | 1 (Ref.) | | 1 (Ref.) | |
| T/C^{a} | 12.4 (5.3, 12.4+) | 0.56 (0.19, 1.68) | | 0.60 (0.19, 1.87) | |
| C/C^{a} | | | | | |
| p53 rs1042522 | | | 0.43 | | 0.29 |
| C/C | 8.7 (5.3, 14.8+) | 1 (Ref.) | | 1 (Ref.) | |
| C/G | 6.3 (4.4, 12.4+) | 1.35 (0.55, 3.35) | | 1.61 (0.64, 4.06) | |
| G/G | 8.4+ (7.2, 8.4+) | 0.41 (0.05, 3.29) | | 0.38 (0.05, 3.11) | |
| | | | 0.75 | | 0.59 |
| C/C | 8.7 (5.3, 14.8+) | 1 (Ref.) | | 1 (Ref.) | |
| Any G | 7.2 (5.4, 15.6+) | 1.15 (0.47, 2.81) | | 1.28 (0.52, 3.19) | |
| Pin1 rs2233678 | | | 0.033 | | 0.015 |
| G/G | 8.3 (6.3, 15.6+) | 1 (Ref.) | | 1 (Ref.) | |
| G/C | 4.2 (2.7, 5.7+) | 4.23 (0.92, 19.42) | | 7.21 (1.47, 35.33) | |
| Pin1 rs2233679 | | | 0.86 | | 0.99 |
| C/C | 7.2 (4.9, 15.6+) | 1 (Ref.) | | 1 (Ref.) | |
| C/T | 8.3 (6.3, 12.4+) | 1.05 (0.41, 2.65) | | 1.04 (0.41, 2.64) | |
| T/T | 8.1 (3.1, 14.8+) | 0.74 (0.20, 2.75) | | 1.08 (0.24, 4.91) | |
| | | | 0.90 | | 0.92 |
| C/C | 7.2 (4.9, 15.6+) | 1 (Ref.) | | 1 (Ref.) | |
| Any T | 8.3 (6.3, 14.8+) | 0.95 (0.40, 2.26) | | 1.05 (0.43, 2.54) | |
| CHEK2 | | | 0.88 | | 0.52 |
| rs22236142 | | | | | |
| G/G | 8.1 (4.4, 14.8+) | 1 (Ref.) | | 1 (Ref.) | |
| G/C | 8.3 (4.9, 12.4+) | 0.78 (0.29, 2.11) | | 0.55 (0.19, 1.58) | |
| C/C | 7.5 (3.3, 15.6+) | 0.93 (0.31, 2.81) | | 0.88 (0.28, 2.77) | |
| | | | 0.69 | | 0.37 |
| G/G | 8.1 (4.4, 14.8+) | 1 (Ref.) | | 1 (Ref.) | |
| Any C | 8.3 (5.4, 15.6+) | 0.84 (0.35, 2.00) | | 0.66 (0.26, 1.65) | |
| PCNA rs25406 | | | 0.59 | | 0.42 |
| G/G | 8.3 (2.7, 14.8+) | 1 (Ref.) | | 1 (Ref.) | |
| G/A | 8.1 (5.7, 15.6+) | 0.62 (0.22, 1.71) | | 0.54 (0.19, 1.52) | |
| A/A | 8.7 (5.3, 10.7+) | 0.67 (0.21, 2.19) | | 0.55 (0.17, 1.84) | |
| | | | 0.30 | | 0.19 |
| G/G | 8.3 (2.7, 14.8+) | 1 (Ref.) | | 1 (Ref.) | |
| Any A | 8.1 (5.7, 15.6+) | 0.64 (0.26, 1.56) | | 0.54 (0.22, 1.35) | |
| p21 rs1801270 | | | 0.44 | | 0.21 |
| A/A | 7.2 (3.3, 8.7) | 1 (Ref.) | | 1 (Ref.) | |
| A/C | 12.4 (5.3, 15.6+) | 0.53 (0.19, 1.47) | | 0.39 (0.13, 1.11) | |
| C/C | 6.3 (3.1, 10.7) | 0.85 (0.29, 2.48) | | 0.72 (0.24, 2.15) | |
| | | | 0.32 | | 0.14 |
| A/A | 7.2 (3.3, 8.7) | 1 (Ref.) | | 1 (Ref.) | |
| Any C | 12.4 (5.4, 15.6+) | 0.64 (0.27, 1.56) | | 0.50 (0.20, 1.25) | |
| p21 rs1059234 | | | 0.56 | | 0.26 |
| T/T | 7.2 (3.3, 8.7) | 1 (Ref.) | | 1 (Ref.) | |
| T/C | 12.4 (5.3, 15.6+) | 0.59 (0.22, 1.57) | | 0.42 (0.15, 1.19) | |
| C/C | 6.3 (2.7, 10.7+) | 0.76 (0.25, 2.35) | | 0.67 (0.21, 2.12) | |
| | | | 0.32 | | 0.14 |
| T/T | 7.2 (3.3, 8.7) | 1 (Ref.) | | 1 (Ref.) | |
| Any C | 12.4 (5.4, 15.6+) | 0.64 (0.27, 1.56) | | 0.50 (0.20, 1.25) | |

†*P value was based on the log-rank test in the univariate analysis and Wald test in the multivariable analysis within Cox regression model (‡) adjusted for liver metastasis and adjuvant history in the evaluation cohort; age group.
^{a}Grouped together for analysis.

TABLE M
Gene polymorphism and clinical outcome in the validation
cohort treated with TAS-102

| | | | Disease Control Rate | | | |
|---|---|---|---|---|---|---|
| | | | | | | P |
| | | N | SD | | PD | value* |
| Pin1 | | | | | | 1.00 |
| rs2233678 | | | | | | |
| G/G | | 91 | 26 (29%) | | 65 (71%) | |
| G/C a | | 35 | 11 (30%) | | 26 (70%) | |
| C/C a | | 3 | | | | |
| ATR | | | | | | 0.12 |
| rs2227928 | | | | | | |
| G/G | | 39 | 12 (31%) | | 27 (69%) | |
| G/A | | 65 | 14 (22%) | | 50 (78%) | |
| A/A | | 25 | 11 (44%) | | 14 (56%) | |
| | | | | | | 0.83 |
| G/G | | 39 | 12 (31%) | | 27 (69%) | |
| Any A | | 90 | 25 (28%) | | 64 (72%) | |
| | | Progression-Free Survival | | | | |
| | | Median, | | | | |
| | | months | HR | | HR | P |
| | | (95% CI) | (95% CI) † | P value* | (95% CI)‡ | value* |
| Pin1 | | | | 0.98 | | 0.45 |
| rs2233678 | | | | | | |
| G/G | 91 | 2.0 | 1 | | 1 | |
| | | (1.9, 2.1) | (Ref.) | | (Ref.) | |
| G/C ^{a} | 35 | 2.0 | 1.00 | | 1.18 | |
| | | (1.9, 2.3) | (0.66, 1.54) | | (0.76, 1.84) | |
| C/C ^{a} | 3 | | | | | |
| ATR | | | | 0.19 | | 0.20 |
| rs2227928 | | | | | | |
| G/G | 39 | 2.1 | 1 | | 1 | |
| | | (1.9, 2.5) | (Ref.) | | (Ref.) | |
| G/A | 65 | 2.0 | 1.26 | | 1.45 | |
| | | (1.8, 2.0) | (0.81, 1.95) | | (0.92, 2.28) | |
| A/A | 25 | 2.4 | 0.79 | | 1.03 | |
| | | (1.8, 6.4+) | (0.43, 1.43) | | (0.55, 1.90) | |
| | | | | 0.61 | | 0.20 |
| G/G | 39 | 2.1 | 1 | | 1 | |
| | | (1.9, 2.5) | (Ref.) | | (Ref.) | |
| Any A | 90 | 2.0 | 1.11 | | 1.33 | |
| | | (1.9, 2.1) | (0.73, 1.68) | | (0.86, 2.05) | |
| | | Overall Survival | | | | |
| | | Median, | | | | |
| | | months | HR | | HR | P |
| | | (95% CI) | (95% CI) † | P value* | (95% CI) ‡ | value* |
| Pin1 | | | | 0.75 | | 0.93 |
| rs2233678 | | | | | | |
| G/G | 91 | 5.8 | 1 | | 1 | |
| | | (4.1, 8.7+) | (Ref.) | | (Ref.) | |
| G/C ^{a} | 35 | 5.4 | 0.91 | | | 0.97 |
| | | (3.9, 9.0+) | (0.50, 1.66) | | (0.53, 1.80) | |
| C/C ^{a} | 3 | | | | | |
| ATR | | | | 0.74 | | 0.62 |
| rs2227928 | | | | | | |
| G/G | 39 | 8.1+ | 1 | | 1 | |
| | | (3.9, 8.1+) | (Ref.) | | (Ref.) | |
| G/A | 65 | 5.4 | 1.20 | | 1.36 | |
| | | (3.9, 9.0+) | (0.65, 2.21) | | (0.73, 2.52) | |
| A/A | 25 | 8.8+ | 0.94 | | 1.32 | |
| | | (3.6, 8.8+) | (0.41, 2.12) | | (0.56, 3.10) | |
| | | | | 0.70 | | 0.33 |
| G/G | 39 | 8.1+ | 1 | | 1 | |
| | | (3.9, 8.1+) | (Ref.) | | (Ref.) | |
| Any A | 90 | 5.5 | 1.12 | | 1.35 | |
| | | (4.1, 9.0+) | (0.62, 2.02) | | (0.74, 2.44) | |

†* P value was based on the log-rank test in the univariate analysis and Wald test in the multivariable analysis within Cox regression model (‡) adjusted for liver metastasis and adjuvant history in the evaluation cohort; age group. ^{a} Grouped together for analysis. + Estimates not reached yet.

Graphs depicting the OS, survival functions, and PFS for the Japanese TAS-102 cohort and the ITA REGORA cohort demonstrate that the ATM rs609429 (C/C), the XRCC3 rs861539 (G/G) genotypes are significantly associated with poor OS or PFS in the Japanese TAS-102 treated cohort but are not significantly correlated in the regorafenib cohort (FIGS. 3-5). Additional results are presented in Tables N-P below:

TABLE N
Overall Survival by ATM rs609429

| | Median OS | Log-rank P |
|---|---|---|
| ATM rs609429 | (months) | value |
| JPN TAS-102 cohort | | 0.022 |
| C/C (n = 14) | 4.4 | |
| G/C or G/G (n = 38) | 8.7 | |
| ITA REGORA | | 0.459 |
| cohort | | |
| C/C (n = 28) | 4.5 | |
| G/C or G/G (n = 24) | 5.9 | |

TABLE O
Progression-Free Survival by XRCC3 rs861539

| | Median PFS | Log-rank P |
|---|---|---|
| XRCC3 rs861539 | (months) | value |
| JPN TAS-102 cohort | | 0.024 |
| G/G (n = 35) | 2.3 | |
| G/A (n = 17) | 3.8 | |
| ITA REGORA | | 0.801 |
| cohort | | |
| G/G (n = 15) | 1.7 | |
| G/A (n = 37) | 1.9 | |

TABLE P
Overall Survival by by XRCC3 rs861539

| | Median OS | Log-rank P |
|---|---|---|
| XRCC3 rs861539 | (months) | value |
| JPN TAS-102 cohort | | 0.012 |
| G/G (n = 35) | 6.3 | |
| G/A (n = 17) | 15.6+ | |
| ITA REGORA | | 0.28 |
| cohort | | |
| G/G (n = 15) | 4.1 | |
| G/A (n = 37) | 5.9 | |

SNPs and TAS-102-Related Toxicity with Clinical Outcome

Well known TAS-102 related grade (Gr) 3≤toxicities including neutropenia, leukopenia, anemia, anorexia, nausea and diarrhea were analyzed for association with clinical outcomes. In univariate analysis for the evaluation cohort, neutropenia Gr3≤(n=20) showed longer PFS and OS compared to that less than grade 3 (n=32), but it was not statistically significant (PFS, 3.4 vs. 2.1 months, HR 0.68, 95% CI: 0.37-1.28, P=0.21; OS, 8.3 vs. 6.3 months, HR 0.59, 95% CI: 0.24-1.43, P=0.22). These findings were confirmed in the validation cohort in PFS (2.3 vs. 1.9 months, HR 0.50, 95% CI: 0.32-0.77, P<0.001) and OS (8.8 vs. 3.7 months, HR 0.21, 95% CI: 0.10-0.44, P<0.001) (Table 17). Gr3≤neutropenia was more frequent in patients with the any G allele (n=16/38) in ATM rs609429 compared to those with the C/C variant (n=4/14) (42% vs. 29%, p=0.52) though no significance was observed in the evaluation cohort. In the validation cohort, patients with the any G allele (n=34/78) in ATM rs609429 were marginal significantly associated with high incidence of Gr3≤neutropenia compared to those with the C/C variant (n=13/51) (44% vs. 26%, P=0.060). There was no significant association between other SNPs and toxicity (Table 18).

TABLE Q
Toxicities and clinical outcome in the evaluation and validation
cohort treated with TAS-102

| | | | |
|---|---|---|---|
| | Median, months | | P |
| N | (95% CI) | HR (95% CI) | value* |

Progression-free Survival

| | | | | |
|---|---|---|---|---|
| Evaluation cohort | | | | |
| Neutropenia | | | | 0.21 |
| <3 | 32 | 2.1 (1.8, 3.0) | 1 (Ref.) | |
| ≥3 | 20 | 3.4 (1.8, 4.9) | 0.68 (0.37, 1.28) | |
| Leukopenia | | | | 0.83 |
| <3 | 40 | 2.5 (1.9, 3.5) | 1 (Ref.) | |
| ≥3 | 12 | 3.4 (1.6, 4.6) | 0.93 (0.45, 1.90) | |
| Anemia | | | | 0.35 |
| <3 | 40 | 2.5 (2.0, 3.4) | 1 (Ref.) | |
| ≥3 | 12 | 3.4 (1.5, 6.0+) | 0.70 (0.32, 1.53) | |
| Anorexia | | | | 0.33 |
| <3 | 44 | 2.9 (2.1, 3.4) | 1 (Ref.) | |
| ≥3 | 8 | 2.7 (0.9, 11.3) | 0.67 (0.27, 1.69) | |
| Nausea | | | | 0.48 |
| <3 | 35 | 3.3 (2.3, 3.7) | 1 (Ref.) | |
| ≥3 | 17 | 1.9 (1.6, 2.7) | 1.24 (0.62, 2.49) | |
| Diarrhea | | | | 0.79 |
| <3 | 45 | 2.5 (2.0, 3.4) | 1 (Ref.) | |
| ≥3 | 7 | 2.9 (1.8, 6.0+) | 0.88 (0.35, 2.26) | |
| Validation cohort | | | | |
| Neutropenia | | | | <0.001 |
| <3 | 81 | 1.9 (1.7, 1.9) | 1 (Ref.) | |
| ≥3 | 47 | 2.3 (2.0, 3.7) | 0.50 (0.32, 0.77) | |

Overall Survival

| | | | | |
|---|---|---|---|---|
| Evaluation cohort | | | | |
| Neutropenia | | | | 0.22 |
| <3 | 32 | 6.3 (3.4, 15.6+) | 1 (Ref.) | |
| ≥3 | 20 | 8.3 (6.3, 14.8+) | 0.59 (0.24, 1.43) | |
| Leukopenia | | | | 0.53 |
| <3 | 40 | 8.3 (4.9, 15.6+) | 1 (Ref.) | |
| ≥3 | 12 | 8.1 (5.7, 10.7+) | 0.73 (0.26, 2.01) | |
| Anemia | | | | 0.16 |
| <3 | 40 | 8.3 (5.7, 15.6+) | 1 (Ref.) | |
| ≥3 | 12 | 6.3 (2.0, 14.8+) | 1.87 (0.74, 4.74) | |
| Anorexia | | | | 0.76 |
| <3 | 44 | 8.1 (5.7, 12.4+) | 1 (Ref.) | |
| ≥3 | 8 | 8.7 (1.4, 15.6+) | 0.85 (0.27, 2.69) | |
| Nausea | | | | 0.28 |
| <3 | 35 | 8.1 (6.3, 14.8+) | 1 (Ref.) | |
| ≥3 | 17 | 5.7 (2.7, 12.4) | 1.58 (0.65, 3.83) | |
| Diarrhea | | | | 0.46 |
| <3 | 45 | 8.3 (6.3, 12.4) | 1 (Ref.) | |
| ≥3 | 7 | 5.7 (2.0, 14.8+) | 1.49 (0.49, 4.53) | |
| Validation cohort | | | | |
| Neutropenia | | | | <0.001 |
| <3 | 81 | 3.7 (3.3, 5.1) | 1 (Ref.) | |
| ≥3 | 47 | 8.8+ | 0.21 (0.10, 0.44) | |

*Based on the log-rank test in the univariate analysis

TABLE R
SNPs and grade 3≤ neutropenia in the evaluation and
validation cohort treated with TAS-102

| | | Neutropenia | | |
|---|---|---|---|---|
| | N | <3 | ≥3 | P value* |
| Evaluation cohort | | | | |
| ATM rs609429 | | | | 0.52 |
| C/C | 14 | 10 (71%) | 4 (29%) | |
| Any G | 38 | 22 (58%) | 16 (42%) | |
| BRCA1 rs799917 | | | | 0.78 |
| G/G | 23 | 15 (65%) | 8 (35%) | |
| Any A | 29 | 17 (59%) | 12 (41%) | |
| BRCA1 rs16941 | | | | 0.78 |
| T/T | 23 | 15 (65%) | 8 (35%) | |
| Any C | 29 | 17 (59%) | 12 (41%) | |
| BRCA2 rs144848 | | | | 0.77 |
| A/A | 32 | 19 (59%) | 13 (41%) | |
| Any C | 20 | 13 (65%) | 7 (35%) | |
| RAD51 rs1801320 | | | | 0.13 |
| G/G | 44 | 25 (57%) | 19 (43%) | |
| G/C | 8 | 7 (88%) | 1 (13%) | |
| XRCC3 rs861539 | | | | 0.54 |
| G/G | 35 | 23 (66%) | 12 (34%) | |
| G/A | 17 | 9 (53%) | 8 (47%) | |
| FANCD2 | | | | 1.00 |
| rs2272125 | | | | |
| T/T | 46 | 28 (61%) | 18 (39%) | |
| T/G | 6 | 4 (67%) | 2 (33%) | |
| FANCD2 | | | | 1.00 |
| rs6792811 | | | | |
| A/A | 35 | 22 (63%) | 13 (37%) | |
| Any G | 17 | 10 (59%) | 7 (41%) | |
| H2AX rs2509049 | | | | 0.089 |
| T/T | 21 | 16 (76%) | 5 (24%) | |
| Any C | 31 | 16 (52%) | 15 (48%) | |
| H2AX rs643788 | | | | 0.082 |
| C/C | 22 | 17 (77%) | 5 (23%) | |
| Any T | 30 | 15 (50%) | 15 (50%) | |
| Che1 rs1045056 | | | | 1.00 |
| T/T | 41 | 25 (61%) | 16 (39%) | |
| T/C | 11 | 7 (64%) | 4 (36%) | |
| Che1 rs1564796 | | | | 0.75 |
| T/T | 40 | 24 (60%) | 16 (40%) | |
| Any C | 12 | 8 (67%) | 4 (33%) | |
| p53 rs1042522 | | | | 1.00 |
| C/C | 21 | 13 (62%) | 8 (38%) | |
| Any G | 31 | 19 (61%) | 12 (39%) | |
| Pin1 rs2233678 | | | | 1.00 |
| G/G | 50 | 31 (62%) | 19 (38%) | |
| G/C | 2 | 1 (50%) | 1 (50%) | |
| Pin1 rs2233679 | | | | 0.77 |
| C/C | 19 | 11 (58%) | 8 (42%) | |
| Any T | 33 | 21 (64%) | 12 (36%) | |
| CHEK2 rs2267130 | | | | 0.25 |
| T/T | 22 | 16 (73%) | 6 (27%) | |
| Any C | 30 | 16 (53%) | 14 (47%) | |
| CHEK2 rs2236142 | | | | 0.40 |
| G/G | 22 | 12 (55%) | 10 (45%) | |
| Any C | 30 | 20 (67%) | 10 (33%) | |
| p21 rs1801270 | | | | 0.21 |
| A/A | 15 | 7 (47%) | 8 (53%) | |
| Any C | 37 | 25 (68%) | 12 (32%) | |
| p21 rs1059234 | | | | 1.00 |
| C/C | 13 | 8 (62%) | 5 (38%) | |
| Any T | 39 | 24 (62%) | 15 (38%) | |
| PCNA rs25406 | | | | 0.070 |
| G/G | 19 | 15 (79%) | 4 (21%) | |
| Any A | 30 | 15 (50%) | 15 (50%) | |
| ATR rs2227928 | | | | 0.55 |
| A/A | 16 | 11 (69%) | 5 (31%) | |
| Any G | 36 | 21 (58%) | 15 (42%) | |
| CHEK1 rs521102 | | | | 0.56 |
| G/G | 20 | 11 (55%) | 9 (45%) | |
| Any A | 32 | 21 (66%) | 11 (34%) | |
| Validation Cohort | | | | |
| ATM rs609429 | | | | 0.060 |
| C/C | 51 | 37 (74%) | 13 (26%) | |
| Any G | 78 | 44 (56%) | 34 (44%) | |
| XRCC3 rs861539 | | | | 0.18 |
| G/G | 50 | 36 (72%) | 14 (28%) | |
| Any A | 75 | 44 (59%) | 30 (41%) | |
| Pin1 rs2233678 | | | | 0.42 |
| G/G | 91 | 60 (66%) | 31 (34%) | |
| Any C | 38 | 21 (57%) | 16 (43%) | |
| CHEK2 rs2267130 | | | | 0.41 |
| T/T | 35 | 20 (57%) | 15 (43%) | |
| Any C | 94 | 61 (66%) | 32 (34%) | |
| ATR rs2227928 | | | | 1.00 |
| G/G | 39 | 24 (63%) | 14 (37%) | |
| Any A | 90 | 57 (63%) | 33 (37%) | |

*P value based on the Fisher's exact test for toxicity.

### Discussion

Without being bound by theory, this study demonstrates that the incorporation of FTD as the active cytotoxic component of TAS-102 into DNA induces DSBs followed by DNA repair mainly through HR pathway. Furthermore, to Applicant's knowledge, this study is the first to report that HR pathway-related genes polymorphisms, ATM rs609429 and XRCC3 rs861539, are associated with clinical outcomes in mCRC patients receiving TAS-102.

HR is known as a conservative pathway to repair DSBs induced by DNA damaging agents (Mayer et al. [32]), and utilizes a sister DNA strand harboring correct sequence as a homologous pair for reference in the recombinant process of repairs for DSBs (Cheung-Ong et al. [37]). Hence, genes regulating the activity of HR pathway can be molecular targets with regard to prediction of chemo-sensitivity or elucidation of chemo resistance.

ATM is an upper stream gene in the HR pathway and plays as a key activator of the cellular response to DSBs through subsequent phosphorylation of downstream HR-related genes. It is recruited and activated of by the MRN complex acting as sensor of DNA damage. ATM is also responsible for cell cycle checkpoint activation and p53, responsible for cell cycle arrest, is one of its target genes (Bijnsdorp et al. [4]). Thus, HR-derived DNA repair is harmonized with cell cycle progression through ATM activation. A protein expression analysis for ATM, y-H2AX and Ku70 in HR-pathway in stage II/III colorectal cancer tissue reported that loss of ATM expression is independently associated with shorter DFS, and suggests ATM as a negative prognostic biomarker in colorectal cancer (Boggs et al. [20]). Applicant thereby identified the key activator of HR-mediated DNA repair, ATM gene, as a primary candidate that might also influence the efficacy of TAS-102 treatment or prognosis in patients with mCRC.

The role of HR-related gene polymorphisms for the risk of developing colorectal cancer and the chemo-sensitivity to mCRC still remain unclear as contrasted with breast or lung cancer (Goode et al. and Moreno et al. [48,49]). However, an association between oxaliplatin, a third-generation platinum analogue, and SNPs of genes involved in the DNA repair pathways including nucleotide excision repair, base excision repair and mismatch repair was reported within an exploratory study. The results demonstrated that ATM rs1801516 and ERCC5 rs1047768 SNPs were both involved in efficacy of oxaliplatin in patients with advanced CRC (Kweekel et al. [50]).

There have been few studies that mentioned biological function of individual ATM genetic variants in various cancer types. An ATM mRNA transcript from a cell-line carrying the variants demonstrated that the minor allele (G) of ATM rs609429 generates a weak 5′ splice site and decreases gene expression (Angele et al. [53]). An in vitro study that examined the mode of action of FTD-induced DNA damage after its incorporation into DNA, observed ATM phosphorylation in FTD-treated cells indicating presence of double strand DNA breaks (Peng et al. [46]). The dominant C allele is considered to mainly activate ATM phosphorylation corresponding to DSBs induced by FTD incorporation into DNA. In fact, the present study revealed that patients carrying the C/C variant had poor outcome in the evaluation and validation cohorts treated with TAS-102, though the different frequency of the C/C variant in ATM rs609429 (26.9% vs. 39.5%) was observed. Without being bound by theory, these results support the conclusion that this is one of the main pathways for developing chemo-resistance to TAS-102 treatment.

XRCC3 is another crucial member of DNA repair genes encoding the RecA/RAD51-related protein family for the maintenance of genome stability in HR repair for DNA double-strand breaks (Goode et al. [48]). Association between XRCC3 rs861539 polymorphism and cancer risk have been reported in several cancer types including bladder cancer, melanoma and lung cancer [21,27,28,29,30]. Two studies for lung cancer showed no association with the gene variants (Butkiewicz et al. and David-Beabes et al. [54,55]). Different studies demonstrated a statistically significant increased risk of bladder cancer (Matullo et al. [56]) and melanoma (Winsey et al. [57]) in heterozygous or homozygous XRCC3 rs861539 compared to wild type. In addition, some studies for gastrointestinal cancer have been reported including colorectal cancer (Jiang et al. [58,59]) and gastric cancer (Wang et al. [60]). Jiang et al. performed a meta-analysis to evaluate the role of XRCC1 and XRCC3 genotypes in CRC susceptibility, and identified no association between XRCC3 T241M (r5861539) and XRCC1 with colorectal cancer risk [31]. Discordant results were reported in a meta-analysis by Wang et al. describing a possible correlation between XRCC3 T241M (r5861539) T241M Met/Met (A/A) variant and an elevated risk of CRC was reported in an Asian population (Wang et al. [59]). Another research group conducted a similar meta-analysis for gastric cancer (GC) finding that XRCC3 T241M C/C variants are associated with decreased GC risk (Wang et al. [60]). Furthermore, no evidence of association of the SNPs with response to chemotherapy or clinical outcomes was observed in the studies. Without being bound by theory, XRCC3 T241M (r5861539) polymorphism might be involved in cancer development, but its role as a predictive marker remains unknown.

Mutated DNA repair pathway genes are often molecular targets of chemotherapeutic agents, especially biologic agents such as pharmacological inhibitors of the enzyme poly ADP ribose polymerase (PARP) inhibitor that deteriorate the DNA repair in cancer cells leading to cell death (Mayer et al. [32]). In the present study, Applicant observed an ethnic difference in the allele frequency in XRCC3 rs861539 that showed the lack of A/A variant (0% vs. 20.0%) and high frequency of the G/G variant (67.3% vs. 40.0%) in the evaluation cohort compared to those of the validation cohort. However, a trend toward shorter OS in the G/G variant compared with the G/A variant was confirmed in the validation cohort when Applicant excluded the A/A variant from the analysis, which is consistent with those observed in the evaluation cohort; though without statistical significance.

Taken together, Applicant's data demonstrates that the dominant wild-type variants, C/C in ATM rs609429 and G/G in XRCC3 rs861539, affect chemosensitivity to TAS-102 treatment leading to shorter PFS or OS. Without being bound by theory, Applicant believes that this is caused by insufficient DNA repair for DSBs by FTD incorporation into DNA. In addition, Applicant found an association between grade 3≤neutropenia and ATM rs609429. Without being bound by theory, this association can be explained by the maintenance of increased FTD concentration in tumor cells of patients carrying the G allele compared with the concentration level in those with the C/C variant. Applicant has demonstrated more clearly the DNA repair mechanism and identified candidate markers for clinical outcomes in mCRC patients treated with TAS-102 by comparing the three independent cohorts including an evaluation cohort for discovery, a control cohort with comparable clinical characteristics and disease stage and a larger validation cohort with comparable clinical characteristics and receiving the same treatment.

In conclusion, Applicant's study demonstrates the first evidence that genetic variants in the HR pathway, ATM rs609429 and XRCC3 rs861539, may serve as predictive and prognostic markers in refractory mCRC patients receiving TAS-102. These results also provide support for the formulation of new combination treatments with TAS-102.

### Example 3: hENT1 Variants

Decreased expression of human equilibrative nucleoside transporter (hENT1) and thymidine kinase 1 (TK1) results in decreased nuclear intake of FTD. Applicant tested whether gene polymorphisms involved in FTD metabolism are associated with outcomes in patients with refractory metastatic colorectal cancer (mCRC) treated with TAS-102.

Applicant analyzed genomic DNA extracted from 104 blood samples of two different cohorts: an evaluation set of 52 patients receiving TAS-102 (median age 61 years, male 44%, median followup time, 6.4 months), and a control group of 52 patients receiving regorafenib without history of TAS-102 treatment (median age 64 years, male 44%, all patients deceased). Single nucleotide polymorphisms (SNPs) of genes in FTD metabolism (TKJ, hENT1) were analyzed by PCR-based direct sequencing.

The candidate SNPs were selected by their frequency and potential function. Associations between selected SNPs and PFS and OS were evaluated by KaplanMeier and logrank tests in the overall population. Cox proportional hazard regression model was used in multivariate analyses.

Exemplary PCR primers used in the example are provided in Table S below.

TABLE S

| SNP | Forward primer | Reverse primer |
|---|---|---|
| rs760370 | TGGGGGACACTCAGTAGA | AACGTGTATGGTGGGGTTGT |
| | GG (SEQ ID NO: 12) | (SEQ ID NO: 13) |
| rs9394992 | CTGCCTCCTGTGCTCCAT | TGGGAAATGACTGAGCTGTG |
| | (SEQ ID NO: 14) | (SEQ ID NO: 15) |

The amplicon generated comprising, or alternatively consisting essentially of, or yet further consisting of hENT1 rs760370 has the following sequence (R=A or G): 5′TGGGGGACACTCAGTAGAGGGAGGGCAAAAGGAGAGTCCCTGCTCCCAGAGCTGA GAGGAGAGATGGCTCAGGAGGGGCTCCCAGGCTGAGGGGATAGTGACTGTAGTGGA GGGGGGCGCCAAGCTTACTTGGGTGGAGGTGGAGACAGGTTTGC(R)GGAAGGAGTG AAAGACAACCCCACCATACACGTT3′ (SEQ ID NO:24). Thus, the sequence of the amplicon generated for the (A) allele is: 5′TGGGGGACACTCAGTAGAGGGAGGGCAAAAGGAGAGTCCCTGCTCCCAGAGCTGA GAGGAGAGATGGCTCAGGAGGGGCTCCCAGGCTGAGGGGATAGTGACTGTAGTGGA GGGGGGCGCCAAGCTTACTTGGGTGGAGGTGGAGACAGGTTTGCAGGAAGGAGTGA AAGACAACCCCACCATACACGTT3′ (SEQ ID NO: 86) and the sequence generated for the (G) allele is:

(SEQ ID NO: 87)
5′TGGGGGACACTCAGTAGAGGGAGGGCAAAAGGAGAGTCCCTGCTCCCA
GAGCTGAGAGGAGAGATGGCTCAGGAGGGGCTCCCAGGCTGAGGGGATAG
TGACTGTAGTGGAGGGGGGCGCCAAGCTTACTTGGGTGGAGGTGGAGACA
GGTTTGCGGGAAGGAGTGAAAGACAACCCCACCATACACGTT3′.

The amplicon generated comprising, or alternatively consisting essentially of, or yet further consisting of hENT1 rs9394992 has the following sequence (Y=C or T): 5′CTGCCTCCTGTGCTCCATTCCCAGGCTGCCTGCCCATTCTGTCCCCACCTC CGCTCTCCTGTGGGCAGTTCCCTGAAGGCCT(Y)GCCGGCTCCATTTGCCTTATTGC ACAGCTCAGTCATTTCCCA3′ (SEQ ID NO:25). Thus, the sequence of the hENT1 rs9394992 amplicon for the (C) allele is: 5′CTGCCTCCTGTGCTCCATTCCCAGGCTGCCTGCCCATTC TGTCCCCACCTCCGCTCTCCTGTGGGCAGTTCCCTGAAGGCCTCGCCGGCTCCATTT GCCTTATTGCACAGCTCAGTCATTTCCCA3′ (SEQ ID NO:88) and the sequence for the amplicon of the (T) allele is: 5′CTGCCTCCTGTGCTCCATTCCCAGGCTGCCTGCCCATTCT GTCCCCACCTCCGCTCTCCTGTGGGCAGTTCCCTGAAGGCCTTGCCGGCTCCATTTGC CTTATTGCACAGCTCAGTCATTTCCCA3′ (SEQ ID NO:89).

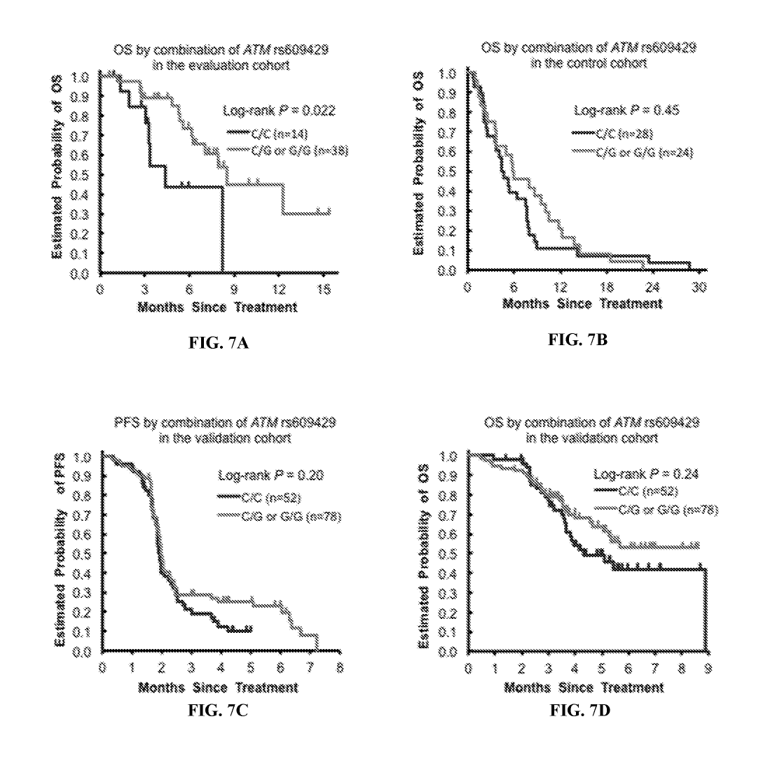

In univariate analysis, median PFS for patients with any hENT1 rs760370 G allele was 3.5 vs. 2.1 months for those carrying the A/A genotype (HR 0.44, 95% CI: 0.230.83, p=0.004). Median OS was 8.7 and 5.3 months in the two groups, respectively (HR 0.27, 95% CI: 0.100.70, p=0.003) (FIG. 6). Among patients with any hENT1 rs9394992 T allele, median PFS was 3.4 vs. 1.9 months for those with the C/C genotype (FIG. 7).

### Example 4: OCT2 and MATE1 Variants

The thymidine phosphorylase inhibitor (TPI) tipiracil is excreted by Organic cation transporter 2 (OCT2, also known as SLC22A2, mRNA: NM_153191, NM_003058) and Multidrug and toxin extrusion (MATE1, also known as SLC47A1, mRNA: NM_018242). Applicant tested whether gene polymorphisms in genes involved in TPI metabolism are associated with outcomes in patients with refractory metastatic colorectal cancer (mCRC) treated with TAS-102.

Single nucleotide polymorphisms (SNPs) were analyzed by PCR-based direct sequencing. Exemplary PCR primers used in the example are provided in Table T below.

TABLE T

| SNP | Forward primer | Reverse primer |
|---|---|---|
| rs316000 | CCAGACCACTCAAGCTTTCTC | AGTCATGTTGAAAGCCAGCA |
| | (SEQ ID NO: 16) | (SEQ ID NO: 17) |
| rs316019 | GAAGGCAGACTTCTTAGCAGAAT | ATACAGTTGGGCTCCTGGTG |
| | (SEQ ID NO: 18) | (SEQ ID NO: 19) |
| rs2289669 | CCAGTTTGTGCTAAGCATCG | ACACCTGGTGGGAAAACTTG |
| | (SEQ ID NO: 20) | (SEQ ID NO: 21) |

The amplicon generated comprising, or alternatively consisting essentially of, or yet further consisting of OCT2 rs316000 has the following sequence (R=A or G):5′CCAGACCACTCAAGCTTTCTCCATATCAGCAATAAGGCTGTTTCATTT CTCATTTGTAGCATTTTTATACTTCCTTCAAGAACTTTTCCTGTGCATTCTC(R)ACTTG GCTACCTGGTACAAGAGTCCTCACTTTCAGCTTATGCTGGCTTTCAACATGACT3′ (SEQ ID NO:26). Thus, the sequence of the OCT2 rs316000 amplicon for the (A) allele is: 5′CCAGACCACTCAAGCTTTCTCCATATCAGCAATAAGGCTGTTTCATTTCTCATTTG TAGCATTTTTATACTTCCTTCAAGAACTTTTCCTGTGCATTCTCAACTTGGCTACCTG GTACAAGAGTCCTCACTTTCAGCTTATGCTGGCTTTCAACATGACT3′ (SEQ ID NO:90) and the sequence for the (G) allele is: 5′CCAGACCACTCAAGCTTTCTCCATATCAGCAATA AGGCTGTTTCATTTCTCATTTGTAGCATTTTTATACTTCCTTCAAGAACTTTTCCTGTG CATTCTCGACTTGGCTACCTGGTACAAGAGTCCTCACTTTCAGCTTATGCTGGCTTTC AACATGACT3′ (SEQ ID NO:91).

The amplicon generated comprising, or alternatively consisting essentially of, or yet further consisting of OCT2 rs316019 has the following sequence (M=A or C):

(SEQ ID NO: 27)
5′GAAGGCAGACTTCTTAGCAGAATAAAATACTTTGATTTATTTCCTTTT
TATTCCAAATGGACTTACCAGTAATAGAGCAAGAAGAAGAAGTTGGGCAG
AG(M)AACTGTGAACTGCAACCACCTCCAGTGAGGAAGTGCGTAAGCCAC
CCCAGCTAGCACCAGGAGCCCAACTGTAT3′

The amplicon generated comprising, or alternatively consisting essentially of, or yet further consisting of MATE1 rs2289669 has the following sequence (R=A or G):

(SEQ ID NO: 28)
5′CCAGTTTGTGCTAAGCATCGTAACCTGGGGCTCAGTTTCCACAGTAGC
GTGG(R)AGTTCCCGGCTAGACAAAGGGGATGTTGCAAATCAGTCTTTTC
AAAACTTTTAGGACCAAGTTTTCCCACCAGGTGT3′

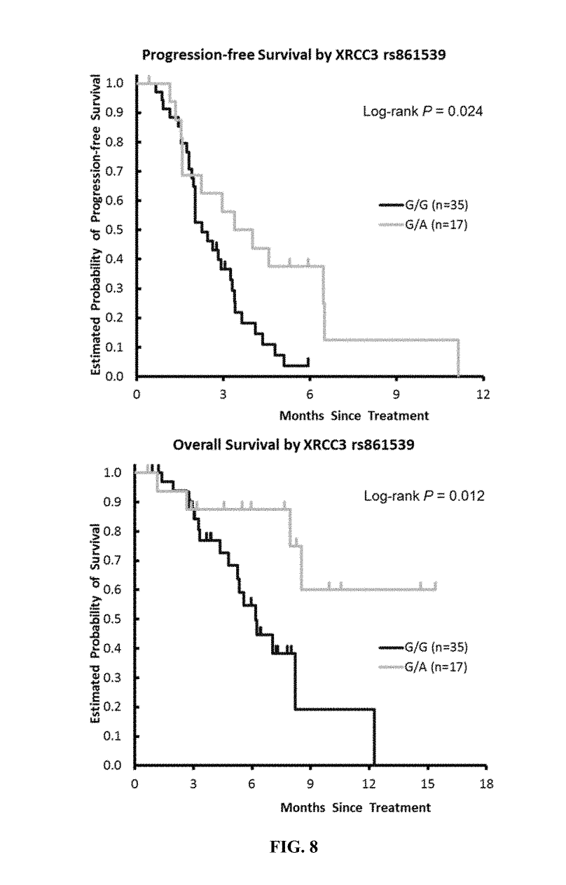

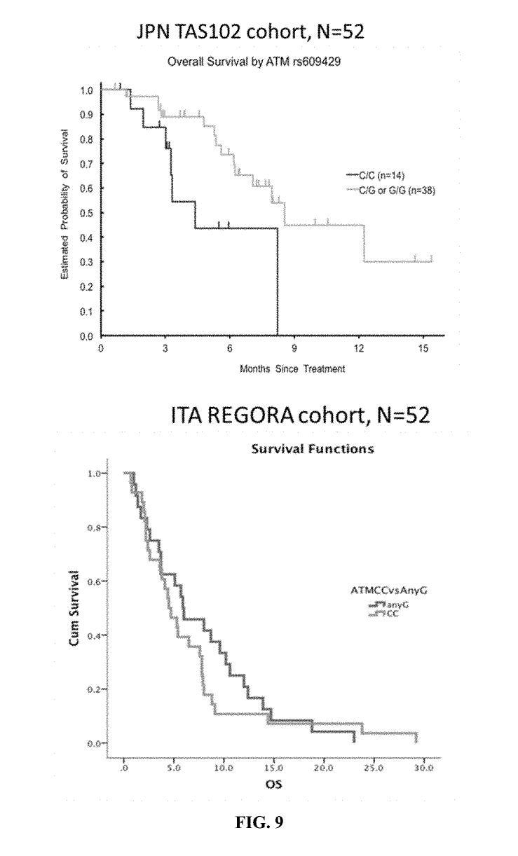

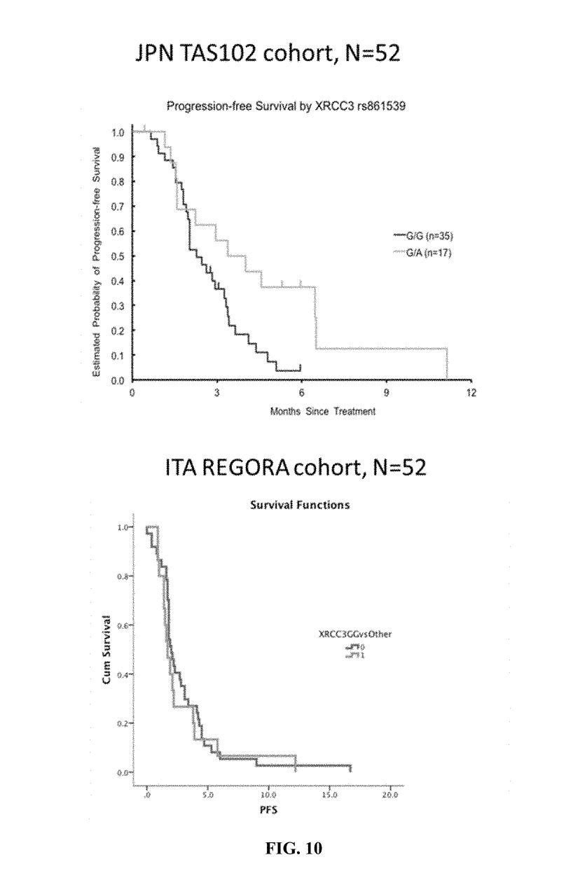

Neither TPI renal clearance (CLren) nor or tubular secretory Clearance (CLsec) were associated with SNPs in MATE1 (rs2289669 or rs2252281) when tested individually (FIG. 8). However, homozygous carriers of the minor allele in rs2289669 showed a trend toward a lower Clrenal reference (G/G): 33.3 l/h versus variant (A/A): 29.6 l/h (P=0.053) and CLsec reference. (FIGS. 8-10). TPI is strongly associated with PFS and OS with regard to renal clearance difference by OCT2 and MATE1 gene-gene interaction.

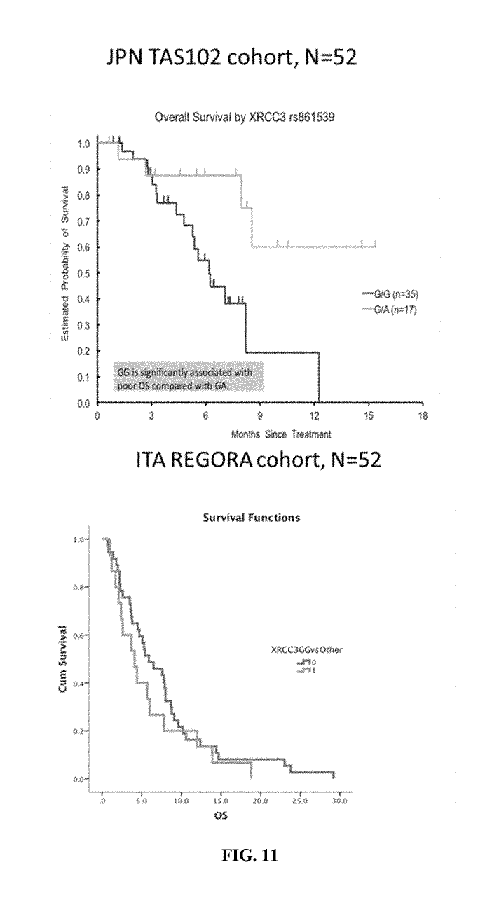

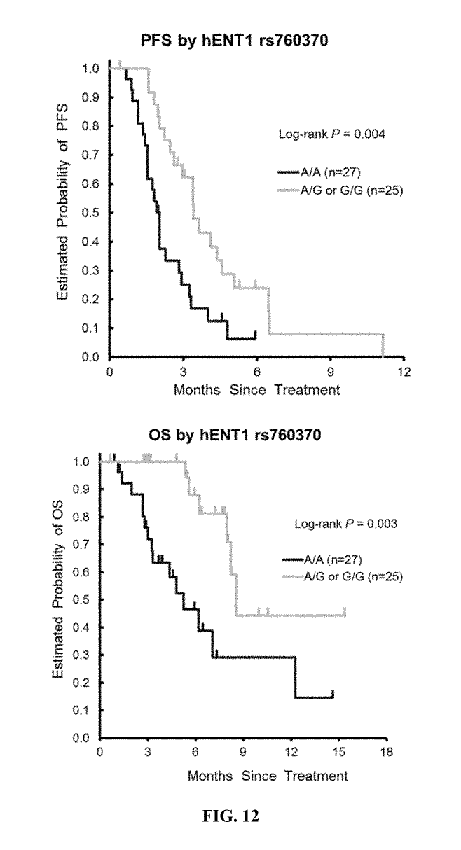

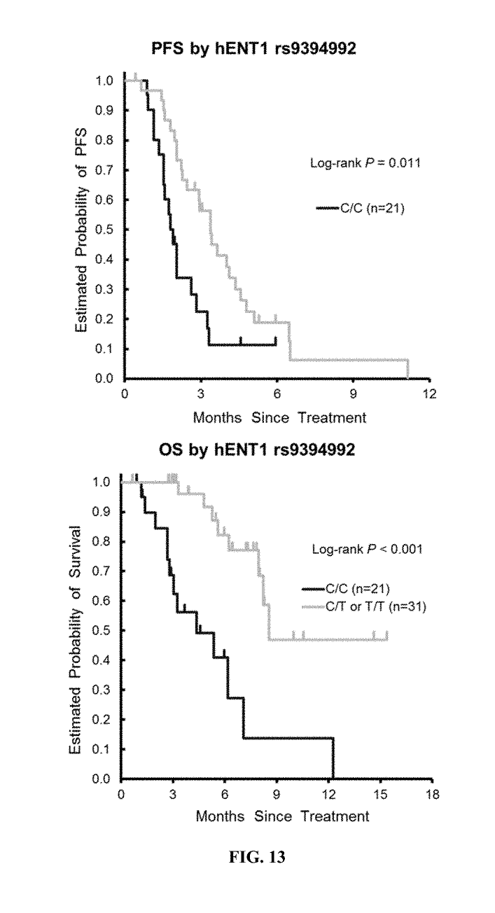

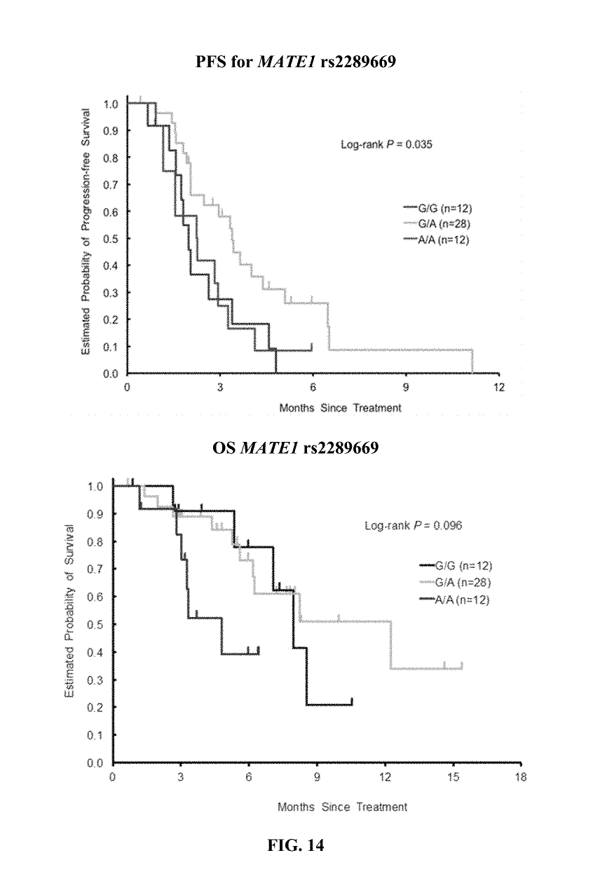

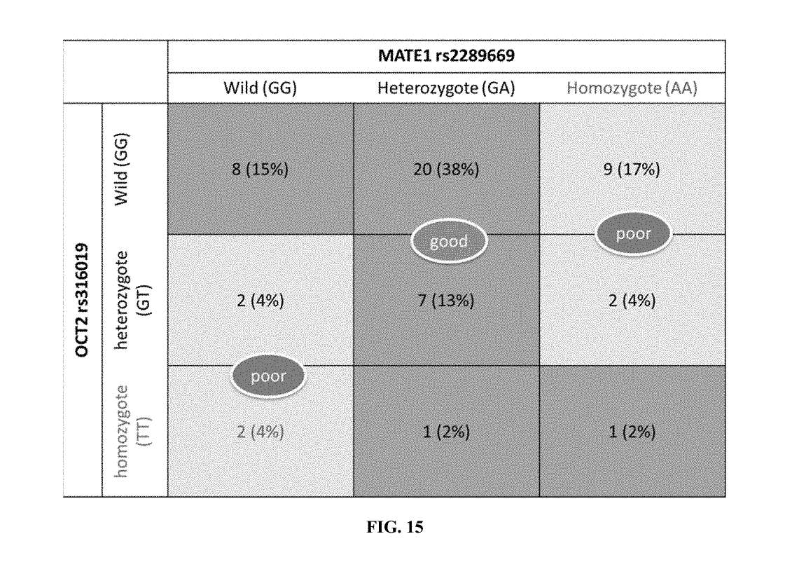

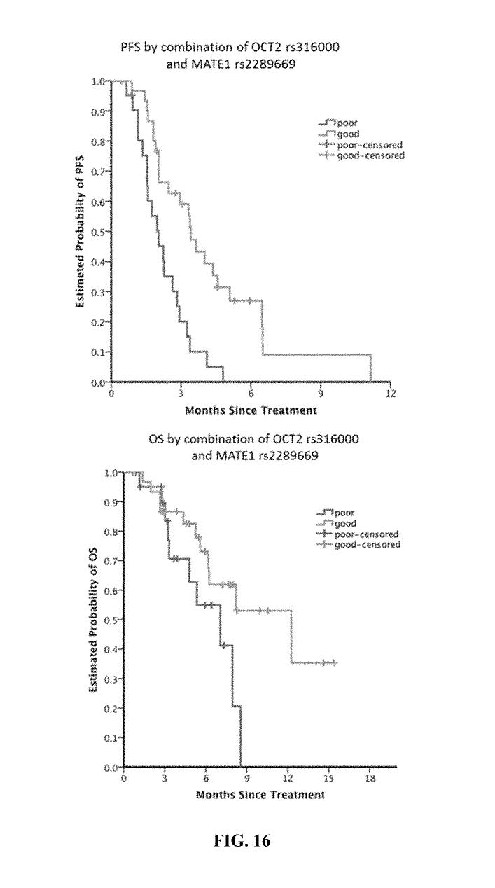

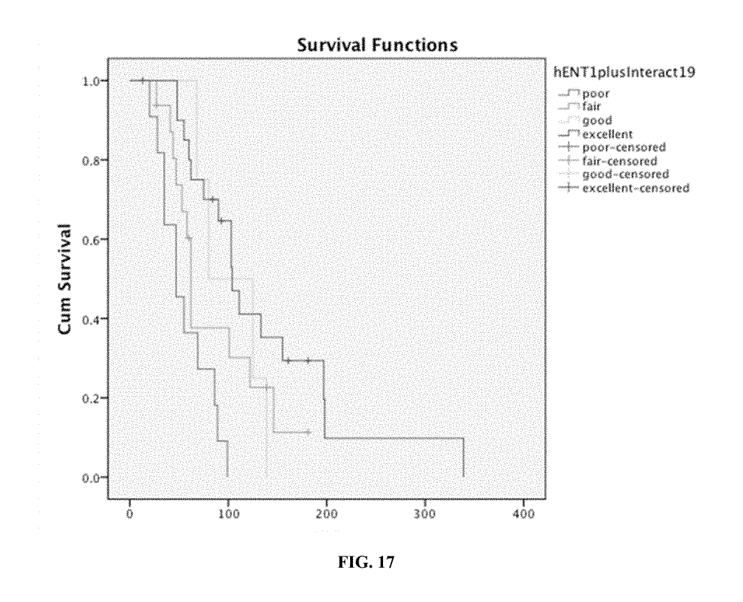

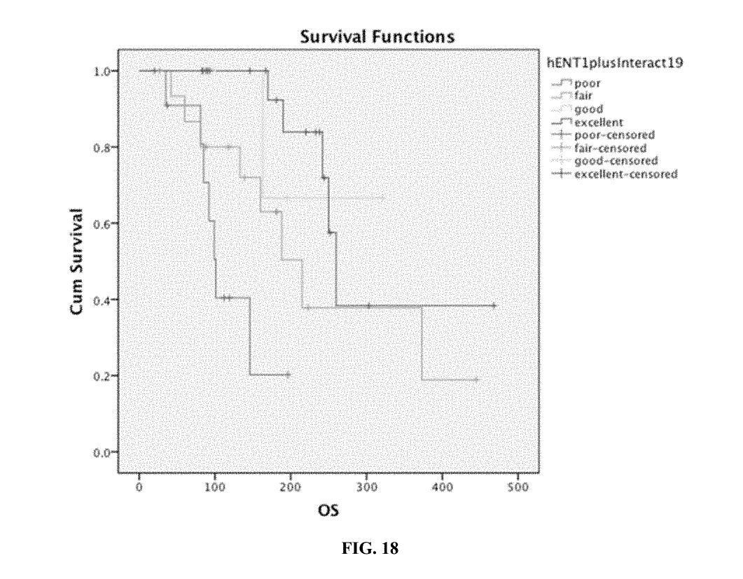

When hENT1 variants were analyzed in combination with the OCT2/MATE1 combination variant analysis, there was a consistent association with PFS and OS irrespective of OCT2/MATE1 combination indicating that the hENT1 variants were stronger predictors of PFS and OS. However, the triple combination mutant variant for hENT1 and OCT2/MATE1 exhibited significantly higher progression free survival (FIG. 11: the PFS (days): poor(11)=47.0, 95% CI: 25.4-68.6; fair (16)=62.0, 95% CI: 57.5-66.5); good(4)=80, 95% CI: 24.1-135.9; excellent(21)=104.0, 95% CI:83.5-124.5; overall(52)=80, 95% CI: 55.7-104.3 with a P value of 0.001) and overall survival (FIG. 12: the PFS (days): poor=101.0, 95% CI: 87.4-114.6; fair=215.0, 95% CI: 146.6-283.4); good=NR; excellent=260.0, 95% CI: 240.2-279.8; overall=242.0, 95% CI: 187.6-296.4 with a P value of 0.001). Thus, gene variants of hENT1 as influx of FTD is well harmonized with OCT2/MATE2 gene variants combination as efflux of TPI. Integrated classification clearly distinguished clinical outcome in mCRC patients receiving TAS-102.

TABLE U

OCT2/MATE1

| | Poor | good |
|---|---|---|
| hENT1 WT | Poor | fair |
| (poor) | | |
| hENT1 MT | Good | excellent |
| (good) | | |

### EQUIVALENTS

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosure embodied therein herein disclosed can be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. Several references are identified by an Arabic number, and the full bibliographic citation or these references are provided below. In case of conflict, the present specification, including definitions, will control.

### REFERENCES

1. Hong D S, Abbruzzese J L, Bogaard K, Lassere Y, Fukushima M, Mita A, Kuwata K, Hoff P M. Phase I study to determine the safety and pharmacokinetics of oral administration of TAS-102 in patients with solid tumors. Cancer 2006; 107: 1383-90.
2. Temmink O H, Emura T, de Bruin M, Fukushima M, Peters G J. Therapeutic potential of the dual-targeted TAS-102 formulation in the treatment of gastrointestinal malignancies. Cancer Sci 2007; 98: 779-89.
3. Bijnsdorp I V, Peters G J, Temmink O H, Fukushima M, Kruyt F A. Differential activation of cell death and autophagy results in an increased cytotoxic potential for trifluorothymidine compared to 5-fluorouracil in colon cancer cells. Int J Cancer 2010; 126: 2457-68.
4. Smith K M, Slugoski M D, Loewen S K, Ng A M, Yao S Y, Chen X Z, Karpinski E, Cass C E, Baldwin S A, Young J D. The broadly selective human Na+/nucleoside cotransporter (hCNT3) exhibits novel cation-coupled nucleoside transport characteristics. J Biol Chem. 2005; 280:25436-49.
5. Okayama T, Yoshisue K, Kuwata K, Komuro M, Ohta S, Nagayama S. Involvement of concentrative nucleoside transporter 1 in intestinal absorption of trifluorothymidine, a novel antitumor nucleoside, in rats. J Pharmacol Exp Ther 2012; 340:457-62.
6. Bonate P L, Arthaud L, Cantrell W R Jr, Stephenson K, Secrist J A 3rd, Weitman S. Discovery and development of clofarabine: a nucleoside analogue for treating cancer. Nat Rev Drug Discov 2006; 5:855-63.
7. Emura T, Suzuki N, Yamaguchi M, Ohshimo H, Fukushima M. A novel combination antimetabolite, TAS-102, exhibits antitumor activity in FU-resistant human cancer cells through a mechanism involving FTD incorporation in DNA. Int J Oncol 2004; 25:571-8.
8. Suzuki N, Nakagawa F, Nukatsuka M, Fukushima M. Trifluorothymidine exhibits potent antitumor activity via the induction of DNA double-strand breaks. Exp Ther Med 2011; 2: 393-97.
9. Emura T, Nakagawa F, Fujioka A, Ohshimo H, Yokogawa T, Okabe H, Kitazato K. D An optimal dosing schedule for a novel combination antimetabolite, TAS-102, based on its intracellular metabolism and its incorporation into DNA. Int J Mol Med 2004; 13:249-55.
10. Sakamoto K, Yokogawa T, Ueno H, Oguchi K, Kazuno H, Ishida K, Tanaka N, Osada A, Yamada Y, Okabe H, Matsuo K. Crucial roles of thymidine kinase 1 and deoxyUTPase in incorporating the antineoplastic nucleosides trifluridine and 2′-deoxy-5-fluorouridine into DNA. Int J Oncol 2015; 46:2327-34.
11. Temmink O H, Bijnsdorp I V, Prins H J, Losekoot N, Adema A D, Smid K, Honeywell R J, Ylstra B, Eijk P P, Fukushima M, Peters G J. Trifluorothymidine resistance is associated with decreased thymidine kinase and equilibrative nucleoside transporter expression or increased secretory phospholipase A2. Mol Cancer Ther 2010; 9:1047-57.
12. Doi T, Ohtsu A, Yoshino T, Boku N, Onozawa Y, Fukutomi A, Hironaka S, Koizumi W, Sasaki T. Phase I study of TAS-102 treatment in Japanese patients with advanced solid tumours. Br J Cancer 2012; 107:429-34.
13. Yoshisue K, Takahashi K, Okayama T, Yamashita F, Chiba M. INVESTIGATION OF TRANSPORTERS THAT PLAY AN IMPORTANT ROLE IN URINARY SECRETION OF THYMIDINE PHOSPHORYLASE INHIBITOR COMBINED WITH A NOVEL ANTICANCER AGENT OF TAS-102. Drug Metab Rev 2015; 47(S1): 263
14. Strobel J, Müller F, Zolk O, Endreß B, König J, Fromm M F, Maas R. Transport of asymmetric dimethylarginine (ADMA) by cationic amino acid transporter 2 (CAT2), organic cation transporter 2 (OCT2) and multidrug and toxin extrusion protein 1 (MATE1). Amino Acids 2013; 45:989-1002.
15. Valeri L, Vanderweele T J. Mediation analysis allowing for exposure-mediator interactions and causal interpretation: theoretical assumptions and implementation with SAS and SPSS macros. Psychol Methods 2013; 18:137-50.
16. Christensen M M, Pedersen R S, Stage T B, Brasch-Andersen C, Nielsen F, Damkier P, Beck-Nielsen H, Brøsen K. A gene-gene interaction between polymorphisms in the OCT2 and MATE1 genes influences the renal clearance of metformin. Pharmacogenet Genomics 2013; 23:526-34.
17. Grun B, Kiessling M K, Burhenne J, Riedel K D, Weiss J, Rauch G, Haefeli W E, Czock D. Trimethoprim-metformin interaction and its genetic modulation by OCT2 and MATE1 transporters. Br J Clin Pharmacol 2013; 76:787-96.
18. Vanderweele T J, Vansteelandt S. Odds ratios for mediation analysis for a dichotomous outcome. Am J Epidemiol. 2010; 172: 1339-48.
19. Valeri L, Vanderweele T J. Mediation analysis allowing for exposure-mediator interactions and causal interpretation: theoretical assumptions and implementation with SAS and SPSS macros. Psychol Methods. 2013; 18:137-50.
20. Jordheim L P, Durantel D, Zoulim F, Dumontet C. Advances in the development of nucleoside and nucleotide analogues for cancer and viral diseases. Nat Rev Drug Discov 2013; 12:447-64.
21. Spratlin J L, Mackey J R. Human Equilibrative Nucleoside Transporter 1 (hENT1) in Pancreatic Adenocarcinoma: Towards Individualized Treatment Decisions. Cancers (Base1) 2010; 2:2044-54.
22. Damaraju V L, Damaraju S, Young J D, Baldwin S A, Mackey J, Sawyer M B, Cass C E. Nucleoside anticancer drugs: the role of nucleoside transporters in resistance to cancer chemotherapy. Oncogene 2003; 22:7524-36.
23. Spratlin J, Sangha R, Glubrecht D, Dabbagh L, Young J D, Dumontet C, Cass C, Lai R, Mackey J R. The absence of human equilibrative nucleoside transporter 1 is associated with reduced survival in patients with gemcitabine-treated pancreas adenocarcinoma. Clin Cancer Res 2004; 10:6956-61.
24. Okazaki T, Javle M, Tanaka M, Abbruzzese J L, Li D. Single nucleotide polymorphisms of gemcitabine metabolic genes and pancreatic cancer survival and drug toxicity. Clin Cancer Res 2010; 16:320-9.
25. Tanaka M, Javle M, Dong X, Eng C, Abbruzzese J L, Li D. Gemcitabine metabolic and transporter gene polymorphisms are associated with drug toxicity and efficacy in patients with locally advanced pancreatic cancer. Cancer 2010; 116:5325-35.
26. Koepsell H, Lips K, Volk C. Polyspecific organic cation transporters: structure, function, physiological roles, and biopharmaceutical implications. Pharm Res 2007; 24:1227-51.
27. Otsuka M, Matsumoto T, Morimoto R, Arioka S, Omote H, Moriyama Y. A human transporter protein that mediates the final excretion step for toxic organic cations. Proc Natl Acad Sci USA 2005; 102:17923-8.
28. Joerger M, van Schaik R H, Becker M L, Hayoz S, Pollak M, Cathomas R, Winterhalder R, Gillessen S, Rothermundt C. Multidrug and toxin extrusion 1 and human organic cation transporter 1 polymorphisms in patients with castration-resistant prostate cancer receiving metformin (SAKK 08/09). Prostate Cancer Prostatic Dis 2015; 18:167-72.
29. Meyer zu Schwabedissen H E, Verstuyft C, Kroemer H K, Becquemont L, Kim R B. Human multidrug and toxin extrusion 1 (MATE1/SLC47A1) transporter: functional characterization, interaction with OCT2 (SLC22A2), and single nucleotide polymorphisms. Am J Physiol Renal Physiol 2010; 298:F997-F1005.
30. Lee J J, Seraj J, Yoshida K, Mizuguchi H, Strychor S, Fiejdasz J, Faulkner T, Parise R A, Fawcett P, Pollice L, Mason S, Hague J, Croft M, Nugteren J, Tedder C, Sun W, Chu E, Beumer J H. Human mass balance study of TAS-102 using (14)C analyzed by accelerator mass spectrometry. Cancer Chemother Pharmacol 2016; 77:515-26.
31. Nakano K, Ando H, Kurokawa S, Hosohata K, Ushijima K, Takada M, Tateishi M, Yonezawa A, Masuda S, Matsubara K, Inui K, Morita T, Fujimura A. Association of decreased mRNA expression of multidrug and toxin extrusion protein 1 in peripheral blood cells with the development of flutamide-induced liver injury. Cancer Chemother Pharmacol 2015; 75:1191-7.
32. Mayer R J, Van Cutsem E, Falcone A, Yoshino T, Garcia-Carbonero R, Mizunuma N, Yamazaki K, Shimada Y, Tabernero J, Komatsu Y, Sobrero A, Boucher E, Peeters M, Tran B, Lenz H J, Zaniboni A, Hochster H, Cleary J M, Prenen H, Benedetti F, Mizuguchi H, Makris L, Ito M, Ohtsu A; RECOURSE Study Group. Randomized trial of TAS-102 for refractory metastatic colorectal cancer. N Engl J Med 2015; 372: 1909-19.
33. Branzei D, Foiani M. Regulation of DNA repair throughout the cell cycle. Nat Rev Mol Cell Biol. 2008; 9: 297-308.
34. Liu J, Ehmsen K T, Heyer W D, Morrical S W. Presynaptic filament dynamics in homologous recombination and DNA repair. Crit Rev Biochem Mol Biol. 2011; 46: 240-70.
35. Krajewska M, Fehrmann R S, de Vries E G, van Vugt M A. Regulators of homologous recombination repair as novel targets for cancer treatment. Front Genet 2015; 6: 96.
36. Jasin M, Rothstein R. Repair of strand breaks by homologous recombination. Cold Spring Harb Perspect Biol 2013; 5: a012740.
37. Cheung-Ong K, Giaever G, Nislow C. DNA-damaging agents in cancer chemotherapy: serendipity and chemical biology. Chem Biol 2013; 20: 648-59.
38. Plummer R. Perspective on the pipeline of drugs being developed with modulation of DNA damage as a target. Clin Cancer Res 2010; 16: 4527-31.
39. Haynes B, Saadat N, Myung B, Shekhar M P. Crosstalk between translesion synthesis, Fanconi anemia network, and homologous recombination repair pathways in interstrand DNA crosslink repair and development of chemoresistance. Mutat Res Rev Mutat Res 2015; 763: 258-66.
40. Wang H, Shao Z, Shi L Z, Hwang P Y, Truong L N, Berns M W, Chen D J, Wu X. CtIP protein dimerization is critical for its recruitment to chromosomal DNA double-stranded breaks. J Biol Chem 2012; 287: 21471-80.
41. Sone K, Piao L, Nakakido M, Ueda K, Jenuwein T, Nakamura Y, Hamamoto R. Critical role of lysine 134 methylation on histone H2AX for y-H2AX production and DNA repair. Nat Commun 2014; 5: 5691.
42. Cook P J, Ju B G, Telese F, Wang X, Glass C K, Rosenfeld M G. Tyrosine dephosphorylation of H2AX modulates apoptosis and survival decisions. Nature 2009; 458: 591-6.
43. Barroso E, Milne R L, Fernandez L P, Zamora P, Arias J I, Benitez J, Ribas G. FANCD2 associated with sporadic breast cancer risk. Carcinogenesis. 2006; 27: 1930-7.
44. McIlwraith M J, Vaisman A, Liu Y, Fanning E, Woodgate R, West S C. Human DNA polymerase eta promotes DNA synthesis from strand invasion intermediates of homologous recombination. Mol Cell 2005; 20: 783-92.
45. Modesti M, Kanaar R. Homologous recombination: from model organisms to human disease. Genome Biol 2001; 2: REVIEWS 1014.
46. Peng Z, Liao Z, Dziegielewska B, Matsumoto Y, Thomas S, Wan Y, Yang A, Tomkinson A E. Phosphorylation of serine 51 regulates the interaction of human DNA ligase I with replication factor C and its participation in DNA replication and repair. J Biol Chem 2012; 287: 36711-9.
47. Beggs A D, Domingo E, McGregor M, Presz M, Johnstone E, Midgley R, Kerr D, Oukrif D, Novelli M, Abulafi M, Hodgson S V, Fadhil W, Ilyas M, Tomlinson I P. Loss of expression of the double strand break repair protein ATM is associated with worse prognosis in colorectal cancer and loss of Ku70 expression is associated with CIN. Oncotarget 2012; 3: 1348-55.
48. Goode E L, Ulrich C M, Potter J D. Polymorphisms in DNA repair genes and associations with cancer risk. Cancer Epidemiol Biomarkers Prev 2002; 11: 1513-30.
49. Moreno V, Gemignani F, Landi S, Gioia-Patricola L, Chabrier A, Blanco I, González S, Guino E, Capella G, Canzian F. Polymorphisms in genes of nucleotide and base excision repair: risk and prognosis of colorectal cancer. Clin Cancer Res 2006; 12: 2101-8.
50. Kweekel D M, Antonini N F, Nortier J W, Punt C J, Gelderblom H, Guchelaar H J. Explorative study to identify novel candidate genes related to oxaliplatin efficacy and toxicity using a DNA repair array. Br J Cancer 2009; 101: 357-62.
51. Thorstenson Y R, Roxas A, Kroiss R, Jenkins M A, Yu K M, Bachrich T, Muhr D, Wayne T L, Chu G, Davis R W, Wagner, Oefner P J. Contributions of ATM mutations to familial breast and ovarian cancer. Cancer Res 2003; 63: 3325-33.
52. Landi S, Gemignani F, Canzian F, Gaborieau V, Barale R, Landi D, Szeszenia-Dabrowska N, Zaridze D, Lissowska J, Rudnai P, Fabianova E, Mates D, Foretova L, Janout V, Bencko V, Gioia-Patricola L, Hall J, Boffetta P, Hung R J, Brennan P. DNA repair and cell cycle control genes and the risk of young-onset lung cancer. Cancer Res. 2006; 66: 11062-9.
53. Angèle S, Romestaing P, Moullan N, Vuillaume M, Chapot B, Friesen M, Jongmans W, Cox D G, Pisani P, Gérard J P, Hall J. ATM haplotypes and cellular response to DNA damage: association with breast cancer risk and clinical radiosensitivity. Cancer Res 2003; 63: 8717-25.
54. Butkiewicz D, Rusin M, Enewold L, Shields P G, Chorazy M, Harris C C. Genetic polymorphisms in DNA repair genes and risk of lung cancer. Carcinogenesis 2001; 22: 593-7.
55. David-Beabes G L, Lunn R M, London S J. No association between the XPD (Lys751Gln) polymorphism or the XRCC3 (Thr241Met) polymorphism and lung cancer risk. Cancer Epidemiol Biomarkers Prev 2001; 10: 911-2.
56. Matullo G, Guarrera S, Carturan S, Peluso M, Malaveille C, Davico L, Piazza A, Vineis P. DNA repair gene polymorphisms, bulky DNA adducts in white blood cells and bladder cancer in a case-control study. Int J Cancer 2001; 92: 562-7.
57. Winsey S L, Haldar N A, Marsh H P, Bunce M, Marshall S E, Harris A L, Wojnarowska F, Welsh K I. A variant within the DNA repair gene XRCC3 is associated with the development of melanoma skin cancer. Cancer Res 2000; 60: 5612-6.
58. Jiang Z, Li C, Xu Y, Cai S. A meta-analysis on XRCC1 and XRCC3 polymorphisms and colorectal cancer risk. Int J Colorectal Dis 2010; 25: 169-80.
59. Wang Z, Zhang W. Association between XRCC3 Thr241Met polymorphism and colorectal cancer risk. Tumour Biol 2013; 34: 1421-9.
60. Wang Z, Chen X, Liu B, Li S, Liu M, Xue H. Quantitative assessment of the associations between DNA repair gene XRCC3 Thr241Met polymorphism and gastric cancer. Tumour Biol 2014; 35: 1589-98.
61. Hickson I, Zhao Y, Richardson C J, Green S J, Martin N M, Orr A I, Reaper P M, Jackson S P, Curtin N J, Smith G C. dentification and characterization of a novel and specific inhibitor of the ataxia-telangiectasia mutated kinase ATM. Cancer Res 2004; 64: 9152-9.
62. Batey M A, Zhao Y, Kyle S, Richardson C, Slade A, Martin N M, Lau A, Newell D R, Curtin N J. Preclinical evaluation of a novel ATM inhibitor, KU59403, in vitro and in vivo in p53 functional and dysfunctional models of human cancer. Mol Cancer Ther 2013; 12: 959-67.

SEQUENCE LISTING

ATM rs609429 polymorphism (SEQ ID NO: 1)
GAATTGTTATCAATATAGTAA[C/G]
XRCC3 rs861539 polymorphism (SEQ ID NO: 2)
AGGCATCTGCAGTCCCTGGGGGCCA[C/T]GCTGCGTGAGCTGAGCAGTGCCTTC
hENT1 rs760370 polymorphism (SEQ ID NO: 3)
TGGGTGGAGGTGGAGACAGGTTTGC[A/G]GGAAGGAGTGAAAGACAACCCCACC
hENT1 rs9394992 polymorphism (SEQ ID NO: 4)
CCTGTGGGCAGTTCCCTGAAGGCCT[C/T]GCCGGCTCCATTTGCCTTATTGCAC
OCT2 rs316000 polymorphism (SEQ ID NO: 5)
TCAAGAACTTTTCC[A/G]ACTTGGCTACCTGGTACAAGAGTCC
OCT2 rs316019 polymorphism (SEQ ID NO: 6)
CTGGAGGTGGTTGCAGTTCACAGTT[G/T]
MATE1 rs2289669 polymorphism (SEQ ID NO: 7)
GGGCTCAGTTTCCACAGTAGCGTGG[A/G]
ATM rs609429 F (SEQ ID NO: 8)
GCCTTGGGCAAGGGTACTTA
ATM rs609429 R (SEQ ID NO: 9)
CCCACGTCTGCCTATCTGTC
XRCC3 rs861539 F2 (SEQ ID NO: 10)
CCGCATCCTGGCTAAAAATA
XRCC3 rs861539 R (SEQ ID NO: 11)
CCATTCCGCTGTGAATTTG
hENT1 rs760370 F: (SEQ ID NO: 12)
TGGGGGACACTCAGTAGAGG
hENT1 rs760370 F: (SEQ ID NO: 13)
AACGTGTATGGTGGGGTTGT
hENT1 rs9394992 F: (SEQ ID NO: 14)
CTGCCTCCTGTGCTCCAT
hENT1 rs9394992 F: (SEQ ID NO: 15)
TGGGAAATGACTGAGCTGTG
rs316000 F (SEQ ID NO: 16)
CCAGACCACTCAAGCTTTCTC
rs316000 R (SEQ ID NO: 17)
AGTCATGTTGAAAGCCAGCA
rs316019 F (SEQ ID NO: 18)
GAAGGCAGACTTCTTAGCAGAAT
rs316019 R (SEQ ID NO: 19)
ATACAGTTGGGCTCCTGGTG
rs2289669 F (SEQ ID NO: 20)
CCAGTTTGTGCTAAGCATCG
rs2289669 R (SEQ ID NO: 21)
ACACCTGGTGGGAAAACTTG
ATM rs609429 amplicon (S = C or G) (SEQ ID NO: 22)
GCCTTGGGCAAGG(S)TAACTTACTATATATGTACACACAGATACACATACA
XRCC3 rs861539 10F + 11R (R = A or G) (SEQ ID NO: 23)
CCGCATCCTGGCTAAAAATACGAGCTCAGGGGTGCAACCCTGCCTTGGTGCTCAC
CTGGTTGATGCACAGCACAGGGGCAGC(R)TGGCCCCCAGGGACTGCAGATGCCTGG
CCCTGGGGGCGGAGGCCTGGCTGTCAAATTCACAGCGGAATGG
hENT1 rs760370 amplicon (R = A or G) (SEQ ID NO: 24)
TGGGGGACACTCAGTAGAGGGAGGGCAAAAGGAGAGTCCCTGCTCCCAGAGCTGAG
AGGAGAGATGGCTCAGGAGGGGCTCCCAGGCTGAGGGGATAGTGACTGTAGTGGAG
GGGGGCGCCAAGCTTACTTGGGTGGAGGTGGAGACAGGTTTGC(R)GGAAGGAGTGA
AAGACAACCCCACCATACACGTT
hENT1 rs9394992 amplicon (Y = C or T): (SEQ ID NO: 25)
5′CTGCCTCCTGTGCTCCATTCCCAGGCTGCCTGCCCATTCTGTCCCCACCTCCGCTCT
CCTGTGGGCAGTTCCCTGAAGGCCT(Y)GCCGGCTCCATTTGCCTTATTGCACAGCTC
AGTCATTTCCCA3′
OCT2 rs316000 amplicon (R = A or G) (SEQ ID NO: 26)
5′CCAGACCACTCAAGCTTTCTCCATATCAGCAATAAGGCTGTTTCATTTCTCATTTGT
AGCATTTTTATACTTCCTTCAAGAACTTTTCCTGTGCATTCTC(R)ACTTGGCTACCTG
GTACAAGAGTCCTCACTTTCAGCTTATGCTGGCTTTCAACATGACT3′
OCT2 rs316019 amplicon (M = A or C) (SEQ ID NO: 27)
GAAGGCAGACTTCTTAGCAGAATAAAATACTTTGATTTATTTCCTTTTTATTCCAAAT
GGACTTACCAGTAATAGAGCAAGAAGAAGAAGTTGGGCAGAG(M)AACTGTGAACT
GCAACCACCTCCAGTGAGGAAGTGCGTAAGCCACCCCAGCTAGCACCAGGAGCCCA
ACTGTAT
MATE1 rs2289669 amplicon (R = A or G) (SEQ ID NO: 28)
CCAGTTTGTGCTAAGCATCGTAACCTGGGGCTCAGTTTCCACAGTAGCGTGG(R)AGT
TCCCGGCTAGACAAAGGGGATGTTGCAAATCAGTCTTTTCAAAACTTTTAGGACCAA
GTTTTCCCACCAGGTGT
ATM rs609429 (C) allele (SEQ ID NO: 29)
GAATTGTTATCAATATAGTAA
ATM rs609429 (G) allele (SEQ ID NO: 30)
GAATT
XRCC3 rs861539 (C) allele (SEQ ID NO: 31)
AGGCATCTGCAGTCCCTGGGGGCCACGCTGCGTGAG
XRCC3 rs861539 (C) allele (SEQ ID NO: 32)
AGGCATC
hENT1 rs760370 (A) allele (SEQ ID NO: 33)
TGGGTGGAGGTGGAGACAGGTTTGCAGGAAGG
hENT1 rs760370 (G) allele (SEQ ID NO: 34)
TGGGTGGAGGTGGAGACAGGTTTGCGGGAAGG
hENT1 rs9394992 (C) allele (SEQ ID NO: 35)
CCTGTGGGC
hENT1 rs9394992 (T) allele is: (SEQ ID NO: 36)
CCTGTGGGC
OCT2 rs316000 (A) allele (SEQ ID NO: 37)
TCAAGAACTTTTCC
OCT2 rs316000 (G) allele (SEQ ID NO: 38)
TCAAGAACTTTTCC
OCT2 rs316019 (G) allele (SEQ ID NO: 39)
CTGGAGGTGGTTGCAGTTCACAGTTG
OCT2 rs316019 (T) allele of rs316019 (SEQ ID NO: 40)
CTGGAGGTGGTTGCAGTTCACAGTTT
MATE1 rs2289669 (A) allele (SEQ ID NO: 41)
GGGCTCAGTTTCCACAGTAGCGTGGA
MATE1 rs2289669 (G) allele is (SEQ ID NO: 42)
GGGCTCAG
BRCA1 rs799917 F: (SEQ ID NO: 43)
CCAGAGTGGGCAGAGAATGT
BRCA1 rs799917 R: (SEQ ID NO: 44)
AACCACAGTCGGGAAACAAG
BRCA1 rs16941 F: (SEQ ID NO: 45)
GCTTGAATGTTTTCATCACTGG
BRCA1 rs16941 R: (SEQ ID NO: 46)
CAGTGAGCACAATTAGCCGTA
BRCA2 rs144848 F: (SEQ ID NO: 47)
GGAACCAAATGATACTGATCCA
BRCA2 rs144848 R: (SEQ ID NO: 48)
ACCATTCACAGGCCAAAGAC
XRCC3 rs861539 F1: (SEQ ID NO: 49)
CTCACCTGGTTGATGCACAG
FANCD2 rs2272125 F: (SEQ ID NO: 50)
CATGTGCCTCTGCTCAAAAA
FANCD2 rs2272125 R: (SEQ ID NO: 51)
GATCCAAGGCTTACCTGCAA
FANCD2 rs6792811 F: (SEQ ID NO: 52)
CACTGCCATACCACCACTTG
FANCD2 rs6792811 R: (SEQ ID NO: 53)
GATTACAGGCGTGAGCCATT
H2AX rs643788 F: (SEQ ID NO: 54)
TCTGGGACCAGAGAGAGAGG
H2AX rs643788 R: (SEQ ID NO: 55)
TCCAGTCCATTTCTCCTTGC
H2AX rs2509049 F: (SEQ ID NO: 56)
AGACCAAAGGGGTGGAGTCT
H2AX rs2509049 R: (SEQ ID NO: 57)
TCCATTCTCCCTTTGTCAGG
RAD51 rs1801320 F: (SEQ ID NO: 58)
AGCTGGGAACTGCAACTCAT
RAD51 rs1801320 R: (SEQ ID NO: 59)
CGCCTCACACACTCACCTC
ATR rs2227928 F: (SEQ ID NO: 60)
AGCAGAACACAACCTATCTGC
ATR rs2227928 R: (SEQ ID NO: 61)
CAGCTCCTTTGCAGTTGATG
CHEK1 rs521102 F: (SEQ ID NO: 62)
TGGGCTATCAATGGAAGAAAA
CHEK1 rs521102 R: (SEQ ID NO: 63)
AGGTGTGAGCCACAGCCTAT
CHEK2 rs2267130 F: (SEQ ID NO: 64)
GGCTTGGAAGTTCAATCAGG
CHEK2 rs2267130 R: (SEQ ID NO: 65)
TTCATTCCCAGGTAGCATCC
CHEK2 rs2236142 F: (SEQ ID NO: 66)
ACCAATGAGGAGCAGCAGAT
CHEK2 rs2236142 R: (SEQ ID NO: 67)
CTGATTGGCTGGGGAGTC
TP53 rs1042522 F: (SEQ ID NO: 68)
TTCTGGGAAGGGACAGAAGA
TP53 rs1042522 R: (SEQ ID NO: 69)
GAAGACCCAGGTCCAGATGA
CHE1 (AATF) rs1045056 F: (SEQ ID NO: 70)
GCCTTTGAACGCTCAATCTT
CHE1 (AATF) rs1045056 R: (SEQ ID NO: 71)
AACTCTCTGGGACAGGCTGA
CHE1 (AATF) rs1564796 F: (SEQ ID NO: 72)
AGCTGACCCCCTGAAAGTCT
CHE1 (AATF) rs1564796 R: (SEQ ID NO: 73)
AGCAATGAAGGCAGGAGAAA
PIN1 rs2233678 F: (SEQ ID NO: 74)
ACTCTGGGTCCCCAAATACC
PIN1 rs2233678 R: (SEQ ID NO: 75)
GTGCCGACATTGACATTCAT
PIN1 rs2233679 F: (SEQ ID NO: 76)
CCAGACTGCGAGGGAATAAA
PIN1 rs2233679 R: (SEQ ID NO: 77)
TGTTTCCCACAGATGTCCAA
CDKNA1 rs1801270 F: (SEQ ID NO: 78)
GTCCGTCAGAACCCATGC
CDKNA1 rs1801270 R: (SEQ ID NO: 79)
GTGTCTCGGTGACAAAGTCG
CDKNA1 rs1059234 F: (SEQ ID NO: 80)
TGGCTGACTTCTGCTGTCTC
CDKNA1 rs1059234 R: (SEQ ID NO: 81)
AAGATGTAGAGCGGGCCTTT
PCNA rs25406 F: (SEQ ID NO: 82)
GAAGGGCTGTATTTCGAACG
PCNA rs25406 R: (SEQ ID NO: 83)
ATAATGGCATCCTCCAGCAG
ATM rs609429 amplicon (C) (SEQ ID NO: 84)
GCCTTGGGCAAGGTATGTACACACAGATACACATACA
ATM rs609429 amplicon (G) (SEQ ID NO: 85)
GCCTTGGGCAAGGTATGTACACACAGATACACATACA
hENT1 rs760370 amplicon (A) (SEQ ID NO: 86)
TGGGGGACACTCAGTAGAGGGAGGGCAAAAGGAGAGTCCCTGCTCCCAGAGCTGAG
AGGAGAGATGGCTCAGGAGGGGCTCCCAGGCTGAGGGGATAGTGACTGTAGTGGAG
GGGGGCGCCAAGCTTACTTGGGTGGAGGTGGAGACAGGTTTGCAGGAAGGAGTGAA
AGACAACCCCACCATACACGTT
hENT1 rs760370 amplicon (G) (SEQ ID NO: 87)
TGGGGGACACTCAGTAGAGGGAGGGCAAAAGGAGAGTCCCTGCTCCCAGAGCTGAG
AGGAGAGATGGCTCAGGAGGGGCTCCCAGGCTGAGGGGATAGTGACTGTAGTGGAG
GGGGGCGCCAAGCTTACTTGGGTGGAGGTGGAGACAGGTTTGCGGGAAGGAGTGAA
AGACAACCCCACCATACACGTT
hENT1 rs9394992 amplicon (C) (SEQ ID NO: 88)
CTGCCTCCTGTGCTCCATTCCCAGGCTGCCTGCCCA
hENT1 rs9394992 amplicon (T) (SEQ ID NO: 89)
CTGCCTCCTGTGCTCCATTCCCAGGCTGCCTGCCCATT
CGCTCTCCTGTGGGCAGTTCCCTGAAGGCCTTGCCGGCTCCATTTGC
OCT2 rs316000 amplicon (A) (SEQ ID NO: 90)
CCAGACCACTCAAGCTTTCTCCATATCAGCAATAAGGCTGTTTCATTTCTCATTTGTA
GCATTTTTATACTTCCTTCAAGAACTTTTCCTGTGCATTCTCAACTTGGCTACCTGGT
ACAAGAGTCCTCACTTTCAGCTTATGCTGGCTTTCAACATGACT
OCT2 rs316000 amplicon (G) (SEQ ID NO: 91)
CCAGACCACTCAAGCTTTCTCCATATCAGCAATAAGGCTGTTTCATTTCTCATTTGTA
GCATTTTTATACTTCCTTCAAGAACTTTTCCTGTGCATTCTCGACTTGGCTACCTGGT
ACAAGAGTCCTCACTTTCAGCTTATGCTGGCTTTCAACATGACT
OCT2 rs316019 amplicon (A) (SEQ ID NO: 92)
GAAGGCAGACTTCTTAGCAGAATAAAATACTTTGATTTATTTCCTTTTTATTCCAAAT
GGACTTACCAGTAATAGAGCAAGAAGAAGAAGTTGGGCAGAGAAACTGTGAACTGC
AACCACCTCCAGTGAGGAAGTGCGTAAGCCACCCCAGCTAGCACCAGGAGCCCAAC
TGTAT
OCT2 rs316019 amplicon (C) (SEQ ID NO: 93)
GAAGGCAGACTTCTTAGCAGAATAAAATACTTTGATTTATTTCCTTTTTATTCCAAAT
GGACTTACCAGTAATAGAGCAAGAAGAAGAAGTTGGGCAGAGCAACTGTGAACTGC
AACCACCTCCAGTGAGGAAGTGCGTAAGCCACCCCAGCTAGCACCAGGAGCCCAAC
TGTAT
MATE1 rs2289669 amplicon (A) (SEQ ID NO: 94)
CCAGTTTGTGCTAAGCATCGTAACCTGGGGCTCAGTTTCCACAGTAGCGTGGAAGTT
CCCGGCTAGACAAAGGGGATGTTGCAAATCAGTCTTTTCAAAACTTTTAGGACCAAG
TTTTCCCACCAGGTGT
MATE1 rs2289669 amplicon (G) (SEQ ID NO: 95)
CCAGTTTGTGCTAAGCATCGTAACCTGGGGCTCAGTTTCCACAGTAGCGTGGGAGTT
CCCGGCTAGACAAAGGGGATGTTGCAAATCAGTCTTTTCAAAACTTTTAGGACCAAG
TTTTCCCACCAGGTGT
XRCC3 rs861539 10F + 11R (A) (SEQ ID NO: 96)
CCGCATCCTGGCTAAAAATACGAGCTCAGGGGTGCAACCCTGCCTTGGTGCTCAC
CTGGTTGATGCACAGCACAGGGGCAGCATGGCCCCCAGGGACTGCAGATGCCTGG
CCCTGGGGGCGGAGGCCTGGCTGTCAAATTCACAGCGGAATGG
XRCC3 rs861539 10F + 11R (G) (SEQ ID NO: 97)
CCGCATCCTGGCTAAAAATACGAGCTCAGGGGTGCAACCCTGCCTTGGTGCTCAC
CTGGTTGATGCACAGCACAGGGGCAGCGTGGCCCCCAGGGACTGCAGATGCCTGG
CCCTGGGGGCGGAGGCCTGGCTGTCAAATTCACAGCGGAATGG
XRCC3 rs861539 49F + 11R (R = A or G) (SEQ ID NO: 98)
CTCACCTGGTTGATGCACAGCACAGGG(R)TGGCCCCCAGGGACTGCAGATGCCTGG
CCCTGGGGGCGGAGGCCTGGCTGTCAAATTCACAGCGGAATGG
XRCC3 rs861539 49F + 11R (A) (SEQ ID NO: 99)
CTCACCTGGTTGATGCACAGCACAGGGTGGCCCCCAGGGACTGCAGATGCCTGG
CCCTGGGGGCGGAGGCCTGGCTGTCAAATTCACAGCGGAATGG
XRCC3 rs861539 49F + 11R (G) (SEQ ID NO: 100)
CTCACCTGGTTGATGCACAGCACAGGGTGGCCCCCAGGGACTGCAGATGCCTGG
CCCTGGGGGCGGAGGCCTGGCTGTCAAATTCACAGCGGAATGG

What is claimed is:

1. A method for one or more of:
(a) selecting a cancer patient for a therapy comprising administration of TAS-102 or an equivalent thereof;
(b) identifying whether a cancer patient is likely to experience a relatively longer or shorter progression free survival (PFS) or overall survival (OS) following a therapy comprising administration of TAS-102 or an equivalent thereof;
(c) treating a cancer patient;
(d) detecting a biomarker in a biological sample isolated from a cancer patient;
(e) detecting a polymorphism in a biological sample isolated from a cancer patient or a patient suspected of having cancer;
(f) selecting a cancer patient for a therapy comprising administration of an effective amount of TAS-102;
(g) classifying a cancer patient as eligible for a therapy comprising administration of an effective amount of TAS-102; and
(h) identifying whether a cancer patient is likely to experience TAS-102 related toxicity following a therapy comprising the administration of TAS-102;
the method comprising screening a biological sample isolated from the cancer patient or the patient suspected of having cancer for one or more polymorphism(s) selected from the group consisting of rs760370 (GG or GA); rs9394992 (TT or TC); rs2289669 (GA); rs2289669 (GG) and rs316019 (CC); rs2289669 (GA) and rs316019 (CC); rs2289669 (GA) and rs316019 (CA); rs2289669 (GA) and rs316019 (AA); rs2289669 (AA) and rs316019 (AA); rs760370 (GG or GA) and rs316019/rs2289669 of (CC/GG); rs760370 (GG or GA) and rs316019/rs2289669 of (CC/GA); rs760370 (GG or GA) and rs316019/rs2289669 of (CA/GA); rs760370 (GG or GA) and rs316019/rs2289669 of (AA/GA); rs760370 (GG or GA) and rs316019/rs2289669 of (AA/AA); rs609429 (GC or GG); and rs861539 (AG or AA); and
performing one or more of the following if the biological sample isolated from the cancer patient has the one or more polymorphism(s):
(i) selecting the cancer patient for the therapy comprising administration of TAS-102 or an equivalent thereof;
(j) identifying that the cancer patient or patient suspected of having cancer is likely to experience a longer progression free survival than a cancer patient not having the one or more polymorphism(s);
(k) classifying the cancer patient as eligible for a therapy comprising administration of an effective amount of TAS-102;
(l) identifying that the cancer patient is likely to experience toxicity from a therapy comprising administration of TAS-102 or an equivalent thereof; and
(m) administering an effective amount of TAS-102 to the cancer patient or the patient suspected of having cancer.

2. The method of claim 1, comprising classifying the cancer patient as not eligible for therapy comprising administration of TAS-102 if the one or more polymorphism(s) are not in the biological sample isolated from the cancer patient.

3. The method of claim 1, wherein the cancer patient is a colorectal cancer patient.

4. The method of claim 1, wherein the toxicity comprises one or more of the group of: febrile neutropenia, leukopenia, stomatitis, neutropenia, hand-foot syndrome, cardiac ischemia, thrombocytopenia, increase in alanine aminotransferase level, increase in aspartate aminotransferase level, increase in total bilirubin, increase in alkaline phosphatase level, increase in creatinine level, anemia, anorexia, nausea, vomiting, decreased appetite, fatigue, abdominal pain, fever, asthenia, or diarrhea.

5. A method for treating a cancer patient selected for treatment based on the presence of one or more polymorphism(s) in a biological sample isolated from the patient, wherein the one or more polymorphism(s) is selected from the group consisting of rs760370 (GG or GA);
rs9394992 (TT or TC); rs2289669 (GA); rs2289669 (GG) and rs316019 (CC); rs2289669 (GA) and rs316019 (CC); rs2289669 (GA) and rs316019 (CA); rs2289669 (GA) and rs316019 (AA); rs2289669 (AA) and rs316019 (AA); rs760370 (GG or GA) and rs316019/rs2289669 of (CC/GG); rs760370 (GG or GA) and rs316019/rs2289669 of (CC/GA); rs760370 (GG or GA) and rs316019/rs2289669 of (CA/GA); rs760370 (GG or GA) and rs316019/rs2289669 of (AA/GA); rs760370 (GG or GA) and rs316019/rs2289669 of (AA/AA); rs609429 (GC or GG); and rs861539 (AG or AA),
the method comprising administering to the cancer patient a therapy comprising a therapeutically effective amount of TAS-102.

6. A method for increasing the progression-free and/or overall survival of a cancer patient, comprising:
screening a biological sample isolated from the cancer patient for one or more polymorphism(s) is selected from the group consisting of rs760370 (GG or GA); rs9394992 (TT or TC); rs2289669 (GA); rs2289669 (GG) and rs316019 (CC); rs2289669 (GA) and rs316019 (CC); rs2289669 (GA) and rs316019 (CA); rs2289669 (GA) and rs316019 (AA); rs2289669 (AA) and rs316019 (AA); rs760370 (GG or GA) and rs316019/rs2289669 of (CC/GG); rs760370 (GG or GA) and rs316019/rs2289669 of (CC/GA); rs760370 (GG or GA) and rs316019/rs2289669 of (CA/GA); rs760370 (GG or GA) and rs316019/rs2289669 of (AA/GA); rs760370 (GG or GA) and rs316019/rs2289669 of (AA/AA); rs609429 (GC or GG); and rs861539 (AG or AA), and
classifying the patient as eligible for a therapy comprising administration of TAS-102 if the one or more polymorphism(s) is present in the biological sample isolated from the cancer patient or not eligible for a therapy comprising administration of TAS-102 if the one or more polymorphism(s) is not present in the biological sample isolated from the cancer patient.

7. The method of claim 1, wherein the detecting comprises contacting the biological sample isolated from the cancer patient or nucleic acid isolated from the biological sample with a labeled nucleic acid probe that specifically binds to a nucleic acid having the sequence of any of SEQ ID NO:1-4 and overlaps a rs609429, rs861539, rs760370, or rs9394992 polymorphic site.

8. The method of claim 7, wherein the nucleic acid probe comprises about 5, about 10, about 15, about 20, about 25, about 30, about 35, about 40 or more contiguous nucleotides of any of SEQ ID NO:1-4.

9. The method of claim 7, wherein the label is a fluorophore.

10. The method of claim 7, wherein detecting further comprises amplifying nucleic acid containing the rs609429, rs861539, rs760370, or rs9394992 polymorphism to generate an amplicon containing the rs609429, rs861539, rs760370, or rs9394992 polymorphism.

11. The method of claim 10, wherein said amplifying is performed with a forward primer having the sequence of SEQ ID NO:8 and a reverse primer having the sequence of SEQ ID NO:9, a forward primer having the sequence of SEQ ID NO:10 and a reverse primer having the sequence of SEQ ID NO:11, a forward primer having the sequence of SEQ ID NO:49 and a reverse primer having the sequence of SEQ ID NO:11, a forward primer having the sequence of SEQ ID NO:12 and a reverse primer having the sequence of SEQ ID NO:13, or a forward primer having the sequence of SEQ ID NO:14 and a reverse primer having the sequence of SEQ ID NO:15.

12. The method of claim 1, wherein the cancer of the patient or patient suspected of having cancer is a cancer having a KRAS phenotype and BRAF wild-type phenotype.

13. The method of claim 1, wherein the cancer is a cancer selected from gastrointestinal cancer, colon cancer, rectal cancer, or colorectal cancer.

14. The method of claim 13, wherein the cancer is non-metastatic colorectal cancer or metastatic colorectal cancer.

15. The method of claim 1, wherein the biological sample isolated from the cancer patient is a cell or a tissue sample.

16. The method of claim 1, wherein the biological sample isolated from the cancer patient comprises at least one of a tumor cell, a normal cell adjacent to a tumor, a normal cell corresponding to the tumor tissue type, a blood cell, a peripheral blood lymphocyte, or combinations thereof.

17. The method of claim 1, wherein the biological sample isolated from the cancer patient is at least one of blood, plasma, an original sample recently isolated from the patient, a fixed tissue, a frozen tissue, a biopsy tissue, a resection tissue, a microdissected tissue, or combinations thereof.

18. The method of claim 1, wherein the detecting is by a method comprising PCR, PCR-RFLP, whole genome sequencing, sequencing, or microarray.

19. The method of claim 1, wherein the cancer patient or patient suspected of having cancer is a human patient.

20. A kit for performing the method of claim 1, comprising reagents to identify or determine the genotype of the biological sample isolated from the cancer patient and instructions for use.

21. The kit of claim 20, comprising a forward primer having the sequence of SEQ ID NO:8 and a reverse primer having the sequence of SEQ ID NO:9, a forward primer having the sequence of SEQ ID NO:10 and a reverse primer having the sequence of SEQ ID NO:11, a forward primer having the sequence of SEQ ID NO:49 and a reverse primer having the sequence of SEQ ID NO:11, a forward primer having the sequence of SEQ ID NO:12 and a reverse primer having the sequence of SEQ ID NO:13, or a forward primer having the sequence of SEQ ID NO:14 and a reverse primer having the sequence of SEQ ID NO:15.

22. The kit of claim 21, comprising a nucleic acid probe having about 5, about 10, or about 20, or about 25, or about 30, about 35, about 40 or more contiguous nucleotides of SEQ ID NO:1-4 and overlapping the rs609429, rs861539, rs760370, or rs9394992 polymorphic site.
